(19) United States
(12) Patent Application Publication
(10) Pub. No.: US 2024/0132628 A1
(43) Pub. Date: Apr. 25, 2024

(54) OLIGONUCLEOTIDE ANALOGUE THERAPEUTICS FOR TREATMENT OF NEUROMUSCULAR DISEASE

(71) Applicant: NeuBase Therapeutics, Inc., Pittsburgh, PA (US)

(72) Inventors: Dietrich A. STEPHAN, Pittsburgh, PA (US); Dani STOLTZFUS, Pittsburgh, PA (US); Barry BADEAU, Pittsburgh, PA (US); Ramesh BATWAL, Pune (IN); Austin KILGORE, Pittsburgh, PA (US)

(21) Appl. No.: 18/266,970

(22) PCT Filed: Dec. 15, 2021
(86) PCT No.: PCT/US2021/010058

§ 371 Date: Jun. 13, 2023

Related U.S. Application Data
(60) Provisional application No. 63/125,513, filed on Dec. 15, 2020.
(60) Provisional application No. 63/158,176, filed on Mar. 8, 2021.
(60) Provisional application No. 63/197,816, filed on Jun. 7, 2021.
(60) Provisional application No. 63/250,117, filed on Sep. 29, 2021.

(51) Int. Cl. C07K 19/00

(52) U.S. Cl. CPC C07K 19/00

(57) ABSTRACT

The present disclosure relates to compounds useful for the detection or modulation of target nucleic acids, including DNA and RNA. The present disclosure further relates to methods for treatment of trinucleotide repeat disorders, which can include administration of oligonucleotid analogues that can bind pathogenic nucleotide repeats in DNA or RNA.

### CROSS REFERENCE

This application is a 371 national stage entry of International Application No. PCT/US2021/010058, which claims the benefit of U.S. Provisional Application No. 63/125,513, filed Dec. 15, 2020, U.S. Provisional Application No. 63/158,176, filed Mar. 8, 2021, U.S. Provisional Application No. 63/197,816, filed Jun. 7, 2021, and U.S. Provisional Application No. 63/250,117, filed Sep. 29, 2021, each of which is entirely incorporated herein by reference.

### SEQUENCE LISTING

The instant application contains a Sequence Listing which has been submitted electronically in ASCII format and is hereby incorporated by reference in its entirety. Said ASCII copy, created on Jun. 13, 2023, is named 54344-717_831_SL.txt and is 44,792 bytes in size.

### BACKGROUND

Muscular dystrophies are a group of diseases characterized by progressive skeletal muscle loss and weakness over time. Myotonic dystrophy (DM) is the most common form of muscular dystrophy, and can be further classified into type 1 myotonic dystrophy (DM1) and type 2 myotonic dystrophy (DM2). There is presently no cure for DM.

### SUMMARY

In some embodiments, the present disclosure provides a compound comprising an oligomeric sequence, wherein the oligomeric sequence comprises a repeating unit of formula:

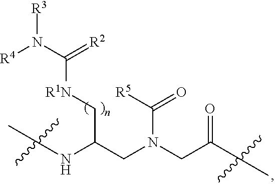

or an ionized form thereof, wherein:
R¹ is H, alkyl, or a nitrogen atom protecting group;
R² is O, NH, N(alkyl), or N(Pg^{N}), wherein Pg^{N} is a nitrogen atom protecting group;
R³ is H, alkyl, or a nitrogen atom protecting group;
R⁴ is H, alkyl, or a nitrogen atom protecting group;
R⁵ is alkyl or O-alkyl, any of which is unsubstituted or substituted; and
n is 1, 2, 3, 4, 5, 6, 7, 8, 9, or 10,
   or a pharmaceutically-acceptable salt or ionized form thereof.

In some embodiments, the present disclosure provides a compound comprising a structure that is:

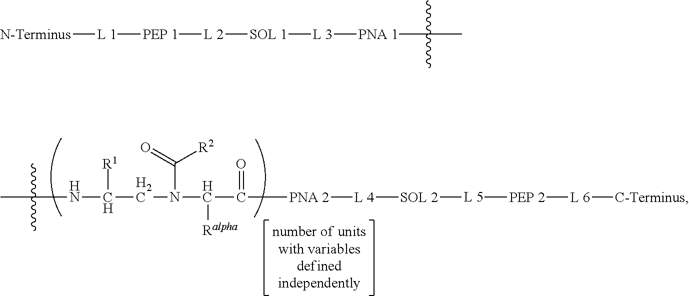

wherein:
the number of units with variables defined independently is at least 11;
N-Terminus is H, acyl, a group that together with the nitrogen atom to which the N-Terminus is bound forms a carbamate, a probe, a metal chelator, or a biological agent;
each R¹ is independently alkyl that is unsubstituted or substituted or H, wherein at least one iteration of R¹ is a hydroxyalkyl group;
each R^{alpha} is independently alkyl that is unsubstituted or substituted or H;
each R² is independently methyl substituted with a heterocycle;
C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof.

In some embodiments, the present disclosure provides a compound of formula:

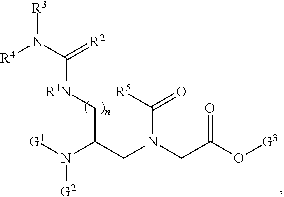

wherein:
R¹ is H, alkyl, or a nitrogen atom protecting group;
R² is NH, N(alkyl), or N(Pg^{N}), wherein Pg^{N} is a nitrogen atom protecting group;
R³ is H, alkyl, or a nitrogen atom protecting group;
R⁴ is H, alkyl, or a nitrogen atom protecting group;
R⁵ is alkyl or O-alkyl, any of which is unsubstituted or substituted;
each of G¹ and G² is independently a nitrogen atom protecting group or hydrogen;
G³ is substituted or unsubstituted alkyl, substituted or unsubstituted alkenyl, haloalkyl, or hydrogen; and
n is 1, 2, 3, 4, 5, 6, 7, 8, 9, or 10,
   or a salt or ionized form thereof.

In some embodiments, the present disclosure provides a compound of formula:

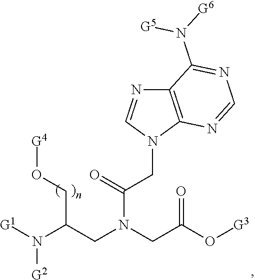

wherein:
G¹ is H or a nitrogen atom protecting group;
G² is H or a nitrogen atom protecting group;
G³ is H or carboxylate protecting group;
G⁴ is H or a hydroxyl protecting group;
G⁵ is H;
G⁶ is H or a nitrogen atom protecting group; and
n is 1, 2, 3, or 4,
   or a salt or ionized form thereof.

In some embodiments, the present disclosure provides a compound of formula:

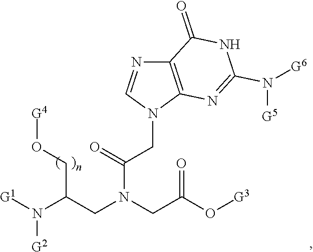

wherein:
G¹ is H or a nitrogen atom protecting group;
G² is H or a nitrogen atom protecting group;
G³ is H or carboxylate protecting group;
G⁴ is H or a hydroxyl protecting group;
G⁵ is H or a nitrogen atom protecting group;
G⁶ is H or a nitrogen atom protecting group; and
n is 1, 2, 3, or 4,
   or a salt or ionized form thereof.

### INCORPORATION BY REFERENCE

Each patent, publication, and non-patent literature cited in the application is hereby incorporated by reference in its entirety as if each was incorporated by reference individually.

### BRIEF DESCRIPTION OF THE DRAWINGS

FIG. 1 is a chart summarizing the mutational mechanism of Myotonic Dystrophy Type 1 (DM1). A genetic mutation results in generalized mis-splicing of transcripts and haploinsufficiency of the DMPK protein which both contribute to the disease.

FIG. 2 is a chart summarizing total DMPK mRNA in DM1 patient-derived human cells that carry the DMPK 3′-UTR repeat expansion, either: (i) treated in tissue culture with Compound 1, or (ii) untreated (labelled as “DM1”). The horizontal line indicates the level of DMPK transcript in normal human cells that lack the DMPK 3′-UTR repeat expansion. In DM1 cells treated with Compound 1, DMPK mRNA levels were normalized within 24 hours. These results indicate that Compound 1 crossed the cell membrane, escaped the endosome, penetrated the nucleus, and disrupted the nuclear aggregates to allow the mutant transcript to translocate to the cytoplasm and normalize the overexpression of DMPK.

FIG. 3 illustrates percent of exon inclusion across two established mis-spliced transcripts (MBNL1 and MBNL2) in untreated DM1 cells (“DM1”), DM1 cells treated with Compound 1 (“treated”), and healthy cells. By day 2 post treatment, an effect on splicing rescue was observed, with the correct adult exon usage further improved at later time points and approaching full restoration by day 5-9 in each case.

FIG. 4 illustrates a composite score of global splice rescue after administration of Compound 1 to cells. Correction of global splicing across multiple transcripts (hDSI) was observed at day 5. The transcriptome in human DM1 patient cells was sequenced, and the change in transcript percent splice inclusion (ΔPSI) between affected cells and healthy cells at the p<0.05 level was determined. Calculation of hDSI (degree of average ΔPSI across all mis-spliced transcripts) indicated that treatment rescued normal splicing at a level of p=1.84×10-7 over affected cells.

FIG. 5 is a chart that depicts normalized percent splice inclusion (PSI) between treated vs. healthy human cells for 188 unique human transcripts (x-axis) at 5 days post treatment with Compound 1. A statistically-significant difference in PSI was observed between healthy and DM1 splice inclusion, greater than >20% PSI and robust fold-coverage. A statistically-significant p-value was observed between treated and untreated (DM1) PSI. Restoration of normal splicing was observed after Compound 1 treatment across 188 significantly mis-spliced human transcripts by day 5.

FIG. 6 is a chart summarizing percent splice inclusion levels (PSI) for selected transcripts in cells 5 days after treatment with Compound 1.

FIG. 7 illustrates ACTA1 mRNA levels in untreated HSA^{LR} mice and HSA^{LR} mice 1 day post administration of 29 mg/kg Compound 1 injected into the tail vein. These results indicate that Compound 1 can exit the vasculature and penetrate into Tibialis anterior muscle nuclei to engage target HSA (skeletal muscle actin) mRNA within 24 hours. mRNA levels remained decreased until 13 days (last timepoint tested). This result illustrates that Compound 1 can maintain sufficient time in circulation to enter target tissue and to engage with the target to elicit a therapeutic effect.

FIG. 8 illustrates a composite score of global splice rescue after administration of Compound 1 into the tail vein of HSA^{LR} mice. Significant correction of global splicing across many transcripts (mDSI) in Tibialis anterior skeletal muscle was observed at day 13.

FIG. 9 illustrates normalized percent splice inclusion (PSI) between treated HSA^{LR} mice vs. FVB background strain mice for 56 unique murine Tibialis anterior muscle transcripts (x-axis) at 13 days post IV administration of Compound 1 for which were observed (i) a statistically-significant difference in PSI between healthy and HSA^{LR} exon inclusion, (ii) greater than >20% PSI fold-coverage, and (iii) a statistically-significant p-value between treated and HSA^{LR} PSI. Restoration of normal splicing was observed across 56 mis-spliced murine Tibialis anterior skeletal muscle transcripts at 13 days post intravenous administration of Compound 1.

FIG. 10 illustrates percent splice inclusion (PSI) in selected murine Tibialis anterior muscle transcripts at day 13 after intravenous administration of Compound 1.

FIG. 11 illustrates normalized DMPK protein levels in untreated DM1 patient-derived cells and DM1 patient-derived cells 5 days after treatment with Compound 1. 74 kD DMPK protein levels in treated cells remained unchanged relative to untreated cells 5 days after initial dosing.

FIG. 12 is a chart summarizing total ACTA1 transgene mRNA levels in the tibialis anterior muscle of HSA^{LR} transgenic mice as measured by qRT-PCR 24 hours after administration of a 30 mg/kg single IV dose of Compound 1. The target ACTA1 transgene contained a ˜220 trinucleotide repeat and mirrored DM1 muscle phenotype. These results show that ACTA1 transgene mRNA levels were lower (p=0.021) in mice treated with Compound 1, and further indicate that Compound 1 penetrated myofiber, escaped the endosome, localized to nuclei, and invaded ACTA1 mRNA 3′ UTR hairpins.

FIG. 13 illustrates reduction in nuclear inclusions by FISH in HSA^{LR} mouse muscle using Compound 1.

FIG. 14 illustrates in vivo mis-splicing correction of the myotonia-causing transcripts using Compound 1.

FIG. 15A shows restoration of CLCN1 protein levels by western blot for HSA^{LR} mice that received a single dose of Compound 1.

FIG. 15B shows restoration of CLCN1 protein levels by western blot for HSA^{LR} mice that received a single dose of Compound 1 or daily doses of Compound 1.

FIG. 15C shows correction of Clcn1 splicing for mice treated daily with 3 mg/kg of Compound 1.

FIG. 16A illustrates in vivo functional rescue of myotonia by muscle relaxation tests using Compound 2.

FIG. 16B illustrates in vivo functional rescue of myotonia by muscle relaxation tests using Compound 2 or Compound 3.

FIG. 17A shows reduced levels of pathogenic DMPK mRNA in DM1 patient-derived fibroblasts treated with Compound 1 at 1 μM.

FIG. 17B illustrates global splice rescue for DM1 patient-derived fibroblasts treated with Compound 1 for 5 days. Approximately 90% of mis-spliced transcripts were rescued after 5 days of treatment with Compound 1 at 1 μM.

FIG. 18 shows reduced levels of pathogenic mRNA (hACTA1 mRNA) in HSA^{LR} mice that received a single 0.03 mg/kg dose via the intramuscular route of compounds of the disclosure.

FIG. 19A shows reduced levels of pathogenic hACTA1 mRNA in HSA^{LR} mice that were administered four 0.3 mg/kg doses of Compound 1, seven days apart, via the intramuscular route. Tissues were processed six weeks after the final treatment.

FIG. 19B shows near complete Clcn1 splicing rescue in HSA^{LR} mice that were administered four 0.3 mg/kg doses of Compound 1, seven days apart, via the intramuscular route. Tissues were processed six weeks after the final treatment.

FIG. 20 is a chart that shows DMPK mRNA in DM1 model cells treated with Compounds 1, 34, 35, 36 or 90.

FIG. 21 depicts nonlimiting examples of oligonucleotide backbones, where R is a nucleobase (e.g. natural, modified, or non-natural nucleobases) or hydrogen.

FIG. 22 illustrates the structure of Compound 1.

FIG. 23 illustrates the structure of Compound 2.

FIG. 24 illustrates the structure of Compound 3.

FIG. 25 illustrates the structure of Compound 4.

FIG. 26 illustrates the structure of Compound 5.

FIG. 27 illustrates the structure of Compound 6.

FIG. 28 illustrates the structure of Compound 7.

FIG. 29 illustrates the structure of Compound 8.

FIG. 30 illustrates the structure of Compound 9.

FIG. 31 illustrates the structure of Compound 10.

FIG. 32 illustrates the structure of Compound 11.

### DETAILED DESCRIPTION

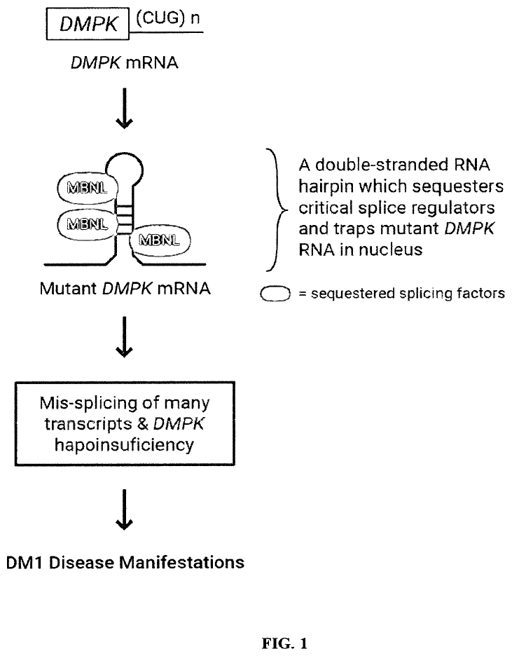

Myotonic Dystrophy Type 1 (DM1) is an autosomal dominant repeat expansion disorder characterized by progressive muscle wasting and weakness. DM1 can also affect the central nervous system (CNS) and heart. FIG. 1 is a chart summarizing the mutational mechanism of DM1. A genetic mutation results in generalized mis-splicing of transcripts and haploinsufficiency of the DMPK protein which both contribute to the disease.

DM1 is caused by an expansion of CTG nucleic acid repeats in the DMPK gene that produce a hairpin structure in transcribed DMPK mRNA (as CUG repeats in the 3′ UTR of the transcript). The hairpin structure sequesters splice regulators and results in mis-splicing of multiple gene transcripts. For example, the hairpins can form aggregates that sequester MBNL1 and/or MBNL2 transcripts, resulting in widespread mis-splicing of pre-mRNAs. Furthermore, binding of splice regulators and aggregation can trap the mutant DMPK mRNA in the nucleus and result in DMPK protein haploinsufficiency, which can exacerbate the CNS and cardiac symptoms that can characterize DM1. Mis-spliced transcripts can also result in altered protein products that are dysfunctional.

Mild DM1 is characterized by cataract development, mild myotonia (sustained muscle contraction), and normal lifespan. Classic DM1 is characterized by muscle weakness and wasting, myotonia, cataract, and often cardiac conduction abnormalities. Adults with classic DM1 can become physically disabled and can have a shortened life span. Congenital DM1 is characterized by hypotonia and severe generalized weakness at birth, often with respiratory insufficiency and early death. Intellectual disability is common. The severity of DM1 can correlate with the number of CTG nucleic acid repeats in the DMPK gene.

mRNAs with a CUG repeat expansion can fold into a hairpin structure with a double stranded region containing GC base pairs and bulged, unpaired U residues. In some embodiments, a compound disclosed herein (e.g., Compound 1, Compound 2, or Compound 3) opens a double-stranded RNA transcript that contains a CUG repeat, sterically displaces a Muscleblind Like Splicing Regulator (e.g., MBNL1 and/or MBNL2) that is sequestered by the transcript, and/or resolves splicing to form a normal or closer to normal complement of mRNAs and/or proteins. In some embodiments, administration of a compound disclosed herein (e.g., Compound 1, Compound 2, or Compound 3) to a cell does not reduce levels of DMPK protein in the cell.

### Compounds of the Disclosure

Provided herein are compounds that reduce expression of the disease-causing DM1 gene and compositions thereof. Selected compounds of the disclosure are provided in TABLE 1. Residue structures, pendant nucleobase identities (when present), and monomer chemical names associated with the symbols used in the structure codes of TABLE 1 and TABLE 2 are provided in TABLE 3. Compounds can be provided as a pharmaceutically-acceptable salt, tautomer, or ionized form thereof.

TABLE 1

| Cpd # | Structure Code(N to C terminus)^{a} | [Z]^{b} | SEQ ID NO^{c} |
|---|---|---|---|
| 1 | [Z] | PNA SEQ NO 27 | — |
| 2 | {RHxRRBxRRHxRFQILYRHxRBxRHxRBx}[Z] | PNA SEQ NO 1 | 128 |
| 3 | {ASSLNIAHxBxR*R*R*}[Z] | PNA SEQ NO 1 | 129 |
| 4 | VeSx[Z]{RHxRRBxRRHxRRBxRHxBx} | PNA SEQ NO 1 | 130 |
| 5 | VeSx[Z]{RHxRRBxRRHxRFQILYRHxRBxRHxRBx} | PNA SEQ NO 1 | 128 |
| 6 | YgYgYgYgYgYgYgPe&&Pe[Z]{PKKKRKV} | PNA SEQ NO 1 | 1 |
| 7 | YgYgYgYgYgYgYgPe&&Pe[Z] | PNA SEQ NO 1 | — |
| 8 | YgYgYgYgYgYgYgEVCt[Z] | PNA SEQ NO 1 | — |
| 9 | [Z] | PNA SEQ NO 43 | — |
| 10 | [Z] | PNA SEQ NO 44 | — |
| 11 | [Z] | PNA SEQ NO 42 | — |
| 12 | KEVCtQxQx[Z]QxQxRQxRR | PNA SEQ NO 1 | — |
| 13 | KEVCtQxQx[Z]QxQxRRQxR | PNA SEQ NO 1 | — |
| 14 | KEVCtQxQx[Z]QxQxRQxQxRQxR | PNA SEQ NO 1 | — |
| 15 | KEVCtQxQx[Z]QxQxRQxQxRR | PNA SEQ NO 1 | — |
| 16 | KEVCtQxQx[Z]QxQxRRQxQxR | PNA SEQ NO 1 | — |
| 17 | KEVCtQxQx[Z]QxQxRQxRQxQxR | PNA SEQ NO 1 | — |
| 18 | KEVCtQxQx[Z]QxQxRRQxRR | PNA SEQ NO 1 | — |
| 19 | KEVCtQxQx[Z]QxQxRRQxRQxR | PNA SEQ NO 1 | — |
| 20 | KEVCtQxQx[Z]QxQxRQxRQxRQxR | PNA SEQ NO 1 | — |
| 21 | QxQx[Z]QxQxYgYgYg | PNA SEQ NO 1 | — |
| 22 | [Z]QxQxYgYgYg | PNA SEQ NO 1 | — |
| 23 | [Z] | PNA SEQ NO 33 | — |
| 24 | QxQx[Z] | PNA SEQ NO 33 | — |
| 25 | KEVCtQxQx[Z]QxQxRR | PNA SEQ NO 1 | — |
| 26 | KEVCtQxQx[Z]QxQxRRR | PNA SEQ NO 1 | — |
| 27 | KEVCtQxQx[Z]QxQxRQxRQxR | PNA SEQ NO 1 | — |
| 28 | KEVCtQxQx[Z]QxQxRQxQxRQxQxR | PNA SEQ NO 1 | — |
| 29 | {C(&)RTIGPSVC(&)}[Z]R*R*R* | PNA SEQ NO 1 | 82 |
| 30 | {T*H*R*P*P*M*W*S*P*V*W*P*}[Z]R*R*R* | PNA SEQ NO 1 | 85 |
| 31 | {P*W*V*P*S*W*M*P*P*R*H*T*}[Z]R*R*R* | PNA SEQ NO 1 | 83 |
| 32 | {H*R*P*Y*I*A*H*}[Z]R*R*R* | PNA SEQ NO 1 | 84 |
| 33 | [Z] | PNA SEQ NO 34 | — |
| 34 | [Z] | PNA SEQ NO 28 | — |
| 35 | [Z] | PNA SEQ NO 29 | — |
| 36 | [Z] | PNA SEQ NO 30 | — |
| 37 | Hd[Z]R*R*R* | PNA SEQ NO 1 | — |
| 38 | LcSx[Z]{ASSLNIAHxBxR*R*R*} | PNA SEQ NO 1 | 129 |
| 39 | Lh[Z]{RHxRRBxRRHxRFQILYRHxRBxRHxRBx} | PNA SEQ NO 1 | 128 |
| 40 | Lh[Z]{RHxRRBxRRHxRRBxRHxBx} | PNA SEQ NO 1 | 130 |
| 41 | Lh[Z]{ASSLNIAHxBxR*R*R*} | PNA SEQ NO 1 | 129 |
| 42 | VeSx[Z]{ASSLNIAHxBxR*R*R*} | PNA SEQ NO 1 | 129 |
| 43 | Hd[Z] | PNA SEQ NO 32 | — |
| 44 | LcSx[Z]R*R*R* | PNA SEQ NO 1 | — |
| 45 | VeSx[Z] | PNA SEQ NO 32 | — |
| 46 | Lh[Z]R*R*R* | PNA SEQ NO 1 | — |
| 47 | VeSx[Z]R*R*R* | PNA SEQ NO 1 | — |
| 48 | Hd[Z]{RHxRRBxRRHxRFQILYRHxRBxRHxRBx} | PNA SEQ NO 1 | 128 |
| 49 | Hd[Z]{RHxRRBxRRHxRRBxRHxBx} | PNA SEQ NO 1 | 130 |
| 50 | Hd[Z]{ASSLNIAHxBxR*R*R*} | PNA SEQ NO 1 | 129 |
| 51 | LcSx[Z]{RHxRRBxRRHxRFQILYRHxRBxRHxRBx} | PNA SEQ NO 1 | 128 |
| 52 | LcSx[Z]{RHxRRBxRRHxRRBxRHxBx} | PNA SEQ NO 1 | 130 |
| 53 | [Z] | PNA SEQ NO 35 | — |
| 54 | [Z] | PNA SEQ NO 36 | — |
| 55 | YgYgYgYgYgYgYgQxQx[Z] | PNA SEQ NO 3 | — |
| 56 | [Z] | PNA SEQ NO 37 | — |
| 57 | YgYgYgYgYgYgYgQxQx[Z] | PNA SEQ NO 5 | — |
| 58 | [Z] | PNA SEQ NO 38 | — |
| 59 | [Z] | PNA SEQ NO 39 | — |
| 60 | [Z] | PNA SEQ NO 30 | — |
| 61 | YgYgYgYgYgYgYgQxQx[Z] | PNA SEQ NO 8 | — |
| 62 | [Z] | PNA SEQ NO 41 | — |
| 63 | [Z] | PNA SEQ NO 10 | — |
| 64 | [Z] | PNA SEQ NO 11 | — |
| 65 | [Z] | PNA SEQ NO 12 | — |
| 66 | [Z] | PNA SEQ NO 13 | — |
| 67 | [Z] | PNA SEQ NO 14 | — |
| 68 | [Z] | PNA SEQ NO 15 | — |
| 69 | [Z] | PNA SEQ NO 16 | — |
| 70 | [Z] | PNA SEQ NO 17 | — |
| 71 | [Z] | PNA SEQ NO 18 | — |
| 72 | [Z] | PNA SEQ NO 19 | — |
| 73 | [Z] | PNA SEQ NO 9 | — |
| 74 | [Z] | PNA SEQ NO 20 | — |
| 75 | [Z] | PNA SEQ NO 21 | — |
| 76 | [Z] | PNA SEQ NO 22 | — |
| 77 | [Z] | PNA SEQ NO 23 | — |
| 78 | [Z] | PNA SEQ NO 24 | — |
| 79 | [Z] | PNA SEQ NO 25 | — |
| 80 | [Z] | PNA SEQ NO 1 | — |
| 81 | {RGRGRGRGRGRGRG}[Z] | PNA SEQ NO 1 | 93 |
| 82 | [Z] | PNA SEQ NO 26 | — |
| 83 | {RHxRRBxRRHxRYQFLIRHxRBxRHxRBx}[Z] | PNA SEQ NO 1 | 131 |
| 84 | {RHxRRBxRRHxRIQFLIRHxRBxRHxRBx}[Z] | PNA SEQ NO 1 | 132 |
| 85 | {RHxRRBxRRHxYRFLIRHxRBxRHxRBx}[Z] | PNA SEQ NO 1 | 133 |
| 86 | {RHxRRBxRRHxRRBxRHxBx}[Z] | PNA SEQ NO 1 | 130 |
| 87 | {RRWWRRWRR}[Z] | PNA SEQ NO 1 | 76 |
| 88 | {RRWQW}[Z] | PNA SEQ NO 1 | 89 |
| 89 | {GRKKRRQRRRPQ}Px{GFWFG}[Z] | PNA SEQ NO 1 | 92 and 91 |
| 90 | [Z] | PNA SEQ NO 31 | |

ªUnless otherwise noted, all C-termini are amidated.^{b}The structure code corresponding to eachPNA SEQ NO is provided in TABLE 2. For example,the structure code of Compound 19 is KEVCtQxQx[Z]QxQxRRQxRQxR, which isKEVCtQxQxCsAnGsCnAsGnCsAnGsCnAsGnCsAnQxQxRRQxRQxR.^{c}Portion of structure code within braces(e.g., {PKKKRKV}” (SEQ ID NO: 1)), when present,
corresponds to SEQ ID NO provided in this column.

TABLE 2

| PNA | |
|---|---|
| SEQ NO | SEQ |
| 1 | CsAnGsCnAsGnCsAnGsCnAsGnCsAn |
| 2 | AsGnCsAnGsCnCsGnCsAnGsCnAsAn |
| 3 | CsAnGsCnAsGnCsAnGsCn |
| 4 | CsAsGnCsAsGnCsAsGnCsAsGnCsAs |
| 5 | CsAsGnCsAsGnCsAsGnCs |
| 6 | CsAnGsCnAsGnCsAnGsCnAsGn |
| 7 | CsAsGnCsAsGnCsAsGnCsAsGn |
| 8 | CsAnGnCsAnGnCsAnGnCsAnGnCsAn |
| 9 | CsAnGsCnAsGnCsAnGsCnAsGnCsAnGsCnAs |
| 10 | CsAnGsCnAsGnCsAnGsCnAsGnCsAnGs |
| 11 | GsCnAsGnCsAnGsCnAsGnCsAnGsCn |
| 12 | GsCnAsGnCsAnGsCnAsGnCsAnGsCnAs |
| 13 | GsCnAsGnCsAnGsCnAsGnCsAnGsCnAsGn |
| 14 | GsCnAsGnCsAnGsCnAsGnCsAnGsCnAsGnCs |
| 15 | CsAnGsTnCsGnAsGnGsCnCsAnAsAn |
| 16 | CsAnGsTnCsGnAsGnGsCnCsAnAsAnGs |
| 17 | CsAnGsTnCsGnAsGnGsCnCsAnAsAnGsAn |
| 18 | CsAnGsTnCsGnAsGnGsCnCsAnAsAnGsAnAs |
| 19 | CsAnGsCnAsGnCsAnGsCnAsGnCsAnGsCn |
| 20 | AnGsCnAsGnCsAnGsCnAsGnCsAn |
| 21 | AnGsCnAsGnCsAnGsCnAsGnCsAnGs |
| 22 | AnGsCnAsGnCsAnGsCnAsGnCsAnGsCn |
| 23 | AnGsCnAsGnCsAnGsCnAsGnCsAnGsCnAs |
| 24 | AnGsCnAsGnCsAnGsCnAsGnCsAnGsCnAsGn |
| 25 | GsCnAsGnCsAnGsCnAsGnCsAnGs |
| 26 | CsAnGsTnCsGnAsGnGsCnCsAnAsAnGsAnAsGn |
| 27 | YgYgYgYgYgYgYgCsAnGsCnAsGnCsAnGsCnAsGnCsAn |
| 28 | YgYgYgYgYgYgCsAnGsCnAsGnCsAnGsCnAsGnCsAn |
| 29 | YgYgYgYgYgCsAnGsCnAsGnCsAnGsCnAsGnCsAn |
| 30 | YgYgYgYgCsAnGsCnAsGnCsAnGsCnAsGnCsAn |
| 31 | YgYgYgCsAnGsCnAsGnCsAnGsCnAsGnCsAn |
| 32 | CsAnGsCnAsGnCsAnGsCnAsGnCsAnYgYgYgYgYgYgYg |
| 33 | CsAnGsCnAsGnCsAnGsCnAsGnCsAnYgYgYg |
| 34 | YgYgYgYgYgYgYgAsGnCsAnGsCnCsGnCsAnGsCnAsAn |
| 35 | YgYgYgYgYgYgYgCsAnGsCnAsGnCsAnGsCn |
| 36 | YgYgYgYgYgYgYgCsAsGnCsAsGnCsAsGnCsAsGnCsAs |
| 37 | YgYgYgYgYgYgYgCsAsGnCsAsGnCsAsGnCs |
| 38 | YgYgYgYgYgYgYgCsAnGsCnAsGnCsAnGsCnAsGn |
| 39 | YgYgYgYgYgYgYgCsAsGnCsAsGnCsAsGnCsAsGn |
| 40 | YgYgYgYgYgYgYgCsAnGnCsAnGnCsAnGnCsAnGnCsAn |
| 41 | YgYgYgYgYgYgYgCsAnGsCnAsGnCsAnGsCnAsGnCsAn |
| | GsCnAs |
| 42 | YgYgYgYgYgYgYgCsAnGsCnAsGnCsAnGsCnAsGnCsAn |
| | Gs |
| 43 | YgYgYgYgYgYgYgCsAnGsCnAsGnCsAnGsCnAsGnCsAn |
| | GsCn |
| 44 | YgYgYgYgYgYgYgCsAnGsCnAsGnCsAnGsCnAsGnCsAn |
| | GsCnAsGn |

TABLE 3

| | | | Monomer Chemical |
|---|---|---|---|
| Code^{a,b,c} | Residue Structure | Nucleobase | Name^{d} |
| An | 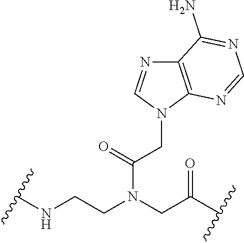 [SMILES: *NCCN(CC(*)=O)C(=O)Cn1cnc2c(N)ncnc21] | adenine | N-(2-(6-amino-9H-purin- 9-yl)acetyl)-N-(2- aminoethyl)glycine |
| Cn | 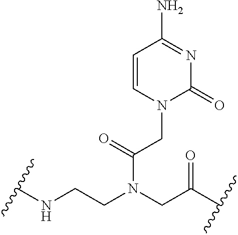 [SMILES: *NCCN(CC(*)=O)C(=O)Cn1ccc(N)nc1=O] | cytosine | N-(2-(4-amino-2- oxopyrimidin-1(2H)- yl)acetyl)-N-(2- aminoethyl)glycine |
| Gn | 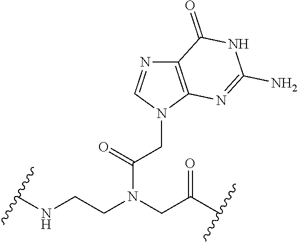 [SMILES: *NCCN(CC(*)=O)C(=O)Cn1cnc2c(=O)[nH]c(N)nc21] | guanine | N-(2-(2-amino-6-oxo-1,6- dihydro-9H-purin-9- yl)acetyl)-N-(2- aminoethyl)glycine |
| Tn | 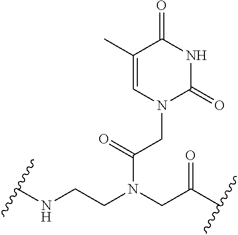 [SMILES: *NCCN(CC(*)=O)C(=O)Cn1cc(C)c(=O)[nH]c1=O] | thymine | N-(2-aminoethyl)-N-(2-(5- methyl-2,4-dioxo-3,4- dihydropyrimidin-1(2H)- yl)acetyl)glycine |
| Dn | 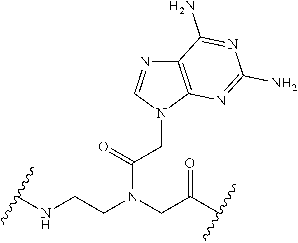 [SMILES: *NCCN(CC(*)=O)C(=O)Cn1cnc2c(N)nc(N)nc21] | 2,6- diaminopurine | N-(2-aminoethyl)-N-(2- (2,6-diamino-9H-purin-9- yl)acetyl)glycine |
| Zn | 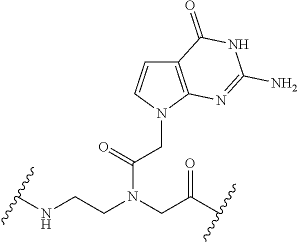 [SMILES: *NCCN(CC(*)=O)C(=O)Cn1ccc2c(=O)[nH]c(N)nc21] | 7- deazaguanine | N-(2-(2-amino-4-oxo-3,4- dihydro-7H-pyrrolo[2,3- d]pyrimidin-7-yl)acetyl)- N-(2-aminoethyl)glycine |
| Sn | 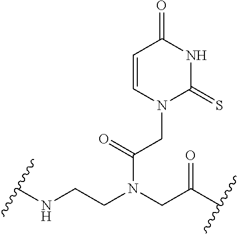 [SMILES: *NCCN(CC(*)=O)C(=O)Cn1ccc(=O)[nH]c1=S] | 2-thiouracil | N-(2-aminoethyl)-N-(2-(4- oxo-2-thioxo-3,4- dihydropyrimidin-1(2H)- yl)acetyl)glycine |
| Yn | 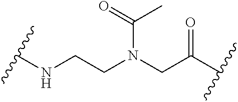 [SMILES: *NCCN(CC(*)=O)C(C)=O] | — | N-acetyl-N-(2- aminoethyl)glycine |
| As | 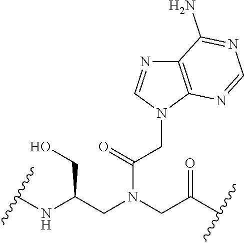 [SMILES: *N[C@@H](CO)CN(CC(*)=O)C(=O)Cn1cnc2c(N)ncnc21] | adenine | (R)-N-(2-amino-3- hydroxypropyl)-N-(2-(6- amino-9H-purin-9- yl)acetyl)glycine |
| Cs | 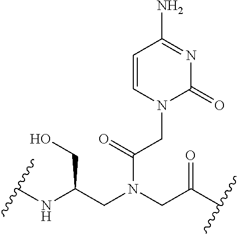 [SMILES: *N[C@@H](CO)CN(CC(*)=O)C(=O)Cn1ccc(N)nc1=O] | cytosine | (R)-N-(2-(4-amino-2- oxopyrimidin-1(2H)- yl)acetyl)-N-(2-amino-3- hydroxypropyl)glycine |
| Gs | 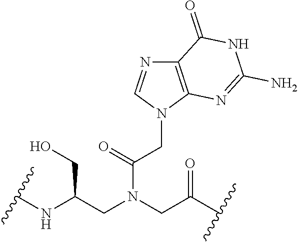 [SMILES: *N[C@@H](CO)CN(CC(*)=O)C(=O)Cn1cnc2c(=O)[nH]c(N)nc21] | guanine | (R)-N-(2-amino-3- hydroxypropyl)-N-(2-(2- amino-6-oxo-1,6-dihydro- 9H-purin-9- yl)acetyl)glycine |
| Ts | 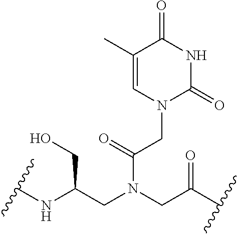 [SMILES: *N[C@@H](CO)CN(CC(*)=O)C(=O)Cn1cc(C)c(=O)[nH]c1=O] | thymine | (R)-N-(2-amino-3- hydroxypropyl)-N-(2-(5- methyl-2,4-dioxo-3,4- dihydropyrimidin-1(2H)- yl)acetyl)glycine |
| Ds | 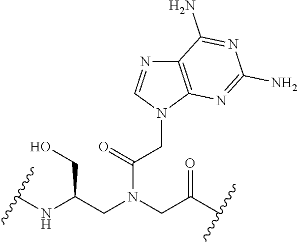 [SMILES: *N[C@@H](CO)CN(CC(*)=O)C(=O)Cn1cnc2c(N)nc(N)nc21] | 2,6- diaminopurine | (R)-N-(2-amino-3- hydroxypropyl)-N-(2-(2,6- diamino-9H-purin-9- yl)acetyl)glycine |
| Zs | 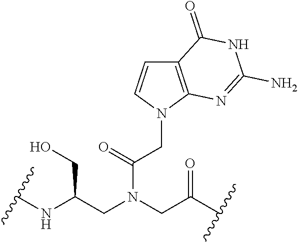 [SMILES: *N[C@@H](CO)CN(CC(*)=O)C(=O)Cn1ccc2c(=O)[nH]c(N)nc21] | 7- deazaguanine | (R)-N-(2-amino-3- hydroxypropyl)-N-(2-(2- amino-4-oxo-3,4-dihydro- 7H-pyrrolo[2,3- d]pyrimidin-7- yl)acetyl)glycine |
| Ss | 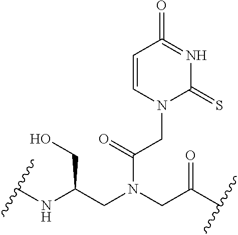 | 2-thiouracil | (R)-N-(2-amino-3- hydroxypropyl)-N-(2-(4- oxo-2-thioxo-3,4- dihydropyrimidin-1(2H)- yl)acetyl)glycine |
| Ys | 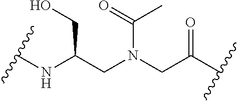 [SMILES: *N[C@@H](CO)CN(CC(*)=O)C(C)=O] | — | (R)-N-acetyl-N-(2-amino- 3-hydroxypropyl)glycine |
| Av | 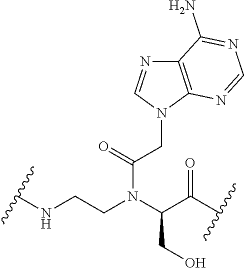 [SMILES: *NCCN(C(=O)Cn1cnc2c(N)ncnc21)[C@H](CO)C(*)=O] | adenine | N-(2-(6-amino-9H-purin- 9-yl)acetyl)-N-(2- aminoethyl)-D-serine |
| Cv | 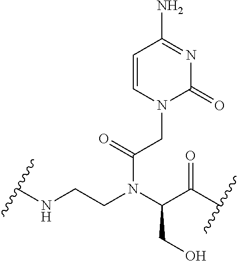 [SMILES: *NCCN(C(=O)Cn1ccc(N)nc1=O)[C@H](CO)C(*)=O] | cytosine | N-(2-(4-amino-2- oxopyrimidin-1(2H)- yl)acetyl)-N-(2- aminoethyl)-D-serine |
| Gv | 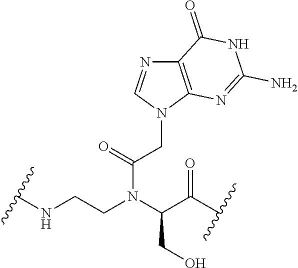 [SMILES: *NCCN(C(=O)Cn1cnc2c(=O)[nH]c(N)nc21)[C@H](CO)C(*)=O] | guanine | N-(2-(2-amino-6-oxo-1,6- dihydro-9H-purin-9- yl)acetyl)-N-(2- aminoethyl)-D-serine |
| Tv | 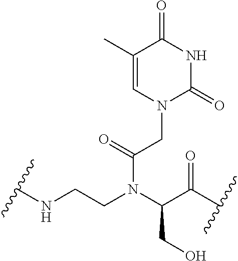 [SMILES: *NCCN(C(=O)Cn1cc(C)c(=O)[nH]c1=O)[C@H](CO)C(*)=O] | thymine | N-(2-aminoethyl)-N-(2-(5- methyl-2,4-dioxo-3,4- dihydropyrimidin-1(2H)- yl)acetyl)-D-serine |
| Dv | 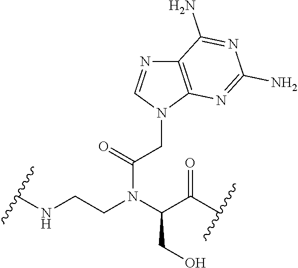 [SMILES: *NCCN(C(=O)Cn1cnc2c(N)nc(N)nc21)[C@H](CO)C(*)=O] | 2,6- diaminopurine | N-(2-aminoethyl)-N-(2- (2,6-diamino-9H-purin-9- yl)acetyl)-D-serine |
| Zv | 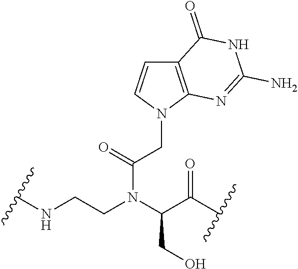 [SMILES: *NCCN(C(=O)Cn1ccc2c(=O)[nH]c(N)nc21)[C@H](CO)C(*)=O] | 7- deazaguanine | N-(2-(2-amino-4-oxo-3,4- dihydro-7H-pyrrolo[2,3- d]pyrimidin-7-yl)acetyl)- N-(2-aminoethyl)-D-serine |
| Sv | 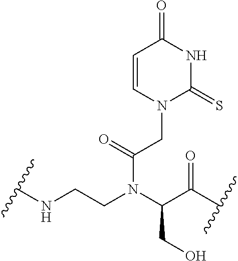 [SMILES: *NCCN(C(=O)Cn1ccc(=O)[nH]c1=S)[C@H](CO)C(*)=O] | 2-thiouracil | N-(2-aminoethyl)-N-(2-(4- oxo-2-thioxo-3,4- dihydropyrimidin-1(2H)- yl)acetyl)-D-serine |
| Yv | 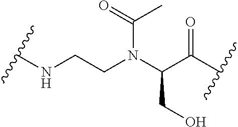 [SMILES: *NCCN(C(C)=O)[C@H](CO)C(*)=O] | — | N-acetyl-N-(2- aminoethyl)-D-serine |
| Ag | 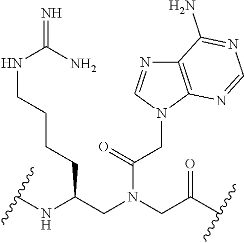 [SMILES: *N[C@@H](CCCCNC(=N)N)CN(CC(*)=O)C(=O)Cn1cnc2c(N)ncnc21] | adenine | (S)-N-(2-amino-6- guanidinohexyl)-N-(2-(6- amino-9H-purin-9- yl)acetyl)glycine |
| Cg | 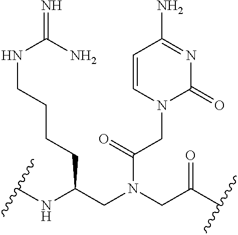 [SMILES: *N[C@@H](CCCCNC(=N)N)CN(CC(*)=O)C(=O)Cn1ccc(N)nc1=O] | cytosine | (S)-N-(2-(4-amino-2- oxopyrimidin-1(2H)- yl)acetyl)-N-(2-amino-6- guanidinohexyl)glycine |
| Gg | 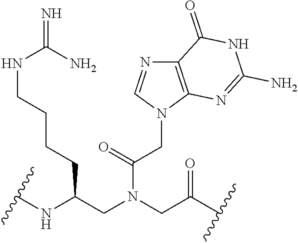 [SMILES: *N[C@@H](CCCCNC(=N)N)CN(CC(*)=O)C(=O)Cn1cnc2c(=O)[nH]c(N)nc21] | guanine | (S)-N-(2-amino-6- guanidinohexyl)-N-(2-(2- amino-6-oxo-1,6-dihydro- 9H-purin-9- yl)acetyl)glycine |
| Tg | 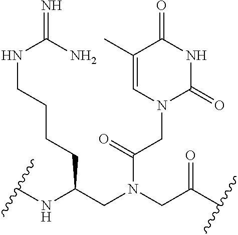 [SMILES: *N[C@@H](CCCCNC(=N)N)CN(CC(*)=O)C(=O)Cn1cc(C)c(=O)[nH]c1=O] | thymine | (S)-N-(2-amino-6- guanidinohexyl)-N-(2-(5- methyl-2,4-dioxo-3,4- dihydropyrimidin-1(2H)- yl)acetyl)glycine |
| Dg | 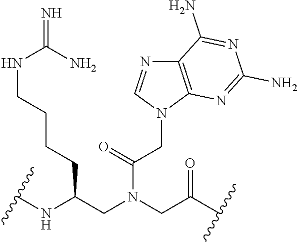 [SMILES: *N[C@@H](CCCCNC(=N)N)CN(CC(*)=O)C(=O)Cn1cnc2c(N)nc(N)nc21] | 2,6- diaminopurine | (S)-N-(2-amino-6- guanidinohexyl)-N-(2- (2,6-diamino-9H-purin-9- yl)acetyl)glycine |
| Zg | 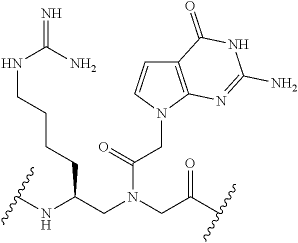 [SMILES: *N[C@@H](CCCCNC(=N)N)CN(CC(*)=O)C(=O)Cn1ccc2c(=O)[nH]c(N)nc21] | 7- deazaguanine | (S)-N-(2-(2-amino-4-oxo- 3,4-dihydro-7H- pyrrolo[2,3-d]pyrimidin-7- yl)acetyl)-N-(2-amino-6- guanidinohexyl)glycine |
| Sg | 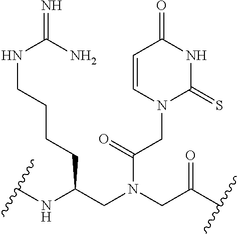 [SMILES: *N[C@@H](CCCCNC(=N)N)CN(CC(*)=O)C(=O)Cn1ccc(=O)[nH]c1=S] | 2-thiouracil | (S)-N-(2-amino-6- guanidinohexyl)-N-(2-(4- oxo-2-thioxo-3,4- dihydropyrimidin-1(2H)- yl)acetyl)glycine |
| Yg | 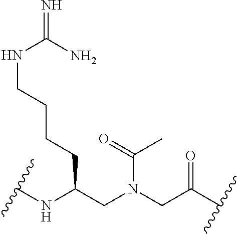 [SMILES: *N[C@@H](CCCCNC(=N)N)CN(CC(*)=O)C(C)=O] | — | (S)-N-acetyl-N-(2-amino- 6-guanidinohexyl)glycine |
| Ap | 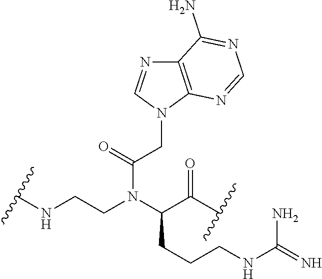 [SMILES: *NCCN(C(=O)Cn1cnc2c(N)ncnc21)[C@H](CCCNC(=N)N)C(*)=O] | adenine | N²-(2-(6-amino-9H-purin- 9-yl)acetyl)-N²-(2- aminoethyl)-D-arginine |
| Cp | 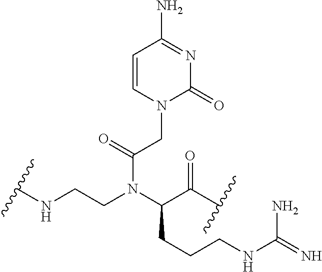 [SMILES: *NCCN(C(=O)Cn1ccc(N)nc1=O)[C@H](CCCNC(=N)N)C(*)=O] | cytosine | N²-(2-(4-amino-2- oxopyrimidin-1(2H)- yl)acetyl)-N²-(2- aminoethyl)-D-arginine |
| Gp | 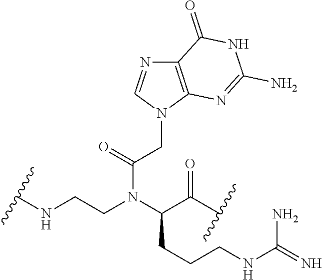 [SMILES: *NCCN(C(=O)Cn1cnc2c(=O)[nH]c(N)nc21)[C@H](CCCNC(=N)N)C(*)=O] | guanine | N²-(2-(2-amino-6-oxo-1,6- dihydro-9H-purin-9- yl)acetyl)-N²-(2- aminoethyl)-D-arginine |
| Tp | 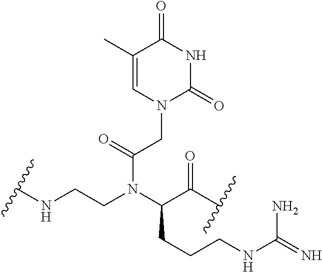 [SMILES: *NCCN(C(=O)Cn1cc(C)c(=O)[nH]c1=O)[C@H](CCCNC(=N)N)C(*)=O] | thymine | N²-(2-aminoethyl)-N²-(2- (5-methyl-2,4-dioxo-3,4- dihydropyrimidin-1(2H)- yl)acetyl)-D-arginine |
| Dp | 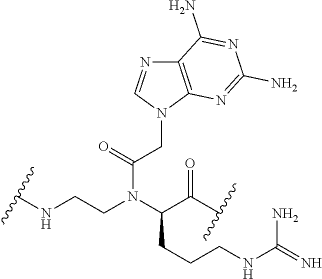 [SMILES: *NCCN(C(=O)Cn1cnc2c(N)nc(N)nc21)[C@H](CCCNC(=N)N)C(*)=O] | 2,6- diaminopurine | N²-(2-aminoethyl)-N²-(2- (2,6-diamino-9H-purin-9- yl)acetyl)-D-arginine |
| Zp | 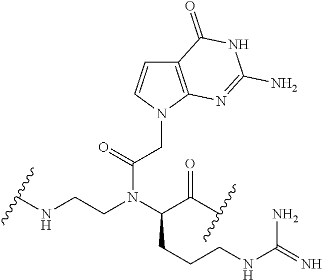 [SMILES: *NCCN(C(=O)Cn1ccc2c(=O)[nH]c(N)nc21)[C@H](CCCNC(=N)N)C(*)=O] | 7- deazaguanine | N²-(2-(2-amino-4-oxo-3,4- dihydro-7H-pyrrolo[2,3- d]pyrimidin-7-yl)acetyl)- N²-(2-aminoethyl)-D- arginine |
| Sp | 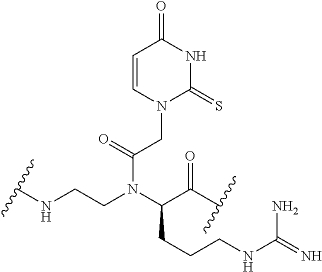 [SMILES: *NCCN(C(=O)Cn1ccc(=O)[nH]c1=S)[C@H](CCCNC(=N)N)C(*)=O] | 2-thiouracil | N²-(2-aminoethyl)-N²-(2- (4-oxo-2-thioxo-3,4- dihydropyrimidin-1(2H)- yl)acetyl)-D-arginine |
| Yp | 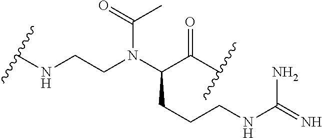 [SMILES: *NCCN(C(C)=O)[C@H](CCCNC(=N)N)C(*)=O] | — | N²-acetyl-N²-(2- aminoethyl)-D-arginine |
| Ct | 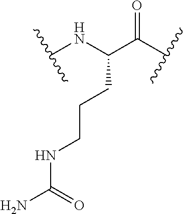 [SMILES: *N[C@@H](CCCNC(N)=O)C(*)=O] | — | L-citrulline |
| Qx | 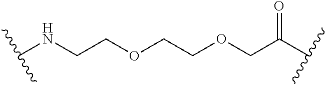 [SMILES: *NCCOCCOCC(*)=O] | — | 2-(2-(2- aminoethoxy)ethoxy)acetic acid |
| Pe&&Pe | 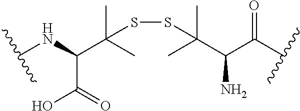 [SMILES: *N[C@H](C(=O)O)C(C)(C)SSC(C)(C)[C@H](N)C(*)=O] | — | (2R,2′R)-3,3′- disulfanediylbis(2-amino- 3-methylbutanoic acid) |
| Hx | 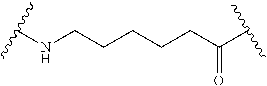 [SMILES: *NCCCCCC(*)=O] | — | 6-aminohexanoic acid |
| Bx | 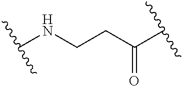 [SMILES: *NCCC(*)=O] | — | 3-aminopropanoic acid |
| Sx | 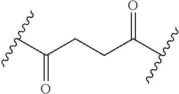 [SMILES: *C(=O)CCC(*)=O] | — | propanedioic acid |
| Px | 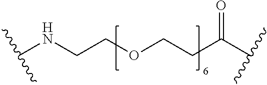 | — | Amino-PEG6-acid |
| Ve | 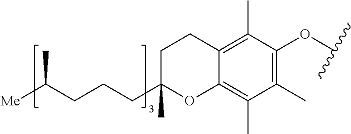 | — | Vitamin E |
| Lc | 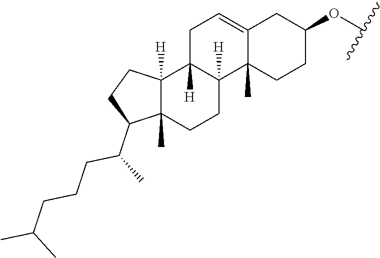 | — | Cholesterol |
| Lh | 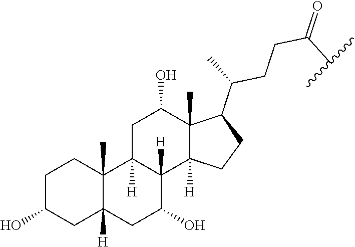 | — | Cholic acid |
| Hd | 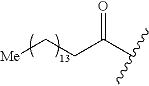 | — | Palmitic acid |

^{a}Proteinogenic amino acid residues in compounds provided in TABLE 1 are represented by the following one-letter codes: A: L-alanine, R: L-arginine, N: L-asparagine, D: L-aspartic acid, C: L-cysteine, E: L-glutamic acid, Q: L-glutamine, G: glycine, H: L-histidine, I: L-isoleucine, L: L-leucine , K: L-lysine, M: L-methionine, F: L-phenylalanine, P: L-proline, S: L-serine, T: L-threonine, W: L-tryptophan, Y: L-tyrosine, V: L-valine.
^{b}An antipode of a chiral residue presented in TABLE 3 is represented in TABLE 1 by the code of the chiral residue followed by an asterisk (*). For example, R* represents D-arginine.
^{c}In TABLE 1, C(&) represents an L-cysteine residue that is covalently bound via the sulfur atom of its side chain to a sulfur atom of another L-cysteine residue represented by C(&). For example, a compound having the sequence code GC(&)GGGGGC(&)G (SEQ ID NO: 134) comprises two cysteine residues that are bound to each other via an intrachain disulfide bond.
^{d}For each residue, a chemical name is provided for the corresponding unincorporated monomer.

In some embodiments, the disclosure provides a compound comprising a structure, wherein the structure binds to a mRNA sequence transcribed from a gene associated with a neuromuscular disease phenotype, wherein the structure is attached to a chain of atoms bearing a series of side chains, wherein the series of side chains has a sub-series of three consecutive side chains that are: i) guanidinoalkyl; ii) C(O)-alkyl; and iii) guanidinoalkyl.

In some embodiments, the disclosure provides a compound comprising a structure, wherein the structure binds to a mRNA sequence transcribed from a gene associated with a neuromuscular disease phenotype, wherein the structure is attached to a chain of atoms, wherein carbon atoms of the chain of atoms bear a series of side chains, wherein the series of side chains has two consecutive side chains that are each independently guanidinoalkyl.

In some embodiments, the disclosure provides a compound comprising a structure, wherein the structure binds to a mRNA sequence transcribed from a gene associated with a neuromuscular disease phenotype, wherein the structure is attached to a chain of atoms, wherein carbon atoms of the chain of atoms bear a series of side chains, wherein the series of side chains has six consecutive side chains that each independently bear a positive charge at physiological pH.

In some embodiments, the structure is a peptide nucleic acid structure. In some embodiments, the neuromuscular disease phenotype is a DM1 disease phenotype.

In some embodiments, the disclosure provides a compound comprising a structure, wherein the structure binds to a mRNA sequence that contains a subsequence that is (CUG)_{z}, wherein z is an integer from 1-100 (SEQ ID NO: 135), wherein the structure is attached to a chain of atoms bearing a series of side chains, wherein the series of side chains has a sub-series of three consecutive side chains that are: i) guanidinoalkyl; ii) C(O)-alkyl; and iii) guanidinoalkyl.

In some embodiments, the disclosure provides a compound comprising a structure, wherein the structure binds to a mRNA sequence that contains a subsequence that is (CUG)_{z}, wherein z is an integer from 1-100 (SEQ ID NO: 135), wherein the structure is attached to a chain of atoms, wherein carbon atoms of the chain of atoms bear a series of side chains, wherein the series of side chains has two consecutive side chains that are each independently guanidinoalkyl.

In some embodiments, the disclosure provides a compound comprising a structure, wherein the structure binds to a mRNA sequence that contains a subsequence that is (CUG)_{z}, wherein z is an integer from 1-100 (SEQ ID NO: 135), wherein the structure is attached to a chain of atoms, wherein carbon atoms of the chain of atoms bear a series of side chains, wherein the series of side chains has six consecutive side chains that each independently bear a positive charge at physiological pH. wherein the structure binds to the mRNA sequence at the subsequence that is (CUG)_{z}.

In some embodiments, the disclosure provides a composition comprising a compound that is therapeutically-effective for treatment of a neuromuscular disease, wherein the compound comprises a structure that is:

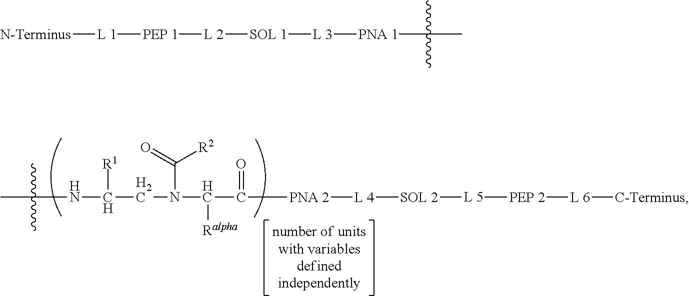

wherein:
the number of units with variables defined independently is at least 11;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle;
C-Terminus is OH, OMe, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof.

In some embodiments, PEP1 is absent. In some embodiments, PEP1 is the peptide sequence. In some embodiments, the peptide sequence of PEP1 is a nuclear localization sequence. In some embodiments, PEP1 is a sequence selected from the group consisting of -Pro-Lys-Lys-Lys-Arg-Lys-Val- (SEQ ID NO: 1), -Ala-Lys-Ala-Ser-Ser-Leu-Asn-Ile-Ala- (SEQ ID NO:77), -Ala-Ser-Ser-Leu-Asn-Ile-Ala- (SEQ ID NO: 78), -Arg-Arg-, -Arg-Phe-Gln-Ile-Leu-Tyr-Arg- (SEQ ID NO: 86), -(D-Arg)-(D-Arg)-(D-Arg)-, -Arg-Gly-Arg-Gly-Arg-Gly-Arg-Gly-Arg-Gly-Arg-Gly-Arg-Gly- (SEQ ID NO: 93), -Arg-Tyr-Gln-Phe-Leu-Ile-Arg- (SEQ ID NO: 87), -Arg-Ile-Gln-Phe-Leu-Ile-Arg- (SEQ ID NO: 88), -Arg-Arg-Trp-Trp-Arg-Arg-Trp-Arg-Arg- (SEQ ID NO: 76), (D-Thr)-(D-His)-(D-Arg)-(D-Pro)-(D-Pro)-(D-Met)-(D-Trp)-(D-Ser)-(D-Pro)-(D-Val)-(D-Trp)-(D-Pro-) (SEQ ID NO: 85), -(D-Pro)-(D-Trp)-(D-Val)-(D-Pro)-(D-Ser)-(D-Trp)-(D-Met)-(D-Pro)-(D-Pro)-(D-Arg)-(D-His)-(D-Thr)- (SEQ ID NO: 83), -(D-His)-(D-Arg)-(D-Pro)-(D-Tyr)-(D-Ile)-(D-Ala)-(D-His)- (SEQ ID NO: 84), and -Arg-Arg-Trp-Gln-Trp- (SEQ ID NO: 89).

In some embodiments, PEP1 is -Cys¹-Arg-Thr-Ile-Gly-Pro-Ser-Val-Cys²-, wherein Cys¹ and Cys² are bound to one another via an intrachain disulfide bond (SEQ ID NO: 82).

In some embodiments, PEP2 is absent. In some embodiments, PEP2 is the peptide sequence. In some embodiments, the peptide sequence of PEP2 is a nuclear localization sequence. In some embodiments, PEP2 is a sequence selected from the group consisting of -Pro-Lys-Lys-Lys-Arg-Lys-Val- (SEQ ID NO: 1), -Ala-Lys-Ala-Ser-Ser-Leu-Asn-Ile-Ala- (SEQ ID NO:77), -Ala-Ser-Ser-Leu-Asn-Ile-Ala- (SEQ ID NO: 78), -Arg-Arg-,-Arg-Phe-Gln-Ile-Leu-Tyr-Arg- (SEQ ID NO: 86), -(D-Arg)-(D-Arg)-(D-Arg)-, -Arg-Gly-Arg-Gly-Arg-Gly-Arg-Gly-Arg-Gly-Arg-Gly-Arg-Gly- (SEQ ID NO: 93), -Arg-Tyr-Gln-Phe-Leu-Ile-Arg- (SEQ ID NO: 87), -Arg-Ile-Gln-Phe-Leu-Ile-Arg- (SEQ ID NO: 88), -Arg-Arg-Trp-Trp-Arg-Arg-Trp-Arg-Arg- (SEQ ID NO: 76), (D-Thr)-(D-His)-(D-Arg)-(D-Pro)-(D-Pro)-(D-Met)-(D-Trp)-(D-Ser)-(D-Pro)-(D-Val)-(D-Trp)-(D-Pro-) (SEQ ID NO: 85), -(D-Pro)-(D-Trp)-(D-Val)-(D-Pro)-(D-Ser)-(D-Trp)-(D-Met)-(D-Pro)-(D-Pro)-(D-Arg)-(D-His)-(D-Thr)- (SEQ ID NO: 83), -(D-His)-(D-Arg)-(D-Pro)-(D-Tyr)-(D-Ile)-(D-Ala)-(D-His)- (SEQ ID NO: 84), and -Arg-Arg-Trp-Gln-Trp- (SEQ ID NO: 89).

In some embodiments, SOL1 is absent. In some embodiments, SOL1 is the water-solubilizing group. In some embodiments, the water-solubilizing group of SOL1 is a peptide sequence. In some embodiments, the water-solubilizing group of SOL1 is a group that contains multiple electrical charges at physiological pH. In some embodiments, the water-solubilizing group of SOL1 is a group that contains multiple positive charges at physiological pH. In some embodiments, the water-solubilizing group of SOL1 is a polyethyleneglycol group. In some embodiments, the water-solubilizing group of SOL1 is -Arg-Arg-NH(CH₂)₂C(O)-Arg-Arg- (SEQ ID NO: 136).

In some embodiments, the water-solubilizing group of SOL1 is a group of formula:

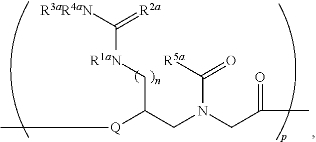

wherein
R^{1a} is H, alkyl, or a nitrogen atom protecting group;
R^{2a} is O, NH, N(alkyl), or N(Pg^{N}), wherein Pg^{N} is a nitrogen atom protecting group;
R^{1a} is H, alkyl, or a nitrogen atom protecting group;
R^{4a} is H, alkyl, or a nitrogen atom protecting group;
R^{5a} is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
n is 1, 2, 3, 4, 5, 6, 7, 8, 9, or 10; and
p is an integer that is 1-1,000.

In some embodiments, the water-solubilizing group of SOL1 is a group of formula:

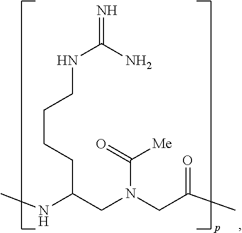

wherein p is an integer that is 1-1,000.

In some embodiments, the water-solubilizing group of SOL1 is a group of formula:

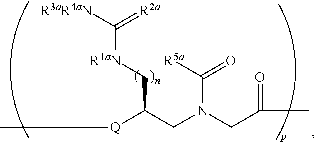

wherein
R^{1a} is H, alkyl, or a nitrogen atom protecting group;
R^{2a} is O, NH, N(alkyl), or N(Pg^{N}), wherein Pg^{N} is a nitrogen atom protecting group;
R^{1a} is H, alkyl, or a nitrogen atom protecting group;
R^{4a} is H, alkyl, or a nitrogen atom protecting group;
R^{5a} is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
n is 1, 2, 3, 4, 5, 6, 7, 8, 9, or 10; and
p is an integer that is 1-1,000.

In some embodiments, the water-solubilizing group of SOL1 is a group of formula:

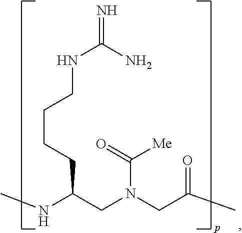

wherein p is an integer that is 1-1,000.

In some embodiments, p is an integer that is 1-100 or an integer that is 1-50. In some embodiments, p is an integer that is 1, 2, 3, 4, 5, 6, 7, 8, 9, 10, 11, 12, 13, 14, 15, 16, 17, 18, 19, or 20. In some embodiments, p is an integer that is 1, 2, 3, 4, 5, 6, 7, 8, 9, 10, 11, or 12. In some embodiments, p is an integer that is 3, 4, 5, 6, 7, 8, 9, or 10. In some embodiments, p is an integer that is 5, 6, 7, 8, or 9. In some embodiments, p is an integer that is 6, 7, or 8. In some embodiments, p is an integer that is 7.

In some embodiments, SOL1 is absent. In some embodiments, SOL1 is the water-solubilizing group. In some embodiments, the water-solubilizing group of SOL1 is a peptide sequence. In some embodiments, the water-solubilizing group of SOL1 is a group that contains multiple electrical charges at physiological pH. In some embodiments, the water-solubilizing group of SOL1 is a group that contains multiple positive charges at physiological pH. In some embodiments, the water-solubilizing group of SOL1 is a polyethyleneglycol group. In some embodiments, the water-solubilizing group of SOL1 is -Arg-Arg-NH(CH₂)₂C(O)-Arg-Arg- (SEQ ID NO: 136).

In some embodiments, the water-solubilizing group of SOL2 is a group of formula:

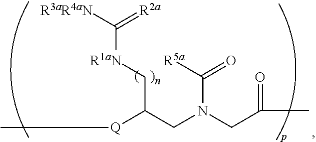

wherein
R^{1a} is H, alkyl, or a nitrogen atom protecting group;
R^{2a} is O, NH, N(alkyl), or N(Pg^{N}), wherein Pg^{N} is a nitrogen atom protecting group;
R^{1a} is H, alkyl, or a nitrogen atom protecting group;
R^{4a} is H, alkyl, or a nitrogen atom protecting group;
R^{5a} is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
n is 1, 2, 3, 4, 5, 6, 7, 8, 9, or 10; and
p is an integer that is 1-1,000.

In some embodiments, the water-solubilizing group of SOL2 is a group of formula:

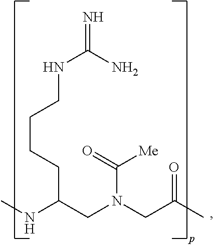

wherein p is an integer that is 1-1,000.

In some embodiments, the water-solubilizing group of SOL2 is a group of formula:

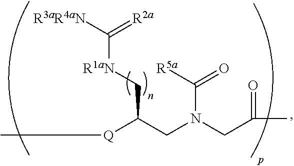

wherein
R^{1a} is H, alkyl, or a nitrogen atom protecting group;

R^{2a} is O, NH, N(alkyl), or N(Pg^{N}), wherein Pg^{N} is a nitrogen atom protecting group;
R^{1a} is H, alkyl, or a nitrogen atom protecting group;
R^{4a} is H, alkyl, or a nitrogen atom protecting group;
R^{5a} is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
n is 1, 2, 3, 4, 5, 6, 7, 8, 9, or 10; and
p is an integer that is 1-1,000.

In some embodiments, the water-solubilizing group of SOL2 is a group of formula:

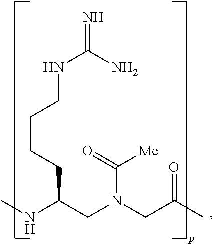

wherein p is an integer that is 1-1,000.

In some embodiments, p is an integer that is 1-100 or an integer that is 1-50. In some embodiments, p is an integer that is 1, 2, 3, 4, 5, 6, 7, 8, 9, 10, 11, 12, 13, 14, 15, 16, 17, 18, 19, or 20. In some embodiments, p is an integer that is 1, 2, 3, 4, 5, 6, 7, 8, 9, 10, 11, or 12. In some embodiments, p is an integer that is 3, 4, 5, 6, 7, 8, 9, or 10. In some embodiments, p is an integer that is 5, 6, 7, 8, or 9. In some embodiments, p is an integer that is 6, 7, or 8. In some embodiments, p is an integer that is 7.

In some embodiments, PNA1 is the peptide nucleic acid sequence. In some embodiments, PNA2 is the peptide nucleic acid sequence.

In some embodiments, L1 is the linker group. In some embodiments, the linker group of L1 is cleavable. In some embodiments, the linker group of L1 is —NHCH(COOH)C(CH₃)₂S—SC(CH₃)₂CH(NH₂)C(O)— or —NHCH(COOH)C(CH₃)₂S—SCH₂CH(NH₂)C(O)—.

In some embodiments, the linker group of L1 is a peptide sequence. In some embodiments, the linker group of L1 is a peptide sequence that is -Glu-Val- Citrulline-, -Lys-, or -(D-Arg)-(D-Arg)-(D-Arg)-.

In some embodiments, the linker group of L1 is a residue of an omega-amino fatty acid. In some embodiments, the linker group of L1 is a residue of an omega-amino caproic acid. In some embodiments, the linker group of L1 is a residue of a dicarboxylic acid. In some embodiments, the linker group of L1 is a residue of oxalic acid or a residue of succinic acid. In some embodiments, the linker group of L1 is a polyamine sequence. In some embodiments, the linker group of L1 is a polyamide sequence. In some embodiments, the linker group of L1 is non-cleavable.

In some embodiments, the linker group of L1 is -Arg-NH(CH₂)₅C(O)—, —NH(CH₂)₅C(O)—, —NH(CH₂)₂C(O)-Arg-NH(CH₂)₅C(O)NH(CH₂)₂C(O)—, —NH(CH₂)₅C(O)-Arg-NH(CH₂)₂C(O)-Arg-NH(CH₂)₅C(O)-Arg-NH(CH₂)₂C(O), -Arg-NH(CH₂)₅C(O)-Arg-Arg-NH(CH₂)₂C(O)-Arg-Arg-NH(CH₂)₅C(O)— (SEQ ID NO: 137), or —NH(CH₂)₅C(O)NH(CH₂)₂-(D-arginine)-(D-arginine)-(D-arginine).

In some embodiments, the linker group of L1 is —NH(CH₂CH₂O)₂CH₂C(O)—, —NH(CH₂CH₂O)₂CH₂C(O)-Arg-, —NH(CH₂CH₂O)₂CH₂C(O)—NH(CH₂CH₂O)₂CH₂C(O)—, —NH(CH₂CH₂O)₂CH₂C(O)—NH(CH₂CH₂O)₂CH₂C(O)-Arg-, —NH(CH₂CH₂O)₂CH₂C(O)—NH(CH₂CH₂O)₂CH₂C(O)-Arg-Arg-, or —NH(CH₂CH₂O)₂CH₂C(O)-Arg-NH(CH₂CH₂O)₂CH₂C(O)-Arg-.

In some embodiments, the linker group of L2 is a peptide sequence. In some embodiments, the linker group of L2 is a peptide sequence that is -Glu-Val- Citrulline-, -Lys-, or -(D-Arg)-(D-Arg)-(D-Arg)-.

In some embodiments, the linker group of L2 is a residue of an omega-amino fatty acid. In some embodiments, the linker group of L2 is a residue of an omega-amino caproic acid. In some embodiments, the linker group of L2 is a residue of a dicarboxylic acid. In some embodiments, the linker group of L2 is a residue of oxalic acid or a residue of succinic acid. In some embodiments, the linker group of L2 is a polyamine sequence. In some embodiments, the linker group of L2 is a polyamide sequence. In some embodiments, the linker group of L2 is non-cleavable.

In some embodiments, the linker group of L2 is -Arg-NH(CH₂)₅C(O)—, —NH(CH₂)₅C(O)—, —NH(CH₂)₂C(O)-Arg-NH(CH₂)₅C(O)NH(CH₂)₂C(O)—, —NH(CH₂)₅C(O)-Arg-NH(CH₂)₂C(O)-Arg-NH(CH₂)₅C(O)-Arg-NH(CH₂)₂C(O), -Arg-NH(CH₂)₅C(O)-Arg-Arg-NH(CH₂)₂C(O)-Arg-Arg-NH(CH₂)₅C(O)— (SEQ ID NO: 137), or —NH(CH₂)₅C(O)NH(CH₂)₂-(D-arginine)-(D-arginine)-(D-arginine).

In some embodiments, the linker group of L2 is —NH(CH₂CH₂O)₂CH₂C(O)—, —NH(CH₂CH₂O)₂CH₂C(O)-Arg-, —NH(CH₂CH₂O)₂CH₂C(O)—NH(CH₂CH₂O)₂CH₂C(O)—, —NH(CH₂CH₂O)₂CH₂C(O)—NH(CH₂CH₂O)₂CH₂C(O)-Arg-, —NH(CH₂CH₂O)₂CH₂C(O)—NH(CH₂CH₂O)₂CH₂C(O)-Arg-Arg-, or —NH(CH₂CH₂O)₂CH₂C(O)-Arg-NH(CH₂CH₂O)₂CH₂C(O)-Arg-.

In some embodiments, the linker group of L3 is a peptide sequence. In some embodiments, the linker group of L1 is a peptide sequence that is -Glu-Val- Citrulline-, -Lys-, or -(D-Arg)-(D-Arg)-(D-Arg)-.

In some embodiments, the linker group of L3 is a residue of an omega-amino fatty acid. In some embodiments, the linker group of L3 is a residue of an omega-amino caproic acid. In some embodiments, the linker group of L3 is a residue of a dicarboxylic acid. In some embodiments, the linker group of L3 is a residue of oxalic acid or a residue of succinic acid. In some embodiments, the linker group of L3 is a polyamine sequence. In some embodiments, the linker group of L3 is a polyamide sequence. In some embodiments, the linker group of L3 is non-cleavable.

In some embodiments, the linker group of L3 is -Arg-NH(CH₂)₅C(O)—, —NH(CH₂)₅C(O)—, —NH(CH₂)₂C(O)-Arg-NH(CH₂)₅C(O)NH(CH₂)₂C(O)—, —NH(CH₂)₅C(O)-Arg-NH(CH₂)₂C(O)-Arg-NH(CH₂)₅C(O)-Arg-NH(CH₂)₂C(O), -Arg-NH(CH₂)₅C(O)-Arg-Arg-NH(CH₂)₂C(O)-Arg-Arg-NH(CH₂)₅C(O)— (SEQ ID NO: 137), or —NH(CH₂)₅C(O)NH(CH₂)₂-(D-arginine)-(D-arginine)-(D-arginine).

In some embodiments, the linker group of L3 is —NH(CH₂CH₂O)₂CH₂C(O)—, —NH(CH₂CH₂O)₂CH₂C(O)-Arg-, —NH(CH₂CH₂O)₂CH₂C(O)—NH(CH₂CH₂O)₂CH₂C(O)—, —NH(CH₂CH₂O)₂CH₂C(O)—NH(CH₂CH₂O)₂CH₂C(O)-Arg-, —NH(CH₂CH₂O)₂CH₂C(O)—NH(CH₂CH₂O)₂CH₂C(O)-Arg-Arg-, or —NH(CH₂CH₂O)₂CH₂C(O)-Arg-NH(CH₂CH₂O)₂CH₂C(O)-Arg-.

In some embodiments, the linker group of L4 is a peptide sequence. In some embodiments, the linker group of L4 is a peptide sequence that is -Glu-Val- Citrulline-, -Lys-, or -(D-Arg)-(D-Arg)-(D-Arg)-.

In some embodiments, the linker group of L4 is a residue of an omega-amino fatty acid. In some embodiments, the linker group of L4 is a residue of an omega-amino caproic acid. In some embodiments, the linker group of L4 is a residue of a dicarboxylic acid. In some embodiments, the linker group of L4 is a residue of oxalic acid or a residue of succinic acid. In some embodiments, the linker group of L4 is a polyamine sequence. In some embodiments, the linker group of L4 is a polyamide sequence. In some embodiments, the linker group of L4 is non-cleavable.

In some embodiments, the linker group of L4 is -Arg-NH(CH₂)₅C(O)—, —NH(CH₂)₅C(O)—, —NH(CH₂)₂C(O)-Arg-NH(CH₂)₅C(O)NH(CH₂)₂C(O)—, —NH(CH₂)₅C(O)-Arg-NH(CH₂)₂C(O)-Arg-NH(CH₂)₅C(O)-Arg-NH(CH₂)₂C(O), -Arg-NH(CH₂)₅C(O)-Arg-Arg-NH(CH₂)₂C(O)-Arg-Arg-NH(CH₂)₅C(O)— (SEQ ID NO: 137), or —NH(CH₂)₅C(O)NH(CH₂)₂-(D-arginine)-(D-arginine)-(D-arginine).

In some embodiments, the linker group of L4 is —NH(CH₂CH₂O)₂CH₂C(O)—, —NH(CH₂CH₂O)₂CH₂C(O)-Arg-, —NH(CH₂CH₂O)₂CH₂C(O)—NH(CH₂CH₂O)₂CH₂C(O)—, —NH(CH₂CH₂O)₂CH₂C(O)—NH(CH₂CH₂O)₂CH₂C(O)-Arg-, —NH(CH₂CH₂O)₂CH₂C(O)—NH(CH₂CH₂O)₂CH₂C(O)-Arg-Arg-, or —NH(CH₂CH₂O)₂CH₂C(O)-Arg-NH(CH₂CH₂O)₂CH₂C(O)-Arg-.

In some embodiments, the linker group of L5 is a peptide sequence. In some embodiments, the linker group of L5 is a peptide sequence that is -Glu-Val- Citrulline-, -Lys-, or -(D-Arg)-(D-Arg)-(D-Arg)-.

In some embodiments, the linker group of L5 is a residue of an omega-amino fatty acid. In some embodiments, the linker group of L5 is a residue of an omega-amino caproic acid. In some embodiments, the linker group of L5 is a residue of a dicarboxylic acid. In some embodiments, the linker group of L5 is a residue of oxalic acid or a residue of succinic acid. In some embodiments, the linker group of L5 is a polyamine sequence. In some embodiments, the linker group of L5 is a polyamide sequence. In some embodiments, the linker group of L5 is non-cleavable.

In some embodiments, the linker group of L5 is -Arg-NH(CH₂)₅C(O)—, —NH(CH₂)₅C(O)—, —NH(CH₂)₂C(O)-Arg-NH(CH₂)₅C(O)NH(CH₂)₂C(O)—, —NH(CH₂)₅C(O)-Arg-NH(CH₂)₂C(O)-Arg-NH(CH₂)₅C(O)-Arg-NH(CH₂)₂C(O), -Arg-NH(CH₂)₅C(O)-Arg-Arg-NH(CH₂)₂C(O)-Arg-Arg-NH(CH₂)₅C(O)— (SEQ ID NO: 137), or —NH(CH₂)₅C(O)NH(CH₂)₂-(D-arginine)-(D-arginine)-(D-arginine).

In some embodiments, the linker group of L5 is —NH(CH₂CH₂O)₂CH₂C(O)—, —NH(CH₂CH₂O)₂CH₂C(O)-Arg-, —NH(CH₂CH₂O)₂CH₂C(O)—NH(CH₂CH₂O)₂CH₂C(O)—, —NH(CH₂CH₂O)₂CH₂C(O)—NH(CH₂CH₂O)₂CH₂C(O)-Arg-, —NH(CH₂CH₂O)₂CH₂C(O)—NH(CH₂CH₂O)₂CH₂C(O)-Arg-Arg-, or —NH(CH₂CH₂O)₂CH₂C(O)-Arg-NH(CH₂CH₂O)₂CH₂C(O)-Arg-.

In some embodiments, the linker group of L6 is a peptide sequence. In some embodiments, the linker group of L6 is a peptide sequence that is -Glu-Val- Citrulline-, -Lys-, or -(D-Arg)-(D-Arg)-(D-Arg)-.

In some embodiments, the linker group of L6 is a residue of an omega-amino fatty acid. In some embodiments, the linker group of L6 is a residue of an omega-amino caproic acid. In some embodiments, the linker group of L6 is a residue of a dicarboxylic acid. In some embodiments, the linker group of L6 is a residue of oxalic acid or a residue of succinic acid. In some embodiments, the linker group of L6 is a polyamine sequence. In some embodiments, the linker group of L6 is a polyamide sequence. In some embodiments, the linker group of L6 is non-cleavable.

In some embodiments, the linker group of L6 is -Arg-NH(CH₂)₅C(O)—, —NH(CH₂)₅C(O)—, —NH(CH₂)₂C(O)-Arg-NH(CH₂)₅C(O)NH(CH₂)₂C(O)—, —NH(CH₂)₅C(O)-Arg-NH(CH₂)₂C(O)-Arg-NH(CH₂)₅C(O)-Arg-NH(CH₂)₂C(O), -Arg-NH(CH₂)₅C(O)-Arg-Arg-NH(CH₂)₂C(O)-Arg-Arg-NH(CH₂)₅C(O)— (SEQ ID NO: 137), or —NH(CH₂)₅C(O)NH(CH₂)₂-(D-arginine)-(D-arginine)-(D-arginine).

In some embodiments, the linker group of L6 is —NH(CH₂CH₂O)₂CH₂C(O)—, —NH(CH₂CH₂O)₂CH₂C(O)-Arg-, —NH(CH₂CH₂O)₂CH₂C(O)—NH(CH₂CH₂O)₂CH₂C(O)—, —NH(CH₂CH₂O)₂CH₂C(O)—NH(CH₂CH₂O)₂CH₂C(O)-Arg-, —NH(CH₂CH₂O)₂CH₂C(O)—NH(CH₂CH₂O)₂CH₂C(O)-Arg-Arg-, or —NH(CH₂CH₂O)₂CH₂C(O)-Arg-NH(CH₂CH₂O)₂CH₂C(O)-Arg-.

In some embodiments, the structure is:

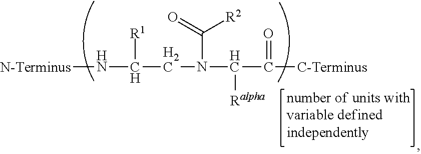

wherein:
the number of units with variables defined independently is at least 11;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle; and
C-Terminus is OH, OMe, NH₂, or a peptide sequence,
   or a pharmaceutically-acceptable salt or ionized form thereof.

In some embodiments, the structure is:

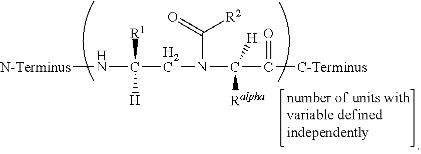

In some embodiments, the structure binds to a mRNA sequence transcribed from a gene associated with a DM1 disease phenotype. In some embodiments, the structure binds to a mRNA sequence transcribed from a gene associated with a DM1 disease phenotype by interactions between the heterocycles of the R² groups and nucleobases of the DM1 gene. In some embodiments, the DM1 gene is a non-wild type DM1 gene. In some embodiments, the non-wild type DM1 gene differs from a wild type DM1 gene in a repeat expansion mutation.

In some embodiments, the structure binds to a mRNA sequence that contains a subsequence that is (CUG)_{z}, wherein z is an integer from 1-100, an integer from 1-50, an integer from 1-40, an integer from 1-30, an integer from 1-25, an integer from 1-20, an integer from 1-15, or an integer from 1-10 (SEQ ID NO: 135). In some embodiments, the structure binds to the mRNA sequence at the subsequence that is (CUG)_{z}.

In some embodiments, the number of units with variables defined independently is 11-1,000, 11-100, or 11-50. In some embodiments, the number of units with variables defined independently is 11, 12, 13, 14, 15, 16, 17, 18, 19, 20, 21, 22, 23, 24, 25, 26, 27, 28, 29, or 30.

In some embodiments, each R¹ is independently alkyl that is unsubstituted. In some embodiments, each alkyl that is unsubstituted is independently methyl, ethyl, prop-1-yl, prop-2-yl, 2-methylprop-1-yl, but-1yl, but-2-yl, or pent-1-yl. In some embodiments, each alkyl that is unsubstituted is independently methyl, prop-2-yl, 2-methylprop-1-yl, or but-2-yl. In some embodiments, each R¹ is independently alkyl that is substituted. In some embodiments, each alkyl that is substituted is independently substituted with —OH, —SH, —SMe, —NH₂, a heterocycle, an aryl group, a carboxylic acid, a guanidino group, a N-methylguanidino group, or an amido group. In some embodiments, each alkyl that is substituted is independently hydroxymethyl, 1-hydroxyeth-1-yl, sulfhydrylmethyl, 2-thiomethyleth-1-yl, 4-aminobut-1-yl, 3-aminoprop-1-yl, 1-H-imidazol-4-ylmethyl, 1-H-indol-3-ylmethyl, benzyl, 4-hydroxyphen-1-ylmethyl, 2-carboxylatoeth-1-yl, 3-carboxylatoprop-1-yl, 3-guanidinoprop-1-yl, 4-guanidinobut-1-yl, 2-carbamoyleth-1-yl, or 3-carbamoylprop-1-yl.

In some embodiments, each R¹ is independently H, hydroxylmethyl, or 4-guanidinobut-1-yl. In some embodiments, at least one iteration of R¹ is hydroxylmethyl. In some embodiments, at least a third of the iterations of R¹ are hydroxylmethyl. In some embodiments, at least half the iterations of R¹ are hydroxylmethyl.

In some embodiments, each R^{alpha} is independently alkyl that is unsubstituted. In some embodiments, each alkyl that is unsubstituted is independently methyl, ethyl, prop-1-yl, prop-2-yl, 2-methylprop-1-yl, but-1yl, but-2-yl, or pent-1-yl. In some embodiments, each alkyl that is unsubstituted is independently methyl, prop-2-yl, 2-methylprop-1-yl, or but-2-yl.

In some embodiments, each R^{alpha} is independently alkyl that is substituted. In some embodiments, each alkyl that is substituted is independently substituted with —OH, —SH, —SMe, —NH₂, a heterocycle, an aryl group, a carboxylic acid, a guanidino group, a N-methylguanidino group, or an amido group. In some embodiments, each alkyl that is substituted is independently hydroxymethyl, 1-hydroxyeth-1-yl, sulfhydrylmethyl, 2-thiomethyleth-1-yl, 4-aminobut-1-yl, 3-aminoprop-1-yl, 1-H-imidazol-4-ylmethyl, 1-H-indol-3-ylmethyl, benzyl, 4-hydroxyphen-1-ylmethyl, 2-carboxylatoeth-1-yl, 3-carboxylatoprop-1-yl, 3-guanidinoprop-1-yl, 4-guanidinobut-1-yl, 2-carbamoyleth-1-yl, or 3-carbamoylprop-1-yl.

In some embodiments, each R^{alpha} is independently H, 3-guanidinoprop-1-yl, or 4-guanidinobut-1-yl. In some embodiments, at least one iteration of R^{alpha} is 3-guanidinoprop-1-yl. In some embodiments, at least a third of the iterations of R^{alpha} are 3-guanidinoprop-1-yl. In some embodiments, at least half the iterations of R^{alpha} are 3-guanidinoprop-1-yl.

In some embodiments, of the units with variables defined independently, counting from N-Terminus, the first, third, sixth, ninth, eleventh, thirteenth, sixteenth, nineteenth, and twenty-second units, independently if present, each have 3-guanidinoprop-1-yl at R^{alpha}.

In some embodiments, at least a third of the R² groups in the structure are methyl substituted with a heterocycle. In some embodiments, at least half of the R² groups in the structure are methyl substituted with a heterocycle. In some embodiments, the heterocycles of the R² groups are nucleobases or analogues of nucleobases. In some embodiments, at least one of the heterocycles of the R² groups is a divalent nucleobase. In some embodiments, the heterocycles of the R² groups are divalent nucleobases.

In some embodiments, the heterocycles of the R² groups are each independently:

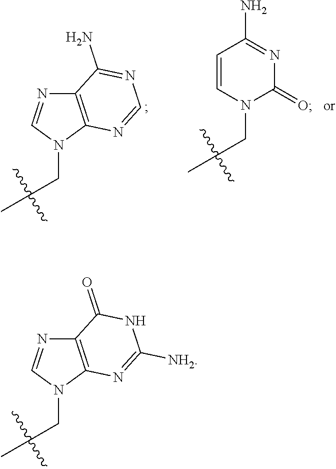

In some embodiments, each R² is independently: methyl,

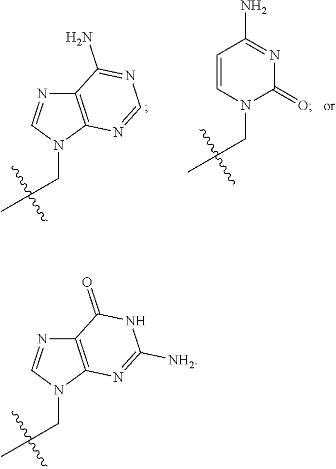

In some embodiments, N-Terminus is H. In some embodiments, N-Terminus is acyl. In some embodiments, N-Terminus is the biological agent. In some embodiments, the biological agent is a vitamin E group. In some embodiments, the biological agent is an O-bound tocopherol group. In some embodiments, C-Terminus is NH₂. In some embodiments, C-Terminus is a peptide sequence according to SEQ ID NO: 1, wherein the C-terminal residue of the peptide sequence is amidated. In some embodiments, the neuromuscular disease is DM1.

In some embodiments, the disclosure provides a compound comprising a structure that is:

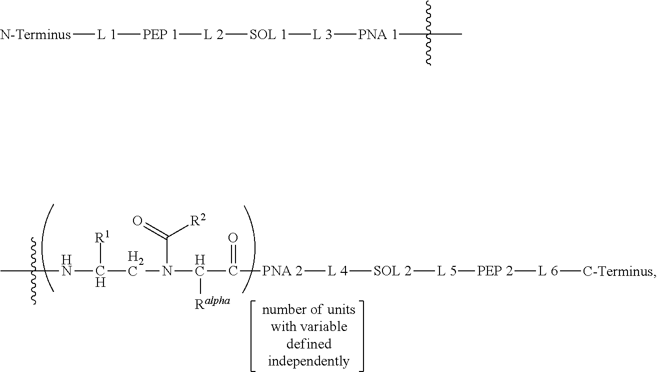

wherein:
the number of units with variables defined independently is at least 11;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H, wherein at least one iteration of R¹ is a hydroxyalkyl group;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle;
C-Terminus is OH, OMe, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof.

In some embodiments, the structure is:

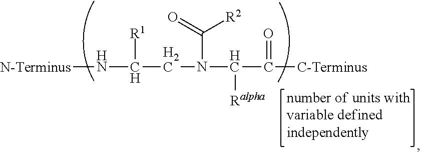

wherein:
the number of units with variables defined independently is at least 11; N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H, wherein at least one iteration of R¹ is a hydroxyalkyl group;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle; and
C-Terminus is OH, OMe, NH₂, or a peptide sequence,
   or a pharmaceutically-acceptable salt or ionized form thereof.

In some embodiments, the disclosure provides a compound comprising a structure that is:

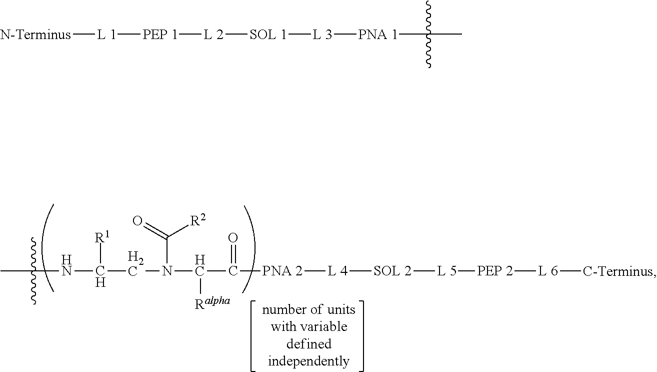

wherein:
the number of units with variables defined independently is at least 3;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H, wherein at least one iteration of R¹ is a hydroxyalkyl group;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle;
C-Terminus is OH, OMe, or NH₂;
PEP1 is a peptide sequence or absent;
PEP2 is a peptide sequence or absent;
   wherein at least one of PEP1 and PEP2 is a peptide sequence of at least three amino acid residues,
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof.

In some embodiments, the disclosure provides a compound comprising a structure that is:

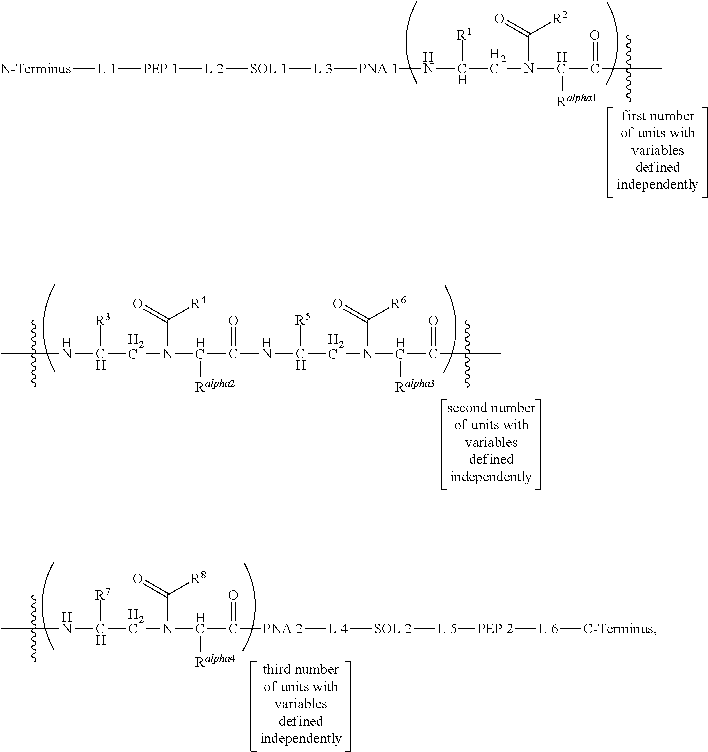

wherein:
the first number of units with variables defined independently is at least zero;
the second number of units with variables defined independently is at least 3;
the third number of units with variables defined independently is at least zero;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R¹ is independently alkyl, O-alkyl, or methyl substituted with a heterocycle;
each R³ is independently a hydroxyalkyl group;
each R⁴ is independently R²;
each R⁵ is independently a group that is not hydroxyalkyl;
each R⁶ is independently R²;
each R⁷ is independently alkyl that is unsubstituted or substituted or H;
each R⁸ is independently R²,
wherein at least two R² groups in the structure are independently methyl substituted with a heterocycle;
each R^{alpha1} is independently alkyl that is unsubstituted or substituted or H;
each R^{alpha2} is independently alkyl that is unsubstituted or substituted or H;
each R^{alpha3} is independently alkyl that is unsubstituted or substituted or H;
each R^{alpha4} is independently alkyl that is unsubstituted or substituted or H;
C-Terminus is OH, OMe, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof.

In some embodiments, the first number of units with variables defined independently is 3-1,000, 3-100, or 3-50. In some embodiments, the first number of units with variables defined independently is 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, the first number of units with variables defined independently is at least 11. In some embodiments, the first number of units with variables defined independently is 11-1,000, 11-100, or 11-50. In some embodiments, the first number of units with variables defined independently is 11, 12, 13, 14, 15, 16, 17, 18, 19, 20, 21, 22, 23, 24, 25, 26, 27, 28, 29, or 30.

In some embodiments, the second number of units with variables defined independently is 3-1,000, 3-100, or 3-50. In some embodiments, the second number of units with variables defined independently is 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, the second number of units with variables defined independently is 3, 4, 5, 6, 7, 8, 9, 10, 11, 12, 13, 14, 15, 16, 17, 18, 19, or 20. In some embodiments, the second number of units with variables defined independently is 3, 4, 5, 6, 7, 8, 9, or 10.

In some embodiments, the third number of units with variables defined independently is 3-1,000, 3-100, or 3-50. In some embodiments, the third number of units with variables defined independently is 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, the third number of units with variables defined independently is at least 11. In some embodiments, the third number of units with variables defined independently is 11-1,000, 11-100, or 11-50. In some embodiments, the third number of units with variables defined independently is 11, 12, 13, 14, 15, 16, 17, 18, 19, 20, 21, 22, 23, 24, 25, 26, 27, 28, 29, or 30.

In some embodiments, each R³ is hydroxymethyl. In some embodiments, each R⁵ is H.

In some embodiments, the disclosure provides a compound comprising a repeating unit of formula:

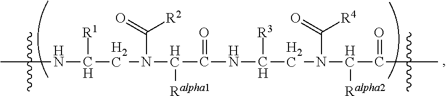

wherein:
each R¹ is independently a hydroxyalkyl group;
each R² is independently alkyl, O-alkyl, or methyl substituted with a heterocycle;
each R³ is independently a group that is not hydroxyalkyl;
each R⁴ is independently alkyl, O-alkyl, or methyl substituted with a heterocycle;
each R^{alpha1} is independently alkyl that is unsubstituted or substituted or H; and
each R^{alpha2} is independently alkyl that is unsubstituted or substituted or H,
   wherein the repeating unit occurs at least twice consecutively in the compound.

In some embodiments, the disclosure provides a compound comprising:
1) a region that comprises a structure that interferes with expression of a gene associated with a neuromuscular disease phenotype; and
2) connected to the region that comprises the structure that interferes with expression of the gene associated with a neuromuscular disease phenotype, an oligomeric sequence, wherein the oligomeric sequence comprises a repeating unit of formula:

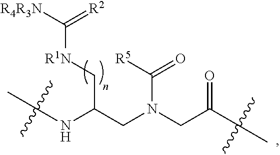

or an ionized form thereof, wherein:
   R¹ is H, alkyl, or a nitrogen atom protecting group;
   R² is O, NH, N(alkyl), or N(Pg^{N}), wherein Pg^{N} is a nitrogen atom protecting group;
   R³ is H, alkyl, or a nitrogen atom protecting group;
   R⁴ is H, alkyl, or a nitrogen atom protecting group;
   R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; and
   n is 1, 2, 3, 4, 5, 6, 7, 8, 9, or 10,
      or a pharmaceutically-acceptable salt or ionized form thereof.

In some embodiments, the disclosure provides a compound comprising:
1) a region that comprises a structure that interferes with expression of a gene associated with a neuromuscular disease phenotype; and
2) connected to the region that comprises the structure that interferes with expression of the gene associated with a neuromuscular disease phenotype, an oligomeric sequence, wherein the oligomeric sequence comprises a repeating unit of formula:

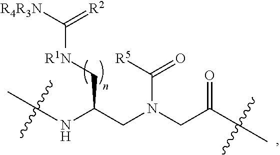

or an ionized form thereof, wherein:
   R¹ is H, alkyl, or a nitrogen atom protecting group;
   R² is O, NH, N(alkyl), or N(Pg^{N}), wherein Pg^{N} is a nitrogen atom protecting group;
   R³ is H, alkyl, or a nitrogen atom protecting group;
   R⁴ is H, alkyl, or a nitrogen atom protecting group;
   R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; and
   n is 1, 2, 3, 4, 5, 6, 7, 8, 9, or 10,
      or a pharmaceutically-acceptable salt or ionized form thereof.

In some embodiments, the neuromuscular disease phenotype is a DM1 disease phenotype. In some embodiments, the structure binds to a mRNA sequence transcribed from a gene associated with a DM1 disease phenotype. In some embodiments, the structure binds to a mRNA sequence transcribed from a gene associated with a DM1 disease phenotype by interactions between the heterocycles of the R² groups and nucleobases of the DM1 gene. In some embodiments, the DM1 gene is a non-wild type DM1 gene. In some embodiments, the non-wild type DM1 gene differs from a wild type DM1 gene in a repeat expansion mutation.

In some embodiments, the structure binds to a mRNA sequence that contains a subsequence that is (CUG)_{z}, wherein z is an integer from 1-100, an integer from 1-50, an integer from 1-40, an integer from 1-30, an integer from 1-25, an integer from 1-20, an integer from 1-15, or an integer from 1-10 (SEQ ID NO: 135). In some embodiments, the structure binds to the mRNA sequence at the subsequence that is (CUG)_{z}.

In some embodiments, the compound is:

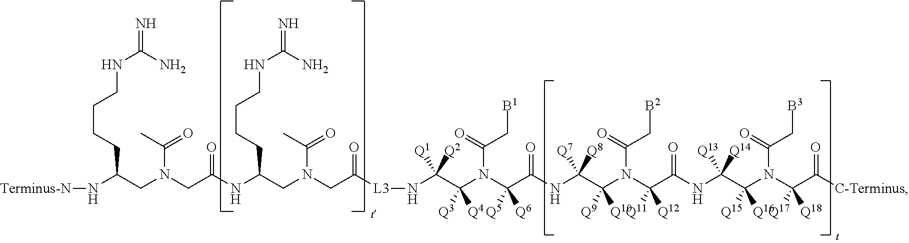

wherein:
each instance of B¹, B², and B³ is independently a heterocycle;
each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl, heteroalkyl, aryl, or heteroaryl, each of which is independently substituted or unsubstituted, or hydrogen;
L3 is a linker group or absent;
N-Terminus is H, acyl, a group that together with the nitrogen atom to which the N-Terminus is bound forms a carbamate, a probe, a fluorophore, a lipid, a metal chelator, or a biological agent;
C-Terminus is -T or —N(H)-J, wherein T is OH, O-alkyl, a peptide sequence, a fluorophore, a lipid, a biological agent, or NH₂, and J is H, acyl, a group that together with the nitrogen atom to which J is bound forms a carbamate, a probe, a fluorophore, a lipid, a metal chelator, or a biological agent;
t is an integer that is from 1 to 30; and
t′ is an integer that is from 2 to 9,
   or a pharmaceutically acceptable salt or ionized form thereof.

In some embodiments, wherein L3 is absent. In some embodiments, N-Terminus is H. In some embodiments, C-Terminus is -T. In some embodiments, C-Terminus is —N(H)-J. In some embodiments, C-Terminus is NH₂. In some embodiments, each instance of Q¹, Q², Q³, Q⁴, Q⁵, Q⁶, Q⁷, Q⁸, Q⁹, Q¹⁰, Q n, Q¹², Q¹³, Q¹⁴, Q¹⁵, Q¹⁶, Q¹⁷ and Q¹⁸ is independently an amino acid side chain, alkyl that is substituted or unsubstituted, or hydrogen.

In some embodiments, the compound is:

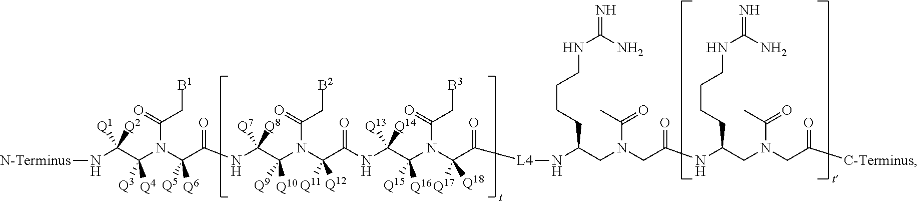

wherein:
each instance of B¹, B², and B³ is independently a heterocycle;
each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl, heteroalkyl, aryl, or heteroaryl, each of which is independently substituted or unsubstituted, or hydrogen;
L4 is a linker group or absent;
N-Terminus is H, acyl, a group that together with the nitrogen atom to which the N-Terminus is bound forms a carbamate, a probe, a fluorophore, a lipid, a metal chelator, or a biological agent;
C-Terminus is -T or —N(H)-J, wherein T is OH, O-alkyl, a peptide sequence, a fluorophore, a lipid, a biological agent, or NH₂, and J is H, acyl, a group that together with the nitrogen atom to which J is bound forms a carbamate, a probe, a fluorophore, a lipid, a metal chelator, or a biological agent;
t is an integer that is from 1 to 30; and
t′ is an integer that is from 2 to 9,
   or a pharmaceutically acceptable salt or ionized form thereof.

In some embodiments, wherein L4 is absent. In some embodiments, C-Terminus is -T. In some embodiments, C-Terminus is —N(H)-J. In some embodiments, C-Terminus is NH₂. In some embodiments, N-Terminus is H. In some embodiments, each instance of Q¹, Q², Q³, Q⁴, Q⁵, Q⁶, Q⁷, Q⁸, Q⁹, Q¹⁰, Q¹¹, Q¹², Q¹³, Q′4, Q¹⁵, Q¹⁶, Q′ 7, and Q¹⁸ is independently an amino acid side chain, alkyl that is substituted or unsubstituted, or hydrogen.

In some embodiments, the disclosure provides a compound comprising:
1) a region that comprises a structure that interferes with expression of a gene associated with a DM1 disease phenotype; and
2) connected to the region that comprises the structure that interferes with expression of a gene associated with a neuromuscular disease phenotype, a compound comprising a repeating unit, wherein the repeating unit comprises:
   a) a guanidino group;
   b) a nitrogen atom that is bound to a —C(O)Me group and a —CH₂C(O)— group; and
   c) a chain of carbon atoms,
      wherein the guanidino group is attached to the chain of carbon atoms at a first point of attachment;
      wherein the nitrogen atom is attached to the chain of carbon atoms at a second point of attachment; and
      wherein the first point of attachment and the second point of attachment are separated by 3-12 carbon atoms,
         or a pharmaceutically-acceptable salt or ionized form thereof.

In some embodiments, the neuromuscular disease phenotype is a DM1 disease phenotype.

In some embodiments, the disclosure provides a compound comprising:
1) a region that comprises a structure that interferes with expression of a gene associated with a neuromuscular disease phenotype; and
2) connected to the region that comprises the structure that interferes with expression of a gene associated with a neuromuscular disease phenotype, a compound of formula:

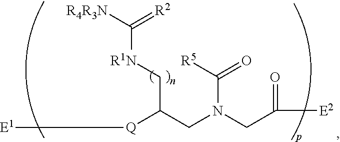

wherein:
   R¹ is H, alkyl, or a nitrogen atom protecting group;
   R² is O, NH, N(alkyl), or N(Pg^{N}), wherein Pg^{N} is a nitrogen atom protecting group;
   R³ is H, alkyl, or a nitrogen atom protecting group;
   R⁴ is H, alkyl, or a nitrogen atom protecting group;
   R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
   Q is O, NH, N(alkyl), or N(Pg^{N});
   E¹ is a chemical moiety;
   E² is a chemical moiety;
   n is 1, 2, 3, 4, 5, 6, 7, 8, 9, or 10; and
   p is an integer that is 1-1,000,
      or a pharmaceutically-acceptable salt or ionized form thereof.

In some embodiments, the disclosure provides a compound comprising:
1) a region that comprises a structure that interferes with expression of a gene associated with a neuromuscular disease phenotype; and
2) connected to the region that comprises the structure that interferes with expression of a gene associated with a neuromuscular disease phenotype, a compound of formula:

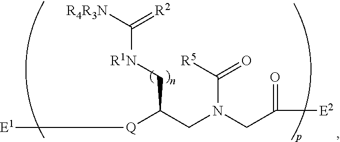

wherein:
   R¹ is H, alkyl, or a nitrogen atom protecting group;
   R² is O, NH, N(alkyl), or N(Pg^{N}), wherein Pg^{N} is a nitrogen atom protecting group;
   R³ is H, alkyl, or a nitrogen atom protecting group;
   R⁴ is H, alkyl, or a nitrogen atom protecting group;
   R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
   Q is O, NH, N(alkyl), or N(Pg^{N});
   E¹ is a chemical moiety;
   E² is a chemical moiety;
   n is 1, 2, 3, 4, 5, 6, 7, 8, 9, or 10; and
   p is an integer that is 1-1,000,
      or a pharmaceutically-acceptable salt or ionized form thereof.

In some embodiments, the neuromuscular disease phenotype is a DM1 disease phenotype.

In some embodiments, the number of units with variables defined independently is 18, wherein:
a first unit is present, and in the first unit:
R¹ is H or —CH₂OH; and R² is

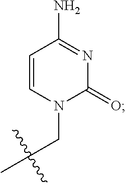 [SMILES: *Cn1ccc(N)nc1=O]

a second unit is present, and in the second unit:
R¹ is H or —CH₂OH; and R² is

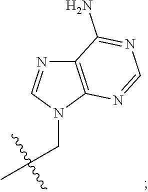 [SMILES: *Cn1cnc2c(N)ncnc21]

a third unit is present, and in the third unit:
R¹ is H or —CH₂OH; and R² is

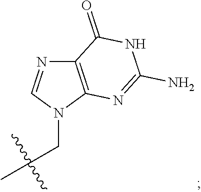 [SMILES: *Cn1cnc2c(=O)[nH]c(N)nc21]

a fourth unit is present, and in the fourth unit:
R¹ is H or —CH₂OH; and R² is

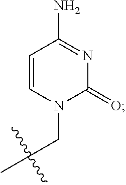 [SMILES: *Cn1ccc(N)nc1=O]

a fifth unit is present, and in the fifth unit:

R¹ is H or —CH₂OH; and R² is

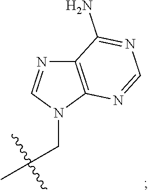 [SMILES: *Cn1cnc2c(N)ncnc21]

a sixth unit is present, and in the sixth unit:

R¹ is H or —CH₂OH; and R² is

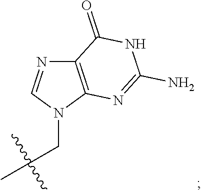 [SMILES: *Cn1cnc2c(=O)[nH]c(N)nc21]

a seventh unit is present, and in the seventh unit:

R¹ is H or —CH₂OH; and R² is

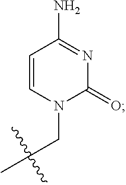 [SMILES: *Cn1ccc(N)nc1=O]

an eighth unit is present, and in the eighth unit:

R¹ is H or —CH₂OH; and R² is

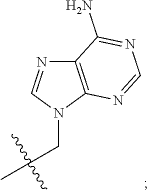 [SMILES: *Cn1cnc2c(N)ncnc21]

a ninth unit is present, and in the ninth unit:

R¹ is H or —CH₂OH; and R² is

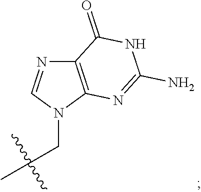 [SMILES: *Cn1cnc2c(=O)[nH]c(N)nc21]

a tenth unit is present, and in the tenth unit:

R¹ is H or —CH₂OH; and R² is

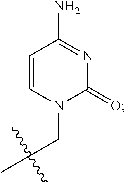 [SMILES: *Cn1ccc(N)nc1=O]

an eleventh unit is present, and in the eleventh unit:

R¹ is H or —CH₂OH; and R² is

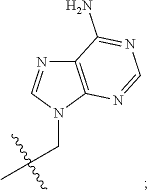 [SMILES: *Cn1cnc2c(N)ncnc21]

a twelfth unit is present, and in the twelfth unit:

R¹ is H or —CH₂OH; and R² is

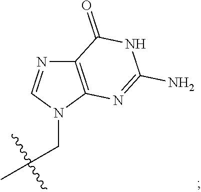 [SMILES: *Cn1cnc2c(=O)[nH]c(N)nc21]

a thirteenth unit is present, and in the thirteenth unit:

R¹ is H or —CH₂OH; and R² is

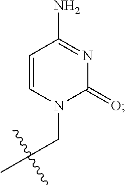 [SMILES: *Cn1ccc(N)nc1=O]

a fourteenth unit is present, and in the fourteenth unit:

R¹ is H or —CH₂OH; and R² is

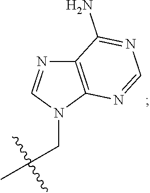 [SMILES: *Cn1cnc2c(N)ncnc21]

a fifteenth unit is present or absent, and in the fifteenth unit:

R¹ is H or —CH₂OH; and R² is

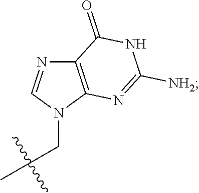 [SMILES: *Cn1cnc2c(=O)[nH]c(N)nc21]

a sixteenth unit is present or absent, and in the sixteenth unit:

R¹ is H or —CH₂OH; and R² is

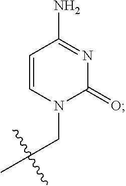 [SMILES: *Cn1ccc(N)nc1=O]

a seventeenth unit is present or absent, and in the seventeenth unit:
R¹ is H or —CH₂OH; and R² is

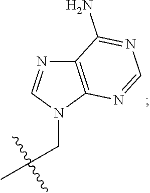 [SMILES: *Cn1cnc2c(N)ncnc21]

and
an eighteenth unit is present or absent, and in the eighteenth unit:
R¹ is H or —CH₂OH; and R² is

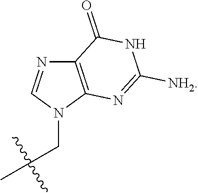 [SMILES: *Cn1cnc2c(=O)[nH]c(N)nc21]

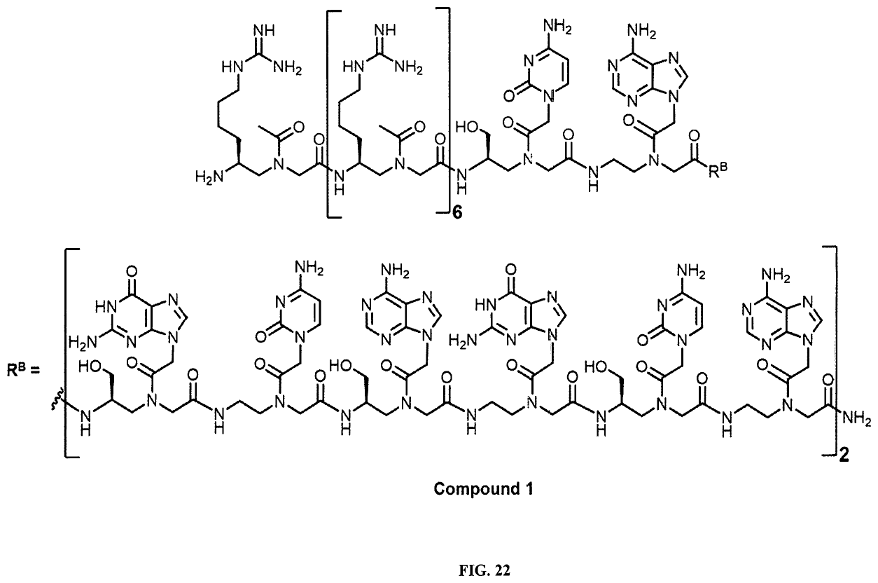

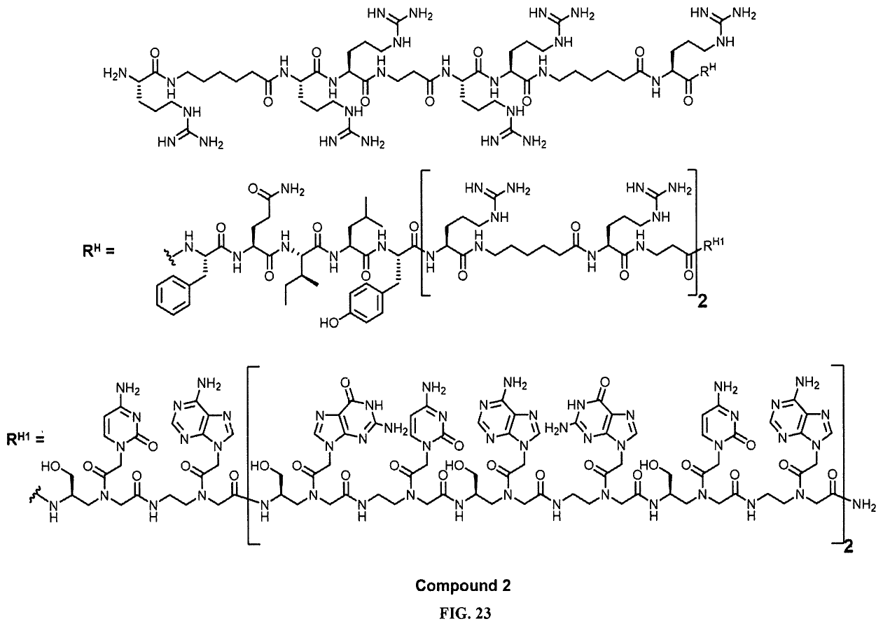

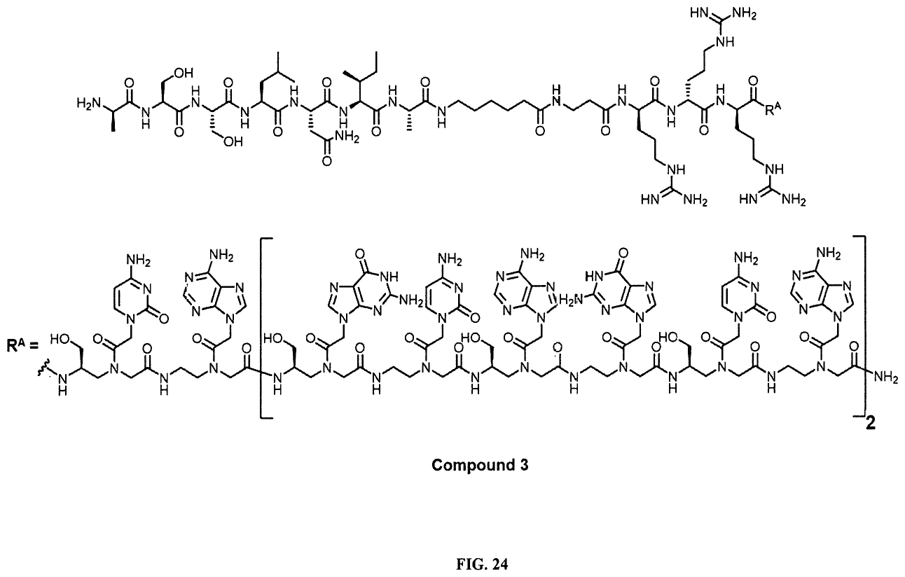

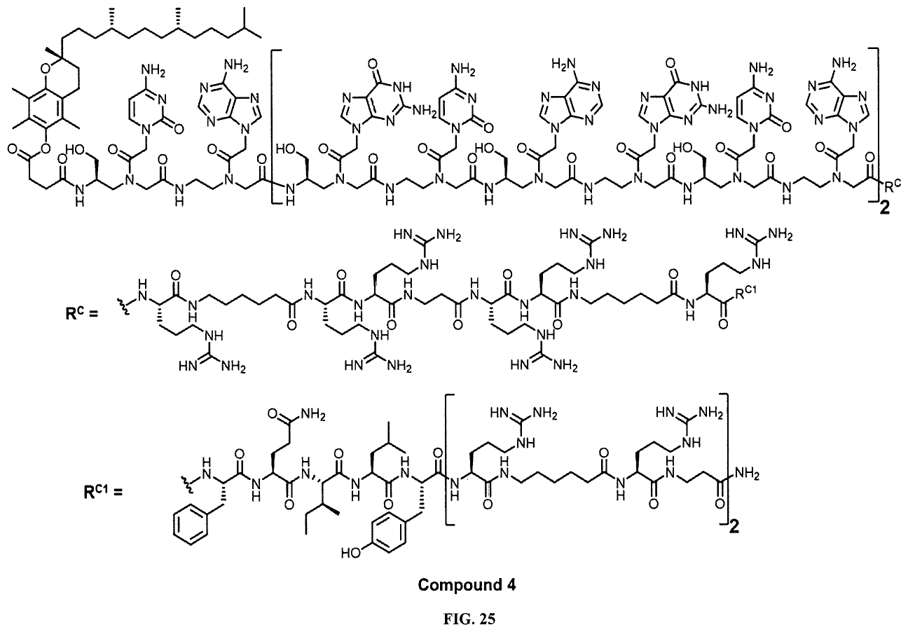

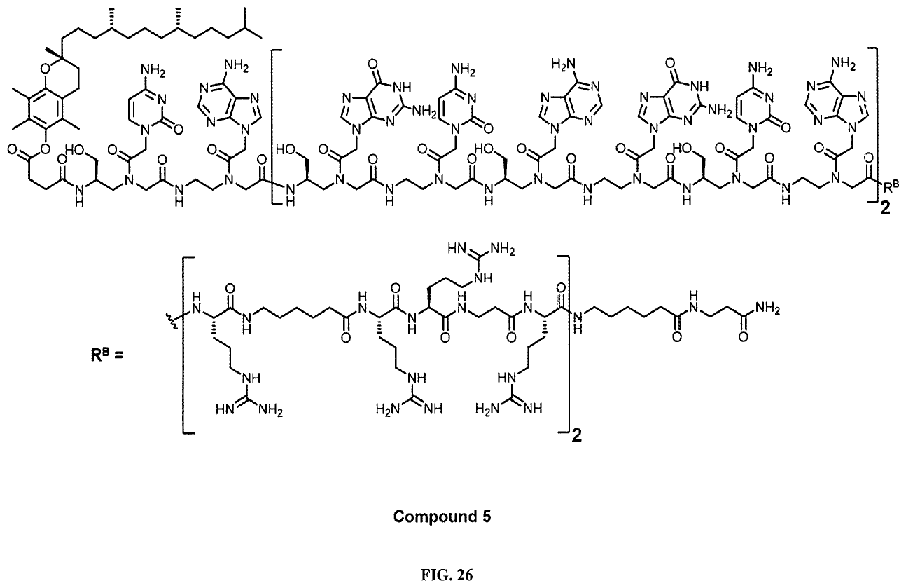

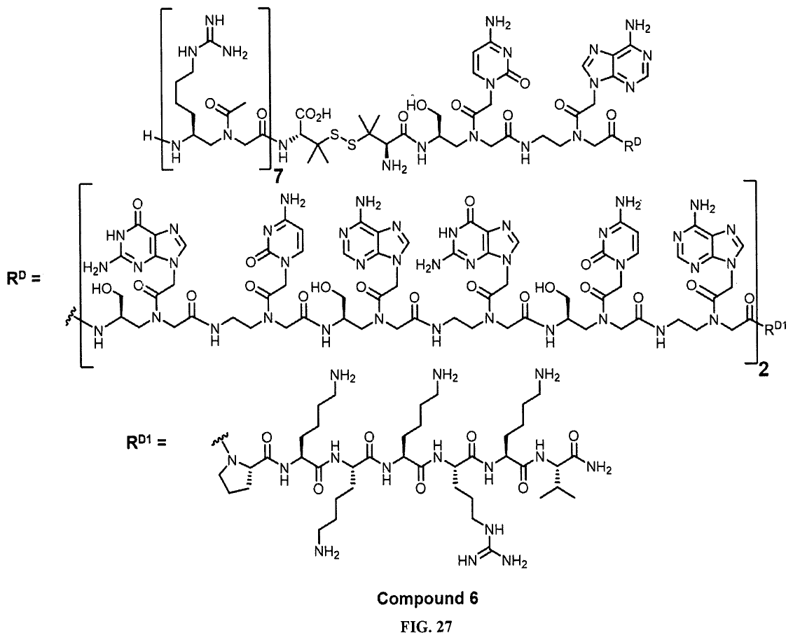

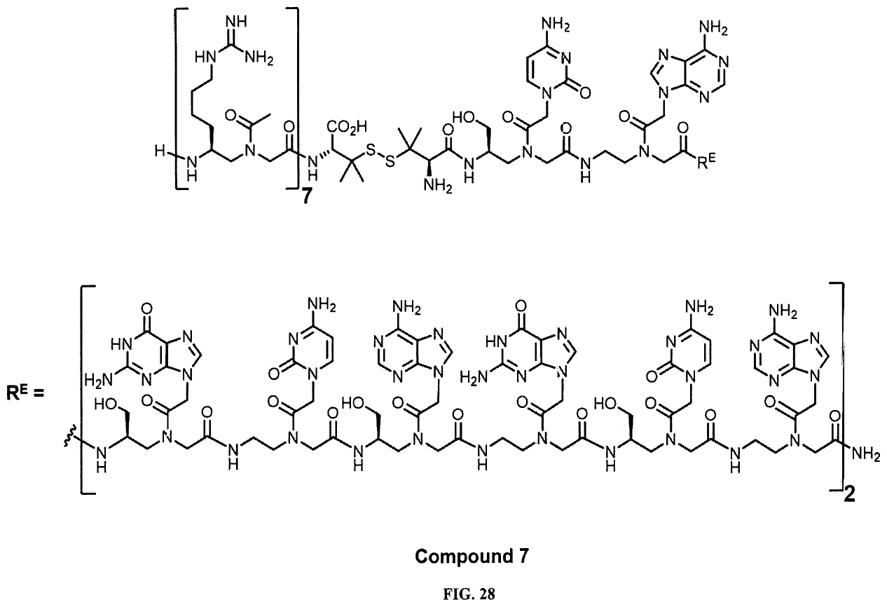

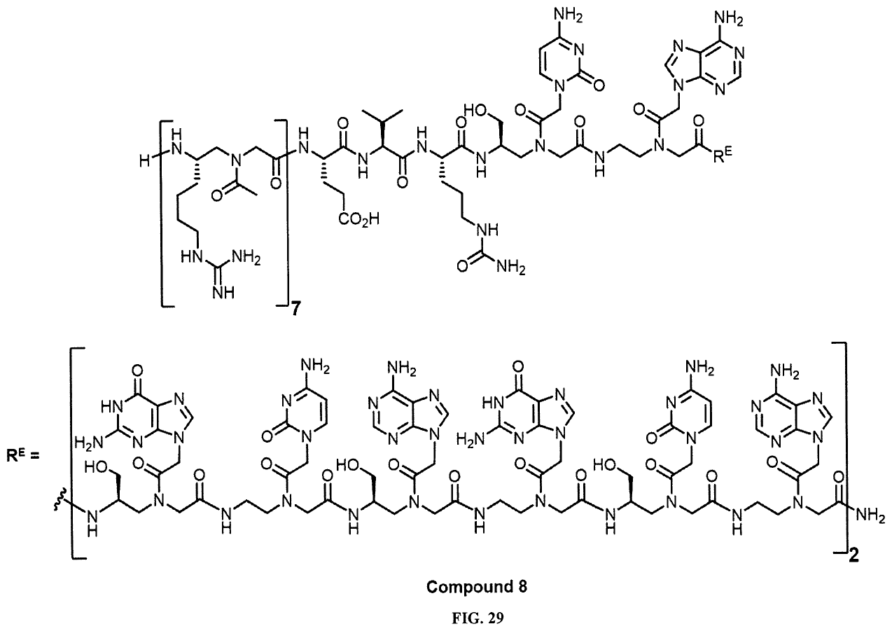

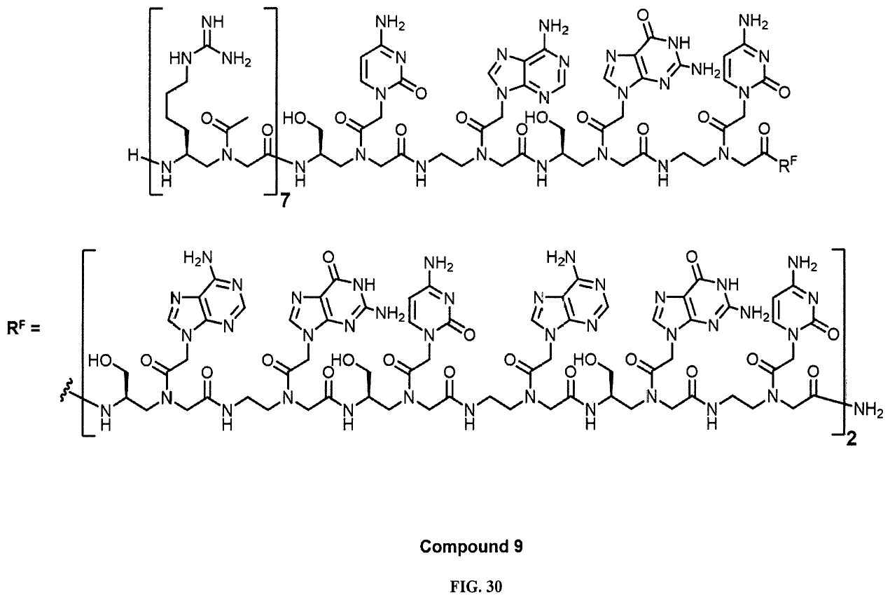

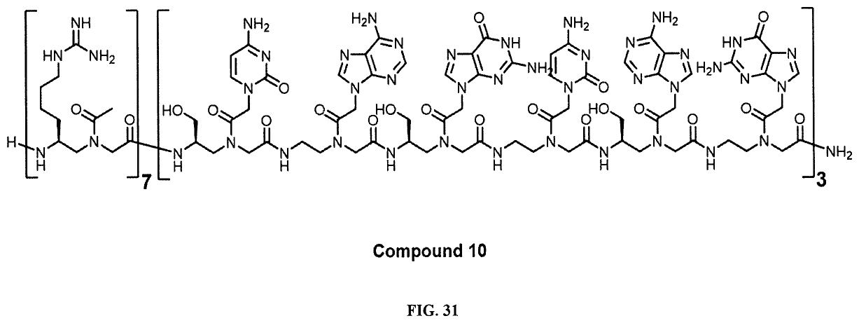

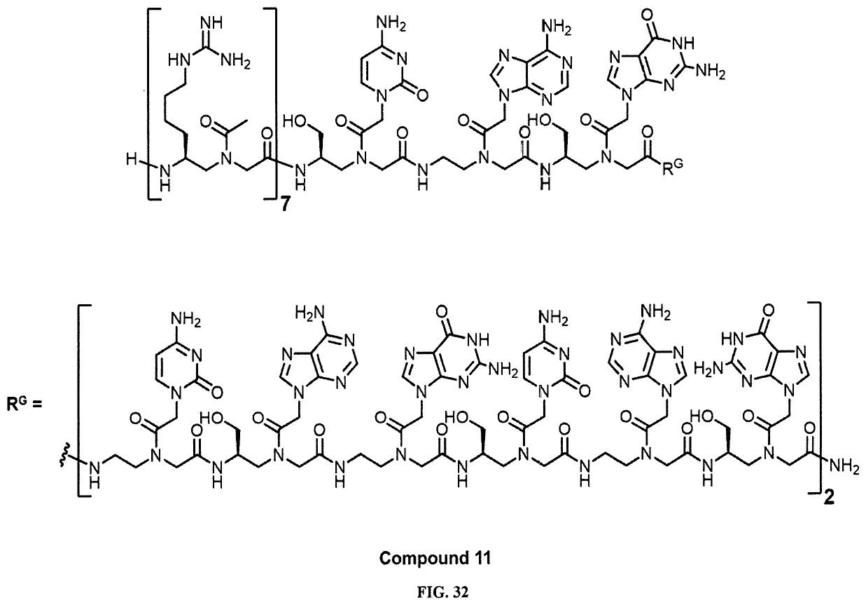

Two-dimensional molecular structures of selected compounds of the disclosure are provided in FIGS. 22-32. FIG. 22 illustrates the structure of Compound 1. FIG. 23 illustrates the structure of Compound 2. FIG. 24 illustrates the structure of Compound 3. FIG. 25 illustrates the structure of Compound 4. FIG. 26 illustrates the structure of Compound 5. FIG. 27 illustrates the structure of Compound 6. FIG. 28 illustrates the structure of Compound 7. FIG. 29 illustrates the structure of Compound 8. FIG. 30 illustrates the structure of Compound 9. FIG. 31 illustrates the structure of Compound 10. FIG. 32 illustrates the structure of Compound 11.

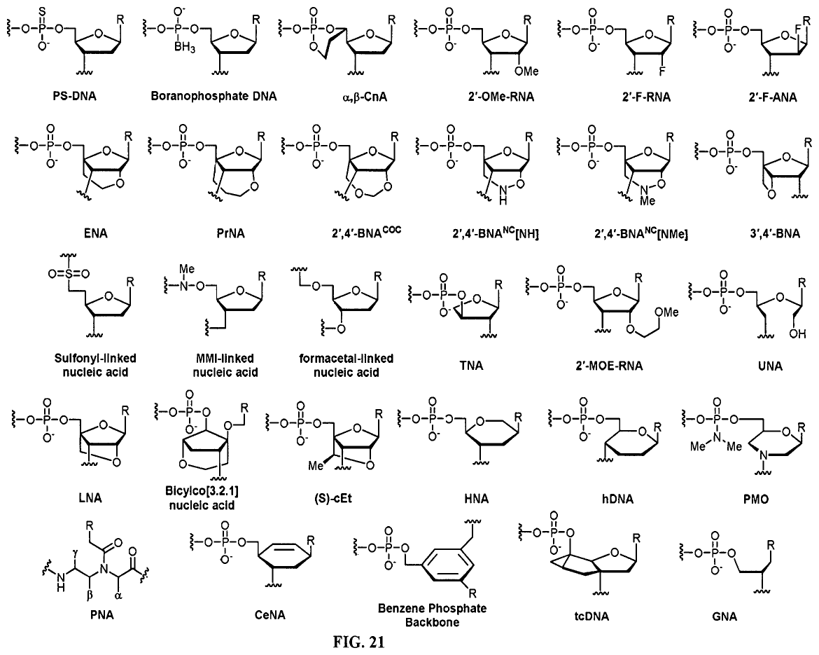

FIG. 21 depicts nonlimiting examples of oligonucleotide backbones, where R is a nucleobase (e.g. natural, modified, or non-natural nucleobases), or hydrogen. One or more oligonucleotide residues of a compound of the disclosure may be independently replaced with a residue comprising an alternative oligonucleotide backbone bearing an identical nucleobase. Nonlimiting examples of oligonucleotide backbones suitable for use in the present disclosure include phosphorothioate deoxyribonucleic acid (PS-DNA), boranophosphate DNA, alpha-,beta-constrained nucleic acid (α,β-CnA), 2′-methoxyribonucleic acid (2′-OMe-RNA), 2′-fluororibonucleic acid (2′-F-RNA), 2′-fluoroarabinonucleic acid (2′-F-ANA), sulfonyl-linked nucleic acid, methylene(methylimino) (MMI) linked, formacetal-linked nucleic acid, threose nucleic acid (TNA), 2′-methoxyribonucleic acid (2′-OMe-RNA), 2′-O-(2-methoxyethyl)ribonucleic acid (2′-MOE-RNA), unlocked nucleic acid (UNA), 2′-O,4′-C-ethylene-bridged nucleic acid (ENA), 2′-O,4′-C-propylene bridged nucleic acid (PrNA), bridged nucleic acids (e.g., 2′,4′-BNA^{COC}, 2′,4′-BNA^{NC}[NH], 2′,4′-BNA^{NC}[NMe], 3′,4′-BNA), locked nucleic acid (LNA), bicylco[3.2.1]nucleic acid, (S)-constrained ethyl nucleic acid ((S)-cEt), hexitol nucleic acid (HNA), homo-deoxyribonucleic acid (hNDA), phosphorodiamidate morpholino oligomer (PMO), peptide nucleic acid (PNA), cyclohexene nucleic acid (CeNA), benzene phosphate backbone, tricyclo-DNA(tcDNA), glycol nucleic acid (GNA), and epimers and diastereomers thereof.

A compound of the disclosure (e.g. a peptide nucleic acid) can be conjugated to one or more polypeptides, such as a cell penetrating peptide or other polypeptide that can facilitate uptake or cellular intake. Nonlimiting examples of cell-penetrating peptides include SV40 NLS (SEQ ID NO 1: PKKKRKV), c-Myc NLS (SEQ ID NO 2: PAAKRVKLD), nuleoplasmin (SEQ ID NO. 3: KRPAATKKAGQAKKKL), NF-Kb NLS (SEQ ID NO 4: VQRKRQKLMP), TFIIE beta NLS (SEQ ID NO 5: SKKKKTKV), Oct-6 NLS (SEQ ID NO 6: GRKRKKRT), HATF-3 NLS (SEQ ID NO 7: ERKKRRRE), SDC3 NLS (SEQ ID NO 8: FKKFRKF), DPV3 (SEQ ID NO 9: RKKRRRESRKKRRRES), DPV6 (SEQ ID NO 10: GRPRESGKKRKRKRLKP), DPV7 (SEQ ID NO 11: GKRKKKGKLGKKRDP), DPV7b (SEQ ID NO 12: GKRKKKGKLGKKRPRSR), DPV3/10 (SEQ ID NO 13: RKKRRRESRRARRSPRHL), DPV10/6 (SEQ ID NO 14: SRRARRSPRESGKKRKRKR), DPV1047 (SEQ ID NO 15: VKRGLKLRHVRPRVTRMDV), DPV 10 (SEQ ID NO 16: SRRARRSPRHLGSG), DPV15 (SEQ ID NO 17: LRRERQSRLRRERQSR), DPV15b (SEQ ID NO 18: GAYDLRRRERQSRLRRRERQSR), HIV-1 Tat (SEQ ID NO 19: RKKRRQRRR), FHV coat (SEQ ID NO 20: RRRRNRTRRNRRRVR), HIV-1 Rev (SEQ ID NO 21: TRQARRNRRRRWRERQR), HTLV-II Rex (SEQ ID NO 22: TRRQRTRRARRNR), BMV Gag (SEQ ID NO 23: KMTRAQRRAAARRNRWTAR, P22 N (SEQ ID NO 24 NAKTRRHERRRKLAIER), λN(1-22) (SEQ ID NO 25: MDAQTRRRERRAEKQAQWKAAN), φ21N(12-29) (SEQ ID NO 26: TAKTRYKARRAELIAERR), Yeast Prp6 (SEQ ID NO 27: TRRNKRNRIQEQLNRK), Protamine 1 (SEQ ID NO 28: PRRRRSSSRPVRRRRRPRVSRRRRRRGGRRRR), Human cJun (SEQ ID NO 29: RIKAERKRMRNRIAASKSRKRKLERIAR), Human cFos (SEQ ID NO 30: KRRIRRERNKMAAAKSRNRRRELTDT), Yeast GCN4 (SEQ ID NO 31: KRARNTEAARRSRARKLQRMKQ), Penetratin (SEQ ID NO 32: RQIKIWFQNRRMKWKK), Islet-1 (SEQ ID NO 33: RVIRVWFQNKRCKDKK), Fushi-tarazu (SEQ ID NO 34: SKRTRQTYTRYQTLELEKEFHFNRYITRRRRIDIANALSLSERQIKIWFQNRRMKSKKDR), Engrailed-2 (SEQ ID NO 35: SQIKIWFQNKRAKIKK), HoxA-13 (SEQ ID NO 36: RQVTIWFQNRRVKEKK), Knotted-1 (SEQ ID NO 37: KQINNWFINQRKRHWK), PDX-1 (SEQ ID NO 38: RHIKIWFQNRRMKWKK), MPG (SEQ ID NO 39: GLAFLGFLGAAGSTMGAWSQPKKKRKV), Bac7 (SEQ ID NO 40: RRIRPRPPRLPRPRPRPLPFPRPG), S413-PVrev (SEQ ID NO 41: ALWKTLLKKVLKAPKKKRKV), HRSV (SEQ ID NO 42: RRIPNRRPRR), L-2 (SEQ ID NO 43: HARIKPTFRRLKWKYKGKFW), Melittin (SEQ ID NO 44: GIGAVLKVLTTGLPALISWIKRKRQQ), SynB1 (SEQ ID NO 45: RGGRLSYSRRRFSTSTGR), IVV-14 (SEQ ID NO 46: KLWMRWYSPTTRRYG), AIP6 (SEQ ID NO 47: RLRWR), CAYH (SEQ ID NO 48: CAYHRLRRC), SVM4 (SEQ ID NO 49: LYKKGPAKKGRPPLRGWFH), SVM3 (SEQ ID NO 50: KGTYKKKLMRIPLKGT), SVM2 (SEQ ID NO 51: RASKRDGSWVKKLHRILE), Buforin 2 (SEQ ID NO 52: TRSSRAGLQWPVGRVHRLLRK), SVM1 (SEQ ID NO 53: FKIYDKKVRTRVVKH), SAP (SEQ ID NO 54: VRLPPPVRLPPPVRLPPP), 435b (SEQ ID NO 55: GPFHFYQFLFPPV), Peptl (SEQ ID NO 56: PLILLRLLRGQF), YTA2 (SEQ ID NO 57: YTAIAWVKAFIRKLRK), Pep-1 (SEQ ID NO 58: KETWWETWWTEWSQPKKRKV), EB-1 (SEQ ID NO 59: LIRLWSHLIHIWFQNRRLKWKKK), Pyrrho-coricin (SEQ ID NO 60: VDKGSYLPRPTPPRPIYNRN), 439a (SEQ ID NO 61: GSPWGLQHHPPRT), MAP (SEQ ID NO 62: KLALKALKALKAALKLA), Bip (1) (SEQ ID NO 63: IPALK), Bip (2) (SEQ ID NO 64: VPALR), pVEC (SEQ ID NO 65: LLIILRRRIRKQAHAHSK), YTA4 (SEQ ID NO 66: IAWVKAFIRKLRKGPLG), K-FGF+NLS (SEQ ID NO 67: AAVLLPVLLAAPVQRKRQKLP), HN-1 (SEQ ID NO 68: TSPLNIHNGQKL), Bip (3) (SEQ ID NO 69: VPTLK), Bip (4) (SEQ ID NO 70: VSALK), VT5 (SEQ ID NO 71: DPKGDPKGVTVTVTVTVTGKGDPKPD), Transportan 10 (SEQ ID NO 72: AGYLLGKINLKALAALAKKIL), SAP(E) (SEQ ID NO 73: VELPPPVELPPPVELPPP), CADY (SEQ ID NO 74: GLWRALWRLLRSLWRLLWRA), PreS2-TLM (SEQ ID NO 75: PLSSIFSRIGDP), R/W (SEQ ID NO 76: RRWWRRWRR), or combinations of any of the preceding.

In some embodiments, a compound of the disclosure is conjugated to a peptide that targets specific tissue, such as a muscle-targeting peptides. Suitable muscle targeting peptides can include, for example, MSP1 (SEQ ID NO 77: AKASSLNIA), MSP2 (SEQ ID NO 78: ASSLNIA), and A2G80 (SEQ ID NO: 79: VQLRNGFPYFSY).

Other peptides suitable for conjugation with a compound of the disclosure include transferrin receptor binders, such as THR (SEQ ID NO: 80: THRPPMWSPVWP) and HAI (SEQ ID NO: 81: HAIYPRH), as well as peptides that bind transferrin receptor-transferrin complex, such as CRT (SEQ ID NO 82: C(&)RTIGPSVC(&)).

Retro-enantio analogues of any peptide disclosed herein are also suitable for conjugation to a compound of the present disclosure. A retro-enantio analogue can mimic the natural function of a corresponding parent peptide while exhibiting increased resistance to degradation. A retro-enantio analogue includes a peptide analogue where, relative to a parent peptide, both the linear peptide sequence and alpha-carbon chirality are inverted. For example, a retro-enantio analogue of THR (SEQ ID NO: 80: THRPPMWSPVWP) can be THRre (SEQ ID NO: 83: pwvpswmpprht), and a retro-enantio analogue of HAI (SEQ ID NO: 81: HAIYPRH) can be HAIre (SEQ ID NO: 84: hrpyiah), where lowercase one letter codes denote D-amino acid residues.

Enantiomers of any peptide disclosed herein are also contemplated, which enantiomers can include, for example, D-THR (SEQ ID NO: 85: thrppmwspvwp).

Other peptides suitable for conjugation with a compound of the disclosure include peptides consisting of or comprising sequences such as RFQILYR (SEQ ID NO: 86), RYQFLIR (SEQ ID NO: 87), RIQFLIR (SEQ ID NO: 88), RRWQW (SEQ ID NO: 89), GWWG (SEQ ID NO: 90), GFWFG (SEQ ID NO: 91), and GRKKRRQRRRPQ (SEQ ID NO: 92). Peptides comprising repeating units of charged residues are also contemplated, such as sequences comprising repeating units of contiguous arginine and glycine residues, such as (RG) e where e is from 1 to 50 (SEQ ID NO: 138) (e.g. SEQ ID NO 93: RGRGRGRGRGRGRG), polyarginine comprising from 2 to 100 contiguous arginine residues (SEQ ID NO: 139), (e.g. SEQ ID NO 94: RRRRRRRRRRRR), and repeating units of proline-proline-arginine, such as (PPR)_{f} where f is from 2 to 50 (SEQ ID NO: 140) (e.g., SEQ ID NO 95: PPRPPRPPRPPR).

In some embodiments, a compound of the disclosure is complementary to a nucleic acid sequence selected from the group consisting of: CUG, CUGC, CUGCU, CUGCUG, CUGCUGC, CUGCUGCU, CUGCUGCUG, CUGCUGCUGC (SEQ ID NO: 96), CUGCUGCUGCU (SEQ ID NO: 97), CUGCUGCUGCUG (SEQ ID NO: 98), CUGCUGCUGCUGC (SEQ ID NO: 99), CUGCUGCUGCUGCU (SEQ ID NO: 100), CUGCUGCUGCUGCUG (SEQ ID NO: 101), CUGCUGCUGCUGCUGC (SEQ ID NO: 102), CUGCUGCUGCUGCUGCU (SEQ ID NO: 103), CUGCUGCUGCUGCUGCUG (SEQ ID NO: 104), CUGCUGCUGCUGCUGCUGC (SEQ ID NO: 105), CUGCUGCUGCUGCUGCUGCU (SEQ ID NO: 106), CUGCUGCUGCUGCUGCUGCUG (SEQ ID NO: 107), CUGCUGCUGCUGCUGCUGCUGC (SEQ ID NO: 108), CUGCUGCUGCUGCUGCUGCUGCU (SEQ ID NO: 109), and CUGCUGCUGCUGCUGCUGCUGCUG (SEQ ID NO: 110).

In some embodiments, sequence variants of the sequences described herein are contemplated. A variant typically differs from a sequence specifically disclosed herein in one or more substitutions, deletions, additions and/or insertions. Such variants can be naturally occurring or can be synthetically generated, for example, by modifying one or more of sequences of the disclosure and evaluating one or more biological activities of the compounds as described herein.

Such modifications include, for example, deletions from, and/or insertions into and/or substitutions of residues within the amino acid and/or nucleic acid sequences of the compound. Any combination of deletion, insertion, and substitution can be made to arrive at the final construct, provided that the final construct possesses the desired characteristics, e.g., modulation of a genetic target.

Percent (%) sequence identity with respect to a reference polypeptide or oligonucleotide sequence is the percentage of amino acid residues, nucleoside residues, and/or nucleoside analogue residues in a candidate sequence that are identical with residues in the reference sequence, after aligning the sequences and introducing gaps, if necessary, to achieve the maximum percent sequence identity, and not considering any conservative substitutions as part of the sequence identity. Appropriate parameters for aligning sequences are able to be determined, including algorithms needed to achieve maximal alignment over the full length of the sequences being compared. In some embodiments, the degree of sequence identity between two sequences can be determined, for example, by comparing the two sequences using computer programs designed for this purpose, such as global or local alignment algorithms. Non-limiting examples include BLAST, BLAST-2, ALIGN, Megalign (DNASTAR), BLASTp, BLASTn, Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, SSEARCH2SEQ, or another suitable method, software or algorithm. A global alignment algorithm, such as a Needleman and Wunsch algorithm, can be used to align two sequences over their entire length, maximizing the number of matches and minimizes the number of gaps. Default settings can be used. In some embodiments, % sequence identity values can be generated using the sequence comparison computer program ALIGN-2. The ALIGN-2 program can be compiled for use on a UNIX operating system, including digital UNIX V4.0D. Sequences that can be compared by these algorithms include, for example, peptides, oligonucleotides, PNAs, and analogues of any of the foregoing.

When comparing two compounds that each have nucleobases or amino acid side chains but do not have the same backbone motif, the percent identity determination can be made based on comparison of the nucleobases or amino acid side chains. Residues in the two molecules being compared can be considered to share identity for the purpose of the percent identity analysis if the residues share a common nucleobase or amino acid side chain even if the residues have non-identical backbone structures. For example, Compound 58 and Compound 59 can be considered to share 100% nucleobase sequence identity.

Compounds disclosed herein can additionally comprise non-proteogenic acids in place of one or more proteogenic amino acids amino acids. Such non-proteogenic acids can include, for example, β-alanine, cystine, cystathionine, lanthionine, t-leucine, norleucine, homonorleucine, ornithine, allothreonine, homocysteine, citrulline, homoserine, isovaline, norvaline, sarcosine, N-ethyl glycine, N-propyl glycine, N-isopropyl glycine, N-methyl alanine, N-ethyl alanine, N-methyl β-alanine, N-ethyl β-alanine, and isoserine.

Compounds described herein can be associated with modifications of one or more amino acids of the compounds. Non-limiting examples of modifications include phosphorylation, acylation including acetylation and formylation, glycosylation (including N-linked and O-linked), amidation, hydroxylation, alkylation including methylation and ethylation, ubiquitination, addition of pyrrolidone carboxylic acid, formation of disulfide bridges, sulfation, myristoylation, palmitoylation, isoprenylation, farnesylation, geranylation, glypiation, lipoylation and iodination.

The nucleobases within a PNA subunit can be naturally occurring or non-naturally occurring. Non-limiting examples of nucleobases include adenine, guanine, thymine, cytosine, uracil, pseudoisocytosine, 2-thiopseudoisocytosine, 5-methylcytosine, 5-hydroxymethylcytosine, xanthine, hypoxanthine, 2-aminoadenine (or 2,6-diaminopurine), 2-thiouracil, 2-thiothymine, 2-thiocytosine, 5-chlorouracil, 5-bromouracil, 5-iodouracil, 5-chlorocytosine,5-bromocytosine, 5-iodocytosine, 5-propynyl uracil, 5-propynyl cytosine, 6-azo uracil, 6-azo cytosine, 6-azo thymine, 7-methylguanine, 7-methyladenine, 8-azaguanine, 8-azaadenine, 7-deazaguanine, 7-deazaadenine, 3-deazaguanine, 3-deazaadenine, 7-deaza-8-aza guanine, 7-deaza-8-aza adenine, 5-propynyl uracil and 2-thio-5-propynyl, pyridazin-3(2H)-one (E), pyrimidin-2(1H)-one (P) and 2-aminopyridine (M), and tautomeric forms thereof.

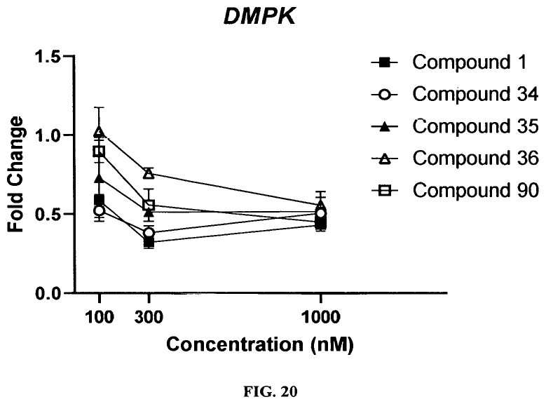

Compounds disclosed herein can comprise divalent nucleobases. A divalent nucleobase can simultaneously bind specifically to two nucleic acid strands, whether or not the two strands are independent strands, two portions of a single strand (e.g., in a hairpin), or contain mismatches in the sense that at one or more positions within the two strands at the site of binding to the genetic recognition reagents, the bases are not able to base pair according to traditional Watson-Crick base pairing (A-T/U, T/U-A, G-C or C-G). Divalent nucleobases can be incorporated into a oligonucleotide analogue backbone such as those described in FIG. 20 (e.g. PNA monomer), which can then be incorporated into an oligomer of monomers with a desired sequence of nucleobases. TABLE 4 provides example divalent bases and their binding specificities, where R¹ is hydrogen or a nitrogen protecting group and X is N or CH.

TABLE 4

| | Target | |
|---|---|---|
| | nucleobase | |
| Nucleobase | pair | Nucleobase residue |
| JB1 | T/D* | 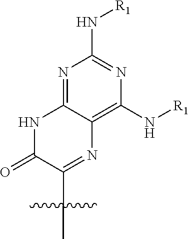 |
| JB1b | T(U)/D* | 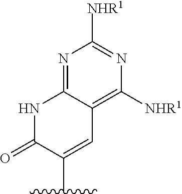 |
| JB1c | T(U)/D* | 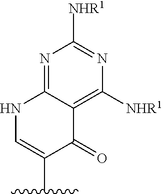 |
| JB1d | T(U)/D* | 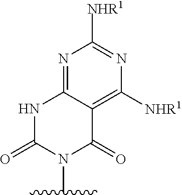 |
| JB2 | D/T | 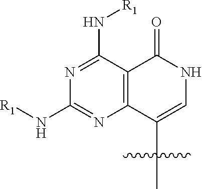 |
| JB2b | D/T(U) | 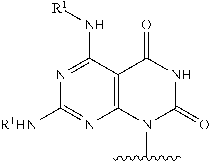 |
| JB3 | G/C | 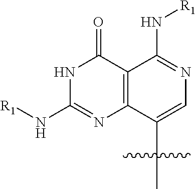 |
| JB3b | G/C | 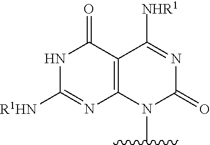 |
| JB4 | C/G | 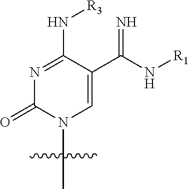 |
| JB4b | C/G | 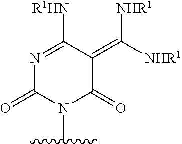 |
| JB4c | C/G | 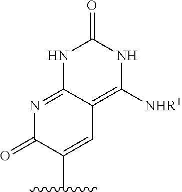 |
| JB4d | C/G | 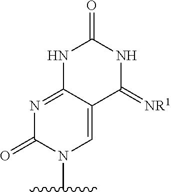 |
| JB4e | C/G | 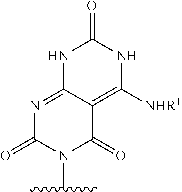 |
| JB5 | C/C | 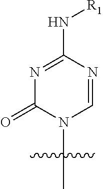 |
| JB5b | C/C | 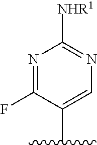 |
| JB5c | C/C | 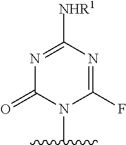 |
| JB5d | C/C | 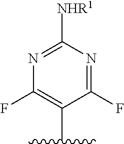 |
| JB6 | U/U | 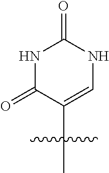 [SMILES: *c1c[nH]c(=O)[nH]c1=O] |
| JB6b | T(U)/T(U) | 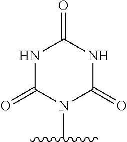 [SMILES: *n1c(=O)[nH]c(=O)[nH]c1=O] |
| JB7 | G/G | 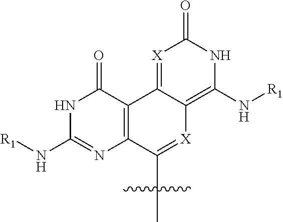 |
| JB7e | G/G | 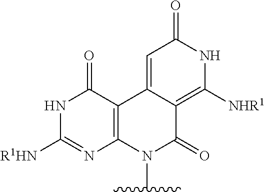 |
| JB7f | G/G | 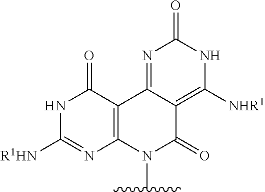 |
| JB8 | D/D | 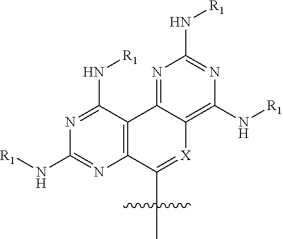 |
| JB8b | D/D | 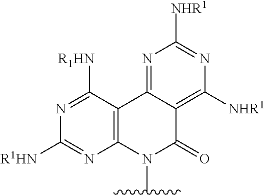 |
| JB9 | A/C | 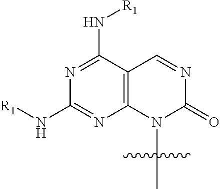 |
| JB9b | A/C | 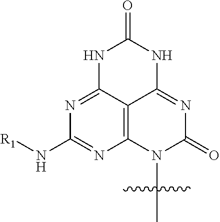 |
| JB9c | A/C | 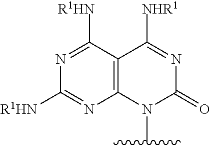 |
| JB10 | C/A | 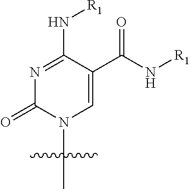 |
| JB10b | C/A | 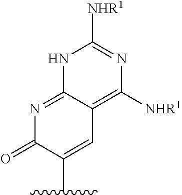 |
| JB10c | C/A | 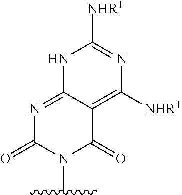 |
| JB11 | U/G | 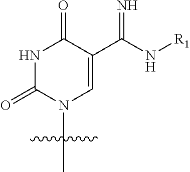 |
| JB11b | T(U)/G | 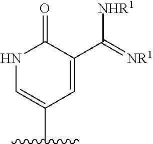 |
| JB11c | T(U)/G | 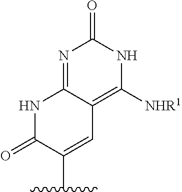 |
| JB11d | T(U)/G | 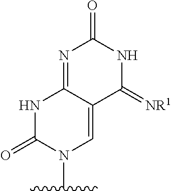 |
| JB11e | T(U)/G | 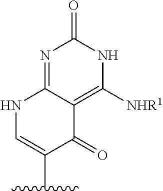 |
| JB12 | G/U | 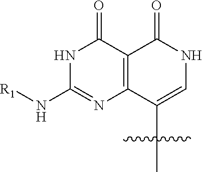 |
| JB12b | G/T(U) | 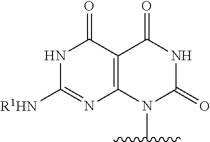 |
| JB13 | C/U | 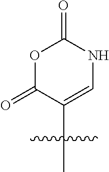 [SMILES: *c1c[nH]c(=O)oc1=O] |
| JB13b | C/T(U) | 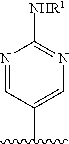 |
| JB13d | C/T(U) | 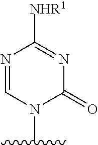 |
| JB13f | C/T(U) | 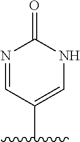 [SMILES: *c1cnc(=O)[nH]c1] |
| JB13g | C/T(U) | 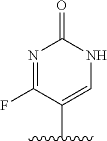 [SMILES: *c1c[nH]c(=O)nc1F] |
| JB13h | C/T(U) | 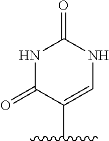 [SMILES: *c1c[nH]c(=O)[nH]c1=O] |
| JB14 | U/C | 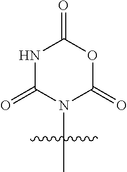 [SMILES: *n1c(=O)[nH]c(=O)oc1=O] |
| JB15 | G/D | 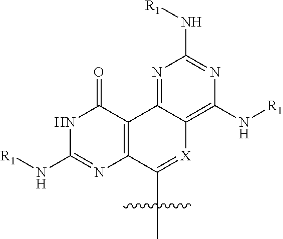 |
| JB16 | D/G | 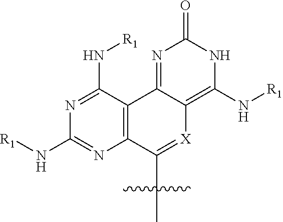 |

Compounds described herein (e.g., PNA subunits and PNA oligomers) can comprise one or more isotopic substitutions. For example, hydrogen can be in any isotopic form, including ¹H (protium), ²H (D or deuterium), and ³H (T or tritium). Carbon can be in any isotopic form, including ¹²C, ¹³C, and ¹⁴C. Oxygen can be in any isotopic form, including ¹⁶O and ¹⁸O.

Compounds described herein (e.g., PNA subunits and PNA oligomers) can comprise one or more asymmetric centers, and can exist in various isomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods including chiral high-performance liquid chromatography (HPLC), selective crystallization as chiral salts, or in the presence of chiral hosts, or from chiral solvents, and through enrichment using enzymes or chemical processes such as dynamic kinetic resolution. A single isomer can be prepared by asymmetric synthesis. The disclosure additionally encompasses compounds described herein as individual isomers substantially free of other isomers, and alternatively, as mixtures of various isomers.

### Chemical Groups.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, hydrocarbyl groups, acyloxy groups, carbamate groups, amide groups, and ester groups.

Non-limiting examples of alkyl and alkylene groups include straight, branched, and cyclic alkyl and alkylene groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl.

An aryloxy group can be, for example, an oxygen atom substituted with any aryl group, such as phenoxy.

An aralkyl group can be, for example, any alkyl group substituted with any aryl group, such as benzyl.

An arylalkoxy group can be, for example, an oxygen atom substituted with any aralkyl group, such as benzyloxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include nucleobases, pyrrole, pyrrolidine, pyridine, piperidine, succinamide, maleimide, morpholine, imidazole, thiophene, furan, tetrahydrofuran, pyran, and tetrahydropyran.

An acyl group can be, for example, a carbonyl group substituted with hydrocarbyl, alkyl, hydrocarbyloxy, alkoxy, aryl, aryloxy, aralkyl, arylalkoxy, or a heterocycle. Non-limiting examples of acyl include acetyl, benzoyl, benzyloxycarbonyl, phenoxycarbonyl, methoxycarbonyl, and ethoxycarbonyl.

An acyloxy group can be an oxygen atom substituted with an acyl group. An ester or an ester group comprises an acyloxy group. A non-limiting example of an acyloxy group, or an ester group, is acetate.

A carbamate group can be an oxygen atom substituted with a carbamoyl group, wherein the nitrogen atom of the carbamoyl group is unsubstituted, monosubstituted, or disubstituted with one or more of hydrocarbyl, alkyl, aryl, heterocyclyl, or aralkyl. When the nitrogen atom is disubstituted, the two substituents together with the nitrogen atom can form a heterocycle.

A hydrocarbyl group can be any group consisting of carbon and hydrogen atoms, and can include alkyl groups, alkenyl groups, alkynyl groups, and aryl groups. A hydrocaryl group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group.

A hydrocarbylcarbonyl group can be a carbonyl group substituted with a hydrocarbyl group, which can be, for example, benzoyl, acetyl, propanoyl, butanoyl, pentanoyl, hexanoyl, heptanoyl, octanoyl, nonanoyl, decanoyl, undencanoyl, dodecanoyl, tridencanoyl, myristoyl, pentadecenoyl, palmitoyl, heptadecanoyl, stearoyl, nondecanoyl, arachidoyl, as well as acyl groups derived from saturated, monounsaturated, and polyunsaturated fatty acids, such as myristoleoyl, palmitoleoyl, sapienoyl, oleoyl, elaidoyl, vaccenoyl, linoleoyl, linoelaidoyl, α-linolenoyl, or arachidonoyl. A hydrocarylcarbonyl group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group.

An aminoalkylene group can be an alkyl group substituted with an amino group, such as, for example, aminomethyl, 2-aminoeth-1-yl, 3-aminoprop-1-yl, 2-aminoprop-1-yl, 4-aminobut-1-yl, 3-aminobut-1-yl, 2-aminobut-1-yl, 5-aminopent-1-yl, 4-aminopent-1-yl, 4-aminopent-1-yl, 3-aminopent-1-yl, 2-aminopent-1-yl, a lysine side chain, or an ornithine side chain.

A guanidinoalkylene group can be an alkyl group substituted with a guanidino group, such as, for example, guanidinomethyl, 2-guanidinoeth-1-yl, 3-guanidinoprop-1-yl, 2-guanidinoprop-1-yl, 4-guanidinobut-1-yl, 3-guanidinobut-1-yl, 2-guanidinobut-1-yl, 5-guanidinopenty-1-1,4-guanidinopent-1-yl, 4-guanidinopent-1-yl, 3-guanidinopent-1-yl, 2-guanidinopent-1-yl, an arginine side chain, or a homoarginine side chain.

Polypeptides and proteins disclosed herein (including functional portions and functional variants thereof) can comprise synthetic amino acids in place of one or more naturally-occurring amino acids. Such synthetic amino acids can include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

In some embodiments, a compound of a disclosure bears one or more nitrogen protecting groups. Nonlimiting examples of nitrogen protecting groups include methyl, formyl, ethyl, acetyl, benzyl, benzoyl, carbamate, trifluoroacetyl, diphenylmethyl, triphenylmethyl, benzyloxymethyl, benzyloxycarbonyl, 2-nitrobenzoyl, t-Boc (tert-butyloxycarbonyl), 4-methylbenzyl, 4-nitrophenyl, 2-chlorobenzyloxycarbonyl, 2-bromobenzyloxycarbonyl, trichlorophenyl, thioanizyl, thiocresyl, cbz (carbobenzyloxy), p-methoxybenzyl carbonyl, 9-fluorenylmethyloxycarbonyl (Fmoc), pentafluorophenyl, p-methoxybenzyl 3,4-dimethozybenzyl p-methoxyphenyl, 4-toluenesulfonyl, p-nitrobenzenesulfonates, 9-fluorenylmethyloxycarbonyl, 2-nitrophenylsulfenyl, 2,2,5,7,8-pentamethyl-chroman-6-sulfonyl, 2-(4-Nitrophenyl)sulfonylethoxycarbonyl (Nsc), 1,1-Dioxobenzo[b]thiophene-2-ylmethyloxycarbonyl (Bsmoc), 1,1-Dioxonaphtho[1,2-b]thiophene-2-methyloxycarbonyl (α-Nsmoc), 3,3-Dioxonaphtho[2,1-b]thiophene-2-methyloxycarbonyl (β-Nsmoc), 1-(4,4-dimethyl-2,6-dioxocyclohex-1-ylidene)-3-methylbutyl (ivDde), 2,7-di-tert-butyl-9-fluorenylmethoxycarbonyl (Fmoc*), 2-monoisooctyl-9-fluorenylmethoxycarbonyl (mio-Fmoc), 2,7-diisooctyl-9-fluorenylmethoxycarbonyl, tetrachlorophthaloyl (TCP), 2-fluoro-9-fluorenylmethoxycarbonyl (Fmoc(2F)), 2-[Phenyl(methyl)sulfonio]ethyloxycarbonyl tetrafluoroborate (Pms), ethanesulfonylethoxycarbonyl (Esc), 2-(4-sulfophenylsulfonyl)ethoxycarbonyl (Sps), N,N-dimethylaminocarbonyl (Dmaoc), and p-bromobenzenesulfonyl.

### Pharmaceutically-Acceptable Salts.

The disclosure provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, imidazole, or pyrazine.

In some embodiments, an ammonium salt is a triethyl amine salt, a trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, trifluoroacetic acid, mandelic acid, cinnamic acid, aspartic acid, stearic acid, palmitic acid, glycolic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a trifluoroacetate salt, a mandelate salt, a cinnamate salt, an aspartate salt, a stearate salt, a palmitate salt, a glycolate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

In some embodiments, the compounds of the disclosure show non-lethal toxicity.

### Therapeutic Methods

Compounds, compositions, and methods of the disclosure can be used to treat a condition in a subject in need thereof, for example, a repeat expansion disorder, such as trinucleotide repeat expansion disorder. The methods can comprise administering to a subject in need thereof a therapeutically-effective amount of a compound disclosed herein.

A condition to be treated can be a repeat expansion disorder, such as a trinucleotide repeat expansion disorder. The repeat can be present, for example, in a 5′ UTR, an intron, an exon, or a 3′ UTR of a gene. A trinucleotide repeat expansion disorder can be a disorder that involves an expansion of a CTG repeat that is translated into a CUG repeat (a CTG or CUG repeat expansion disorder). Non-limiting examples of disorders that comprise an expansion of a CTG repeat include myotonic dystrophy (e.g., myotonic dystrophy type 1 (DM1), with a repeat expansion in the 3′ UTR of DMPK), the HSA^{LR} mouse model of DM1, with a repeat expansion the 3′ UTR of ACTA1 mRNA (which can lead to the formation of toxic hairpins and impaired splicing due to MBNL1 sequestration, as seen in myotonic dystrophy with DMPK), spinocerebellar ataxia type 8 (repeat expansion in SCA8 locus), and Fuchs corneal dystrophy (repeat expansion in TCF4). In some embodiments, a pharmaceutical composition, compound, or method of the disclosure is used for treating an animal model of DM1, such as an HSA^{LR} mouse model of DM1. In some embodiments, a composition, compound, or method of the disclosure is used for treating DM1. In some embodiments, a composition, compound, or method of the disclosure is used for treating spinocerebellar ataxia type 8. In some embodiments, a composition, compound, or method of the disclosure is used for treating Fuchs corneal dystrophy. In some embodiments, a repeat expansion disorder is a CCTG repeat expansion, for example, an expansion of CCTG in intron 1 of CNBP. Myotonic dystrophy type 2 (DM2) is an example of a CCTG repeat expansion disorder. In some embodiments, a composition, compound, or method of the disclosure is used for treating DM2.

In some embodiments, a condition to be treated is a neurological condition. In some embodiments, the condition is a neurodegenerative condition. In some embodiments, a condition to be treated is a neuromuscular condition. In some embodiments, the condition is a central nervous system condition and/or a peripheral nervous system condition. In some embodiments, the condition is a multisystem degenerative disorder. In some embodiments, a condition to be treated is a muscular dystrophy. In some embodiments, the condition is associated with aging. In some embodiments, the condition comprises or is associated with cognitive impairment or intellectual disability. In some embodiments, the condition comprises or is associated with deterioration of motor skills. In some embodiments, the condition comprises or is associated with progressive muscle wasting and/or weakness. In some embodiments, the condition comprises or is associated with cataract development. In some embodiments, the condition comprises or is associated with reduced lifespan. In some embodiments, the condition comprises or is associated with myotonia (sustained muscle contraction, e.g., an inability to relax muscles at will). In some embodiments, the condition comprises or is associated with cardiac pathology, such as cardiac conduction abnormalities. In some embodiments, the condition comprises or is associated with arrhythmia. In some embodiments, the condition is a congenital condition. In some embodiments, the condition comprises or is associated with hypotonia. In some embodiments, the condition comprises or is associated with severe generalized weakness. In some embodiments, the condition comprises or is associated with respiratory insufficiency.

In some embodiments, the condition is a genetic disorder. In some embodiments, the condition is an autosomal dominant genetic disorder. In some embodiments, the condition comprises or is associated with mis-splicing of gene transcripts. In some embodiments, the condition comprises or is associated with altered protein products that are dysfunctional as a result of mis-splicing. In some embodiments, the condition comprises or is associated with sequestration of splice regulators. In some embodiments, the condition comprises or is associated with aggregation of mRNA in the nucleus. In some embodiments, the condition comprises or is associated with protein haploinsufficiency, such as DMPK protein haploinsufficiency. In some embodiments, the severity of the condition is associated with the number of trinucleotide repeats in a trinucleotide repeat expansion. In some embodiments, the condition is a monogenic disorder, e.g., comprises or is associated with an inherited defect in a single gene.

In some embodiments, the condition is DM1. In some embodiments, the condition is congenital DM1. In some embodiments, the condition is childhood-onset DM1. In some embodiments, the condition is adult-onset DM1.

In some embodiments, the disclosure provides a method of treating DM1, the method comprising contacting a cell of a subject (e.g., patient) suffering from DM1 with a compound disclosed herein (e.g., Compound 1 or Compound 2 of TABLE 1). Upon contacting a compound disclosed herein (e.g., Compound 1) with the cell, the compound can penetrate the cell membrane, endosome, and nucleus, engage DMPK mRNA, and restore DMPK mRNA (e.g., to normal levels). In some embodiments, the disclosure provides a method of treating an animal model of DM1, the method comprising contacting a cell of a subject (e.g., HSA^{LR} mouse) with a compound disclosed herein (e.g., Compound 1 or Compound 2). Upon contacting a compound disclosed herein (e.g., Compound 1) with the cell, the compound can penetrate the cell membrane, endosome, and nucleus, engage ACTA1 mRNA, and restore ACTA1 mRNA (e.g., to normal levels).

A compound or composition of the disclosure can be administered on the basis of the number of CTG repeats in a gene, for example, in the DMPK gene, JPH3 gene, TCF4 gene, ACTA1 gene, or SCA8 locus. In some embodiments, the efficacy of a compound, composition, or method of the disclosure can vary based on the number of CTG repeats in a gene, for example, in the DMPK gene, JPH3 gene, TCF4 gene, ACTA1 gene, or SCA8 locus. The number of CTG repeats in a gene can be variable in the general population, for example, the number of CTG repeats in DMPK can be in a range of about 5 to about 37 repeats in subjects that do not have DM1. The number of CTG repeats in subjects with DM1 can be at least 50 and in some cases upwards of 3000. In some embodiments, the efficacy of a compound, composition, or method of the disclosure can vary based on the number of repeats in the gene or locus.

In some embodiments, a compound or composition is administered to a subject or contacted to a cell having a gene (e.g., DMPK gene, JPH3 gene, TCF4 gene, ACTA1 gene, or SCA8 locus) that comprises at least about 30, at least about 40, at least about 45, at least about 50, at least about 55, at least about 60, at least about 65, at least about 70, at least about 75, at least about 80, at least about 90, at least about 100, at least about 110, at least about 120, at least about 130, at least about 140, at least about 150, at least about 160, at least about 170, at least about 180, at least about 190, at least about 200, at least about 250, at least about 300, at least about 350, at least about 400, at least about 450, at least about 500, at least about 600, at least about 700, at least about 800, at least about 900, at least about 1000, at least about 1500, at least about 2000, at least about 2500, or at least about 3000 trinucleotide repeats, for example, CTG repeats or CUG repeats. In some embodiments, the gene is DMPK. In some embodiments, the gene is not DMPK. In some embodiments, the gene is Junctophilin-3 (JPH3). In some embodiments, the gene is not JPH3. In some embodiments, the gene is transcription factor four (TCF4). In some embodiments, the gene is not TCF4. In some embodiments, the gene is ACTA1. In some embodiments, the gene is not ACTA1. In some embodiments, the gene is the SCA8 locus. In some embodiments, the gene not the SCA8 locus. In some embodiments the gene has at most about 100, at most about 250, at most about 500, at most about 1000, at most about 2000, at most about 3000, or at most about 5000 trinucleotide repeats.

In some embodiments, a trinucleotide repeat expansion comprises at least about 40, at least about 45, at least about 50, at least about 55, at least about 60, at least about 65, at least about 70, at least about 75, at least about 80, at least about 90, at least about 100, at least about 110, at least about 120, at least about 130, at least about 140, at least about 150, at least about 160, at least about 170, at least about 180, at least about 190, at least about 200, at least about 250, at least about 300, at least about 350, at least about 400, at least about 450, at least about 500, at least about 600, at least about 700, at least about 800, at least about 900, at least about 1000, at least about 1500, at least about 2000, at least about 2500, or at least about 3000 trinucleotide repeats, for example, CTG repeats or CUG repeats. In some embodiments the trinucleotide repeat expansion has at most about 100, at most about 250, at most about 500, at most about 1000, at most about 2000, at most about 3000, or at most about 5000 trinucleotide repeats.

In some embodiments, a cell, population of cells, or subject that does not have a trinucleotide repeat expansion contains at most 5, at most 10, at most 15, at most 20, at most 25, at most 30, at most 35, at most 36, at most 37, at most 38, at most 39, at most 40, at most 45, at most 50, at most 60, at most 70, at most 80, at most 90, or at most 100 copies of the trinucleotide (e.g., CTG or CUG) in the repeat region of the gene (e.g., DMPK gene, JPH3 gene, TCF4 gene, ACTA1 gene, or SCA8 locus).

In some embodiments, the disclosure provides a method of treating a nucleotide repeat expansion disorder in a subject, comprising administering a compound disclosed herein (e.g., Compound 1 or Compound 2) to the subject. In some embodiments, the nucleotide repeat expansion disorder comprises expression of CUG-repeat-containing mRNA by the subject. In some embodiments, the subject is a mammal. In some embodiments, the subject is human. In some embodiments, the subject is a mouse.

In some embodiments, a compound or composition is administered to a subject or contacted to a cell having a gene (e.g., DMPK gene, SCA8 locus, JPH3 gene, TCF4 gene, or ACTA1 gene) that comprises at least about 30, least about 40, at least about 45, at least about 50, at least about 55, at least about 60, at least about 65, at least about 70, at least about 75, at least about 80, at least about 90, at least about 100, at least about 110, at least about 120, at least about 130, at least about 140, at least about 150, at least about 160, at least about 170, at least about 180, at least about 190, at least about 200, at least about 250, at least about 300, at least about 350, at least about 400, at least about 450, at least about 500, at least about 600, at least about 700, at least about 800, at least about 900, at least about 1000, at least about 1500, at least about 2000, at least about 2500, or at least about 3000 repeats, for example, CTG repeats or CUG repeats. In some embodiments the gene has at most about 100, at most about 250, at most about 500, at most about 1000, at most about 2000, at most about 3000, or at most about 5000 repeats.

In some embodiments, a repeat expansion comprises at least about 40, at least about 45, at least about 50, at least about 55, at least about 60, at least about 65, at least about 70, at least about 75, at least about 80, at least about 90, at least about 100, at least about 110, at least about 120, at least about 130, at least about 140, at least about 150, at least about 160, at least about 170, at least about 180, at least about 190, at least about 200, at least about 250, at least about 300, at least about 350, at least about 400, at least about 450, at least about 500, at least about 600, at least about 700, at least about 800, at least about 900, at least about 1000, at least about 1500, at least about 2000, at least about 2500, or at least about 3000 repeats, for example, CTG or CUG repeats. In some embodiments the repeat expansion has at most about 100, at most about 250, at most about 500, at most about 1000, at most about 2000, at most about 3000, or at most about 5000 repeats.

In some embodiments, a cell, population of cells, or subject that does not have a repeat expansion contains at most 5, at most 10, at most 15, at most 20, at most 25, at most 30, at most 35, at most 36, at most 37, at most 38, at most 39, at most 40, at most 45, at most 50, at most 60, at most 70, at most 80, at most 90, or at most 100 copies of the repeat (e.g., CTG, CUG) in the repeat region of the gene (e.g., DMPK gene, JPH3 gene, TCF4 gene, ACTA1 gene, or SCA8 locus).

In some embodiments, the disclosure provides a method of treating a nucleotide repeat expansion disorder in a subject, comprising administering a compound disclosed herein (e.g., Compound 1 or Compound 2) to the subject. In some embodiments, the repeat expansion disorder comprises expression of repeat-containing mRNA by the subject. In some embodiments, the subject is a mammal. In some embodiments, the subject is human. In some embodiments, the subject is a mouse.

In some embodiments, administration of a compound provided herein does not exhibit or substantially does not exhibit immunogenicity. In some embodiments, administration of a compound provided herein does not promote or substantially does not promote generation of neutralizing antibodies, complement factors, pro-inflammatory cytokines, or type 1 interferons upon or after administration of the compound to a subject. In some embodiments, a compound does not activate or substantially does not activate the TLR9 receptor and is not presented or is minimally presented by MHCI or MHCII complexes to the immune system.

Compounds provided herein can be locally or systemically administered to a subject in need thereof as a therapeutically-effective amount of a compound that binds to a repeat codon. The subject can comprise a bloodstream, a brain, and a blood-brain-barrier. The compound that binds to the repeat codon can enter the brain by passing from the bloodstream through the blood-brain-barrier into the brain.

### Therapeutic Effects

A compound, composition, or method of the disclosure can reduce nuclear aggregates or inclusions in a cell that comprises a trinucleotide repeat expansion. In some embodiments, a compound, composition, or method of the disclosure can reduce nuclear aggregates or inclusions in a cell that comprises a trinucleotide repeat expansion by at least about 5%, at least about 10%, at least about 15%, at least about 20%, at least about 25%, at least about 30%, at least about 35%, at least about 40%, at least about 45%, at least about 50%, at least about 55%, at least about 60%, at least about 70%, at least about 80%, at least about 90%, at least about 95%, at least about 97%, or at least about 99%, for example, as determined by a fluorescence in situ hybridization (FISH) assay. In some embodiments, a compound, composition, or method of the disclosure can reduce nuclear aggregates or inclusions in a cell that comprises a trinucleotide repeat expansion by at least about 2-fold, at least about 3-fold, at least about 4-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 150-fold, at least about 200-fold, at least about 300-fold, at least about 400-fold, at least about 500-fold, at least about 750-fold, at least about 1000-fold, at least about 1500-fold, at least about 2000-fold, or at least about 5000-fold, for example, as determined by a fluorescence in situ hybridization (FISH) assay. In some embodiments, the level of nuclear aggregates is reduced from a detectable level to below a limit of detection. In some embodiments, the trinucleotide repeat is a CTG DNA repeat. In some embodiments, the trinucleotide repeat is a CUG RNA repeat. The cell can be, for example, a neuron, a muscle cell, or a fibroblast.

In some embodiments, a compound, composition, or method of the disclosure can reduce nuclear aggregates or inclusions in a cell that comprises a trinucleotide repeat expansion to within about 1%, within about 3%, within about 5%, within about 10%, within about 15%, within about 20%, within about 25%, within about 30%, within about 35%, within about 40%, within about 45%, within about 50%, within about 55%, within about 60%, or within about 70% of a level in a control cell that does not have the trinucleotide repeat expansion.

In some embodiments, if, in a study: (i) a first HSA-LR mouse is treated with a single dose of a compound disclosed herein by tail vein (IV) injection at 29 mg/kg, (ii) a second HSA-LR mouse is treated with daily doses of 3 mg/kg the compound for 7 days by tail vein (IV) injection at 3 mg/kg, (iii) a third HSA-LR mouse is injected with PBS by tail vein injection as an untreated control, (a) the mice are sacrificed 20 or 21 days after the first injection, quadricep is harvested and snap frozen in liquid nitrogen, (b) the tissue is fixed and cut in 6 micron sections, (c) fluorescence in situ hybridization (FISH) is performed on 6 micron sections using a CAG repeat probe having the sequence: /5Cy3/CAGCAGCAGCAGCAGCAGCA (SEQ ID NO: 111), with the probe (1 ng/μ1 final concentration) hybridized to sections overnight at 1 ng/μl final concentration, (d) the sections are washed and imaged using confocal fluorescence microscopy, with DAPI as a nuclear counterstain, then reductions in nuclear inclusions are observed for Animals treated with the compound.

Muscleblind Like Splicing Regulator 1 (MBNL1) and Muscleblind Like Splicing Regulator 2 (MBNL2) are transcripts that can be dysregulated in trinucleotide repeat expansion disorders, including CUG repeat expansion disorders, such as DM1. MBNL1 and MBNL2 encode C3H-type zinc finger proteins that modulate alternative splicing of pre-mRNAs and can act either as activators or repressors of splicing on specific pre-mRNA targets. Muscleblind proteins also bind specifically to expanded dsCUG RNA (e.g., with at least 50 repeats) but not to normal size CUG repeats and can thereby play a role in the pathophysiology of some trinucleotide repeat expansion disorders, such as DM1.

In some embodiments a composition, compound, or method of the disclosure can increase or restore a functional activity of MBNL1 and/or MBNL2, for example, in a cell or a subject with a trinucleotide repeat expansion disorder, such as a subject or cell with CUG repeat expansion. Non-limiting examples of functional activities of MBNL1 and/or MBNL2 that can be increased by a composition, compound, or method disclosed herein include: (i) inhibiting cardiac troponin-T (TNNT2) pre-mRNA exon inclusion (e.g., in muscle), (ii) inducing insulin receptor (IR) pre-mRNA exon inclusion (e.g., in muscle), (iii) antagonizing the alternative splicing activity pattern of CELF proteins, (iv) regulating the TNNT2 exon 5 skipping through competition with U2AF2, (v) inhibiting the formation of the spliceosome A complex on intron 4 of TNNT2 pre-mRNA, (vi) binding to the stem-loop structure within the polypyrimidine tract of TNNT2 intron 4 during spliceosome assembly, (vii) binding to the 5′-YGCU(U/G)Y-3′ consensus sequence, (viii) binding to IR RNA, or (ix) any combination thereof.

In some embodiments, a composition, compound, or method of the disclosure increases functional activity of MBNL1 and/or MBNL2 in a cell or subject with a trinucleotide repeat expansion to at least about 10%, at least about 15%, at least about 20%, at least about 25%, at least about 30%, at least about 35%, at least about 40%, at least about 45%, at least about 50%, at least about 55%, at least about 60%, at least about 70%, at least about 80%, at least about 90%, at least about 95%, or about 100% of a level observed in a cell or subject that does not have the trinucleotide repeat expansion.

In some embodiments, a composition, compound, or method of the disclosure increases functional activity of MBNL1 and/or MBNL2 in a cell or subject with a trinucleotide repeat expansion by at least about 10%, at least about 15%, at least about 20%, at least about 25%, at least about 30%, at least about 35%, at least about 40%, at least about 45%, at least about 50%, at least about 55%, at least about 60%, at least about 70%, at least about 80%, at least about 90%, at least about 95%, at least about 2-fold, at least about 3-fold, at least about 4-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 150-fold, at least about 200-fold, at least about 300-fold, at least about 400-fold, or at least about 500-fold, for example, compared to a control a cell or subject with the trinucleotide repeat expansion that is not treated with the composition, compound, or method.

Treatment with a compound disclosed herein (e.g., Compound 1) can rescue mis-splicing across MBNL1 and MBNL2 within two days after initial treatment, and induction of rescue can continue to improve through day 9.

A compound, composition, or method of the disclosure can reduce a level of a mis-spliced mRNA in a cell or population of cells that comprises a trinucleotide repeat expansion. A mis-spliced mRNA can be, for example, a splicing configuration that is common or present in a subject that has the trinucleotide repeat expansion but is rare or absent in a subject that lacks the trinucleotide repeat expansion, or a splicing configuration that comprises exons that are not present in a reference gene or genome database, or a splicing configuration that does not encode a native protein, for example, does not encode a protein that is present in a database such as UniProt. In some embodiments, the trinucleotide repeat is a CTG DNA repeat. In some embodiments, the trinucleotide repeat is a CUG RNA repeat. In some embodiments the cell is a muscle cell. In some embodiments the cell is a neuron. In some embodiments the cell is a fibroblast.

In some embodiments, a compound, composition, or method of the disclosure can reduce a level of a mis-spliced mRNA in a cell that comprises a trinucleotide repeat expansion by at least about 10%, at least about 15%, at least about 20%, at least about 25%, at least about 30%, at least about 35%, at least about 40%, at least about 45%, at least about 50%, at least about 55%, at least about 60%, at least about 70%, at least about 80%, at least about 90%, at least about 95%, at least about 97%, or at least about 99%, for example, as determined by a qPCR assay or an RNAseq assay. In some embodiments, a compound, composition, or method of the disclosure can reduce a level of a mis-spliced mRNA in a cell that comprises a trinucleotide repeat expansion by at least about 2-fold, at least about 3-fold, at least about 4-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 150-fold, at least about 200-fold, at least about 300-fold, at least about 400-fold, at least about 500-fold, at least about 750-fold, at least about 1000-fold, at least about 1500-fold, at least about 2000-fold, or at least about 5000-fold, for example, as determined by a qPCR assay or an RNAseq assay. In some embodiments, a level of the mis-spliced mRNA is reduced from a detectable level to below a limit of detection. The mis-spliced mRNA can be, for example, an mRNA of ADCK1, AGFG1, ATP11C, BCAS3, CA5BP1, CLCN1, COA1, FIRRE, GM29394, HER4, HMGA1, Insulin receptor (IR), MBNL1, MBNL2, MIR100HG, MRPL55, MYO18A, NR2C2AP, SENP5, SKA2, TREF1, or ZNF83.

In some embodiments, a compound, composition, or method of the disclosure can reduce a level of a mis-spliced mRNA in a cell that comprises a trinucleotide repeat expansion to within about 1%, within about 3%, within about 5%, within about 10%, within about 15%, within about 20%, within about 25%, within about 30%, within about 35%, within about 40%, within about 45%, within about 50%, within about 55%, within about 60%, or within about 70% of a level in a control cell or subject that does not have the trinucleotide repeat expansion. The mis-spliced mRNA can be, for example, an mRNA of ADCK1, AGFG1, ATP11C, BCAS3, CA5BP1, CLCN1, COA1, FIRRE, GM29394, HER4, HMGA1, Insulin receptor (IR), MBNL1, MBNL2, MIR100HG, MRPL55, MYO18A, NR2C2AP, SENP5, SKA2, TREF1, or ZNF83.

A compound, composition, or method of the disclosure can increase a level of a correctly spliced mRNA in a cell that comprises a trinucleotide repeat expansion. A correctly spliced mRNA can be, for example, a splicing configuration that is found or is common in a subject that lacks the trinucleotide repeat expansion, or a splicing configuration that comprises only exons that are present in a reference gene or genome database, or a splicing configuration that encodes a native protein, for example, as found in a protein database such as UniProt. In some embodiments, the trinucleotide repeat is a CTG DNA repeat. In some embodiments, the trinucleotide repeat is a CUG RNA repeat. In some embodiments the cell is a muscle cell. In some embodiments the cell is a neuron. In some embodiments, the cell is a fibroblast. The mRNA can encode, for example, CLCN1, ADCK1, AGFG1, ATP11C, BCAS3, CA5BP1, CLCN1, COA1, FIRRE, GM29394, HER4, HMGA1, MBNL1, MBNL2, MIR100HG, MRPL55, MYO18A, NR2C2AP, SENP5, SKA2, TREF1, or ZNF83, or insulin receptor (IR). The mRNA can encode, for example, a protein that is associated with a pathogenic phenotype in subjects that comprise the trinucleotide repeat expansion (for example, low expression or activity level of the protein is associated with the pathogenic phenotype).

In some embodiments, a compound, composition, or method of the disclosure can increase a level of a correctly spliced mRNA in a cell that comprises a trinucleotide repeat expansion by at least about 10%, at least about 15%, at least about 20%, at least about 25%, at least about 30%, at least about 35%, at least about 40%, at least about 45%, at least about 50%, at least about 55%, at least about 60%, at least about 70%, at least about 80%, at least about 90%, at least about 95%, at least about 2-fold, at least about 3-fold, at least about 4-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 150-fold, at least about 200-fold, at least about 300-fold, at least about 400-fold, at least about 500-fold, at least about 750-fold, at least about 1000-fold, at least about 1500-fold, at least about 2000-fold, or at least about 5000-fold, for example, as determined by a qPCR assay or an RNAseq assay. In some embodiments, the level of the correctly spliced mRNA is increased from below a limit of detection to a detectable level. The mRNA can be, for example, an mRNA of ADCK1, AGFG1, ATP11C, BCAS3, CA5BP1, CLCN1, COA1, FIRRE, GM29394, HER4, HMGA1, Insulin receptor (IR), MBNL1, MBNL2, MIR100HG, MRPL55, MYO18A, NR2C2AP, SENP5, SKA2, TREF1, or ZNF83.

In some embodiments, a compound, composition, or method of the disclosure can increase a level of a correctly spliced mRNA in a cell that comprises a trinucleotide repeat expansion to at least about 10%, at least about 15%, at least about 20%, at least about 25%, at least about 30%, at least about 35%, at least about 40%, at least about 45%, at least about 50%, at least about 55%, at least about 60%, at least about 70%, at least about 80%, at least about 90%, at least about 95%, at least about 97%, or about 100% of a level in a cell or population of cells that does not have the trinucleotide repeat expansion. The mRNA can be, for example, an mRNA of ADCK1, AGFG1, ATP11C, BCAS3, CA5BP1, CLCN1, COA1, FIRRE, GM29394, HER4, HMGA1, Insulin receptor (IR), MBNL1, MBNL2, MIR100HG, MRPL55, MYO18A, NR2C2AP, SENP5, SKA2, TREF1, or ZNF83.

A compound, composition, or method of the disclosure can increase a level of a protein that is encoded by a gene or mRNA that comprises a trinucleotide repeat expansion, for example, by rescuing splicing of the mRNA and/or translation of the protein. In some embodiments, the trinucleotide repeat is a CTG DNA repeat. In some embodiments, the trinucleotide repeat is a CUG RNA repeat. In some embodiments, the protein is DMPK. In some embodiments, the protein is not DMPK. In some embodiments, the protein is Junctophilin-3 (JPH3). In some embodiments, the protein is not JPH3. In some embodiments, the protein is transcription factor four (TCF4). In some embodiments, the protein is not TCF4. In some embodiments, the protein is ACTA1. In some embodiments, the protein is not ACTA1. In some embodiments, the level of the protein is increased in a muscle cell, a neuron, a fibroblast, or a population thereof.

For example, a compound, composition, or method of the disclosure can increase a level of a protein that is encoded by a gene or mRNA that comprises a trinucleotide repeat expansion by at least about 10%, at least about 15%, at least about 20%, at least about 25%, at least about 30%, at least about 35%, at least about 40%, at least about 45%, at least about 50%, at least about 55%, at least about 60%, at least about 70%, at least about 80%, at least about 90%, at least about 95%, at least about 2-fold, at least about 3-fold, at least about 4-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 150-fold, at least about 200-fold, at least about 300-fold, at least about 400-fold, at least about 500-fold, at least about 750-fold, at least about 1000-fold, at least about 1500-fold, at least about 2000-fold, or at least about 5000-fold, for example, as determined by a Western Blot assay. In some embodiments, the level of the protein is increased from below a limit of detection to a detectable level.

In some embodiments, a level of a protein that is encoded by a gene or mRNA that comprises a trinucleotide repeat expansion is increased in a cell or population of cells to at least about 10%, at least about 15%, at least about 20%, at least about 25%, at least about 30%, at least about 35%, at least about 40%, at least about 45%, at least about 50%, at least about 55%, at least about 60%, at least about 70%, at least about 80%, at least about 90%, at least about 95%, at least about 97%, or about 100% of a level in a control cell or population of cells that does not have the trinucleotide repeat expansion.

A compound, composition, or method of the disclosure can increase a level of a protein in a cell that is otherwise reduced due to the presence of an mRNA that comprises a trinucleotide repeat expansion, for example, by rescuing splicing of the mRNA and/or translation of the protein. For example, a compound, composition, or method of the disclosure can increase levels of active splicing regulators and/or decrease RNA aggregates, thereby allowing increased splicing and translation of an mRNA that encodes the protein. In some embodiments, the protein is CLCN1. In some embodiments, the protein is insulin receptor (IR). In some embodiments, the protein encoded by ADCK1, AGFG1, ATP11C, BCAS3, CA5BP1, COA1, FIRRE, GM29394, HER4, HMGA1, MBNL1, MBNL2, MIR100HG, MRPL55, MYO18A, NR2C2AP, SENP5, SKA2, TREF1, or ZNF83. In some embodiments, the protein is associated with a pathogenic phenotype in subjects that comprise the trinucleotide repeat expansion (for example, low expression or activity level of the protein is associated with the pathogenic phenotype).

In some embodiments, a compound, composition, or method of the disclosure can increase a level of a protein in a cell that is otherwise reduced due to the presence of an mRNA that comprises a trinucleotide repeat expansion by at least about 10%, at least about 15%, at least about 20%, at least about 25%, at least about 30%, at least about 35%, at least about 40%, at least about 45%, at least about 50%, at least about 55%, at least about 60%, at least about 70%, at least about 80%, at least about 90%, at least about 95%, at least about 2-fold, at least about 3-fold, at least about 4-fold, at least about 5-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 150-fold, at least about 200-fold, at least about 300-fold, at least about 400-fold, at least about 500-fold, at least about 750-fold, at least about 1000-fold, at least about 1500-fold, at least about 2000-fold, or at least about 5000-fold, for example, as determined by a Western Blot assay. The protein can be, for example, a protein encoded by ADCK1, AGFG1, ATP11C, BCAS3, CA5BP1, CLCN1, COA1, FIRRE, GM29394, HER4, HMGA1, Insulin receptor (IR), MBNL1, MBNL2, MIR100HG, MRPL55, MYO18A, NR2C2AP, SENP5, SKA2, TREF1, or ZNF83.

In some embodiments, a compound, composition, or method of the disclosure can increase a level of a protein in a cell or population of cells that is otherwise reduced due to the presence of an mRNA that comprises a trinucleotide repeat expansion to at least about 10%, at least about 15%, at least about 20%, at least about 25%, at least about 30%, at least about 35%, at least about 40%, at least about 45%, at least about 50%, at least about 55%, at least about 60%, at least about 70%, at least about 80%, at least about 90%, at least about 95%, at least about 97%, or about 100% of a level in a control cell or population of cells that does not have the trinucleotide repeat expansion. The protein can be, for example, a protein encoded by ADCK1, AGFG1, ATP11C, BCAS3, CA5BP1, CLCN1, COA1, FIRRE, GM29394, HER4, HMGA1, Insulin receptor (IR), MBNL1, MBNL2, MIR100HG, MRPL55, MYO18A, NR2C2AP, SENP5, SKA2, TREF1, or ZNF83.

In some embodiments, if, in an assay, (i) a first group of 3 HSA-LR mice are injected with a compound disclosed herein (e.g., Compound 1) at a dose of 30 mg/kg via tail vein injection (single dose), (ii) a second group of 3 HSA-LR mice are injected a compound disclosed herein at a dose of 3 mg/kg via tail vein injection (daily doses for 7 days), (iii) a third group of 3 HSA-LR mice are injected with PBS via tail vein injection, (iv) a group of 3 healthy FVB background mice are injected with PBS via tail vein injection, (a) 20 days after the first dose, the tibialis anterior muscles are harvested and processed for RNA seq analysis and western blotting, (b) for Western Blot, about 50-100 mg of muscle tissue is homogenized and lysed using 300 μl M-Per™ buffer, protein extracts are quantified using BCA assay, protein extracts are resolved on SDS-PAGE 4-12% gradient gels with Tris-SDS IVIES Buffer, with gels run at 150 V for 2 hours, (c) after gel electrophoresis, proteins are transferred to a nitrocellulose membrane, (d) primary antibodies specific for CLCN1 (1:1000, Abcam™, Cambridge UK, cat. n #ab189857) and Beta-actin (1:5000, Abcam™ Cambridge, UK, cat. n #ab8227) are used, (e) HRP conjugate anti-mouse or anti-rabbit secondary antibody is used for visualizing proteins using SuperSignal™ West Pico Plus Chemiluminescent Substrate, (f) protein bands are quantified using iBright™ Analysis Software, and bands are normalized according to beta-actin expression, and the CLCN1 expression inhibition is calculated as a relative value to untreated control cells, then, levels of the skeletal muscle chloride channel (CLCN1) protein are increased or restored by day 20 for HSA-LR mice that were injected with the compound, and (g) for RNA seq, total RNA is collected using an RNAeasy kit, with RNA eluted in 30 μl and quantified, (h) libraries for RNA sequencing are created from polyA-selected RNA, with the protocol adjusted for 300 ng total RNA, (i) libraries are amplified for 13 cycles and after clean up, concentration and quality are checked using Qubit and Tape station, (j) sequencing is run on Illumima NextSeq500™ with 2×150 bp sequencing and a read depth of approximately 50M, (k) reads are aligned to reference data with STAR v2.7.1a using the STAR options from rMATS v4.1.0, duplicates are removed with Picard MarkDuplicates v2.18.7, aberrant splice activity is measured with rMATS v4.1.0 run on all pairwise combinations of conditions (healthy-untreated, disease-treated, and disease-untreated) with standard options, the top events from the “Skipped Exons Junction Counts Exon Counts” (‘SE.MATS.JCEC.txt’) from the three outputs is then merged together, any event not present in the healthy-untreated vs. disease-treated comparison is dropped, and only those events with a minimum absolute inclusion difference greater than 0.2 are included, then approximately 99% of Clcn1 transcript splicing is corrected by day 20 at least for mice treated daily with 3 mg/kg of the compound.

In some embodiments, if, in an assay, (i) a first group of HSA-LR mice are injected with a compound disclosed herein at a dose of 0.03 mg/kg via intramuscular injection (single dose), (ii) a second group of HSA-LR mice are injected with PBS via intramuscular injection (single dose), (iii) a healthy FVB background mice are injected with PBS via intramuscular injection (single dose), (a) muscle tissue is harvested and processed for qPCR to quantify transcript levels, (b) total RNA is isolated using 1 mL of Trizol per 100 mg tissue, (c) tissues are homogenized with OMNI TH homogenizer (in Trizol), (d) RNA is extracted using BCP, precipitated using Isopropanol, and resuspended in 100 μL RNase free water, (e) 5 μg of the isolated RNA is treated with DNase to remove genomic DNA, and RNA re-purified using an RNeasy Mini kit, (f) 300 ng of purified total RNA is reverse transcribed using a SuperScriptIII First-Strand Synthesis System for RT-PCR, (g) qPCR reactions are carried out using TaqMan Fast Advanced Master Mix with ACTA1 forward primer GTAGCTACCCGCCCAGAAACT (SEQ ID NO: 112), ACTA1 reverse primer CCAGGCCGGAGCCATT (SEQ ID NO: 113), and custom TaqMan probe ACCACCGCCCTCGTGTGCG (3′ MGBNFQ quencher and 5′ 6FAM dye, IDT) (SEQ ID NO: 114), and a 1:10 dilution of cDNA used as input, (h) for Gtf2b reference gene qPCR, Gene Expression Assay Mm00663250_ml (4331182, Vic, Applied Biosystems) is used and 2 μL of cDNA, (i) qPCR is performed on QuantaStudio5 using fast ramp speed, (j) ACTA1 transgene expression relative to Gtf2b is calculated using the ΔΔCt method, then reduced levels of pathogenic ACTA1 mRNA is observed for animals treated with the compound (hACTA1 mRNA comprises a CUG repeat in the 3′ UTR that leads to the formation of toxic hairpins and impaired splicing due to MBNL1 sequestration in the HAS-LR mouse model of myotonic dystrophy).

A compound, composition, or method of the disclosure can correct splicing of multiple transcripts. For example, a compound, composition, or method of the disclosure can correct global exon inclusion levels in multiple transcripts as measured by a differential splice inclusion (DSI) statistic. A DSI statistic can provide a composite view of splice correction across multiple mis-spliced transcripts. In some embodiments, a compound, composition, or method of the disclosure can correct splicing as indicated by a reduction in DSI statistic of at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, or at least 0.95.

Treatment with a compound disclosed herein (e.g., Compound 1) can induce correction of global exon inclusion levels in transcripts that initially exhibit mis-splicing in DM1 patient-derived cell lines relative to unaffected patient cells, as measured by the human differential splice inclusion (hDSI) statistic. Treatment with a compound disclosed herein (e.g., Compound 1) can also induce rescue of mis-splicing from 100% normalized dysregulation in DM1 cells to 50.3% dysregulation of splicing in compound-treated cells with a p-value=1.39×10-44 five days after initial treatment.

In some embodiments, a subject is a HSA^{LR} mouse. HSA^{LR} mice are transgenic FVB mice that can express a human skeletal actin gene (HAS or hACTA1) gene with ˜220 CUG repeats in the 3′-UTR region. This repeat expansion results in a phenotype that resembles human subjects with a repeat expansion in the 3′-UTR region of DMPK. In some embodiments, a single intravenous (IV) injection of 29 mg/kg of a compound disclosed herein (e.g., Compound 1) results in the compound exiting the vasculature of the subject, penetrating into Tibialis anterior (TA) skeletal muscle cell nuclei of the subject, and engaging target mRNA (e.g., HAS, hACTA1, or DMPK) within 24 hours.

Administration (e.g., intravenous, subcutaneous, or intramuscular administration) of a compound disclosed herein (e.g., Compound 1) can induce correction of global exon inclusion levels in transcripts that initially exhibit mis-splicing in HSA^{LR} TA skeletal muscle relative to the FVB background strain mice, as measured by the murine differential splice inclusion (mDSI) statistic. Treatment with a compound disclosed herein (e.g., Compound 1) can also induce rescue of mis-splicing from 100% normalized dysregulation in HSA^{LR} to 54.5% dysregulation of splicing in compound-treated animals with a p-value=1.39×10-44 at 13 days after treatment with a p-value of p=9.278×10\hat{ }-26.

Administration of a compound disclosed herein (e.g., Compound 1) can normalize levels of splicing in 56 unique murine skeletal muscle transcripts that are initially dysregulated (p<0.05) in HSA^{LR} affected mice relative to unaffected background strain mice. In some embodiments, administration of a compound disclosed herein (e.g., Compound 1) normalizes exon usage of an initially dysregulated transcript in a cell exhibiting a nucleotide repeat expansion disorder.

In some embodiments, if, in an assay, (i) a first group of HSA-LR mice are injected with a compound disclosed herein at a dose of 29 mg/kg via tail vein injection (single dose), (ii) a second group of HSA-LR mice are injected with PBS via tail vein injection, (iii) a group of healthy FVB background mice are injected with PBS via tail vein injection, (a) 13 days after injection, tibialis anterior muscle is collected and processed for RNA sequencing, (b) RNA sequencing is performed, (c) reads are aligned to reference genome (GCF_000001635.27 GRC39) with STAR v2.7.1a using the STAR options from rMATS v4.1.0, with reads aligned to the FASTA file using BWA with a clipping penalty of “100,100”, junctions identified by aligning the exclusion isoform against the inclusion isoform, reads overlapping the identified splice junctions counted using sambamba v0.6.6 with the following filters: “mapping_quality>=60 and not (secondary_alignment or failed_quality_control or duplicate or supplementary or chimeric)”, (d) duplicates are removed with Picard MarkDuplicates v2.18.7 (e) aberrant splice activity is measured with rMATS v4.1.0 run on all pairwise combinations of conditions (healthy-untreated, disease-treated, and disease-untreated) with standard options, (f) the top events from the “Skipped Exons Junction Counts Exon Counts” (‘SE.MATS.JCEC.txt’) from the three outputs are then merged together, (g) any event not present in the healthy-untreated vs. disease-treated comparison is dropped, and only those events with a minimum absolute inclusion difference greater than 0.2 are included, (h) differential splice inclusion (DSI) is calculated by first selecting a set of cassette exons with: a) delta-PSI (absolute difference in Percent Splice Inclusion) between healthy-unrelated and disease-untreated samples, as calculated by rMATS, of >=20%; b) p-value of <=0.05 for differential splicing as calculated by rMATS; and c) read depth >=5× in all healthy-untreated, disease-untreated and disease-treated samples, (i) for each cassette exon a scale is calibrated from 0-1, where 0 is the mean PSI of healthy-untreated samples and 1 is the mean PSI of disease-untreated samples, (j) for each sample, the PSI for each cassette exon is normalized into the scale, and then values for all selected cassette exons are averaged to yield DSI, then a statistically significant broad correction across many transcripts is observed for HSALR mice treated with the single dose of the compound (p<0.0001); approximately 75% of mis-spliced transcripts are rescued within 13 days relative to untreated animals that harbor the mutation, which exhibit a set of inappropriately spliced genes that lead to the disease; and/or the chloride channel CLCN1, IR, and other genes exhibit corrected splicing.

In some embodiments, if, in an assay, (i) a first set of cell culture samples are DM1 human patient-derived fibroblast cell line GM03989, (ii) a second set of cell culture samples are normal fibroblast control cell line GM7492, (a) the cells are plated in 24-well plates at 100,000 cells/well in 1 mL of medium and maintained at 37° C. and 5% CO₂ in MEM supplemented with 15% heat inactivated FBS for a day, (b) medium is replaced with MEM with 3% heat-inactivated FBS prior to treatment, (c) stock solutions of a compound of the disclosure are heated at 80° C. for 10 min before use and are added to the cell cultures at a final concentration of 1 μM, (d) cells are incubated for 5 days in the presence or absence of the compound, with all treatments performed in triplicate, (e) total RNA is collected using an RNAeasy kit, (f) libraries for RNA sequencing are created from polyA-selected RNA with the protocol adjusted for 300 ng total RNA, (g) libraries are amplified for 13 cycles and after clean up, concentration and quality are checked using Qubit and Tape station, (h) sequencing is performed with an Illumima NextSeq500, (g) reads are aligned to reference data with STAR v2.7.1a using the STAR options from rMATS v4.1.0, (h) duplicates are removed with Picard MarkDuplicates v2.18.7, (i) aberrant splice activity is measured with rMATS v4.1.0 run on all pairwise combinations of conditions (healthy-untreated, disease-treated, and disease-untreated) with standard options (j) the top events from the “Skipped Exons Junction Counts Exon Counts” (‘SE.MATS.JCEC.txt’) from the three outputs are then merged together, (k) any event not present in the healthy-untreated vs. disease-treated comparison is dropped, and only those events with a minimum absolute inclusion difference greater than 0.2 are included, (1) differential splice inclusion (DSI) is calculated by first selecting a set of cassette exons with: a) delta-PSI (absolute difference in Percent Splice Inclusion) between healthy-unrelated and disease-untreated samples, as calculated by rMATS, of >=20%; b) p-value of <=0.05 for differential splicing as calculated by rMATS; and c) read depth >=5× in all healthy-untreated, disease-untreated and disease-treated samples, (m) for each cassette exon a scale is calibrated from 0-1 where 0 is the mean PSI of healthy-untreated samples and 1 is the mean PSI of disease-untreated samples, (n) for each sample the PSI for each cassette exon is normalized into the scale, and then values for all selected cassette exons are averaged to yield DSI, then a statistically significant broad correction across many transcripts is observed for DM1 cells treated with the compound for 5 days (p<0.0001), approximately 90% of mis-spliced transcripts exhibited corrected splicing for DM1 cells treated with the compound for 5 days, and/or treatment results in rescue of appropriate adult cassette exon use across many mis-spliced transcripts, including those known to rescue the phenotype such as CLCN1, IR, and others.

A compound, composition, or method of the disclosure can reduce myotonia in a subject in need thereof. In some embodiments, a compound, composition, or method of the disclosure can reduce myotonia, for example, a time to relaxation after contraction is induced, by at least about 5%, at least about 10%, at least about 15%, at least about 20%, at least about 25%, at least about 30%, at least about 35%, at least about 40%, at least about 45%, at least about 50%, at least about 55%, at least about 60%, at least about 70%, at least about 80%, at least about 90%, or at least about 95%, e.g., as determined by a muscle relaxation assay, such as a nerve-evoked muscle function assay with a relaxation threshold of about 60%, about 70%, about 80%, or about 90%.

In some embodiments, a compound, composition, or method of the disclosure can reduce myotonia (e.g., a time to relaxation after contraction is induced) in a subject with a trinucleotide repeat expansion to within about 1%, within about 3%, within about 5%, within about 10%, within about 15%, within about 20%, within about 25%, within about 30%, within about 35%, within about 40%, within about 45%, within about 50%, within about 55%, within about 60%, or within about 70% of a level in a control subject that does not have the trinucleotide repeat expansion.

In some embodiments, if, in an assay, (i) a first group of HSA-LR mice are injected a compound disclosed herein at a dose of 3 mg/kg via subcutaneous injection, weekly for four weeks, (ii) a second group of HSA-LR mice are injected with PBS via subcutaneous injection, weekly for four weeks, (iii) a group of healthy FVB background mice are injected with PBS via subcutaneous injection, weekly for four weeks, (a) at 35 days after the first injection, a plantar flexor torque assay is performed; muscle performance is measured using a nerve-evoked muscle function assay that provides a measurement of the specific force of the plantarflexor muscle group, muscle contraction evoked by direct electrical stimulation, (b) myotonia is measured as the delay of muscle relaxation after the maximal isometric tetanic force, (c) muscle performance is measured with a 305C muscle lever system on anesthetized mice, the mice are placed on a thermostatically controlled table and anesthesia maintained via nose-cone (˜3% isoflurane, or to effect), (d) the knee is isolated using a pin pressed against the tibial head and the foot firmly fixed to a footplate on the motor shaft, (e) for the plantar flexor muscle group, contractions are elicited by percutaneous electrical stimulation of the sciatic nerve; no stimulations are performed prior to initiation of the test contractions, (f) optimal stimulus intensity is determined using a wild-type FVB/N mouse, then increased 30%, (g) ten (10) contractions (0.2 ms pulse, 500 ms train duration) are elicited at 30 second intervals and relaxation is recorded for 5 seconds, (h) the force and relaxation curves are normalized to maximal force prior to analysis, then an approximately 70% reduction of myotonia is observed for mice injected with the compound, and/or, a significant reduction in time to relaxation is observed for mice injected with the compound.

In some embodiments, the present disclosure provides a compound comprising: a) a therapeutically-effective warhead region; and b) attached to the therapeutically-effective warhead region, a contiguous sequence of a number of residues of N-acyl-N-(2-aminoethyl)glycine or N-acyl-N-(2-hydroxyethyl)glycine, wherein each residue is independently substituted with a side chain that bears a positive charge at physiological pH, wherein if, in an assay: 1) an experiment is performed using a 100 nM formulation of the compound wherein the number of peptide nucleic acid residues in the contiguous sequence is 3, and in the experiment: i) 3989-DM1 fibroblast cells are treated for five days with the formulation of the compound; ii) at conclusion of the five days, total cellular RNA is extracted from the DM1 fibroblast cells; iii) the total cellular RNA that was extracted from the DM1 fibroblast cells is used as a template for cDNA synthesis by real-time quantitative reverse transcription PCR (qRT-PCR) using a primer for dystrophia myotonica protein kinase (DMPK) gene synthesis, thereby synthesizing cDNA containing the DMPK gene; and iv) DMPK induction by the cDNA that was synthesized is quantified by delta-delta-Ct using untreated DM1 fibroblast cells as a control with expression normalized to glyceraldehyde 3-phosphate dehydrogenase (GADPH) expression; 2) the experiment is repeated using a 300 nM formulation of the compound wherein the number of peptide nucleic acid residues in the contiguous sequence is 3; 3) the experiment is repeated using a 1,000 nM formulation of the compound wherein the number of peptide nucleic acid residues in the contiguous sequence is 3; 4) the experiment is repeated using a 100 nM formulation of the compound wherein the number of peptide nucleic acid residues in the contiguous sequence is 4; 5) the experiment is repeated using a 300 nM formulation of the compound wherein the number of peptide nucleic acid residues in the contiguous sequence is 4; 6) the experiment is repeated using a 1,000 nM formulation of the compound wherein the number of peptide nucleic acid residues in the contiguous sequence is 4; 7) the experiment is repeated using a 100 nM formulation of the compound wherein the number of peptide nucleic acid residues in the contiguous sequence is 5; 8) the experiment is repeated using a 300 nM formulation of the compound wherein the number of peptide nucleic acid residues in the contiguous sequence is 5; 9) the experiment is repeated using a 1,000 nM formulation of the compound wherein the number of peptide nucleic acid residues in the contiguous sequence is 5; 10) the experiment is repeated using a 100 nM formulation of the compound wherein the number of peptide nucleic acid residues in the contiguous sequence is 6; 11) the experiment is repeated using a 300 nM formulation of the compound wherein the number of peptide nucleic acid residues in the contiguous sequence is 6; 12) the experiment is repeated using a 1,000 nM formulation of the compound wherein the number of peptide nucleic acid residues in the contiguous sequence is 6; 13) the experiment is repeated using a 100 nM formulation of the compound wherein the number of peptide nucleic acid residues in the contiguous sequence is 7; 14) the experiment is repeated using a 300 nM formulation of the compound wherein the number of peptide nucleic acid residues in the contiguous sequence is 7; and 15) the experiment is repeated using a 1,000 nM formulation of the compound wherein the number of peptide nucleic acid residues in the contiguous sequence is 7; then: A) when the number of peptide nucleic acid residues in the contiguous sequence is 7, a fold change from the control for DMPK induction is lesser than 0.6 for each of the 100 nm formulation, the 300 nm formulation, and the 1,000 nm formulation; B) when the number of peptide nucleic acid residues in the contiguous sequence is 6, a fold change from the control for DMPK induction is lesser than 0.6 for each of the 100 nm formulation, the 300 nm formulation, and the 1,000 nm formulation; C) when the number of peptide nucleic acid residues in the contiguous sequence is 5, a fold change from the control for DMPK induction is lesser than 0.8 for the 100 nm formulation and lesser than 0.6 for each of the 300 nm formulation and the 1,000 formulation; D) when the number of peptide nucleic acid residues in the contiguous sequence is 4, a fold change from the control for DMPK induction is up to 1 for the 100 nm formulation, lesser than 0.8 for the 300 nm formulation, and lesser than 0.6 for the 1,000 formulation; and E) when the number of peptide nucleic acid residues in the contiguous sequence is 3, a fold change from the control for DMPK induction is up to 1 for the 100 nm formulation, lesser than 0.8 for the 300 nm formulation, and lesser than 0.6 for the 1,000 formulation.

In some embodiments, the therapeutically-effective warhead region and the contiguous sequence of the number of peptide nucleic acid residues form:

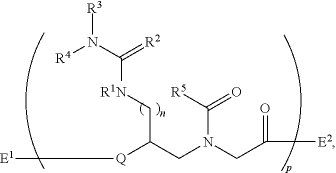

wherein: R¹ is H, alkyl, or a nitrogen atom protecting group; R² is O, NH, N(alkyl), or N(Pg^{N}), wherein Pg^{N} is a nitrogen atom protecting group; R³ is H, alkyl, or a nitrogen atom protecting group; R⁴ is H, alkyl, or a nitrogen atom protecting group; R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; Q is O, NH, N(alkyl), or N(Pg^{N}); n is 1, 2, 3, 4, 5, 6, 7, 8, 9, or 10; p is the number of peptide nucleic acid residues in the contiguous sequence; and E¹ is hydrogen, acyl, a group that together with the nitrogen atom to which E¹ is bound forms a carbamate, a peptide sequence, a linker, a probe, a metal chelator, or an imaging agent, and E² is the therapeutically-effective warhead region; or E¹ is the therapeutically-effective warhead region, and E² is OH, O-alkyl, NH₂, a peptide sequence, a linker, a probe, a metal chelator, or an imaging agent, or a pharmaceutically-acceptable salt or ionized form thereof. In some embodiments, each of R¹, R³, and R⁴ is hydrogen. In some embodiments, R² is NH or N(Pg^{N}). In some embodiments, each of R¹, R³, and R⁴ is hydrogen; and R² is NH or N(Pg^{N}). In some embodiments, R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, or heterocyclyl. In some embodiments, R⁵ is linear alkyl, branched alkyl, or cyclic alkyl. In some embodiments, R⁵ is linear alkyl. In some embodiments, R⁵ is methyl. In some embodiments, Q is NH or N(Pg^{N}). In some embodiments, n is 3. In some embodiments, n is 4. In some embodiments, p is 3, 4, 5, 6, or 7. In some embodiments, E¹ is the therapeutically-effective warhead region, and E² is OH, OMe, NH₂, a peptide sequence, a linker, a probe, a metal chelator, or an imaging agent. In some embodiments, E² is OH or NH₂. In some embodiments, E¹ is hydrogen, acyl, a group that together with the nitrogen atom to which E¹ is bound forms a carbamate, a peptide sequence, a linker, a probe, a metal chelator, or an imaging agent, and E² is the therapeutically-effective warhead region. In some embodiments, E¹ is hydrogen.

In some embodiments, the therapeutically-effective warhead region and the contiguous sequence of the number of peptide nucleic acid residues form:

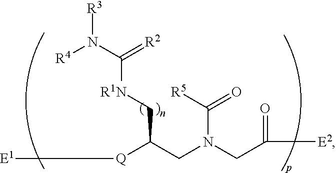

wherein: R¹ is H, alkyl, or a nitrogen atom protecting group; R² is O, NH, N(alkyl), or N(Pg^{N}), wherein Pg^{N} is a nitrogen atom protecting group; R³ is H, alkyl, or a nitrogen atom protecting group; R⁴ is H, alkyl, or a nitrogen atom protecting group; R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; Q is O, NH, N(alkyl), or N(Pg^{N}); n is 1, 2, 3, 4, 5, 6, 7, 8, 9, or 10; p is the number of peptide nucleic acid residues in the contiguous sequence; and E¹ is hydrogen, acyl, a group that together with the nitrogen atom to which E¹ is bound forms a carbamate, a peptide sequence, a linker, a probe, a metal chelator, or an imaging agent, and E² is the therapeutically-effective warhead region; or E¹ is the therapeutically-effective warhead region, and E² is OH, O-alkyl, NH₂, a peptide sequence, a linker, a probe, a metal chelator, or an imaging agent, or a pharmaceutically-acceptable salt or ionized form thereof. In some embodiments, each of R¹, R³, and R⁴ is hydrogen. In some embodiments, R² is NH or N(Pg^{N}). In some embodiments, each of R¹, R³, and R⁴ is hydrogen; and R² is NH or N(Pg^{N}). In some embodiments, R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, or heterocyclyl. In some embodiments, R⁵ is linear alkyl, branched alkyl, or cyclic alkyl. In some embodiments, R⁵ is linear alkyl. In some embodiments, R⁵ is methyl. In some embodiments, Q is NH or N(Pg^{N}). In some embodiments, n is 3. In some embodiments, n is 4. In some embodiments, p is 3, 4, 5, 6, or 7. In some embodiments, E¹ is the therapeutically-effective warhead region, and E² is OH, OMe, NH₂, a peptide sequence, a linker, a probe, a metal chelator, or an imaging agent. In some embodiments, E² is OH or NH₂. In some embodiments, E¹ is hydrogen, acyl, a group that together with the nitrogen atom to which E¹ is bound forms a carbamate, a peptide sequence, a linker, a probe, a metal chelator, or an imaging agent, and E² is the therapeutically-effective warhead region. In some embodiments, E¹ is hydrogen.

In some embodiments, the therapeutically-effective warhead region comprises a structure that interferes with expression of a gene associated with a neuromuscular disease phenotype. In some embodiments, the neuromuscular disease phenotype is a DM1 disease phenotype. In some embodiments, the therapeutically-effective warhead region is an oligonucleotide or oligonucleotide analogue. In some embodiments, the therapeutically-effective warhead region is a peptide nucleic acid. In some embodiments, the therapeutically-effective warhead region binds to a mRNA sequence transcribed from a gene associated with a DM1 disease phenotype. In some embodiments, the DM1 gene is a non-wild type DM1 gene. In some embodiments, the non-wild type DM1 gene differs from a wild type DM1 gene in a repeat expansion mutation. In some embodiments, the therapeutically-effective warhead region binds to a mRNA sequence that contains a subsequence that is (CUG)_{z}, wherein z is an integer from 1-100 (SEQ ID NO: 135). In some embodiments, the therapeutically-effective warhead region binds to the mRNA sequence at the subsequence that is (CUG)_{z}.

### Modes of Administration

A compound provided herein or a composition comprising a compound provided herein (e.g., a pharmaceutical composition) can be administered to a subject in various forms and by various suitable routes of administration.

A compound provided herein or a composition comprising a compound provided herein (e.g., a pharmaceutical composition) can be administered in a local manner, for example, via injection of the compound directly into an organ, optionally in a depot or sustained release formulation or implant. A compound provided herein or a composition comprising a compound provided herein (e.g., a pharmaceutical composition) can be administered in a systemic manner.

In some embodiments, a compound provided herein or a composition comprising a compound provided herein (e.g., a pharmaceutical composition) is administered parenterally. Parenteral administration can be, for example, by bolus injection or by gradual infusion or perfusion over time. Administration can also be by surgical deposition of a bolus or positioning of a medical device.

In some embodiments, a compound provided herein or a composition comprising a compound provided herein (e.g., a pharmaceutical composition) is administered orally. In some embodiments, a compound provided herein or a composition comprising a compound provided herein (e.g., a pharmaceutical composition) is administered by an intravenous, intratumoral, subcutaneous, intramuscular, intracerebral, intracerebroventricular, intra-articular, intraperitoneal, intracranial, intrathecal, intranasal, buccal, sublingual, oral, or rectal administration route. In some embodiments, a compound provided herein or a composition comprising a compound provided herein (e.g., a pharmaceutical composition) is administered by intravenous administration. In some embodiments, a compound provided herein or a composition comprising a compound provided herein (e.g., a pharmaceutical composition) is administered by subcutaneous administration. In some embodiments, a compound provided herein or a composition comprising a compound provided herein (e.g., a pharmaceutical composition) is administered by intramuscular administration. In some embodiments, a compound provided herein or a composition comprising a compound provided herein (e.g., a pharmaceutical composition) is administered by intracerebroventricular administration. In some embodiments, a compound provided herein or a composition comprising a compound provided herein (e.g., a pharmaceutical composition) is administered by oral administration. In some embodiments, a compound provided herein or a composition comprising a compound provided herein (e.g., a pharmaceutical composition) is administered by intrathecal administration.

Any aforementioned route of administration can be combined with another route of administration. For example, a compound provided herein can be delivered by a first route of administration, and one or more subsequent maintenance doses of the compound can be delivered by the same or a different route of administration. In some embodiments, a compound provided herein or a composition comprising a compound provided herein (e.g., a pharmaceutical composition) is administered by intramuscular administration, and one or more subsequent maintenance doses of the compound or the composition comprising the compound are delivered by subcutaneous administration or intravenous administration.

Non-limiting examples of suitable modes and routes of administration include oral, topical, parenteral, intravenous injection, intravenous infusion, subcutaneous injection, subcutaneous infusion, intramuscular injection, intramuscular infusion, intradermal injection, intradermal infusion, intraperitoneal injection, intraperitoneal infusion, intracerebral injection, intracerebral infusion, subarachnoid injection, subarachnoid infusion, intraocular injection, intraspinal injection, intrasternal injection, ophthalmic administration, endothelial administration, local administration, intranasal administration, intrapulmonary administration, rectal administration, intraarterial administration, intrathecal administration, inhalation, intralesional administration, intradermal administration, transdermal administration (e.g., via emulsion/liposome-mediated methods of delivery with the compound optionally packaged into liposomes), epidural administration, absorption through epithelial or mucocutaneous linings (e.g., oral mucosa, rectal and intestinal mucosa), intracapsular administration, subcapsular administration, intracardiac administration, transtracheal administration, subcuticular administration, subarachnoid administration, subcapsular administration, intraspinal administration, and intrasternal administration.

A compound provided herein or a composition comprising a compound provided herein (e.g., a pharmaceutical composition) can be administered via a non-invasive method. Examples of non-invasive modes of administering can include using a needleless injection device, and topical administration, e.g., eye drops. Multiple administration routes can be employed for efficient delivery.

Depending on the intended mode of administration, the compositions can be in the form of solid, semi solid or liquid dosage forms, such as, e.g., tablets, suppositories, pills, capsules, powders, liquids, suspensions, lotions, creams, or gels, e.g., in unit dosage form suitable for single administration of a precise dosage. The composition can be formulated into any suitable dosage form for administration, e.g., aqueous dispersions, liquids, gels, syrups, elixirs, slurries, and suspensions, for administration to a subject or a patient.

Solid compositions include, e.g., powders, tablets, dispersible granules, capsules, and cachets. Liquid compositions include, e.g., solutions in which a compound is dissolved, emulsions comprising a compound, or a solution containing liposomes, micelles, or nanoparticles comprising a compound as disclosed herein. Semi-solid compositions include, e.g., gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

In some embodiments, the composition is formulated into solutions (e.g., for IV administration). In some cases, the pharmaceutical composition is formulated as an infusion. In some cases, the pharmaceutical composition is formulated as an injection.

A compound provided herein or a composition comprising a compound provided herein (e.g., a pharmaceutical composition) can be administered in the form of a rapid release formulation, in the form of an extended release formulation, or in the form of an intermediate release formulation. A rapid release form can provide an immediate release. An extended release formulation can provide a controlled release or a sustained delayed release.

A composition comprising a compound provided herein can be, e.g., an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements, or has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some embodiments, a controlled release formulation is a delayed release form. A delayed release form can be formulated to delay a compound's action for an extended period of time. A delayed release form can be formulated to delay the release of an effective dose of one or more compounds, e.g., for about 4, about 8, about 12, about 16, or about 24 hours. A controlled release formulation can be a sustained release form. A sustained release form can be formulated to sustain, e.g., the compound's action over an extended period of time. A sustained release form can be formulated to provide an effective dose of any compound described herein (e.g., provide a physiologically-effective blood profile) over about 4, about 8, about 12, about 16, or about 24 hours.

A pharmaceutical composition disclosed herein can be targeted to any suitable tissue or cell type. Modes, routes, and compositions provided herein can be suitable to target a compound provided herein to a particular tissue, or a subset of tissues. Non-limiting examples of tissues that can be targeted include kidney (e.g., kidney cortex), joints, cartilage, liver, salivary glands, bone (e.g., bone surface), skin, lung, muscle, pancreas, hair follicles, large intestine mucosa, aortic wall, small intestine mucosa, adrenal gland, stomach mucosa, spleen, bone marrow, lymph nodes, thymus, brain, cerebellum, olfactory bulb, thalamus, caudate putamen, cerebral cortex, substantia nigra, lateral ventricle, choroid plexus, and combinations thereof.

Compounds can be introduced into cells by, e.g., transfection, electroporation, fusion, liposomes, colloidal polymeric particles, and viral and non-viral vectors. Compounds provided herein can also be delivered using, e.g., methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery.

The method of delivery can depend at least on the cells to be treated and the location of the cells. For instance, localization can be achieved by liposomes with specific markers on the surface to direct the liposome, direct injection into tissue containing target cells, specific receptor mediated uptake, or viral vectors.

In some embodiments, a compound disclosed herein is delivered via an implantable device, e.g., synthetic implant design.

Compounds provided herein can be administered in any physiologically and/or pharmaceutically acceptable vehicle or carrier. Non-limiting examples of pharmaceutically acceptable carriers include saline, phosphate buffered saline (PBS), water, aqueous ethanol, emulsions, such as oil/water emulsions or triglyceride emulsions, tablets, and capsules. The choice of suitable physiologically acceptable carrier can vary depending upon the chosen mode of administration. A pharmaceutically acceptable carrier can include solvents, dispersion media, coatings, antibacterial and antifungal agents, isotonic and absorption delaying agents, and the like, compatible with pharmaceutical administration.

Further provided are prodrugs of a compound provided herein. Prodrugs can be covalently bonded carriers that release a compound in vivo when administered to a subject. Prodrugs can be prepared by modifying functional groups in a way such that the modification is cleaved, either by routine manipulation or in vivo, to yield the biologically active compound. Non-limiting examples of prodrugs include acetate, formate, and benzoate derivatives of alcohol and amine functional groups of compounds provided herein. Further, in the case of a carboxylic acid functional group (—COOH), esters can be used, such as methyl esters and ethyl esters.

In some embodiments, liposomes can be used to facilitate uptake of a compound provided herein into cells. Hydrogels can also be used as vehicles for compound administration. Alternatively, a compound provided herein can be administered in microspheres or microparticles. Alternatively, the use of gas-filled microbubbles complexed with a compound provided herein can enhance delivery to target tissues. Sustained release compositions can also be used, including, e.g., semipermeable polymeric matrices in the form of shaped articles such as films or microcapsules.

In some embodiments, a compound provided herein is administered to a mammalian subject, e.g., human or domestic animal that is exhibiting the symptoms of a polynucleotide repeat expansion disorder. Compounds provided herein can selectively reduce expression of a mutant protein in the subject. In some embodiments, the subject is a human subject, e.g., a patient diagnosed as having a polynucleotide repeat disease. In some embodiments, a compound provided herein is contained in a pharmaceutically acceptable carrier and is delivered orally. In some embodiments, a compound provided herein is contained in a pharmaceutically acceptable carrier and is delivered intravenously.

In some embodiments, the subject is a vertebrate. In some embodiments, the subject is a mammal. In some embodiments, the subject is a human. In some embodiments, the subject is a primate, ape, monkey, sheep, equine, bovine, porcine, minipig, canine, feline, goat, camelid, rodent, rabbit, mouse, rat, hamster, gerbil, hamster, chinchilla, fancy rat, guinea pig, C57BL6J mouse, Beagle dog, Gottingen minipig, or Cynomolgus monkey. In some embodiments, a subject is a non-human subject. In some embodiments, a subject is a veterinary subject.

In some embodiments, the patient is a vertebrate. In some embodiments, the patient is a mammal. In some embodiments, the patient is a human. In some embodiments, the patient is a primate, ape, monkey, sheep, equine, bovine, porcine, minipig, canine, feline, goat, camelid, rodent, rabbit, mouse, rat, hamster, gerbil, hamster, chinchilla, fancy rat, guinea pig, C57BL6J mouse, Beagle dog, Gottingen minipig, or Cynomolgus monkey. In some embodiments, a patient is a non-human patient. In some embodiments, a patient is a veterinary patient.

In some embodiments, a patient and a subject are the same species. In some embodiments, a subject and a patient are human.

In some embodiments, a patient and a subject are different species. In some embodiments, a subject is human and a patient is a non-human, for example, a non-human vertebrate, non-human mammal, non-human primate, ape, monkey, sheep, equine, bovine, porcine, minipig, canine, feline, goat, camelid, rodent, rabbit, mouse, rat, hamster, gerbil, hamster, chinchilla, fancy rat, or guinea pig. In some embodiments, a patient is human and a subject is a non-human, for example, a non-human vertebrate, non-human mammal, non-human primate, ape, monkey, sheep, equine, bovine, porcine, minipig, canine, feline, goat, camelid, rodent, rabbit, mouse, rat, hamster, gerbil, hamster, chinchilla, fancy rat, or guinea pig.

An effective in vivo treatment regimen using the compounds provided herein can vary according to the duration, dose, frequency, and route of administration, as well as the condition of the subject under treatment (i.e., prophylactic administration versus administration in response to localized or systemic infection). Accordingly, such in vivo therapy can require monitoring by tests appropriate to the particular type of disorder under treatment, and corresponding adjustments in the dose or treatment regimen, in order to achieve an optimal therapeutic outcome.

The efficacy of an in vivo administered compound provided herein can be determined from biological samples (e.g., tissue, blood, urine) taken from a subject prior to, during, and subsequent to administration of the compound. Assays of such samples can include (1) monitoring the presence or absence of heteroduplex formation with target and non-target sequences, e.g., by an electrophoretic gel mobility assay; and (2) monitoring the amount of a mutant mRNA or protein in relation to a reference wild-type mRNA or protein as determined by standard techniques such as RT-PCR, Northern blotting, ELISA, or Western blotting.

In some embodiments, the compound provided herein is actively taken up by mammalian cells. In further embodiments, the compound provided herein can be conjugated to a transport moiety (e.g., transport peptide) as described herein to facilitate such uptake.

Compounds provided herein can be administered to subjects to treat (prophylactically or therapeutically) disorders associated with aberrant expression of a mRNA or protein produced from a mutant polynucleotide repeat containing allele. In conjunction with such treatment, pharmacogenomics (i.e., the study of the relationship between an individual's genotype and the individual's response to a foreign compound or drug) can be considered. Differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a therapeutic agent as well as tailoring the dosage and/or therapeutic regimen of treatment with the therapeutic agent.

### Dosing

Pharmaceutical compositions described herein can be in unit dosage forms suitable for single administration of precise dosages. In unit dosage form, the formulation is divided into unit doses containing appropriate quantities of one or more compound. The dosage (e.g., therapeutically-effective amount) for a compound described herein can be in any amount necessary.

A compound described herein can be present in a composition or a unit dose in a range of from about 1 mg to about 2000 mg; from about 5 mg to about 1000 mg, from about 10 mg to about 25 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition or a unit dose in a range of from about 1 μg to about 2000 μg; from about 5 μg to about 1000 μg, from about 10 μg to about 25 μg, from about 50 μg to about 250 μg, from about 100 μg to about 200 μg, from about 1 μg to about 50 μg, from about 50 μg to about 100 μg, from about 100 μg to about 150 μg, from about 150 μg to about 200 μg, from about 200 μg to about 250 μg, from about 250 μg to about 300 μg, from about 300 μg to about 350 μg, from about 350 μg to about 400 μg, from about 400 μg to about 450 μg, from about 450 μg to about 500 μg, from about 500 μg to about 550 μg, from about 550 μs to about 600 μg, from about 600 μg to about 650 μg, from about 650 μg to about 700 μg, from about 700 μg to about 750 μg, from about 750 μg to about 800 μg, from about 800 μg to about 850 μg, from about 850 μg to about 900 μg, from about 900 μg to about 950 μg, or from about 950 μg to about 1000 μg.

A compound described herein can be present in a composition or a unit dose in an amount of about 0.001 mg, about 0.002 mg, about 0.003 mg, about 0.004 mg, about 0.005 mg, about 0.006 mg, about 0.007 mg, about 0.008 mg, about 0.009 mg, about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a composition is present in a composition or a unit dose in an amount that is at least about 0.001 mg, at least about 0.002 mg, at least about 0.003 mg, at least about 0.004 mg, at least about 0.005 mg, at least about 0.006 mg, at least about 0.007 mg, at least about 0.008 mg, at least about 0.009 mg, at least about 0.01 mg, at least about 0.02 mg, at least about 0.03 mg, at least about 0.04 mg, at least about 0.05 mg, at least about 0.06 mg, at least about 0.07 mg, at least about 0.08 mg, at least about 0.09 mg, at least about 0.1 mg, at least about 0.2 mg, at least about 0.3 mg, at least about 0.4 mg, at least about 0.5 mg, at least about 0.6 mg, at least about 0.7 mg, at least about 0.8 mg, at least about 0.9 mg, at least about 1 mg, at least about 2 mg, at least about 3 mg, at least about 4 mg, at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 125 mg, at least about 150 mg, at least about 175 mg, at least about 200 mg, at least about 250 mg, at least about 300 mg, at least about 350 mg, at least about 400 mg, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, at least about 1000 mg, at least about 1050 mg, at least about 1100 mg, at least about 1150 mg, at least about 1200 mg, at least about 1250 mg, at least about 1300 mg, at least about 1350 mg, at least about 1400 mg, at least about 1450 mg, at least about 1500 mg, at least about 1550 mg, at least about 1600 mg, at least about 1650 mg, at least about 1700 mg, at least about 1750 mg, at least about 1800 mg, at least about 1850 mg, at least about 1900 mg, at least about 1950 mg, or at least about 2000 mg.

In some embodiments, a composition is present in a composition or a unit dose in an amount that is at most about 0.001 mg, at most about 0.002 mg, at most about 0.003 mg, at most about 0.004 mg, at most about 0.005 mg, at most about 0.006 mg, at most about 0.007 mg, at most about 0.008 mg, at most about 0.009 mg, at most about 0.01 mg, at most about 0.02 mg, at most about 0.03 mg, at most about 0.04 mg, at most about 0.05 mg, at most about 0.06 mg, at most about 0.07 mg, at most about 0.08 mg, at most about 0.09 mg, at most about 0.1 mg, at most about 0.2 mg, at most about 0.3 mg, at most about 0.4 mg, at most about 0.5 mg, at most about 0.6 mg, at most about 0.7 mg, at most about 0.8 mg, at most about 0.9 mg, at most about 1 mg, at most about 2 mg, at most about 3 mg, at most about 4 mg, at most about 5 mg, at most about 10 mg, at most about 15 mg, at most about 20 mg, at most about 25 mg, at most about 30 mg, at most about 35 mg, at most about 40 mg, at most about 45 mg, at most about 50 mg, at most about 55 mg, at most about 60 mg, at most about 65 mg, at most about 70 mg, at most about 75 mg, at most about 80 mg, at most about 85 mg, at most about 90 mg, at most about 95 mg, at most about 100 mg, at most about 125 mg, at most about 150 mg, at most about 175 mg, at most about 200 mg, at most about 250 mg, at most about 300 mg, at most about 350 mg, at most about 400 mg, at most about 450 mg, at most about 500 mg, at most about 550 mg, at most about 600 mg, at most about 650 mg, at most about 700 mg, at most about 750 mg, at most about 800 mg, at most about 850 mg, at most about 900 mg, at most about 950 mg, at most about 1000 mg, at most about 1050 mg, at most about 1100 mg, at most about 1150 mg, at most about 1200 mg, at most about 1250 mg, at most about 1300 mg, at most about 1350 mg, at most about 1400 mg, at most about 1450 mg, at most about 1500 mg, at most about 1550 mg, at most about 1600 mg, at most about 1650 mg, at most about 1700 mg, at most about 1750 mg, at most about 1800 mg, at most about 1850 mg, at most about 1900 mg, at most about 1950 mg, or at most about 2000 mg.

In some embodiments, a dose (e.g., a unit dose) is about 0.001 mg/kg, about 0.002 mg/kg, about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 125 mg/kg, about 150 mg/kg, about 175 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, about 1000 mg/kg, about 1050 mg/kg, about 1100 mg/kg, about 1150 mg/kg, about 1200 mg/kg, about 1250 mg/kg, about 1300 mg/kg, about 1350 mg/kg, about 1400 mg/kg, about 1450 mg/kg, about 1500 mg/kg, about 1550 mg/kg, about 1600 mg/kg, about 1650 mg/kg, about 1700 mg/kg, about 1750 mg/kg, about 1800 mg/kg, about 1850 mg/kg, about 1900 mg/kg, about 1950 mg/kg, or about 2000 mg/kg based on body mass of a subject or a patient.

In some embodiments, a dose (e.g., a unit dose) is at least about 0.001 mg/kg, at least about 0.002 mg/kg, at least about 0.003 mg/kg, at least about 0.004 mg/kg, at least about 0.005 mg/kg, at least about 0.006 mg/kg, at least about 0.007 mg/kg, at least about 0.008 mg/kg, at least about 0.009 mg/kg, at least about 0.01 mg/kg, at least about 0.02 mg/kg, at least about 0.03 mg/kg, at least about 0.04 mg/kg, at least about 0.05 mg/kg, at least about 0.06 mg/kg, at least about 0.07 mg/kg, at least about 0.08 mg/kg, at least about 0.09 mg/kg, at least about 0.1 mg/kg, at least about 0.2 mg/kg, at least about 0.3 mg/kg, at least about 0.4 mg/kg, at least about 0.5 mg/kg, at least about 0.6 mg/kg, at least about 0.7 mg/kg, at least about 0.8 mg/kg, at least about 0.9 mg/kg, at least about 1 mg/kg, at least about 2 mg/kg, at least about 3 mg/kg, at least about 4 mg/kg, at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, at least about 100 mg/kg, at least about 125 mg/kg, at least about 150 mg/kg, at least about 175 mg/kg, at least about 200 mg/kg, at least about 250 mg/kg, at least about 300 mg/kg, at least about 350 mg/kg, at least about 400 mg/kg, at least about 450 mg/kg, at least about 500 mg/kg, at least about 550 mg/kg, at least about 600 mg/kg, at least about 650 mg/kg, at least about 700 mg/kg, at least about 750 mg/kg, at least about 800 mg/kg, at least about 850 mg/kg, at least about 900 mg/kg, at least about 950 mg/kg, at least about 1000 mg/kg, at least about 1050 mg/kg, at least about 1100 mg/kg, at least about 1150 mg/kg, at least about 1200 mg/kg, at least about 1250 mg/kg, at least about 1300 mg/kg, at least about 1350 mg/kg, at least about 1400 mg/kg, at least about 1450 mg/kg, at least about 1500 mg/kg, at least about 1550 mg/kg, at least about 1600 mg/kg, at least about 1650 mg/kg, at least about 1700 mg/kg, at least about 1750 mg/kg, at least about 1800 mg/kg, at least about 1850 mg/kg, at least about 1900 mg/kg, at least about 1950 mg/kg, or at least about 2000 mg/kg based on body mass of a subject or a patient.

In some embodiments, a dose (e.g., a unit dose) is at most about 0.001 mg/kg, at most about 0.002 mg/kg, at most about 0.003 mg/kg, at most about 0.004 mg/kg, at most about 0.005 mg/kg, at most about 0.006 mg/kg, at most about 0.007 mg/kg, at most about 0.008 mg/kg, at most about 0.009 mg/kg, at most about 0.01 mg/kg, at most about 0.02 mg/kg, at most about 0.03 mg/kg, at most about 0.04 mg/kg, at most about 0.05 mg/kg, at most about 0.06 mg/kg, at most about 0.07 mg/kg, at most about 0.08 mg/kg, at most about 0.09 mg/kg, at most about 0.1 mg/kg, at most about 0.2 mg/kg, at most about 0.3 mg/kg, at most about 0.4 mg/kg, at most about 0.5 mg/kg, at most about 0.6 mg/kg, at most about 0.7 mg/kg, at most about 0.8 mg/kg, at most about 0.9 mg/kg, at most about 1 mg/kg, at most about 2 mg/kg, at most about 3 mg/kg, at most about 4 mg/kg, at most about 5 mg/kg, at most about 10 mg/kg, at most about 15 mg/kg, at most about 20 mg/kg, at most about 25 mg/kg, at most about 30 mg/kg, at most about 35 mg/kg, at most about 40 mg/kg, at most about 45 mg/kg, at most about 50 mg/kg, at most about 55 mg/kg, at most about 60 mg/kg, at most about 65 mg/kg, at most about 70 mg/kg, at most about 75 mg/kg, at most about 80 mg/kg, at most about 85 mg/kg, at most about 90 mg/kg, at most about 95 mg/kg, at most about 100 mg/kg, at most about 125 mg/kg, at most about 150 mg/kg, at most about 175 mg/kg, at most about 200 mg/kg, at most about 250 mg/kg, at most about 300 mg/kg, at most about 350 mg/kg, at most about 400 mg/kg, at most about 450 mg/kg, at most about 500 mg/kg, at most about 550 mg/kg, at most about 600 mg/kg, at most about 650 mg/kg, at most about 700 mg/kg, at most about 750 mg/kg, at most about 800 mg/kg, at most about 850 mg/kg, at most about 900 mg/kg, at most about 950 mg/kg, at most about 1000 mg/kg, at most about 1050 mg/kg, at most about 1100 mg/kg, at most about 1150 mg/kg, at most about 1200 mg/kg, at most about 1250 mg/kg, at most about 1300 mg/kg, at most about 1350 mg/kg, at most about 1400 mg/kg, at most about 1450 mg/kg, at most about 1500 mg/kg, at most about 1550 mg/kg, at most about 1600 mg/kg, at most about 1650 mg/kg, at most about 1700 mg/kg, at most about 1750 mg/kg, at most about 1800 mg/kg, at most about 1850 mg/kg, at most about 1900 mg/kg, at most about 1950 mg/kg, or at most about 2000 mg/kg based on body mass of a subject or a patient.

In some embodiments, a dose (e.g., a unit dose) is from about 0.1 mg/kg to about 2000 mg/kg, from about 1 mg/kg to about 2000 mg/kg, from about 5 mg/kg to about 1000 mg/kg, from about 10 mg/kg to about 25 mg/kg, from about 50 mg/kg to about 250 mg/kg, from about 100 mg/kg to about 200 mg/kg, from about 1 mg/kg to about 50 mg/kg, from about 50 mg/kg to about 100 mg/kg, from about 100 mg/kg to about 150 mg/kg, from about 150 mg/kg to about 200 mg/kg, from about 200 mg/kg to about 250 mg/kg, from about 250 mg/kg to about 300 mg/kg, from about 300 mg/kg to about 350 mg/kg, from about 350 mg/kg to about 400 mg/kg, from about 400 mg/kg to about 450 mg/kg, from about 450 mg/kg to about 500 mg/kg, from about 500 mg/kg to about 550 mg/kg, from about 550 mg/kg to about 600 mg/kg, from about 600 mg/kg to about 650 mg/kg, from about 650 mg/kg to about 700 mg/kg, from about 700 mg/kg to about 750 mg/kg, from about 750 mg/kg to about 800 mg/kg, from about 800 mg/kg to about 850 mg/kg, from about 850 mg/kg to about 900 mg/kg, from about 900 mg/kg to about 950 mg/kg, from about 950 mg/kg to about 1000 mg/kg, about 1 μg/kg to about 2000 ng/kg; from about 5 μg/kg to about 1000 ng/kg, from about 10 ng/kg to about 25 ng/kg, from about 50 ng/kg to about 250 ng/kg, from about 100 ng/kg to about 200 ng/kg, from about 1 μg/kg to about 50 ng/kg, from about 50 ng/kg to about 100 ng/kg, from about 100 ng/kg to about 150 ng/kg, from about 150 ng/kg to about 200 ng/kg, from about 200 ng/kg to about 250 ng/kg, from about 250 ng/kg to about 300 ng/kg, from about 300 ng/kg to about 350 ng/kg, from about 350 ng/kg to about 400 ng/kg, from about 400 ng/kg to about 450 ng/kg, from about 450 ng/kg to about 500 ng/kg, from about 500 ng/kg to about 550 ng/kg, from about 550 ng/kg to about 600 ng/kg, from about 600 ng/kg to about 650 ng/kg, from about 650 ng/kg to about 700 ng/kg, from about 700 ng/kg to about 750 ng/kg, from about 750 ng/kg to about 800 ng/kg, from about 800 ng/kg to about 850 ng/kg, from about 850 ng/kg to about 900 ng/kg, from about 900 ng/kg to about 950 ng/kg, or from about 950 ng/kg to about 1000 ng/kg based on body mass of a subject or a patient.

Pharmaceutical compositions and formulations described herein can comprise, for example, a compound provided herein at any suitable concentration. A formulation can comprise a composition provided herein at a concentration of, for example, about 0.001 mg/mL, about 0.002 mg/mL, about 0.003 mg/mL, about 0.004 mg/mL, about 0.005 mg/mL, about 0.006 mg/mL, about 0.007 mg/mL, about 0.008 mg/mL, about 0.009 mg/mL, about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 2 mg/mL, about 3 mg/mL, about 4 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 125 mg/mL, about 150 mg/mL, about 175 mg/mL, about 200 mg/mL, about 250 mg/mL, about 300 mg/mL, about 350 mg/mL, about 400 mg/mL, about 450 mg/mL, about 500 mg/mL, about 550 mg/mL, about 600 mg/mL, about 650 mg/mL, about 700 mg/mL, about 750 mg/mL, about 800 mg/mL, about 850 mg/mL, about 900 mg/mL, about 950 mg/mL, about 1000 mg/mL, about 1050 mg/mL, about 1100 mg/mL, about 1150 mg/mL, about 1200 mg/mL, about 1250 mg/mL, about 1300 mg/mL, about 1350 mg/mL, about 1400 mg/mL, about 1450 mg/mL, about 1500 mg/mL, about 1550 mg/mL, about 1600 mg/mL, about 1650 mg/mL, about 1700 mg/mL, about 1750 mg/mL, about 1800 mg/mL, about 1850 mg/mL, about 1900 mg/mL, about 1950 mg/mL, or about 2000 mg/mL.

In some embodiments, a formulation provided herein comprises a compound provided herein at a concentration of at least about 0.001 mg/mL, at least about 0.002 mg/mL, at least about 0.003 mg/mL, at least about 0.004 mg/mL, at least about 0.005 mg/mL, at least about 0.006 mg/mL, at least about 0.007 mg/mL, at least about 0.008 mg/mL, at least about 0.009 mg/mL, at least about 0.01 mg/mL, at least about 0.02 mg/mL, at least about 0.03 mg/mL, at least about 0.04 mg/mL, at least about 0.05 mg/mL, at least about 0.06 mg/mL, at least about 0.07 mg/mL, at least about 0.08 mg/mL, at least about 0.09 mg/mL, at least about 0.1 mg/mL, at least about 0.2 mg/mL, at least about 0.3 mg/mL, at least about 0.4 mg/mL, at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, at least about 30 mg/mL, at least about 35 mg/mL, at least about 40 mg/mL, at least about 45 mg/mL, at least about 50 mg/mL, at least about 55 mg/mL, at least about 60 mg/mL, at least about 65 mg/mL, at least about 70 mg/mL, at least about 75 mg/mL, at least about 80 mg/mL, at least about 85 mg/mL, at least about 90 mg/mL, at least about 95 mg/mL, at least about 100 mg/mL, at least about 125 mg/mL, at least about 150 mg/mL, at least about 175 mg/mL, at least about 200 mg/mL, at least about 250 mg/mL, at least about 300 mg/mL, at least about 350 mg/mL, at least about 400 mg/mL, at least about 450 mg/mL, at least about 500 mg/mL, at least about 550 mg/mL, at least about 600 mg/mL, at least about 650 mg/mL, at least about 700 mg/mL, at least about 750 mg/mL, at least about 800 mg/mL, at least about 850 mg/mL, at least about 900 mg/mL, at least about 950 mg/mL, at least about 1000 mg/mL, at least about 1050 mg/mL, at least about 1100 mg/mL, at least about 1150 mg/mL, at least about 1200 mg/mL, at least about 1250 mg/mL, at least about 1300 mg/mL, at least about 1350 mg/mL, at least about 1400 mg/mL, at least about 1450 mg/mL, at least about 1500 mg/mL, at least about 1550 mg/mL, at least about 1600 mg/mL, at least about 1650 mg/mL, at least about 1700 mg/mL, at least about 1750 mg/mL, at least about 1800 mg/mL, at least about 1850 mg/mL, at least about 1900 mg/mL, at least about 1950 mg/mL, or at least about 2000 mg/mL.

In some embodiments, a formulation provided herein comprises a compound provided herein at a concentration of at most about 0.002 mg/mL, at most about 0.003 mg/mL, at most about 0.004 mg/mL, at most about 0.005 mg/mL, at most about 0.006 mg/mL, at most about 0.007 mg/mL, at most about 0.008 mg/mL, at most about 0.009 mg/mL, at most about 0.01 mg/mL, at most about 0.02 mg/mL, at most about 0.03 mg/mL, at most about 0.04 mg/mL, at most about 0.05 mg/mL, at most about 0.06 mg/mL, at most about 0.07 mg/mL, at most about 0.08 mg/mL, at most about 0.09 mg/mL, at most about 0.1 mg/mL, at most about 0.2 mg/mL, at most about 0.3 mg/mL, at most about 0.4 mg/mL, at most about 0.5 mg/mL, at most about 0.6 mg/mL, at most about 0.7 mg/mL, at most about 0.8 mg/mL, at most about 0.9 mg/mL, at most about 1 mg/mL, at most about 2 mg/mL, at most about 3 mg/mL, at most about 4 mg/mL, at most about 5 mg/mL, at most about 10 mg/mL, at most about 15 mg/mL, at most about 20 mg/mL, at most about 25 mg/mL, at most about 30 mg/mL, at most about 35 mg/mL, at most about 40 mg/mL, at most about 45 mg/mL, at most about 50 mg/mL, at most about 55 mg/mL, at most about 60 mg/mL, at most about 65 mg/mL, at most about 70 mg/mL, at most about 75 mg/mL, at most about 80 mg/mL, at most about 85 mg/mL, at most about 90 mg/mL, at most about 95 mg/mL, at most about 100 mg/mL, at most about 125 mg/mL, at most about 150 mg/mL, at most about 175 mg/mL, at most about 200 mg/mL, at most about 250 mg/mL, at most about 300 mg/mL, at most about 350 mg/mL, at most about 400 mg/mL, at most about 450 mg/mL, at most about 500 mg/mL, at most about 550 mg/mL, at most about 600 mg/mL, at most about 650 mg/mL, at most about 700 mg/mL, at most about 750 mg/mL, at most about 800 mg/mL, at most about 850 mg/mL, at most about 900 mg/mL, at most about 950 mg/mL, at most about 1000 mg/mL, at most about 1050 mg/mL, at most about 1100 mg/mL, at most about 1150 mg/mL, at most about 1200 mg/mL, at most about 1250 mg/mL, at most about 1300 mg/mL, at most about 1350 mg/mL, at most about 1400 mg/mL, at most about 1450 mg/mL, at most about 1500 mg/mL, at most about 1550 mg/mL, at most about 1600 mg/mL, at most about 1650 mg/mL, at most about 1700 mg/mL, at most about 1750 mg/mL, at most about 1800 mg/mL, at most about 1850 mg/mL, at most about 1900 mg/mL, at most about 1950 mg/mL, or at most about 2000 mg/mL.

In some embodiments, a formulation provided herein comprises a compound provided herein at a concentration of about 1 mg/mL to about 2000 mg/mL; from about 5 mg/mL to about 1000 mg/mL, from about 10 mg/mL to about 25 mg/mL, from about 50 mg/mL to about 250 mg/mL, from about 100 mg/mL to about 200 mg/mL, from about 1 mg/mL to about 50 mg/mL, from about 50 mg/mL to about 100 mg/mL, from about 100 mg/mL to about 150 mg/mL, from about 150 mg/mL to about 200 mg/mL, from about 200 mg/mL to about 250 mg/mL, from about 250 mg/mL to about 300 mg/mL, from about 300 mg/mL to about 350 mg/mL, from about 350 mg/mL to about 400 mg/mL, from about 400 mg/mL to about 450 mg/mL, from about 450 mg/mL to about 500 mg/mL, from about 500 mg/mL to about 550 mg/mL, from about 550 mg/mL to about 600 mg/mL, from about 600 mg/mL to about 650 mg/mL, from about 650 mg/mL to about 700 mg/mL, from about 700 mg/mL to about 750 mg/mL, from about 750 mg/mL to about 800 mg/mL, from about 800 mg/mL to about 850 mg/mL, from about 850 mg/mL to about 900 mg/mL, from about 900 mg/mL to about 950 mg/mL, from about 950 mg/mL to about 1000 mg/mL, about 1 μg/mL to about 2000 μg/mL; from about 5 μg/mL to about 1000 μg/mL, from about 10 μg/mL to about 25 μg/mL, from about 50 μg/mL to about 250 μg/mL, from about 100 μg/mL to about 200 μg/mL, from about 1 μg/mL to about 50 μg/mL, from about 50 μg/mL to about 100 μg/mL, from about 100 μg/mL to about 150 μg/mL, from about 150 μg/mL to about 200 μg/mL, from about 200 μg/mL to about 250 μg/mL, from about 250 μg/mL to about 300 μg/mL, from about 300 μg/mL to about 350 μg/mL, from about 350 μg/mL to about 400 μg/mL, from about 400 μg/mL to about 450 μg/mL, from about 450 μg/mL to about 500 μg/mL, from about 500 μg/mL to about 550 μg/mL, from about 550 μg/mL to about 600 μg/mL, from about 600 μg/mL to about 650 μg/mL, from about 650 μg/mL to about 700 μg/mL, from about 700 μg/mL to about 750 μg/mL, from about 750 μg/mL to about 800 μg/mL, from about 800 μg/mL to about 850 μg/mL, from about 850 μg/mL to about 900 μg/mL, from about 900 μg/mL to about 950 μg/mL, or from about 950 μg/mL to about 1000 μg/mL.

In some embodiments, a formulation of the disclosure delivers about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg AED of a compound of the disclosure. In some embodiments, a formulation of the disclosure delivers about 0.1 mg/kg AED of a compound of the disclosure. In some embodiments, a formulation of the disclosure delivers about 0.2 mg/kg AED of a compound of the disclosure. In some embodiments, a formulation of the disclosure delivers about 0.3 mg/kg AED of a compound of the disclosure.

An approximate dose can be predicted or determined on the basis of data existing in other species. In some embodiments, allometric scaling can be used to exchange a drug dose based on normalization of dose to body surface area. Allometric scaling considers the sizes of individual species based on body surface area, which is related to metabolic rate of an animal that is established through evolutionary adaptation of animals to their size. A no observed adverse effect level (NOAEL) is first determined in an animal species, the NOAEL is converted to a human equivalent dose (HED), an appropriate animal species is selected, a safety factor is applied, and a pharmacologically active dose is determined.

NOAEL, the highest dose level that does not cause significant adverse effects, is a typical index for safety obtained from animal experiments to determine a safe starting dose. NOAEL values can be converted to HED on the basis of the body surface correction factor using appropriate scaling factors from animal species. TABLE 5 lists HED calculation guidelines based on body surface areas. HED is determined using the equation:

HED (mg/kg)=Animal NOAEL (mg/kg)×(Weightₐₙᵢₘₐₗ[kg]/Weightₕᵤₘₐₙ[kgM])^{(1-0.67)}

The HED is divided by a factor value of 10 to increase safety of the first human dose. The safety factor is accountable for differences in physiological and biological processes between human and animal species.

The correction factor (Kₘ) is estimated by dividing the average body weight (kg) of a species to its body surface area (m²). The Kₘ factor values of various animal species of TABLE 5 is used to estimate the HED as:

HED (mg/kg)=Animal doses (mg/kg)×(Animal Kₘ/Human Kₘ); or

HED (mg/kg)=Animal doses (mg/kg)×Kₘ ratio

However, conversion between species based on mg/m² is not supported for drugs administered by topical, nasal, subcutaneous, or intramuscular routes, as well as proteins administered parenterally with molecular weight >100,000 Daltons.

TABLE 5

| | | | | To convert | | |
|---|---|---|---|---|---|---|
| | | | | dose in | To convert animal dose | |
| | | | | mg/kg to | in mg/kg to HED in | |
| | | Working | Body | dose in | mg/kg, either | |
| | Reference | weight | surface | mg/m², | Divide | Multiply |
| | body weight | range | area | multiply by | animal | animal |
| Species | (kg) | (kg) | (m²) | Kₘ | dose by | dose by |
| Human | 60 | — | 1.62 | 37 | — | — |
| Mouse | 0.02 | 0.011-0.034 | 0.007 | 3 | 12.3 | 0.081 |
| Hamster | 0.08 | 0.047-0.157 | 0.016 | 5 | 7.4 | 0.135 |
| Rat | 0.15 | 0.08-0.27 | 0.025 | 6 | 6.2 | 0.162 |
| Ferret | 0.30 | 0.16-0.54 | 0.043 | 7 | 5.3 | 0.189 |
| Guinea Pig | 0.40 | 0.208-0.700 | 0.05 | 8 | 4.6 | 0.216 |
| Rabbit | 1.8 | 0.90-3.0 | 0.15 | 12 | 3.1 | 0.324 |
| Dog | 10 | 5-17 | 0.50 | 20 | 1.8 | 0.541 |
| Monkeys | 3 | 1.4-4.9 | 0.25 | 12 | 3.1 | 0.324 |
| (rhesus) | | | | | | |
| Marmoset | 0.35 | 0.14-0.72 | 0.06 | 6 | 6.2 | 0.162 |
| Squirrel | 0.60 | 0.29-0.97 | 0.09 | 7 | 5.3 | 0.189 |
| Monkey | | | | | | |
| Baboon | 12 | 7-23 | 0.60 | 20 | 1.8 | 0.541 |
| Micro pig | 20 | 10-33 | 0.74 | 27 | 1.4 | 0.730 |
| Mini pig | 40 | 25-64 | 1.14 | 35 | 1.1 | 0.946 |

TABLE 6 provides animal equivalent dose (AED) calculation guidelines based on body surface area. The animal equivalent dose (AED) can also be calculated on the basis of body surface area by either dividing or multiplying the human dose (mg/kg) by the K m ratio provided in TABLE 6. AED can be calculated using the equation:

AED (mg/kg)=Human doses (mg/kg)×Kₘ ratio

TABLE 6

| | | To convert | To convert human | |
|---|---|---|---|---|
| | | dose in mg/ | dose in mg/kg to | |
| | | kg to dose | AED in mg/kg, either | |
| | Reference | in mg/ | Multiply | Divide |
| | body | m², divide | human | human |
| Species | weight (kg) | by Kₘ | dose by | dose by |
| Human | 60 | 37 | — | — |
| Mouse | 0.02 | 3 | 12.3 | 0.081 |
| Hamster | 0.08 | 5 | 7.4 | 0.135 |
| Rat | 0.15 | 6 | 6.2 | 0.162 |
| Ferret | 0.30 | 7 | 5.3 | 0.189 |
| Guinea Pig | 0.40 | 8 | 4.6 | 0.216 |
| Rabbit | 1.8 | 12 | 3.1 | 0.324 |
| Dog | 10 | 20 | 1.8 | 0.541 |
| Monkeys (rhesus) | 3 | 12 | 3.1 | 0.324 |
| Marmoset | 0.35 | 6 | 6.2 | 0.162 |
| Squirrel Monkey | 0.60 | 7 | 5.3 | 0.189 |
| Baboon | 12 | 20 | 1.8 | 0.541 |
| Micro pig | 20 | 27 | 1.4 | 0.730 |
| Mini pig | 40 | 35 | 1.1 | 0.946 |

For parenteral administration, HED conversion (mg/kg) is also based on body surface area normalization. The conversion can be made by dividing the NOAEL in appropriate species by the conversion factor. TABLE 7 provides guidelines for maximum injection volume, by species, site location, and gauge size. Injection volume of parenteral formulation is calculated by the following equation:

Injection volume (mL)=[Animal weight (kg)×Animal doses (mg/kg)]/Concentration (mg/kg)

TABLE 7

| | Route | | | | | | | | |
|---|---|---|---|---|---|---|---|---|---|
| | Subcutaneous | | | Intramuscular | | | Intraperitoneal | | |
| | Max | | | Max | | | Max | | |
| | injection | | | injection | | | injection | | |
| | volume | | Gauge | volume | | Gauge | volume | | Gauge |
| Species | (mL) | Site | size | (mL) | Site | size | (mL) | Site | size |
| Human | <2 | Upper | 25-31 | 2-5 | Deltoid, | 21-23 | 2-5 | Peritoneum | — |
| | | arm, | | | vastus | | | | |
| | | abdomen, | | | lateralis | | | | |
| | | thigh, | | | | | | | |
| | | buttock | | | | | | | |
| Mouse | 1-2 | Back | <20 | <0.1 | Quadriceps, | 25 | 2-3 | Lateral tail | 25 |
| | | (scruff), | | | posterior | | | vein | |
| | | lower | | | thigh | | | | |
| | | abdomen | | | | | | | |
| Hamster | 3-4 | Dorsum | <20 | <0.1 | hind limb, | 25 | 2-3 | Lower left | 25-56 |
| | | between | | | caudal thigh | | | quadrant | |
| | | scapula | | | | | | | |
| Rat | 5-10 | Back | <20 | <0.3 | quadriceps, | 25-56 | 5-10 | Lower left | 24 |
| | | (scruff), | | | hamstrings | | | quadrant | |
| | | lower | | | | | | | |
| | | abdomen | | | | | | | |
| Dog | 100-200 | Dorsum | 22 | 2-5 | hind limb, | 22-25 | 100-200 | Cephalic, | 22-25 |
| | | between | | | caudal thigh | | | saphenous | |
| | | scapula | | | | | | | |
| Guinea | 5-10 | Dorsum | 25 | 0.3 | Lind limb, | 25 | 10-15 | Lower left | 25 |
| pig | | between | | | caudal | | | quadrant | |
| | | scapula | | | thigh, | | | | |
| | | | | | lumbar | | | | |
| | | | | | muscles | | | | |
| Rabbit | 10-50 | Dorsum | 22-25 | <0.5 | Hind limb, | 22-25 | 50-100 | Lower left | 22 |
| | | | | | caudal thigh | | | quadrant | |
| Monkey | 10-30 | Dorsum | 22-23 | 1-3 | Hind limb, | 22-23 | 25-50 | Peritoneum | 20 |
| (Rhesus) | | | | | caudal thigh | | | | |
| Squirrel | 5-10 | Scuff | 20 | 1-3 | Quadriceps, | 20 | 25-50 | Peritoneum | 20 |
| Monkey | | | | | posterior | | | | |
| | | | | | thigh, | | | | |
| | | | | | triceps | | | | |

| | Route | | | | | |
|---|---|---|---|---|---|---|
| | Intravenous | | | Intradermal | | |
| | Max | | | Max | | |
| | injection | | | injection | | |
| | volume | | Gauge | volume | | Gauge |
| Species | (mL) | Site | size | (mL) | Site | size |
| Human | <250 | Vein | 18-20 | <0.10 | Dermis | 25-26 |
| Mouse | <0.2 | Lateral tail vein | 25 | <0.05 | Lateral | — |
| | | | | | abdomen | |
| Hamster | <0.2 | Lateral tarsal vein, | 26 | <0.05 | — | — |
| | | cephalic/lingual | | | | |
| | | vein | | | | |
| Rat | <0.5 | Lateral | 24-27 | <0.05 | Lateral | — |
| | | tail/saphenous vein | | | abdomen | |
| Dog | <100 | Cephalic/saphenous | 21 | — | — | — |
| | | vein | | | | |
| Guinea | 10-15 | Lateral saphenous | 26-27 | — | Dorsum | 25 |
| pig | | vein | | | along | |
| | | | | | flank | |
| Rabbit | <5 | Marginal ear vein | 25 | <0.1 | Dorsum | 25 |
| | | | | | along | |
| | | | | | flank | |
| Monkey | 5-10 | Saphenous vein | 22-23 | — | — | — |
| (Rhesus) | | | | | | |
| Squirrel | 0.5-1 | Femoral vein | 21 | — | — | — |
| Monkey | | | | | | |

### Tolerability and Toxicity

A single dose tolerability study at 30 mg/kg Compound 1 (n=3) and 60 mg/kg Compound 1 (n=2) in FVB mice was conducted. No significant weight of loss was observed over 7 days. No behavior changes were noted over 7 days. No significant blood chemistry changes were observed when compared to control animals over 7 days. A profile of 32 cytokines did not show increased response from control animals (blood chemistry variability was observed in control and treated groups).

In some embodiments, administration of a compound disclosed herein (e.g., Compound 1) does not result in immunogenicity as measured by 32 individually assayed cytokine levels. In some embodiments, administration of a compound disclosed herein (e.g., Compound 1) does not induce weight loss in a subject over at least 7 days post-administration.

In some embodiments, the present disclosure provides a compound comprising a peptide nucleic acid sequence, wherein the peptide nucleic acid sequence binds to a nucleic acid molecule associated with myotonic dystrophy 1.

In some embodiments, if in a study: a) i) a control HSA-LR mouse is administered a vehicle by tail vein injection; ii) 21 days later, the control HSA-LR mouse is sacrificed; iii) a quadricep muscle tissue is harvested from the control HSA-LR mouse post-sacrifice; iv) the quadricep muscle tissue post-harvest is snap frozen in liquid nitrogen; v) after snap freeze, the quadricep muscle tissue is fixed and sectioned into sections having a length of about 6 microns; vi) the sections are subjected overnight to fluorescence in situ hybridization using a fluorescent probe at a concentration of 1 ng/μL to stain nuclear inclusions associated with a myotonic dystrophy 1 disease phenotype, wherein the fluorescent probe is/5Cy3/CAGCAGCAGCAGCAGCAGCA (SEQ ID NO: 111); vii) after subjecting to fluorescence in situ hybridization, the sections are washed; and viii) after being washed, the sections are imaged using confocal fluorescence microscopy using 4′,6-diamidino-2-phenylindole stain to stain cellular nuclei in the sections to provide an image showing the cellular nuclei and the nuclear inclusions associated with the myotonic dystrophy 1 disease phenotype, wherein the cellular nuclei and the nuclear inclusions associated with the myotonic dystrophy 1 disease phenotype are visualized in a control ratio; and b) i) a test HSA-LR mouse is administered the compound in the vehicle by tail vein injection; ii) 21 days later, the test HSA-LR mouse is sacrifice; iii) a quadricep muscle tissue is harvested from the test HSA-LR mouse post-sacrifice; iv) the quadricep muscle tissue post-harvest is snap frozen in liquid nitrogen; v) after snap freeze, the quadricep muscle tissue is fixed and sectioned into sections having a length of about 6 microns; vi) the sections are subjected overnight to fluorescence in situ hybridization using the fluorescent probe at a concentration of 1 ng/μL to stain nuclear inclusions associated with a myotonic dystrophy 1 disease phenotype; vii) after subjecting to fluorescence in situ hybridization, the sections are washed; and viii) after being washed, the sections are imaged using confocal fluorescence microscopy using 4′,6-diamidino-2-phenylindole stain to stain cellular nuclei in the sections to provide an image showing the cellular nuclei and the nuclear inclusions associated with the myotonic dystrophy 1 disease phenotype, wherein the cellular nuclei and the nuclear inclusions associated with the myotonic dystrophy 1 disease phenotype are visualized in a test ratio, then: the test ratio is at least 50% greater than is the control ratio.

In some embodiments, the test ratio is at least 100% greater than is the control ratio. In some embodiments, the test ratio is at least 200% greater than is the control ratio. In some embodiments, the test ratio is at least 500% greater than is the control ratio. In some embodiments, the test ratio is at least 1000% greater than is the control ratio.

In some embodiments, the present disclosure provides a method of treating myotonic dystrophy 1, the method comprising administering to a subject in need thereof a therapeutically-effective amount of a compound, wherein the compound comprises a peptide nucleic acid sequence, wherein the peptide nucleic acid sequence binds to a nucleic acid molecule associated with myotonic dystrophy 1.

In some embodiments, if, in a study: a) i) a control HSA-LR mouse is administered a vehicle by tail vein injection; ii) 21 days later, the control HSA-LR mouse is sacrificed; iii) a quadricep muscle tissue is harvested from the control HSA-LR mouse post-sacrifice; iv) the quadricep muscle tissue post-harvest is snap frozen in liquid nitrogen; v) after snap freeze, the quadricep muscle tissue is fixed and sectioned into sections having a length of about 6 microns; vi) the sections are subjected overnight to fluorescence in situ hybridization using a fluorescent probe at a concentration of 1 ng/μL to stain nuclear inclusions associated with a myotonic dystrophy 1 disease phenotype, wherein the fluorescent probe is/5Cy3/CAGCAGCAGCAGCAGCAGCA (SEQ ID NO: 111); vii) after subjecting to fluorescence in situ hybridization, the sections are washed; and viii) after being washed, the sections are imaged using confocal fluorescence microscopy using 4′,6-diamidino-2-phenylindole stain to stain cellular nuclei in the sections to provide an image showing the cellular nuclei and the nuclear inclusions associated with the myotonic dystrophy 1 disease phenotype, wherein the cellular nuclei and the nuclear inclusions associated with the myotonic dystrophy 1 disease phenotype are visualized in a control ratio; and b) i) a test HSA-LR mouse is administered the compound in the vehicle by tail vein injection; ii) 21 days later, the test HSA-LR mouse is sacrifice; iii) a quadricep muscle tissue is harvested from the test HSA-LR mouse post-sacrifice; iv) the quadricep muscle tissue post-harvest is snap frozen in liquid nitrogen; v) after snap freeze, the quadricep muscle tissue is fixed and sectioned into sections having a length of about 6 microns; vi) the sections are subjected overnight to fluorescence in situ hybridization using the fluorescent probe at a concentration of 1 ng/μL to stain nuclear inclusions associated with a myotonic dystrophy 1 disease phenotype; vii) after subjecting to fluorescence in situ hybridization, the sections are washed; and viii) after being washed, the sections are imaged using confocal fluorescence microscopy using 4′,6-diamidino-2-phenylindole stain to stain cellular nuclei in the sections to provide an image showing the cellular nuclei and the nuclear inclusions associated with the myotonic dystrophy 1 disease phenotype, wherein the cellular nuclei and the nuclear inclusions associated with the myotonic dystrophy 1 disease phenotype are visualized in a test ratio, then: the test ratio is at least 50% greater than is the control ratio.

In some embodiments, the test ratio is at least 100% greater than is the control ratio. In some embodiments, the test ratio is at least 200% greater than is the control ratio. In some embodiments, the test ratio is at least 500% greater than is the control ratio. In some embodiments, the test ratio is at least 1000% greater than is the control ratio.

In some embodiments, the present disclosure provides a compound comprising a peptide nucleic acid sequence, wherein the peptide nucleic acid sequence restores skeletal muscle chloride channel in a model of myotonic dystrophy 1.

In some embodiments, if, in a study: a) i) a first test mouse that possesses a myotonic dystrophy 1 phenotype is intravenously administered the compound in a vehicle by tail vein, wherein the compound is present in the vehicle at a concentration of 29 mg/kg; ii) after a period of time sufficient for distribution of the compound through musculature of the first test mouse, the first test mouse is sacrificed; iii) after sacrifice, 50-100 mg of muscle tissue is extracted from the first mouse; iv) the muscle tissue extracted from the first test mouse is homogenized and lysed using 300 μL extraction buffer to provide a protein extract from the first test mouse; v) the protein extract from the first test mouse is resolved using SDS-PAGE at a 4-12% gradient with running buffer at 150 V for 2 hours to provide resolved proteins from the first test mouse, wherein the resolved proteins from the first test mouse are CLCN1 from the first test mouse and beta-actin from the first test mouse; vi) the resolved proteins from the first test mouse are transferred to a nitrocellulose membrane; vii) the CLCN1 from the first test mouse is contacted on the nitrocellulose membrane with a primary antibody specific for CLCN1; viii) the beta-actin from the first test mouse is contacted on the nitrocellulose membrane with a primary antibody specific for beta-actin; ix) a conjugate of horseradish peroxidase and an anti-mouse secondary antibody is contacted to the resolved proteins from the first test mouse on the nitrocellulose membrane, and a chemiluminescent substrate of horseradish peroxidase is contacted to the nitrocellulose membrane to provide a luminescent band associated with the CLCN1 from the first test mouse and a luminescent band associated with the beta-actin from the first test mouse; x) the luminescent band associated with the CLCN1 from the first test mouse is quantified to provide a quantification of the CLCN1 from the first test mouse; and xi) the luminescent band associated with the beta-actin from the first test mouse is quantified to provide a quantification of the beta-actin from the first test mouse; b) i) a second test mouse that possesses a myotonic dystrophy 1 phenotype is intravenously administered the vehicle by tail vein; ii) after the period of time sufficient for distribution of the compound through musculature of the first test mouse, the second test mouse is sacrificed; iii) after sacrifice, 50-100 mg of muscle tissue is extracted from the second mouse; iv) the muscle tissue extracted from the second test mouse is homogenized and lysed using 300 μL extraction buffer to provide a protein extract from the second test mouse; v) the protein extract from the second test mouse is resolved using SDS-PAGE at a 4-12% gradient with running buffer at 150 V for 2 hours to provide resolved proteins from the second test mouse, wherein the resolved proteins from the second test mouse are CLCN1 from the second test mouse and beta-actin from the second test mouse; vi) the resolved proteins from the second test mouse are transferred to a nitrocellulose membrane; vii) the CLCN1 from the second test mouse is contacted on the nitrocellulose membrane with a primary antibody specific for CLCN1; viii) the beta-actin from the second test mouse is contacted on the nitrocellulose membrane with a primary antibody specific for beta-actin; ix) a conjugate of horseradish peroxidase and an anti-mouse secondary antibody is contacted to the resolved proteins from the second test mouse on the nitrocellulose membrane, and a chemiluminescent substrate of horseradish peroxidase is contacted to the nitrocellulose membrane to provide a luminescent band associated with the CLCN1 from the second test mouse and a luminescent band associated with the beta-actin from the second test mouse; x) the luminescent band associated with the CLCN1 from the second test mouse is quantified to provide a quantification of the CLCN1 from the second test mouse; and xi) the luminescent band associated with the beta-actin from the second test mouse is quantified to provide a quantification of the beta-actin from the second test mouse; c) i) a control mouse that does not possess a myotonic dystrophy 1 phenotype is intravenously administered the vehicle by tail vein; ii) after the period of time sufficient for distribution of the compound through musculature of the first test mouse, the control mouse is sacrificed; iii) after sacrifice, 50-100 mg of muscle tissue is extracted from the control mouse; iv) the muscle tissue extracted from the control mouse is homogenized and lysed using 300 μL extraction buffer to provide a protein extract from the control mouse; v) the protein extract from the control mouse is resolved using SDS-PAGE at a 4-12% gradient with running buffer at 150 V for 2 hours to provide resolved proteins from the control mouse, wherein the resolved proteins from the control mouse are CLCN1 from the control mouse and beta-actin from the control mouse; vi) the resolved proteins from the control mouse are transferred to a nitrocellulose membrane; vii) the CLCN1 from the control mouse is contacted on the nitrocellulose membrane with a primary antibody specific for CLCN1; viii) the beta-actin from the control mouse is contacted on the nitrocellulose membrane with a primary antibody specific for beta-actin; ix) a conjugate of horseradish peroxidase and an anti-mouse secondary antibody is contacted to the resolved proteins from the control mouse on the nitrocellulose membrane, and a chemiluminescent substrate of horseradish peroxidase is contacted to the nitrocellulose membrane to provide a luminescent band associated with the CLCN1 from the control mouse and a luminescent band associated with the beta-actin from the control mouse; x) the luminescent band associated with the CLCN1 from the control mouse is quantified to provide a quantification of the CLCN1 from the control mouse; and xi) the luminescent band associated with the beta-actin from the control mouse is quantified to provide a quantification of the beta-actin from the control mouse; and d) the quantification of the CLCN1 from the first test mouse, the quantification of the CLCN1 from the second test mouse, and the quantification of the CLCN1 from the control mouse are normalized based on the quantification of the beta-actin from the first test mouse, the quantification of the beta-actin from the second test mouse, and the quantification of the beta-actin from the control mouse to provide a normalized amount of the CLCN1 from the first test mouse, a normalized amount of the CLCN1 from the second test mouse, and a normalized amount of the CLCN1 from the control mouse, then the normalized amount of the CLCN1 from the first test mouse is determined to be no more than 2.5 times the normalized amount of the CLCN1 from the control mouse; and the normalized amount of the CLCN1 from the first test mouse is determined to be at least 50% greater than is the normalized amount of the CLCN1 from the second test mouse.

In some embodiments, the present disclosure provides a method of treating myotonic dystrophy 1, the method comprising administering to a subject in need thereof a therapeutically-effective amount of a compound, wherein the compound comprises a peptide nucleic acid sequence, wherein the peptide nucleic acid sequence restores skeletal muscle chloride channel in a model of myotonic dystrophy 1.

In some embodiments, if, in a study: a) i) a first test mouse that possesses a myotonic dystrophy 1 phenotype is intravenously administered 29 mg/kg of the compound in a vehicle by tail vein; ii) after a period of time sufficient for distribution of the compound through musculature of the first test mouse, the first test mouse is sacrificed; iii) after sacrifice, 50-100 mg of muscle tissue is extracted from the first mouse; iv) the muscle tissue extracted from the first test mouse is homogenized and lysed using 300 μL extraction buffer to provide a protein extract from the first test mouse; v) the protein extract from the first test mouse is resolved using SDS-PAGE at a 4-12% gradient with running buffer at 150 V for 2 hours to provide resolved proteins from the first test mouse, wherein the resolved proteins from the first test mouse are CLCN1 from the first test mouse and beta-actin from the first test mouse; vi) the resolved proteins from the first test mouse are transferred to a nitrocellulose membrane; vii) the CLCN1 from the first test mouse is contacted on the nitrocellulose membrane with a primary antibody specific for CLCN1; viii) the beta-actin from the first test mouse is contacted on the nitrocellulose membrane with a primary antibody specific for beta-actin; ix) a conjugate of horseradish peroxidase and an anti-mouse secondary antibody is contacted to the resolved proteins from the first test mouse on the nitrocellulose membrane, and a chemiluminescent substrate of horseradish peroxidase is contacted to the nitrocellulose membrane to provide a luminescent band associated with the CLCN1 from the first test mouse and a luminescent band associated with the beta-actin from the first test mouse; x) the luminescent band associated with the CLCN1 from the first test mouse is quantified to provide a quantification of the CLCN1 from the first test mouse; and xi) the luminescent band associated with the beta-actin from the first test mouse is quantified to provide a quantification of the beta-actin from the first test mouse; b) i) a second test mouse that possesses a myotonic dystrophy 1 phenotype is intravenously administered the vehicle by tail vein; ii) after the period of time sufficient for distribution of the compound through musculature of the first test mouse, the second test mouse is sacrificed; iii) after sacrifice, 50-100 mg of muscle tissue is extracted from the second mouse; iv) the muscle tissue extracted from the second test mouse is homogenized and lysed using 300 μL extraction buffer to provide a protein extract from the second test mouse; v) the protein extract from the second test mouse is resolved using SDS-PAGE at a 4-12% gradient with running buffer at 150 V for 2 hours to provide resolved proteins from the second test mouse, wherein the resolved proteins from the second test mouse are CLCN1 from the second test mouse and beta-actin from the second test mouse; vi) the resolved proteins from the second test mouse are transferred to a nitrocellulose membrane; vii) the CLCN1 from the second test mouse is contacted on the nitrocellulose membrane with a primary antibody specific for CLCN1; viii) the beta-actin from the second test mouse is contacted on the nitrocellulose membrane with a primary antibody specific for beta-actin; ix) a conjugate of horseradish peroxidase and an anti-mouse secondary antibody is contacted to the resolved proteins from the second test mouse on the nitrocellulose membrane, and a chemiluminescent substrate of horseradish peroxidase is contacted to the nitrocellulose membrane to provide a luminescent band associated with the CLCN1 from the second test mouse and a luminescent band associated with the beta-actin from the second test mouse; x) the luminescent band associated with the CLCN1 from the second test mouse is quantified to provide a quantification of the CLCN1 from the second test mouse; and xi) the luminescent band associated with the beta-actin from the second test mouse is quantified to provide a quantification of the beta-actin from the second test mouse; c) i) a control mouse that does not possess a myotonic dystrophy 1 phenotype is intravenously administered the vehicle by tail vein; ii) after the period of time sufficient for distribution of the compound through musculature of the first test mouse, the control mouse is sacrificed; iii) after sacrifice, 50-100 mg of muscle tissue is extracted from the control mouse; iv) the muscle tissue extracted from the control mouse is homogenized and lysed using 300 μL extraction buffer to provide a protein extract from the control mouse; v) the protein extract from the control mouse is resolved using SDS-PAGE at a 4-12% gradient with running buffer at 150 V for 2 hours to provide resolved proteins from the control mouse, wherein the resolved proteins from the control mouse are CLCN1 from the control mouse and beta-actin from the control mouse; vi) the resolved proteins from the control mouse are transferred to a nitrocellulose membrane; vii) the CLCN1 from the control mouse is contacted on the nitrocellulose membrane with a primary antibody specific for CLCN1; viii) the beta-actin from the control mouse is contacted on the nitrocellulose membrane with a primary antibody specific for beta-actin; ix) a conjugate of horseradish peroxidase and an anti-mouse secondary antibody is contacted to the resolved proteins from the control mouse on the nitrocellulose membrane, and a chemiluminescent substrate of horseradish peroxidase is contacted to the nitrocellulose membrane to provide a luminescent band associated with the CLCN1 from the control mouse and a luminescent band associated with the beta-actin from the control mouse; x) the luminescent band associated with the CLCN1 from the control mouse is quantified to provide a quantification of the CLCN1 from the control mouse; and xi) the luminescent band associated with the beta-actin from the control mouse is quantified to provide a quantification of the beta-actin from the control mouse; and d) the quantification of the CLCN1 from the first test mouse, the quantification of the CLCN1 from the second test mouse, and the quantification of the CLCN1 from the control mouse are normalized based on the quantification of the beta-actin from the first test mouse, the quantification of the beta-actin from the second test mouse, and the quantification of the beta-actin from the control mouse to provide a normalized amount of the CLCN1 from the first test mouse, a normalized amount of the CLCN1 from the second test mouse, and a normalized amount of the CLCN1 from the control mouse, then: the normalized amount of the CLCN1 from the first test mouse is determined to be no more than 2.5 times the normalized amount of the CLCN1 from the control mouse; and the normalized amount of the CLCN1 from the first test mouse is determined to be at least 50% greater than is the normalized amount of the CLCN1 from the second test mouse.

In some embodiments, the present disclosure provides a compound comprising a peptide nucleic acid sequence, wherein the peptide nucleic acid sequence restores skeletal muscle movement in a model of myotonic dystrophy 1.

In some embodiments, if, in a study: a) i) a first test mouse that possesses a myotonic dystrophy 1 phenotype is subcutaneously administered 3 mg/kg of the compound in a vehicle once per week over a four-week period; ii) 35 days after first administration of the compound in the vehicle to the first test mouse, the first test mouse is subjected to a muscle lever system while the first test mouse is under anesthesia, wherein a knee of the first test mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the first test mouse for stimulation; iii) stimulating the plantarflexor muscle group of the first test mouse by percutaneous electrical stimulation of a sciatic nerve of the plantarflexor muscle group of the first test mouse with 10 stimulations, each at 150 Hz, with 30 second rest periods between stimulations, thereby promoting contractions in the plantarflexor muscle group of the first test mouse and observing the contractions; and iv) based on observing the contractions in the plantarflexor muscle group of the first test mouse, determining a mean time to relaxation for the contractions in the plantarflexor muscle group of the first test mouse; b) i) a second test mouse that possesses a myotonic dystrophy 1 phenotype is subcutaneously administered the vehicle once per week over a four-week period; ii) 35 days after first administration of the vehicle to the second test mouse, the second test mouse is subjected to a muscle lever system while the second test mouse is under anesthesia, wherein a knee of the second test mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the second test mouse for stimulation; iii) stimulating the plantarflexor muscle group of the second test mouse by percutaneous electrical stimulation of a sciatic nerve of the plantarflexor muscle group of the second test mouse with 10 stimulations, each at 150 Hz, with 30 second rest periods between stimulations, thereby promoting contractions in the plantarflexor muscle group of the second test mouse and observing the contractions; and iv) based on observing the contractions in the plantarflexor muscle group of the second test mouse, determining a mean time to relaxation for the contractions in the plantarflexor muscle group of the second test mouse; c) i) a control mouse that does not possess a myotonic dystrophy 1 phenotype is subcutaneously administered the vehicle once per week over a four-week period; ii) 35 days after first administration of the vehicle to the control mouse, the control mouse is subjected to a muscle lever system while the control mouse is under anesthesia, wherein a knee of the control mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the control mouse for stimulation; iii) stimulating the plantarflexor muscle group of the control mouse by percutaneous electrical stimulation of a sciatic nerve of the plantarflexor muscle group of the control mouse with 10 stimulations, each at 150 Hz, with 30 second rest periods between stimulations, thereby promoting contractions in the plantarflexor muscle group of the control mouse and observing the contractions; and iv) based on observing the contractions in the plantarflexor muscle group of the control mouse, determining a mean time to relaxation for the contractions in the plantarflexor muscle group of the control mouse, then: 1) the mean time to relaxation for the contractions in the plantarflexor muscle group of the first test mouse is determined to be 0.15 to 0.22 seconds; 2) the mean time to relaxation for the contractions in the plantarflexor muscle group of the second test mouse is determined to be 0.37 to 0.44 seconds; and 3) the mean time to relaxation for the contractions in the plantarflexor muscle group of the control mouse is determined to be 0.05 to 0.09 seconds.

In some embodiments, the present disclosure provides a compound comprising a peptide nucleic acid sequence, wherein the peptide nucleic acid sequence restores skeletal muscle movement in a model of myotonic dystrophy 1.

In some embodiments, if, in a study, a) i) a first test mouse that possesses a myotonic dystrophy 1 phenotype is subcutaneously administered 3 mg/kg of the compound in a vehicle once per week over a four-week period; ii) 35 days after first administration of the compound in the vehicle to the first test mouse, the first test mouse is subjected to a muscle lever system while the first test mouse is under anesthesia, wherein a knee of the first test mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the first test mouse for stimulation; iii) stimulating the plantarflexor muscle group of the first test mouse by percutaneous electrical stimulation of a sciatic nerve of the plantarflexor muscle group of the first test mouse with 10 stimulations, each at 150 Hz, with 30 second rest periods between stimulations, thereby promoting contractions in the plantarflexor muscle group of the first test mouse and observing the contractions; and iv) based on observing the contractions in the plantarflexor muscle group of the first test mouse, determining a mean time to relaxation for the contractions in the plantarflexor muscle group of the first test mouse; b) i) a second test mouse that possesses a myotonic dystrophy 1 phenotype is subcutaneously administered the vehicle once per week over a four-week period; ii) 35 days after first administration of the vehicle to the second test mouse, the second test mouse is subjected to a muscle lever system while the second test mouse is under anesthesia, wherein a knee of the second test mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the second test mouse for stimulation; iii) stimulating the plantarflexor muscle group of the second test mouse by percutaneous electrical stimulation of a sciatic nerve of the plantarflexor muscle group of the second test mouse with 10 stimulations, each at 150 Hz, with 30 second rest periods between stimulations, thereby promoting contractions in the plantarflexor muscle group of the second test mouse and observing the contractions; and iv) based on observing the contractions in the plantarflexor muscle group of the second test mouse, determining a mean time to relaxation for the contractions in the plantarflexor muscle group of the second test mouse; c) i) a control mouse that does not possess a myotonic dystrophy 1 phenotype is subcutaneously administered the vehicle once per week over a four-week period; ii) 35 days after first administration of the vehicle to the control mouse, the control mouse is subjected to a muscle lever system while the control mouse is under anesthesia, wherein a knee of the control mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the control mouse for stimulation; iii) stimulating the plantarflexor muscle group of the control mouse by percutaneous electrical stimulation of a sciatic nerve of the plantarflexor muscle group of the control mouse with 10 stimulations, each at 150 Hz, with 30 second rest periods between stimulations, thereby promoting contractions in the plantarflexor muscle group of the control mouse and observing the contractions; and iv) based on observing the contractions in the plantarflexor muscle group of the control mouse, determining a mean time to relaxation for the contractions in the plantarflexor muscle group of the control mouse, then: 1) the mean time to relaxation for the contractions in the plantarflexor muscle group of the first test mouse is determined to be at most 60% of the mean time to relaxation for the contractions in the plantarflexor muscle group of the second test mouse; 2) the mean time to relaxation for the contractions in the plantarflexor muscle group of the control mouse is determined to be at most 30% of the mean time to relaxation for the contractions in the plantarflexor muscle group of the second test mouse; and 3) the mean time to relaxation for the contractions in the plantarflexor muscle group of the control mouse is determined to be at most 50% of the mean time to relaxation for the contractions in the plantarflexor muscle group of the first test mouse.

In some embodiments, the present disclosure provides a compound comprising a peptide nucleic acid sequence, wherein the peptide nucleic acid sequence restores skeletal muscle movement in a model of myotonic dystrophy 1.

In some embodiments, if, in a study, a) i) a first test mouse that possesses a myotonic dystrophy 1 phenotype is subcutaneously administered 3 mg/kg of the compound in a vehicle once per week over a four-week period; ii) 35 days after first administration of the compound in the vehicle to the first test mouse, the first test mouse is subjected to a muscle lever system while the first test mouse is under anesthesia, wherein a knee of the first test mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the first test mouse for stimulation; iii) stimulating the plantarflexor muscle group of the first test mouse by percutaneous electrical stimulation of a sciatic nerve of the plantarflexor muscle group of the first test mouse with 10 stimulations, each at 150 Hz, with 30 second rest periods between stimulations, thereby promoting contractions in the plantarflexor muscle group of the first test mouse and observing the contractions; and iv) based on observing the contractions in the plantarflexor muscle group of the first test mouse, determining a mean time to relaxation for the contractions in the plantarflexor muscle group of the first test mouse; and b) i) a second test mouse that possesses a myotonic dystrophy 1 phenotype is subcutaneously administered the vehicle once per week over a four-week period; ii) 35 days after first administration of the vehicle to the second test mouse, the second test mouse is subjected to a muscle lever system while the second test mouse is under anesthesia, wherein a knee of the second test mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the second test mouse for stimulation; iii) stimulating the plantarflexor muscle group of the second test mouse by percutaneous electrical stimulation of a sciatic nerve of the plantarflexor muscle group of the second test mouse with 10 stimulations, each at 150 Hz, with 30 second rest periods between stimulations, thereby promoting contractions in the plantarflexor muscle group of the second test mouse and observing the contractions; and iv) based on observing the contractions in the plantarflexor muscle group of the second test mouse, determining a mean time to relaxation for the contractions in the plantarflexor muscle group of the second test mouse, then the mean time to relaxation for the contractions in the plantarflexor muscle group of the first test mouse is determined to be at most 60% of the mean time to relaxation for the contractions in the plantarflexor muscle group of the second test mouse.

In some embodiments, the present disclosure provides a method of treating myotonic dystrophy 1 in a subject in need thereof, the method comprising administering to the subject a therapeutically-effective amount of a compound, wherein the compound comprises a peptide nucleic acid sequence, wherein the peptide nucleic acid sequence restores skeletal muscle movement in a model of myotonic dystrophy 1.

In some embodiments, if, in a study, a) i) a first test mouse that possesses a myotonic dystrophy 1 phenotype is subcutaneously administered 3 mg/kg of the compound in a vehicle once per week over a four-week period; ii) 35 days after first administration of the compound in the vehicle to the first test mouse, the first test mouse is subjected to a muscle lever system while the first test mouse is under anesthesia, wherein a knee of the first test mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the first test mouse for stimulation; iii) stimulating the plantarflexor muscle group of the first test mouse by percutaneous electrical stimulation of a sciatic nerve of the plantarflexor muscle group of the first test mouse with 10 stimulations, each at 150 Hz, with 30 second rest periods between stimulations, thereby promoting contractions in the plantarflexor muscle group of the first test mouse and observing the contractions; and iv) based on observing the contractions in the plantarflexor muscle group of the first test mouse, determining a mean time to relaxation for the contractions in the plantarflexor muscle group of the first test mouse; b) i) a second test mouse that possesses a myotonic dystrophy 1 phenotype is subcutaneously administered the vehicle once per week over a four-week period; ii) 35 days after first administration of the vehicle to the second test mouse, the second test mouse is subjected to a muscle lever system while the second test mouse is under anesthesia, wherein a knee of the second test mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the second test mouse for stimulation; iii) stimulating the plantarflexor muscle group of the second test mouse by percutaneous electrical stimulation of a sciatic nerve of the plantarflexor muscle group of the second test mouse with 10 stimulations, each at 150 Hz, with 30 second rest periods between stimulations, thereby promoting contractions in the plantarflexor muscle group of the second test mouse and observing the contractions; and iv) based on observing the contractions in the plantarflexor muscle group of the second test mouse, determining a mean time to relaxation for the contractions in the plantarflexor muscle group of the second test mouse; c) i) a control mouse that does not possess a myotonic dystrophy 1 phenotype is subcutaneously administered the vehicle once per week over a four-week period; ii) 35 days after first administration of the vehicle to the control mouse, the control mouse is subjected to a muscle lever system while the control mouse is under anesthesia, wherein a knee of the control mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the control mouse for stimulation; iii) stimulating the plantarflexor muscle group of the control mouse by percutaneous electrical stimulation of a sciatic nerve of the plantarflexor muscle group of the control mouse with 10 stimulations, each at 150 Hz, with 30 second rest periods between stimulations, thereby promoting contractions in the plantarflexor muscle group of the control mouse and observing the contractions; and iv) based on observing the contractions in the plantarflexor muscle group of the control mouse, determining a mean time to relaxation for the contractions in the plantarflexor muscle group of the control mouse, then:1) the mean time to relaxation for the contractions in the plantarflexor muscle group of the first test mouse is determined to be 0.15 to 0.22 seconds; 2) the mean time to relaxation for the contractions in the plantarflexor muscle group of the second test mouse is determined to be 0.37 to 0.44 seconds; and 3) the mean time to relaxation for the contractions in the plantarflexor muscle group of the control mouse is determined to be 0.05 to 0.09 seconds.

In some embodiments, the present disclosure provides a method of treating myotonic dystrophy 1 in a subject in need thereof, the method comprising administering to the subject a therapeutically-effective amount of a compound, wherein the compound comprises a peptide nucleic acid sequence, wherein the peptide nucleic acid sequence restores skeletal muscle movement in a model of myotonic dystrophy 1.

In some embodiments, if, in a study, a) i) a first test mouse that possesses a myotonic dystrophy 1 phenotype is subcutaneously administered 3 mg/kg of the compound in a vehicle once per week over a four-week period; ii) 35 days after first administration of the compound in the vehicle to the first test mouse, the first test mouse is subjected to a muscle lever system while the first test mouse is under anesthesia, wherein a knee of the first test mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the first test mouse for stimulation; iii) stimulating the plantarflexor muscle group of the first test mouse by percutaneous electrical stimulation of a sciatic nerve of the plantarflexor muscle group of the first test mouse with 10 stimulations, each at 150 Hz, with 30 second rest periods between stimulations, thereby promoting contractions in the plantarflexor muscle group of the first test mouse and observing the contractions; and iv) based on observing the contractions in the plantarflexor muscle group of the first test mouse, determining a mean time to relaxation for the contractions in the plantarflexor muscle group of the first test mouse; b) i) a second test mouse that possesses a myotonic dystrophy 1 phenotype is subcutaneously administered the vehicle once per week over a four-week period; ii) 35 days after first administration of the vehicle to the second test mouse, the second test mouse is subjected to a muscle lever system while the second test mouse is under anesthesia, wherein a knee of the second test mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the second test mouse for stimulation; iii) stimulating the plantarflexor muscle group of the second test mouse by percutaneous electrical stimulation of a sciatic nerve of the plantarflexor muscle group of the second test mouse with 10 stimulations, each at 150 Hz, with 30 second rest periods between stimulations, thereby promoting contractions in the plantarflexor muscle group of the second test mouse and observing the contractions; and iv) based on observing the contractions in the plantarflexor muscle group of the second test mouse, determining a mean time to relaxation for the contractions in the plantarflexor muscle group of the second test mouse; c) i) a control mouse that does not possess a myotonic dystrophy 1 phenotype is subcutaneously administered the vehicle once per week over a four-week period; ii) 35 days after first administration of the vehicle to the control mouse, the control mouse is subjected to a muscle lever system while the control mouse is under anesthesia, wherein a knee of the control mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the control mouse for stimulation; iii) stimulating the plantarflexor muscle group of the control mouse by percutaneous electrical stimulation of a sciatic nerve of the plantarflexor muscle group of the control mouse with 10 stimulations, each at 150 Hz, with 30 second rest periods between stimulations, thereby promoting contractions in the plantarflexor muscle group of the control mouse and observing the contractions; and iv) based on observing the contractions in the plantarflexor muscle group of the control mouse, determining a mean time to relaxation for the contractions in the plantarflexor muscle group of the control mouse, then:

1) the mean time to relaxation for the contractions in the plantarflexor muscle group of the first test mouse is determined to be at most 60% of the mean time to relaxation for the contractions in the plantarflexor muscle group of the second test mouse; 2) the mean time to relaxation for the contractions in the plantarflexor muscle group of the control mouse is determined to be at most 30% of the mean time to relaxation for the contractions in the plantarflexor muscle group of the second test mouse; and 3) the mean time to relaxation for the contractions in the plantarflexor muscle group of the control mouse is determined to be at most 50% of the mean time to relaxation for the contractions in the plantarflexor muscle group of the first test mouse.

In some embodiments, the present disclosure provides a method of treating myotonic dystrophy 1 in a subject in need thereof, the method comprising administering to the subject a therapeutically-effective amount of a compound, wherein the compound comprises a peptide nucleic acid sequence, wherein the peptide nucleic acid sequence restores skeletal muscle movement in a model of myotonic dystrophy 1.

In some embodiments, if, in a study, a) i) a first test mouse that possesses a myotonic dystrophy 1 phenotype is subcutaneously administered 3 mg/kg of the compound in a vehicle once per week over a four-week period; ii) 35 days after first administration of the compound in the vehicle to the first test mouse, the first test mouse is subjected to a muscle lever system while the first test mouse is under anesthesia, wherein a knee of the first test mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the first test mouse for stimulation; iii) stimulating the plantarflexor muscle group of the first test mouse by percutaneous electrical stimulation of a sciatic nerve of the plantarflexor muscle group of the first test mouse with 10 stimulations, each at 150 Hz, with 30 second rest periods between stimulations, thereby promoting contractions in the plantarflexor muscle group of the first test mouse and observing the contractions; and iv) based on observing the contractions in the plantarflexor muscle group of the first test mouse, determining a mean time to relaxation for the contractions in the plantarflexor muscle group of the first test mouse; and b) i) a second test mouse that possesses a myotonic dystrophy 1 phenotype is subcutaneously administered the vehicle once per week over a four-week period; ii) 35 days after first administration of the vehicle to the second test mouse, the second test mouse is subjected to a muscle lever system while the second test mouse is under anesthesia, wherein a knee of the second test mouse is isolated by pressing a pin of the muscle lever system against a head of a tibia connected to the knee and by fixing a foot connected to the tibia to a footplate on a motor shaft of the muscle lever system, thereby presenting a plantarflexor muscle group of the second test mouse for stimulation; iii) stimulating the plantarflexor muscle group of the second test mouse by percutaneous electrical stimulation of a sciatic nerve of the plantarflexor muscle group of the second test mouse with 10 stimulations, each at 150 Hz, with 30 second rest periods between stimulations, thereby promoting contractions in the plantarflexor muscle group of the second test mouse and observing the contractions; and iv) based on observing the contractions in the plantarflexor muscle group of the second test mouse, determining a mean time to relaxation for the contractions in the plantarflexor muscle group of the second test mouse, then the mean time to relaxation for the contractions in the plantarflexor muscle group of the first test mouse is determined to be at most 60% of the mean time to relaxation for the contractions in the plantarflexor muscle group of the second test mouse.

In some embodiments, no histological changes (e.g., in liver or kidney) are observed in animals (e.g., mice) that are administered therapeutic doses of a compound disclosed herein. In some embodiments, no significant changes in cell viability or cytokine activation are observed in blood samples (e.g., human blood samples) treated with a compound disclosed herein.

### Pharmaceutical Compositions

A pharmaceutical composition of the disclosure can be used, for example, before, during, or after treatment of a subject with, for example, another pharmaceutical agent.

Subjects can be, for example, elderly adults, adults, adolescents, pre-adolescents, children, toddlers, infants, neonates, and non-human animals. In some embodiments, a subject is a patient.

A pharmaceutical composition of the disclosure can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compounds of the disclosure can be applied topically to the skin, or a body cavity, for example, oral, vaginal, bladder, cranial, spinal, thoracic, or pelvic cavity of a subject. The compounds of the disclosure can be applied to an accessible body cavity.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Multiple therapeutic agents can be administered in any order or simultaneously. In some embodiments, a compound of the disclosure is administered in combination with, before, or after treatment with another therapeutic agent. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of pharmaceutically active agents suitable for combination with compositions of the disclosure include anti-infectives, i.e., aminoglycosides, antiviral agents, antimicrobials, anticholinergics/antispasmotics, antidiabetic agents, antihypertensive agents, antineoplastics, cardiovascular agents, central nervous system agents, coagulation modifiers, hormones, immunologic agents, immunosuppressive agents, and ophthalmic preparations.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the disclosure can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### EMBODIMENTS

The following non-limiting embodiments in numbered paragraph form provide illustrative examples of the invention, but do not limit the scope of the invention.

A compound comprising a structure, wherein the structure binds to a mRNA sequence transcribed from a gene associated with a neuromuscular disease phenotype, wherein the structure is attached to a chain of atoms bearing a series of side chains, wherein the series of side chains has a sub-series of three consecutive side chains that are: i) guanidinoalkyl; ii) C(O)-alkyl; and iii) guanidinoalkyl.

A compound comprising a structure, wherein the structure binds to a mRNA sequence transcribed from a gene associated with a neuromuscular disease phenotype, wherein the structure is attached to a chain of atoms, wherein carbon atoms of the chain of atoms bear a series of side chains, wherein the series of side chains has two consecutive side chains that are each independently guanidinoalkyl.

A compound comprising a structure, wherein the structure binds to a mRNA sequence that contains a subsequence that is (CUG)_{z}, wherein z is an integer from 1-100 (SEQ ID NO: 135), wherein the structure is attached to a chain of atoms bearing a series of side chains, wherein the series of side chains has a sub-series of three consecutive side chains that are: i) guanidinoalkyl; ii) C(O)-alkyl; and iii) guanidinoalkyl.

A compound comprising a structure, wherein the structure binds to a mRNA sequence that contains a subsequence that is (CUG)_{z}, wherein z is an integer from 1-100 (SEQ ID NO: 135), wherein the structure is attached to a chain of atoms, wherein carbon atoms of the chain of atoms bear a series of side chains, wherein the series of side chains has two consecutive side chains that are each independently guanidinoalkyl.

A compound comprising a structure, wherein the structure binds to a mRNA sequence that contains a subsequence that is (CUG)_{z}, wherein z is an integer from 1-100 (SEQ ID NO: 135), wherein the structure is attached to a chain of atoms, wherein carbon atoms of the chain of atoms bear a series of side chains, wherein the series of side chains has six consecutive side chains that each independently bear a positive charge at physiological pH.

A compound comprising a structure, wherein the structure binds to a mRNA sequence transcribed from a gene associated with a neuromuscular disease phenotype, wherein the structure is attached to a chain of atoms, wherein carbon atoms of the chain of atoms bear a series of side chains, wherein the series of side chains has six consecutive side chains that each independently bear a positive charge at physiological pH.

The compound of paragraph [00348] or paragraph [00350], wherein the sub-series of three consecutive side chains is bound to a sub-series of the chain of atoms comprising a first atom that is bound to a second atom, a third atom that is bound to the second atom, a fourth atom that is bound to the third atom, a fifth atom that is bound to the fourth atom, a sixth atom that is bound to the fifth atom, and a seventh atom that is bound to the sixth atom.

The compound of any one of paragraphs [00348], [00350], and [00354], wherein each of the fifth atom and the sixth atom is not bound to any of guanidinoalkyl and C(O)-alkyl.

The compound of any one of paragraphs [00348], [00350], and [00354], wherein each of the second atom, the fifth atom, and the sixth atom is not bound to any of guanidinoalkyl and C(O)-alkyl.

The compound of any one of paragraphs [00348], [00350], and [00354], wherein each of the first atom, the second atom, the fifth atom, and the sixth atom is not bound to any of guanidinoalkyl and C(O)-alkyl.

The compound of any one of paragraphs [00348], [00350], and [00354], wherein each of the first atom, the fourth atom, the fifth atom, and the sixth atom is not bound to any of guanidinoalkyl and C(O)-alkyl.

The compound of any one of paragraphs [00348], [00350], and [00354], wherein each of the second atom, the fourth atom, the fifth atom, and the sixth atom is not bound to any of guanidinoalkyl and C(O)-alkyl.

The compound of any one of paragraphs [00348], [00350], and [00354]-[00357], wherein the fourth atom is bound to one guanidinoalkyl of the sub-series of three consecutive side chains.

The compound of any one of paragraphs [00348], [00350], [00354]-[00356], and [00358], wherein the second atom is bound to one guanidinoalkyl of the sub-series of three consecutive side chains.

The compound of any one of paragraphs [00348], [00350], [00354]-[00356], and [00359], wherein the first atom is bound to one guanidinoalkyl of the sub-series of three consecutive side chains.

The compound of any one of paragraphs [00348], [00350], [00354]-[00356], [00359], and

, Wherein the seventh atom is bound to one guanidinoalkyl of the sub-series of three consecutive side chains.

The compound of any one of paragraphs [00348], [00350], and [00354]-[00363], wherein the third atom is bound the C(O)-alkyl of the sub-series of three consecutive side chains.

The compound of paragraph [00354] or paragraph [00355], wherein the sub-series of the chain of atoms further comprises a eighth atom that is bound to the seventh atom, a ninth atom that is bound to the eighth atom, a tenth atom that is bound to the ninth atom, an eleventh atom that is bound to the tenth atom, and a twelfth atom that is bound to the eleventh atom.

The compound of paragraph [00365], wherein each of the eleventh atom and the twelfth atom is not bound to any of guanidinoalkyl and C(O)-alkyl.

The compound of paragraph [00365], wherein each of the eighth atom, the eleventh atom, and the twelfth atom is not bound to any of guanidinoalkyl and C(O)-alkyl.

The compound of paragraph [00365], wherein each of the seventh atom, the eighth atom, the eleventh atom, and the twelfth atom is not bound to any of guanidinoalkyl and C(O)-alkyl.

The compound of any one of paragraphs [00365]-[00368], wherein the tenth atom is bound to guanidinoalkyl.

The compound of paragraph [00365], wherein each of the seventh atom, the tenth atom, the eleventh atom, and the twelfth atom is not bound to any of guanidinoalkyl and C(O)-alkyl.

The compound of any one of paragraphs [00365], [00366], and [00370], wherein the eighth atom is bound to guanidinoalkyl.

The compound of paragraph [00365], wherein each of the eighth atom, the tenth atom, the eleventh atom, and the twelfth atom is not bound to any of guanidinoalkyl and C(O)-alkyl.

The compound of any one of paragraphs [00365]-[00372], wherein the ninth atom is bound to C(O)-alkyl.

The compound of any one of paragraphs [00365]-[00373], wherein the first through twelfth atoms of the sub-series of the chain of atoms together form a repeating unit of a polymer backbone.

The compound of paragraph [00374], wherein the polymer backbone is a peptide nucleic acid backbone.

The compound of paragraph [00349] or paragraph [00351], wherein the chain of atoms comprises a first atom that is bound to a second atom, a third atom that is bound to the second atom, a fourth atom that is bound to the third atom, a fifth atom that is bound to the fourth atom, a sixth atom that is bound to the fifth atom, a seventh atom that is bound to the sixth atom, an eighth atom that is bound to the seventh atom, a ninth atom that is bound to the eighth atom, a tenth atom that is bound to the ninth atom, an eleventh atom that is bound to the tenth atom, and a twelfth atom that is bound to the eleventh atom.

The compound of paragraph [00376], wherein each of the third atom, the fifth atom, the sixth atom, the ninth atom, the eleventh atom, and the twelfth atom is not bound to guanidinoalkyl.

The compound of paragraph [00376], wherein each of the second atom, the third atom, the fifth atom, the sixth atom, the eighth atom, the ninth atom, the eleventh atom, and the twelfth atom is not bound to guanidinoalkyl.

The compound of paragraph [00376], wherein each of the first atom, the second atom, the third atom, the fifth atom, the sixth atom, the seventh atom, the eighth atom, the ninth atom, the eleventh atom, and the twelfth atom is not bound to guanidinoalkyl.

The compound of any one of paragraphs [00376]-[00379], wherein the fourth atom is bound to guanidinoalkyl.

The compound of any one of paragraphs [00376]-[00379], wherein the tenth atom is bound to guanidinoalkyl.

The compound of paragraph [00376], wherein each of the second atom, the third atom, the fourth atom, the fifth atom, the sixth atom, the eighth atom, the ninth atom, the tenth atom, the eleventh atom, and the twelfth atom is not bound to guanidinoalkyl.

The compound of any one of paragraphs [00376]-[00378], and [00382], wherein the first atom is bound to guanidinoalkyl.

The compound of any one of paragraphs [00376]-[00378], [00382], and [00383], wherein the seventh atom is bound to guanidinoalkyl.

The compound of paragraph [00376], wherein each of the first atom, the third atom, the fourth atom, the fifth atom, the sixth atom, the seventh atom, the ninth atom, the tenth atom, the eleventh atom, and the twelfth atom is not bound to guanidinoalkyl.

The compound of paragraph [00376] or paragraph [00385], wherein the second atom is bound to guanidinoalkyl.

The compound of any one of paragraphs [00376], [00385], and [00386], wherein the eighth atom is bound to guanidinoalkyl.

The compound of any one of paragraphs [00376]-[00387], wherein the third atom is bound to a C(O)-alkyl group.

The compound of any one of paragraphs [00376]-[00388], wherein the ninth atom is bound to a C(O)-alkyl group.

The compound of any one of paragraphs [00376]-[00389], wherein the first through twelfth atoms together form a repeating unit of a polymer backbone.

The compound of paragraph [00390], wherein the polymer backbone is a peptide nucleic acid backbone.

The compound of paragraph [00352] or paragraph [00353], wherein the chain of atoms comprises a first atom that is bound to a second atom, a third atom that is bound to the second atom, a fourth atom that is bound to the third atom, a fifth atom that is bound to the fourth atom, and a sixth atom that is bound to the fifth atom, wherein the first atom is bound to a side chain of the series of side chains, and each of the second atom, the third atom, the fourth atom, the fifth atom, and the sixth atom is not bound to a side chain of the series of side chains.

The compound of paragraph [00392], wherein the third atom is bound to a C(O)-alkyl group.

The compound of paragraph [00392], wherein the second atom is bound to a C(O)-alkyl group.

The compound of paragraph [00392], wherein the sixth atom is bound to a C(O)-alkyl group.

The compound of any one of paragraphs [00392]-[00395], wherein the first through sixth atoms together form a repeating unit of a polymer backbone.

The compound of paragraph [00396], wherein the polymer backbone is a peptide nucleic acid backbone.

The compound of any one of paragraphs [00352], [00353], and [00392]-[00397], wherein each of the side chains is independently aminoalkyl, guanidinoalkyl, ureidolalkyl, amidinoalkyl, morpholinoalkyl, piperidinylalkyl, piperazinylalkyl, or pyrrolidinylalkyl.

The compound of any one of paragraphs [00352], [00353], and [00392]-[00397], wherein each of the side chains is independently aminoalkyl or guanidinoalkyl.

The compound of any one of paragraphs [00352], [00353], and [00392]-[00397], wherein each of the side chains is independently guanidinoalkyl.

The compound of any one of paragraphs [00348]-[00351], [00354]-[00391], and [00398]-[00400], wherein each guanidinoalkyl is independently H, 3-guanidinoprop-1-yl, or 4-guanidinobut-1-yl.

The compound of any one of paragraphs [00348]-[00351], [00354]-[00391], and [00398]-[00400], wherein each guanidinoalkyl is 3-guanidinoprop-1-yl.

The compound of any one of paragraphs [00348]-[00351], [00354]-[00391], and [00398]-[00400], wherein each guanidinoalkyl is 4-guanidinobut-1-yl.

The compound of any one of paragraphs [00348], [00350], [00354]-[00364], [00366], [00368], [00370], [00372], [00373], [00388], [00389], [00393], [00394], and [00395], wherein each C(O)-alkyl group is independently acetyl.

The compound of any one of paragraphs [00348], [00349], and [00353], wherein the neuromuscular disease phenotype is a DM1 disease phenotype.

The compound of any one of paragraphs [00348], [00349], [00353], and [00405], wherein the mRNA sequence contains a subsequence that is (CUG)_{z}, wherein z is an integer from 1-100 (SEQ ID NO: 135).

The compound of any one of paragraphs [00350], [00351], [00352], and [00406], wherein the structure binds to the mRNA sequence at the subsequence that is (CUG)_{z}.

The compound of any one of paragraphs [00348]-[00407], wherein the structure is a oligonucleotide or oligonucleotide analogue.

The compound of any one of paragraphs [00348]-[00407], wherein the structure is a peptide nucleic acid.

A compound comprising a structure that is:

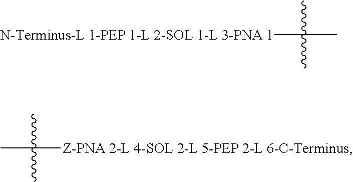

wherein:
the number of units with variables defined independently is at least 3;
N-Terminus is H, acyl, a group that together with the nitrogen atom to which N-Terminus is bound forms a carbamate, a probe, a metal chelator, or a biological agent; C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
Z is a peptide nucleic acid sequence;
PNA2 is a peptide nucleic acid sequence or absent;
L1 is a linker group or absent;
L2 is a linker group or absent;
L3 is a linker group or absent;
L4 is a linker group or absent;
L5 is a linker group or absent;
L6 is a linker group or absent; and
   or a pharmaceutically-acceptable salt or ionized form thereof.

The compound of paragraph [00410], wherein Z is a peptide nucleic acid sequence according to PNA SEQ NO: 1, PNA SEQ NO: 2, PNA SEQ NO: 3, PNA SEQ NO: 4, PNA SEQ NO: 5, PNA SEQ NO: 6, PNA SEQ NO: 7, PNA SEQ NO: 8, PNA SEQ NO: 9, PNA SEQ NO: 10, PNA SEQ NO: 11, PNA SEQ NO: 12, PNA SEQ NO: 13, PNA SEQ NO: 14, PNA SEQ NO: 15, PNA SEQ NO: 16, PNA SEQ NO: 17, PNA SEQ NO: 18, PNA SEQ NO: 19, PNA SEQ NO: 20, PNA SEQ NO: 21, PNA SEQ NO: 22, PNA SEQ NO: 23, PNA SEQ NO: 34, PNA SEQ NO: 25, PNA SEQ NO: 26, PNA SEQ NO: 27, PNA SEQ NO: 28, PNA SEQ NO: 29, PNA SEQ NO: 30, PNA SEQ NO: 31, PNA SEQ NO: 32, PNA SEQ NO: 33, PNA SEQ NO: 34, PNA SEQ NO: 35, PNA SEQ NO: 36, PNA SEQ NO: 37, PNA SEQ NO: 38, PNA SEQ NO: 39, PNA SEQ NO: 40, PNA SEQ NO: 41, PNA SEQ NO: 42, PNA SEQ NO: 43, or PNA SEQ NO: 44.

The compound of paragraph [00410], wherein Z is a peptide nucleic acid sequence according to PNA SEQ NO: 28, PNA SEQ NO: 29, PNA SEQ NO: 30, PNA SEQ NO: 31, PNA SEQ NO: 32, PNA SEQ NO: 33, PNA SEQ NO: 34, PNA SEQ NO: 35, PNA SEQ NO: 36, PNA SEQ NO: 37, PNA SEQ NO: 38, PNA SEQ NO: 39, PNA SEQ NO: 40, PNA SEQ NO: 41, PNA SEQ NO: 42, PNA SEQ NO: 43, or PNA SEQ NO: 44.

The compound of paragraph [00410], wherein Z is a peptide nucleic acid sequence complementary to a nucleic acid sequence selected from the group consisting of: CUG, CUGC, CUGCU, CUGCUG, CUGCUGC, CUGCUGCU, CUGCUGCUG, CUGCUGCUGC (SEQ ID NO: 96), CUGCUGCUGCU (SEQ ID NO: 97), CUGCUGCUGCUG (SEQ ID NO: 98), CUGCUGCUGCUGC (SEQ ID NO: 99), CUGCUGCUGCUGCU (SEQ ID NO: 100), CUGCUGCUGCUGCUG (SEQ ID NO: 101), CUGCUGCUGCUGCUGC (SEQ ID NO: 102), CUGCUGCUGCUGCUGCU (SEQ ID NO: 103), CUGCUGCUGCUGCUGCUG (SEQ ID NO: 104), CUGCUGCUGCUGCUGCUGC (SEQ ID NO: 105), CUGCUGCUGCUGCUGCUGCU (SEQ ID NO: 106), CUGCUGCUGCUGCUGCUGCUG (SEQ ID NO: 107), CUGCUGCUGCUGCUGCUGCUGC (SEQ ID NO: 108), CUGCUGCUGCUGCUGCUGCUGCU (SEQ ID NO: 109), and CUGCUGCUGCUGCUGCUGCUGCUG (SEQ ID NO: 110).

A compound comprising a structure that is:

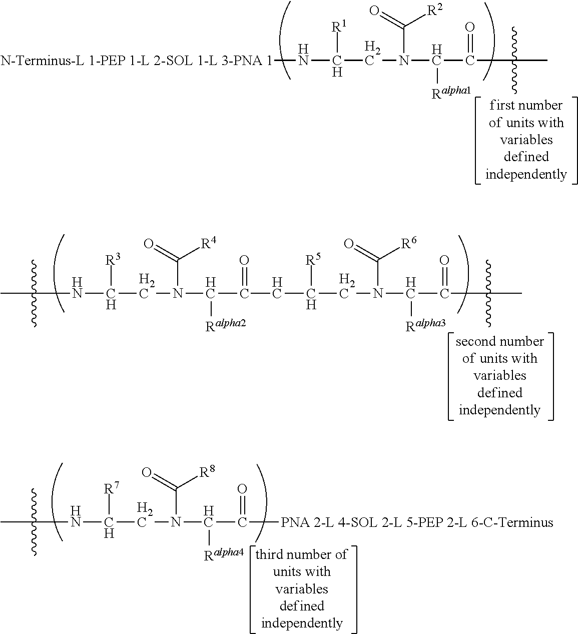

wherein:
the first number of units with variables defined independently is at least zero;
the second number of units with variables defined independently is at least 3;
the third number of units with variables defined independently is at least zero;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R¹ is independently alkyl, O-alkyl, or methyl substituted with a heterocycle; each R³ is independently alkyl that is unsubstituted or substituted or H;
each R⁴ is independently R²;
each R⁵ is independently alkyl that is unsubstituted or substituted or H;
each R⁶ is independently R²;
each R⁷ is independently alkyl that is unsubstituted or substituted or H;
each R⁸ is independently R²,
wherein at least two R² groups in the structure are independently methyl substituted with a heterocycle;
each R^{alpha1} is independently alkyl that is unsubstituted or substituted or H;
each R^{alpha2} is independently alkyl that is unsubstituted or substituted or H;
each R^{alpha3} is independently alkyl that is unsubstituted or substituted or H;
each R^{alpha4} is independently alkyl that is unsubstituted or substituted or H;
C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof.

The compound of paragraph [00414], wherein the first number of units with variables defined independently is 3-1,000.

The compound of paragraph [00414], wherein the first number of units with variables defined independently is 3-100.

The compound of paragraph [00414], wherein the first number of units with variables defined independently is 3-50.

The compound of paragraph [00414], wherein the first number of units with variables defined independently is 3, 4, 5, 6, 7, 8, 9, 10, 11, 12, 13, 14, 15, 16, 17, 18, 19, 20, 21, 22, 23, 24, 25, 26, 27, 28, 29, or 30.

The compound of paragraph [00414], wherein the first number of units with variables defined independently is zero.

The compound of paragraph [00414], wherein the first number of units with variables defined independently is at least 11.

The compound of paragraph [00414], wherein the first number of units with variables defined independently is 11-1,000.

The compound of paragraph [00414], wherein the first number of units with variables defined independently is 11-100.

The compound of paragraph [00414], wherein the first number of units with variables defined independently is 11-50.

The compound of paragraph [00414], wherein the first number of units with variables defined independently is 11, 12, 13, 14, 15, 16, 17, 18, 19, 20, 21, 22, 23, 24, 25, 26, 27, 28, 29, or 30.

The compound of any one of paragraphs [00414]-[00424], wherein the second number of units with variables defined independently is 3-1,000.

The compound of any one of paragraphs [00414]-[00424], wherein the second number of units with variables defined independently is 3-100.

The compound of any one of paragraphs [00414]-[00424], wherein the second number of units with variables defined independently is 3-50.

The compound of any one of paragraphs [00414]-[00424], wherein the second number of units with variables defined independently is 3, 4, 5, 6, 7, 8, 9, 10, 11, 12, 13, 14, 15, 16, 17, 18, 19, 20, 21, 22, 23, 24, 25, 26, 27, 28, 29, or 30.

The compound of any one of paragraphs [00414]-[00424], wherein the second number of units with variables defined independently is 3, 4, 5, 6, 7, 8, 9, 10, 11, 12, 13, 14, 15, 16, 17, 18, 19, or 20.

The compound of any one of paragraphs [00414]-[00424], wherein the second number of units with variables defined independently is 3, 4, 5, 6, 7, 8, 9, or 10.

The compound of any one of paragraphs [00414]-[00430], wherein the third number of units with variables defined independently is 3-1,000.

The compound of any one of paragraphs [00414]-[00430], wherein the third number of units with variables defined independently is 3-100.

The compound of any one of paragraphs [00414]-[00430], wherein the third number of units with variables defined independently is 3-50.

The compound of any one of paragraphs [00414]-[00430], wherein the third number of units with variables defined independently is 3, 4, 5, 6, 7, 8, 9, 10, 11, 12, 13, 14, 15, 16, 17, 18, 19, 20, 21, 22, 23, 24, 25, 26, 27, 28, 29, or 30.

The compound of any one of paragraphs [00414]-[00430], wherein the third number of units with variables defined independently is zero.

The compound of any one of paragraphs [00414]-[00430], wherein the third number of units with variables defined independently is at least 11.

The compound of any one of paragraphs [00414]-[00430], wherein the third number of units with variables defined independently is 11-1,000.

The compound of any one of paragraphs [00414]-[00430], wherein the third number of units with variables defined independently is 11-100.

The compound of any one of paragraphs [00414]-[00430], wherein the third number of units with variables defined independently is 11-50.

The compound of any one of paragraphs [00414]-[00430], wherein the third number of units with variables defined independently is 11, 12, 13, 14, 15, 16, 17, 18, 19, 20, 21, 22, 23, 24, 25, 26, 27, 28, 29, or 30.

The compound of any one of paragraphs [00414]-[00440], wherein each R^{alpha1} is H.

The compound of any one of paragraphs [00414]-[00440], wherein each R^{alpha1} is independently alkyl that is unsubstituted.

The compound of paragraph [00442], wherein each alkyl that is unsubstituted is independently methyl, ethyl, prop-1-yl, prop-2-yl, 2-methylprop-1-yl, but-1yl, but-2-yl, or pent-1-yl.

The compound of paragraph [00442], wherein each alkyl that is unsubstituted is independently methyl, prop-2-yl, 2-methylprop-1-yl, or but-2-yl.

The compound of any one of paragraphs [00414]-[00440], wherein each R^{alpha1} is independently alkyl that is substituted.

The compound of paragraph [00445], wherein each alkyl that is substituted is independently substituted with —OH, —SH, —SMe, —NH₂, a heterocycle, an aryl group, a carboxylic acid, a guanidino group, a N-methylguanidino group, or an amido group.

The compound of paragraph [00445], wherein each alkyl that is substituted is independently hydroxymethyl, 1-hydroxyeth-1-yl, sulfhydrylmethyl, 2-thiomethyleth-1-yl, 4-aminobut-1-yl, 3-aminoprop-1-yl, 1-H-imidazol-4-ylmethyl, 1-H-indol-3-ylmethyl, benzyl, 4-hydroxyphen-1-ylmethyl, 2-carboxylatoeth-1-yl, 3-carboxylatoprop-1-yl, 3-guanidinoprop-1-yl, 4-guanidinobut-1-yl, 2-carbamoyleth-1-yl, or 3-carbamoylprop-1-yl.

The compound of any one of paragraphs [00414]-[00440], wherein each R^{alpha1} is independently a guanidinoalkyl group or a hydroxyalkyl group.

The compound of any one of paragraphs [00414]-[00440], wherein each R^{alpha1} is hydroxymethyl.

The compound of any one of paragraphs [00414]-[00440], wherein each R^{alpha1} is independently H, 3-guanidinoprop-1-yl, or 4-guanidinobut-1-yl.

The compound of any one of paragraphs [00414]-[00440], wherein at least one iteration of R^{alpha1} is 3-guanidinoprop-1-yl.

The compound of any one of paragraphs [00414]-[00440], wherein at least a third of the iterations of R^{alpha1} are 3-guanidinoprop-1-yl.

The compound of any one of paragraphs [00414]-[00440], wherein at least half the iterations of R^{alpha1} are 3-guanidinoprop-1-yl.

The compound of any one of paragraphs [00414]-[00453], wherein each R^{alpha4} is H.

The compound of any one of paragraphs [00414]-[00453], wherein each R^{alpha4} is independently alkyl that is unsubstituted.

The compound of paragraph [00455], wherein each alkyl that is unsubstituted is independently methyl, ethyl, prop-1-yl, prop-2-yl, 2-methylprop-1-yl, but-1yl, but-2-yl, or pent-1-yl.

The compound of paragraph [00455], wherein each alkyl that is unsubstituted is independently methyl, prop-2-yl, 2-methylprop-1-yl, or but-2-yl.

The compound of any one of paragraphs [00414]-[00453], wherein each R^{alpha4} is independently alkyl that is substituted.

The compound of paragraph [00458], wherein each alkyl that is substituted is independently substituted with —OH, —SH, —SMe, —NH₂, a heterocycle, an aryl group, a carboxylic acid, a guanidino group, a N-methylguanidino group, or an amido group.

The compound of paragraph [00458], wherein each alkyl that is substituted is independently hydroxymethyl, 1-hydroxyeth-1-yl, sulfhydrylmethyl, 2-thiomethyleth-1-yl, 4-aminobut-1-yl, 3-aminoprop-1-yl, 1-H-imidazol-4-ylmethyl, 1-H-indol-3-ylmethyl, benzyl, 4-hydroxyphen-1-ylmethyl, 2-carboxylatoeth-1-yl, 3-carboxylatoprop-1-yl, 3-guanidinoprop-1-yl, 4-guanidinobut-1-yl, 2-carbamoyleth-1-yl, or 3-carbamoylprop-1-yl.

The compound of any one of paragraphs [00414]-[00453], wherein each R^{alpha4} is independently a guanidinoalkyl group or a hydroxyalkyl group.

The compound of any one of paragraphs [00414]-[00453], wherein each R^{alpha4} is hydroxymethyl.

The compound of any one of paragraphs [00414]-[00453], wherein each R^{alpha4} is independently H, 3-guanidinoprop-1-yl, or 4-guanidinobut-1-yl.

The compound of any one of paragraphs [00414]-[00453], wherein at least one iteration of R^{alpha4} is 3-guanidinoprop-1-yl.

The compound of any one of paragraphs [00414]-[00453], wherein at least a third of the iterations of R^{alpha4} are 3-guanidinoprop-1-yl.

The compound of any one of paragraphs [00414]-[00453], wherein at least half the iterations of R^{alpha} 4 are 3-guanidinoprop-1-yl.

The compound of any one of paragraphs [00414]-[00466], wherein each R¹ is independently alkyl that is unsubstituted.

The compound of paragraph [00467], wherein each alkyl that is unsubstituted is independently methyl, ethyl, prop-1-yl, prop-2-yl, 2-methylprop-1-yl, but-1yl, but-2-yl, or pent-1-yl.

The compound of paragraph [00467], wherein each alkyl that is unsubstituted is independently methyl, prop-2-yl, 2-methylprop-1-yl, or but-2-yl.

The compound of any one of paragraphs [00414]-[00466], wherein each R¹ is independently alkyl that is substituted.

The compound of paragraph [00470], wherein each alkyl that is substituted is independently substituted with —OH, —SH, —SMe, —NH₂, a heterocycle, an aryl group, a carboxylic acid, a guanidino group, a N-methylguanidino group, or an amido group.

The compound of paragraph [00470], wherein each alkyl that is substituted is independently hydroxymethyl, 1-hydroxyeth-1-yl, sulfhydrylmethyl, 2-thiomethyleth-1-yl, 4-aminobut-1-yl, 3-aminoprop-1-yl, 1-H-imidazol-4-ylmethyl, 1-H-indol-3-ylmethyl, benzyl, 4-hydroxyphen-1-ylmethyl, 2-carboxylatoeth-1-yl, 3-carboxylatoprop-1-yl, 3-guanidinoprop-1-yl, 4-guanidinobut-1-yl, 2-carbamoyleth-1-yl, or 3-carbamoylprop-1-yl.

The compound of any one of paragraphs [00414]-[00466], wherein each R¹ is independently H, hydroxylmethyl, or 4-guanidinobut-1-yl.

The compound of any one of paragraphs [00414]-[00466] and [00470]-[00473], wherein at least one iteration of R¹ is a hydroxyalkyl group.

The compound of any one of paragraphs [00414]-[00466] and [00470]-[00473], wherein at least one iteration of R¹ is hydroxylmethyl.

The compound of any one of paragraphs [00414]-[00466] and [00470]-[00473], wherein at least a third of the iterations of R¹ are hydroxylmethyl.

The compound of any one of paragraphs [00414]-[00466] and [00470]-[00473], wherein at least half the iterations of R¹ are hydroxylmethyl.

The compound of any one of paragraphs [00414]-[00477], wherein each R⁷ is independently alkyl that is unsubstituted.

The compound of paragraph [00478], wherein each alkyl that is unsubstituted is independently methyl, ethyl, prop-1-yl, prop-2-yl, 2-methylprop-1-yl, but-1yl, but-2-yl, or pent-1-yl.

The compound of paragraph [00478], wherein each alkyl that is unsubstituted is independently methyl, prop-2-yl, 2-methylprop-1-yl, or but-2-yl.

The compound of any one of paragraphs [00414]-[00477], wherein each R⁷ is independently alkyl that is substituted.

The compound of paragraph [00481], wherein each alkyl that is substituted is independently substituted with —OH, —SH, —SMe, —NH₂, a heterocycle, an aryl group, a carboxylic acid, a guanidino group, a N-methylguanidino group, or an amido group.

The compound of paragraph [00481], wherein each alkyl that is substituted is independently hydroxymethyl, 1-hydroxyeth-1-yl, sulfhydrylmethyl, 2-thiomethyleth-1-yl, 4-aminobut-1-yl, 3-aminoprop-1-yl, 1-H-imidazol-4-ylmethyl, 1-H-indol-3-ylmethyl, benzyl, 4-hydroxyphen-1-ylmethyl, 2-carboxylatoeth-1-yl, 3-carboxylatoprop-1-yl, 3-guanidinoprop-1-yl, 4-guanidinobut-1-yl, 2-carbamoyleth-1-yl, or 3-carbamoylprop-1-yl.

The compound of any one of paragraphs [00414]-[00477], wherein each R⁷ is independently H, hydroxylmethyl, or 4-guanidinobut-1-yl.

The compound of any one of paragraphs [00414]-[00477] and [00481]-[00483], wherein at least one iteration of R⁷ is a hydroxyalkyl group.

The compound of any one of paragraphs [00414]-[00477] and [00481]-[00483], wherein at least one iteration of R⁷ is hydroxylmethyl.

The compound of any one of paragraphs [00414]-[00477] and [00481]-[00483], wherein at least a third of the iterations of R⁷ are hydroxylmethyl.

The compound of any one of paragraphs [00414]-[00477] and [00481]-[00483], wherein at least half the iterations of R⁷ are hydroxylmethyl.

The compound of any one of paragraphs [00414]-[00488], wherein each R³ is independently an alkyl group that is unsubstituted or substituted, and each R⁵ is independently a group that is not substituted alkyl.

The compound of any one of paragraphs [00414]-[00488], wherein each R³ is independently a guanidinoalkyl group, and each R⁵ is independently a group that is not guanidinoalkyl.

The compound of any one of paragraphs [00414]-[00488], wherein each R³ is independently a hydroxyalkyl group, and each R⁵ is independently a group that is not hydroxyalkyl.

The compound of any one of paragraphs [00414]-[00491], wherein each R³ is hydroxymethyl.

The compound of any one of paragraphs [00414]-[00492], wherein each R⁵ is H. The compound of any one of paragraphs [00414]-[00493], wherein each R^{alpha2} and each Rₐₗₚₕₐ₃ is H.

The compound of any one of paragraphs [00414]-[00488], wherein each R^{alpha2} is independently an alkyl group that is unsubstituted or substituted, and R^{alpha3} is independently a group that is not substituted alkyl.

The compound of any one of paragraphs [00414]-[00488], wherein each R^{alpha2} is independently a guanidinoalkyl group, and each R^{alpha} 3 is independently a group that is not guanidinoalkyl.

The compound of any one of paragraphs [00414]-[00488], wherein each R^{alpha2} is independently a hydroxyalkyl group, and each R^{alpha} 3 is independently a group that is not hydroxyalkyl.

The compound of any one of paragraphs [00495]-[00497], wherein each R^{alpha2} is hydroxymethyl.

The compound of any one of paragraphs [00495]-[00498], wherein each R^{alpha3} is H.

The compound of any one of paragraphs [00495]-[00499], wherein each R³ and each R⁵ is H.

A compound comprising a structure that is:

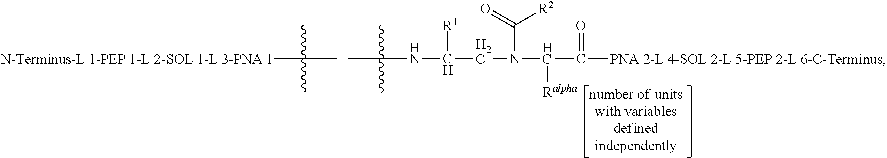

wherein:
the number of units with variables defined independently is at least 3;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle;
C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof.

The compound of paragraph [00501], wherein the number of units with variables defined independently is 11-1,000.

The compound of paragraph [00501], wherein the number of units with variables defined independently is 11-100.

The compound of paragraph [00501], wherein the number of units with variables defined independently is 11-50.

The compound of paragraph [00501], wherein the number of units with variables defined independently is 11, 12, 13, 14, 15, 16, 17, 18, 19, 20, 21, 22, 23, 24, 25, 26, 27, 28, 29, or 30.

The compound of any one of paragraphs [00501]-[00505], wherein each R^{alpha} is H.

The compound of any one of paragraphs [00501]-[00505], wherein each R^{alpha} is independently alkyl that is unsubstituted.

The compound of paragraph [00506], wherein each alkyl that is unsubstituted is independently methyl, ethyl, prop-1-yl, prop-2-yl, 2-methylprop-1-yl, but-1yl, but-2-yl, or pent-1-yl.

The compound of paragraph [00506], wherein each alkyl that is unsubstituted is independently methyl, prop-2-yl, 2-methylprop-1-yl, or but-2-yl.

The compound of any one of paragraphs [00501]-[00505], wherein each R^{alpha} is independently alkyl that is substituted.

The compound of paragraph [00510], wherein each alkyl that is substituted is independently substituted with —OH, —SH, —SMe, —NH₂, a heterocycle, an aryl group, a carboxylic acid, a guanidino group, a N-methylguanidino group, or an amido group.

The compound of paragraph [00510], wherein each alkyl that is substituted is independently hydroxymethyl, 1-hydroxyeth-1-yl, sulfhydrylmethyl, 2-thiomethyleth-1-yl, 4-aminobut-1-yl, 3-aminoprop-1-yl, 1-H-imidazol-4-ylmethyl, 1-H-indol-3-ylmethyl, benzyl, 4-hydroxyphen-1-ylmethyl, 2-carboxylatoeth-1-yl, 3-carboxylatoprop-1-yl, 3-guanidinoprop-1-yl, 4-guanidinobut-1-yl, 2-carbamoyleth-1-yl, or 3-carbamoylprop-1-yl.

The compound of any one of paragraphs [00501]-[00505], wherein each R^{alpha} is independently H, 3-guanidinoprop-1-yl, or 4-guanidinobut-1-yl.

The compound of paragraph [00513], wherein at least one iteration of R^{alpha} is 3-guanidinoprop-1-yl.

The compound of paragraph [00513], wherein at least a third of the iterations of R^{alpha} are 3-guanidinoprop-1-yl.

The compound of paragraph [00513], wherein at least half the iterations of R^{alpha} are 3-guanidinoprop-1-yl.

The compound of any one of paragraphs [00501]-[00516], wherein the number of units with variables defined independently is at least 11; and at least one iteration of R¹ is a hydroxyalkyl group.

The compound of any one of paragraphs [00501]-[00517], wherein the number of units with variables defined independently is 14, 15, 16, 17, or 18, wherein:
a first unit is present, and in the first unit:
R¹ is H or —CH₂OH; and R² is

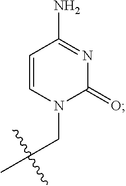 [SMILES: *Cn1ccc(N)nc1=O]

a second unit is present, and in the second unit:
R¹ is H or —CH₂OH; and R² is

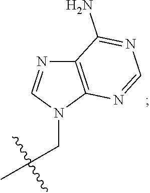 [SMILES: *Cn1cnc2c(N)ncnc21]

a third unit is present, and in the third unit:
R¹ is H or —CH₂OH; and R² is

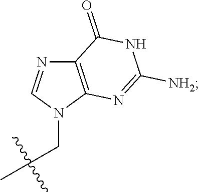 [SMILES: *Cn1cnc2c(=O)[nH]c(N)nc21]

a fourth unit is present, and in the fourth unit:
R¹ is H or —CH₂OH; and R² is

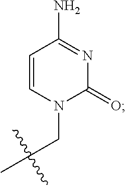 [SMILES: *Cn1ccc(N)nc1=O]

a fifth unit is present, and in the fifth unit:
R¹ is H or —CH₂OH; and R² is

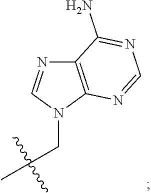 [SMILES: *Cn1cnc2c(N)ncnc21]

a sixth unit is present, and in the sixth unit:
R¹ is H or —CH₂OH; and R² is

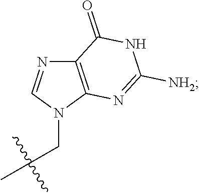 [SMILES: *Cn1cnc2c(=O)[nH]c(N)nc21]

a seventh unit is present, and in the seventh unit:
R¹ is H or —CH₂OH; and R² is

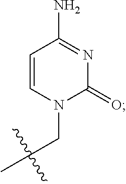 [SMILES: *Cn1ccc(N)nc1=O]

an eighth unit is present, and in the eighth unit:
R¹ is H or —CH₂OH; and R² is

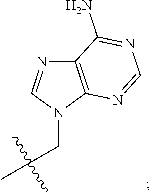 [SMILES: *Cn1cnc2c(N)ncnc21]

a ninth unit is present, and in the ninth unit:

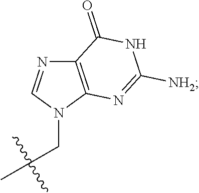 [SMILES: *Cn1cnc2c(=O)[nH]c(N)nc21]

R¹ is H or —CH₂OH; and R² is
a tenth unit is present, and in the tenth unit:
R¹ is H or —CH₂OH; and R² is

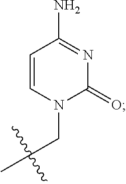 [SMILES: *Cn1ccc(N)nc1=O]

an eleventh unit is present, and in the eleventh unit:
R¹ is H or —CH₂OH; and R² is

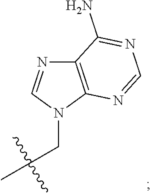 [SMILES: *Cn1cnc2c(N)ncnc21]

a twelfth unit is present, and in the twelfth unit:
R¹ is H or —CH₂OH; and R² is

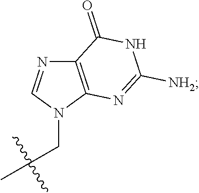 [SMILES: *Cn1cnc2c(=O)[nH]c(N)nc21]

a thirteenth unit is present, and in the thirteenth unit:
R¹ is H or —CH₂OH; and R² is

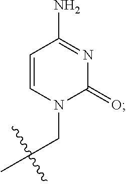 [SMILES: *Cn1ccc(N)nc1=O]

a fourteenth unit is present, and in the fourteenth unit:
R¹ is H or —CH₂OH; and R² is

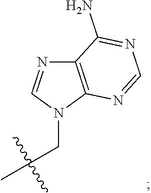 [SMILES: *Cn1cnc2c(N)ncnc21]

a fifteenth unit is present or absent, and in the fifteenth unit:
R¹ is H or —CH₂OH; and R² is

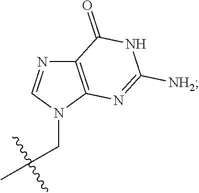 [SMILES: *Cn1cnc2c(=O)[nH]c(N)nc21]

a sixteenth unit is present or absent, and in the sixteenth unit:
R¹ is H or —CH₂OH; and R² is

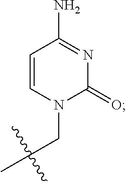 [SMILES: *Cn1ccc(N)nc1=O]

a seventeenth unit is present or absent, and in the seventeenth unit:
R¹ is H or —CH₂OH; and R² is

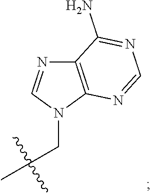 [SMILES: *Cn1cnc2c(N)ncnc21]

and
an eighteenth unit is present or absent, and in the eighteenth unit:
R¹ is H or —CH₂OH; and R² is

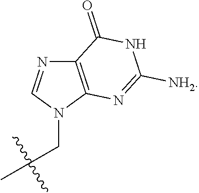 [SMILES: *Cn1cnc2c(=O)[nH]c(N)nc21]

The compound of any one of paragraphs [00414]-[00518], wherein at least a third of the R² groups in the structure are methyl substituted with a heterocycle.

The compound of any one of paragraphs [00414]-[00518], wherein at least half of the R² groups in the structure are methyl substituted with a heterocycle.

The compound of any one of paragraphs [00414]-[00518], wherein the heterocycles of the R² groups are nucleobases or analogues of nucleobases.

The compound of any one of paragraphs [00414]-[00516], wherein at least one of the heterocycles of the R² groups is a divalent nucleobase.

The compound of any one of paragraphs [00414]-[00516], wherein the heterocycles of the R² groups are divalent nucleobases.

The compound of any one of paragraphs [00414]-[00521], wherein the heterocycles of the R² groups are each independently:

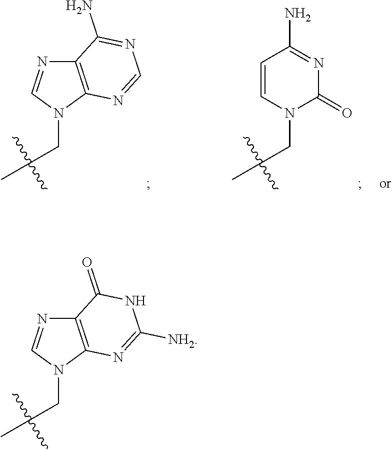 [SMILES: *Cn1cnc2c(N)ncnc21 ; *Cn1ccc(N)nc1=O ; *Cn1cnc2c(=O)[nH]c(N)nc21]

The compound of any one of paragraphs [00414]-[00521], wherein each R² is independently: methyl,

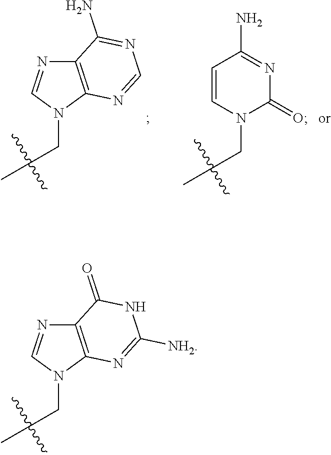

The compound of any one of paragraphs [00501]-[00518], wherein of the units with variables defined independently, counting from N-Terminus, the first, third, sixth, ninth, eleventh, thirteenth, sixteenth, nineteenth, and twenty-second units, independently if present, each have 3-guanidinoprop-1-yl at R^{alpha}.

The compound of any one of paragraphs [00501]-[00516], wherein each R¹ is independently alkyl that is unsubstituted.

The compound of paragraph [00527], wherein each alkyl that is unsubstituted is independently methyl, ethyl, prop-1-yl, prop-2-yl, 2-methylprop-1-yl, but-1yl, but-2-yl, or pent-1-yl.

The compound of paragraph [00527], wherein each alkyl that is unsubstituted is independently methyl, prop-2-yl, 2-methylprop-1-yl, or but-2-yl.

The compound of any one of paragraphs [00501]-[00516], wherein each R¹ is independently alkyl that is substituted.

The compound of paragraph [00530], wherein each alkyl that is substituted is independently substituted with —OH, —SH, —SMe, —NH₂, a heterocycle, an aryl group, a carboxylic acid, a guanidino group, a N-methylguanidino group, or an amido group.

The compound of paragraph [00530], wherein each alkyl that is substituted is independently hydroxymethyl, 1-hydroxyeth-1-yl, sulfhydrylmethyl, 2-thiomethyleth-1-yl, 4-aminobut-1-yl, 3-aminoprop-1-yl, 1-H-imidazol-4-ylmethyl, 1-H-indol-3-ylmethyl, benzyl, 4-hydroxyphen-1-ylmethyl, 2-carboxylatoeth-1-yl, 3-carboxylatoprop-1-yl, 3-guanidinoprop-1-yl, 4-guanidinobut-1-yl, 2-carbamoyleth-1-yl, or 3-carbamoylprop-1-yl.

The compound of any one of paragraphs [00501]-[00516], wherein each R¹ is independently H, hydroxylmethyl, or 4-guanidinobut-1-yl.

The compound of any one of paragraphs [00501]-[00516], wherein at least one iteration of R¹ is a hydroxyalkyl group.

The compound of any one of paragraphs [00501]-[00516], wherein at least one iteration of R¹ is hydroxylmethyl.

The compound of paragraph [00534], wherein at least a third of the iterations of R¹ are hydroxylmethyl.

The compound of paragraph [00534], wherein at least half the iterations of R¹ are hydroxylmethyl.

The compound of any one of paragraphs [00410]-[00537], wherein PEP1 is absent.

The compound of any one of paragraphs [00410]-[00537], wherein PEP1 is the peptide sequence.

The compound of any one of paragraphs [00410]-[00537] and [00539], wherein the peptide sequence of PEP1 is a nuclear localization sequence.

The compound of any one of paragraphs [00410]-[00537] and [00539], wherein at least one of PEP1 and PEP2 is a peptide sequence of at least three amino acid residues.

The compound of any one of paragraphs [00410]-[00537], [00539], and [00541], wherein PEP1 is -Arg-Arg-.

The compound of any one of paragraphs [00410]-[00537], [00539], and [00541], wherein PEP1 is a sequence that is -Pro-Lys-Lys-Lys-Arg-Lys-Val- (SEQ ID NO: 1).

The compound of any one of paragraphs [00410]-[00537], [00539], and [00541], wherein PEP1 is a sequence that is -Ala-Lys-Ala-Ser-Ser-Leu-Asn-Ile-Ala- (SEQ ID NO: 77).

The compound of any one of paragraphs [00410]-[00537], [00539], and [00541], wherein PEP1 is a sequence that is -Ala-Ser-Ser-Leu-Asn-Ile-Ala- (SEQ ID NO: 78).

The compound of any one of paragraphs [00410]-[00537], [00539], and [00541], wherein PEP1 is a sequence that is -Arg-Phe-Gln-Ile-Leu-Tyr-Arg- (SEQ ID NO: 86).

The compound of any one of paragraphs [00410]-[00537], [00539], and [00541], wherein PEP1 is -(D-Arg)-(D-Arg)-(D-Arg)-.

The compound of any one of paragraphs [00410]-[00537], [00539], and [00541], wherein PEP1 is a sequence that is -Cys¹-Arg-Thr-Ile-Gly-Pro-Ser-Val-Cys²-, wherein Cys¹ and Cys² are bound to one another via an intrachain disulfide bond (SEQ ID NO: 82).

The compound of any one of paragraphs [00410]-[00537], [00539], and [00541], wherein PEP1 is a sequence that is -Arg-Gly-Arg-Gly-Arg-Gly-Arg-Gly-Arg-Gly-Arg-Gly-Arg-Gly- (SEQ ID NO: 93).

The compound of any one of paragraphs [00410]-[00537], [00539], and [00541], wherein PEP1 is a sequence that is -Arg-Tyr-Gln-Phe-Leu-Ile-Arg- (SEQ ID NO: 87).

The compound of any one of paragraphs [00410]-[00537], [00539], and [00541], wherein PEP1 is a sequence that is -Arg-Ile-Gln-Phe-Leu-Ile-Arg- (SEQ ID NO: 88).

The compound of any one of paragraphs [00410]-[00537], [00539], and [00541], wherein PEP1 is a sequence that is -Arg-Arg-Trp-Trp-Arg-Arg-Trp-Arg-Arg- (SEQ ID NO: 76).

The compound of any one of paragraphs [00410]-[00537], [00539], and [00541], wherein PEP1 is a sequence that is -Arg-Arg-Trp-Gln-Trp- (SEQ ID NO: 89).

The compound of any one of paragraphs [00410]-[00537], [00539], and [00541], wherein PEP1 is a sequence that is (D-Thr)-(D-His)-(D-Arg)-(D-Pro)-(D-Pro)-(D-Met)-(D-Trp)-(D-Ser)-(D-Pro)-(D-Val)-(D-Trp)-(D-Pro-) (SEQ ID NO: 85).

The compound of any one of paragraphs [00410]-[00537], [00539], and [00541], wherein PEP1 is a sequence that is -(D-Pro)-(D-Trp)-(D-Val)-(D-Pro)-(D-Ser)-(D-Trp)-(D-Met)-(D-Pro)-(D-Pro)-(D-Arg)-(D-His)-(D-Thr)- (SEQ ID NO: 83).

The compound of any one of paragraphs [00410]-[00537], [00539], and [00541], wherein PEP1 is a sequence that is -(D-His)-(D-Arg)-(D-Pro)-(D-Tyr)-(D-Ile)-(D-Ala)-(D-His)- (SEQ ID NO: 84).

The compound of any one of paragraphs [00410]-[00556], wherein PEP2 is absent.

The compound of any one of paragraphs [00410]-[00556], wherein PEP2 is the peptide sequence.

The compound of any one of paragraphs [00410]-[00556] or [00558], wherein the peptide sequence of PEP2 is a nuclear localization sequence.

The compound of any one of paragraphs [00410]-[00556] or [00558], wherein PEP2 is a sequence that is -Pro-Lys-Lys-Lys-Arg-Lys-Val- (SEQ ID NO: 1).

The compound of any one of paragraphs [00410]-[00556] or [00558], wherein PEP2 is -Arg-Arg-.

The compound of any one of paragraphs [00410]-[00556] or [00558], wherein PEP2 is a sequence that is -Arg-Phe-Gln-Ile-Leu-Tyr-Arg- (SEQ ID NO: 86).

The compound of any one of paragraphs [00410]-[00556] or [00558], wherein PEP2 is -(D-Arg)-(D-Arg)-(D-Arg)-.

The compound of any one of paragraphs [00410]-[00556] or [00558], wherein PEP2 is a sequence that is -Cys¹-Arg-Thr-Ile-Gly-Pro-Ser-Val-Cys²-, wherein Cys¹ and Cys² are bound to one another via an intrachain disulfide bond (SEQ ID NO: 82).

The compound of any one of paragraphs [00410]-[00556] or [00558], wherein PEP2 is a sequence that is -Arg-Gly-Arg-Gly-Arg-Gly-Arg-Gly-Arg-Gly-Arg-Gly-Arg-Gly- (SEQ ID NO: 93.

The compound of any one of paragraphs [00410]-[00556] or [00558], wherein PEP2 is a sequence that is -Arg-Tyr-Gln-Phe-Leu-Ile-Arg- (SEQ ID NO: 87).

The compound of any one of paragraphs [00410]-[00556] or [00558], wherein PEP2 is a sequence that is -Arg-Ile-Gln-Phe-Leu-Ile-Arg- (SEQ ID NO: 88).

The compound of any one of paragraphs [00410]-[00556] or [00558], wherein PEP2 is a sequence that is -Arg-Arg-Trp-Trp-Arg-Arg-Trp-Arg-Arg- (SEQ ID NO: 76).

The compound of any one of paragraphs [00410]-[00556] or [00558], wherein PEP2 is a sequence that is -Arg-Arg-Trp-Gln-Trp- (SEQ ID NO: 89).

The compound of any one of paragraphs [00410]-[00556] or [00558], wherein PEP2 is a sequence that is (D-Thr)-(D-His)-(D-Arg)-(D-Pro)-(D-Pro)-(D-Met)-(D-Trp)-(D-Ser)-(D-Pro)-(D-Val)-(D-Trp)-(D-Pro-) (SEQ ID NO: 85).

The compound of any one of paragraphs [00410]-[00556] or [00558], wherein PEP2 is a sequence that is -(D-Pro)-(D-Trp)-(D-Val)-(D-Pro)-(D-Ser)-(D-Trp)-(D-Met)-(D-Pro)-(D-Pro)-(D-Arg)-(D-His)-(D-Thr)- (SEQ ID NO: 83).

The compound of any one of paragraphs [00410]-[00556] or [00558], wherein PEP2 is a sequence that is -(D-His)-(D-Arg)-(D-Pro)-(D-Tyr)-(D-Ile)-(D-Ala)-(D-His)- (SEQ ID NO: 84)-.

The compound of any one of paragraphs [00410]-[00572], wherein SOL1 is absent.

The compound of any one of paragraphs [00410]-[00572], wherein SOL1 is the water-solubilizing group.

The compound of any one of paragraphs [00410]-[00572] and [00574], wherein the water-solubilizing group of SOL1 is a peptide sequence.

The compound of any one of paragraphs [00410]-[00572], [00574], and [00575], wherein the water-solubilizing group of SOL1 is a group that contains multiple electrical charges at physiological pH.

The compound of any one of paragraphs [00410]-[00572] and [00574]-[00576], wherein the water-solubilizing group of SOL1 is a group that contains multiple positive charges at physiological pH.

The compound of any one of paragraphs [00410]-[00572] and [00574], wherein the water-solubilizing group of SOL1 is a polyethyleneglycol group.

The compound of any one of paragraphs [00410]-[00572] and [00574], wherein the water-solubilizing group of SOL1 is -Arg-Arg-NH(CH₂)₂C(O)-Arg-Arg- (SEQ ID NO: 136).

The compound of any one of paragraphs [00410]-[00572] and [00574], wherein the water-solubilizing group of SOL1 is a group of formula:

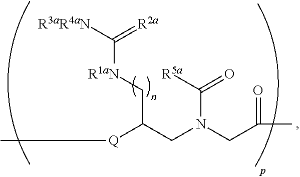

wherein
R^{1a} is H, alkyl, or a nitrogen atom protecting group;
R^{2a} is O, NH, N(alkyl), or N(Pg^{N}), wherein Pg^{N} is a nitrogen atom protecting group;
R^{3a} is H, alkyl, or a nitrogen atom protecting group;
R^{4a} is H, alkyl, or a nitrogen atom protecting group;
R^{5a} is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
n is 1, 2, 3, 4, 5, 6, 7, 8, 9, or 10; and
p is an integer that is 1-1,000.

The compound of any one of paragraphs [00410]-[00572], [00574], and [00580], wherein the water-solubilizing group of SOL1 is a group of formula:

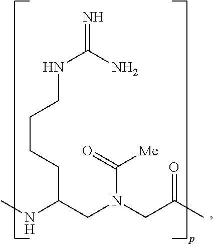

wherein p is an integer that is 1-1,000.

The compound of any one of paragraphs [00410]-[00572] and [00574], wherein the water-solubilizing group of SOL1 is a group of formula:

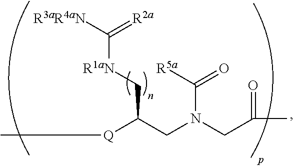

wherein
R^{1a} is H, alkyl, or a nitrogen atom protecting group;
R^{2a} is O, NH, N(alkyl), or N(Pg^{N}), wherein Pg^{N} is a nitrogen atom protecting group;
R^{3a} is H, alkyl, or a nitrogen atom protecting group;
R^{4a} is H, alkyl, or a nitrogen atom protecting group;
R^{5a} is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
n is 1, 2, 3, 4, 5, 6, 7, 8, 9, or 10; and
p is an integer that is 1-1,000.

The compound of any one of paragraphs [00410]-[00572], [00574], and [00580]-[00582], wherein the water-solubilizing group of SOL1 is a group of formula:

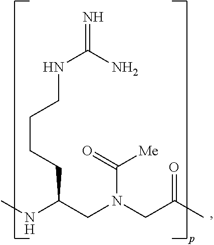

wherein p is an integer that is 1-1,000.

The compound of any one of paragraphs [00580]-[00583], wherein p is an integer that is 1-100.

The compound of any one of paragraphs [00580]-[00583], wherein p is an integer that is 1-50.

The compound of any one of paragraphs [00580]-[00583], wherein p is an integer that is 1, 2, 3, 4, 5, 6, 7, 8, 9, 10, 11, 12, 13, 14, 15, 16, 17, 18, 19, or 20.

The compound of any one of paragraphs [00580]-[00583], wherein p is an integer that is 1, 2, 3, 4, 5, 6, 7, 8, 9, 10, 11, or 12.

The compound of any one of paragraphs [00580]-[00583], wherein p is an integer that is 3, 4, 5, 6, 7, 8, 9, or 10.

The compound of any one of paragraphs [00580]-[00583], wherein p is an integer that is 5, 6, 7, 8, or 9.

The compound of any one of paragraphs [00580]-[00583], wherein p is an integer that is 6, 7, or 8.

The compound of any one of paragraphs [00580]-[00583], wherein p is an integer that is 7.

The compound of any one of paragraphs [00410]-[00590], wherein SOL2 is absent.

The compound of any one of paragraphs [00410]-[00590], wherein SOL2 is the water-solubilizing group.

The compound of any one of paragraphs [00410]-[00590] and [00593], wherein the water-solubilizing group of SOL2 is a peptide sequence.

The compound of any one of paragraphs [00410]-[00590], [00593], and [00594], wherein the water-solubilizing group of SOL2 is a group that contains multiple electrical charges at physiological pH.

The compound of any one of paragraphs [00410]-[00590], and [00593]-[00595], wherein the water-solubilizing group of SOL2 is a group that contains multiple positive charges at physiological pH.

The compound of any one of paragraphs [00410]-[00590] and [00593], wherein the water-solubilizing group of SOL2 is a polyethyleneglycol group.

The compound of any one of paragraphs [00410]-[00590] and [00593], wherein the water-solubilizing group of SOL2 is -Arg-Arg-NH(CH₂)₂C(O)-Arg-Arg- (SEQ ID NO: 136).

The compound of any one of paragraphs [00410]-[00590] and [00593], wherein the water-solubilizing group of SOL2 is a group of formula:

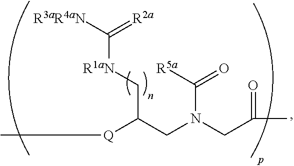

wherein
R^{1a} is H, alkyl, or a nitrogen atom protecting group;

R^{2a} is O, NH, N(alkyl), or N(Pg^{N}), wherein Pg^{N} is a nitrogen atom protecting group;
R^{3a} is H, alkyl, or a nitrogen atom protecting group;
R^{4a} is H, alkyl, or a nitrogen atom protecting group;
R^{5a} is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
n is 1, 2, 3, 4, 5, 6, 7, 8, 9, or 10; and
p is an integer that is 1-1,000.

The compound of any one of paragraphs [00410]-[00590], [00593], and [00599], wherein the water-solubilizing group of SOL2 is a group of formula:

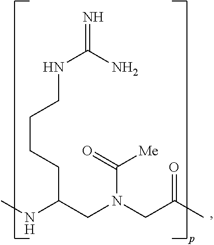

wherein p is an integer that is 1-1,000.

The compound of any one of paragraphs [00410]-[00590] and [00593], wherein the water-solubilizing group of SOL2 is a group of formula:

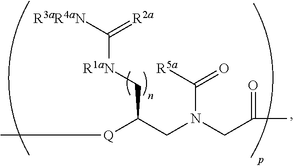

wherein
R^{1a} is H, alkyl, or a nitrogen atom protecting group;

R^{2a} is O, NH, N(alkyl), or N(Pg^{N}), wherein Pg^{N} is a nitrogen atom protecting group;
R^{3a} is H, alkyl, or a nitrogen atom protecting group;
R^{4a} is H, alkyl, or a nitrogen atom protecting group;
R^{5a} is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
n is 1, 2, 3, 4, 5, 6, 7, 8, 9, or 10; and
p is an integer that is 1-1,000.

The compound of any one of paragraphs [00410]-[00590], [00593], and [00599]-[00601], wherein the water-solubilizing group of SOL2 is a group of formula:

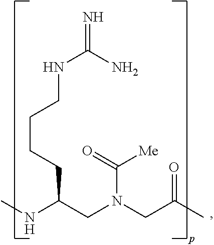

wherein p is an integer that is 1-1,000.

The compound of any one of paragraphs [00599]-[00602], wherein p is an integer that is 1-100.

The compound of any one of paragraphs [00599]-[00602], wherein p is an integer that is 1-50.

The compound of any one of paragraphs [00599]-[00602], wherein p is an integer that is 1, 2, 3, 4, 5, 6, 7, 8, 9, 10, 11, 12, 13, 14, 15, 16, 17, 18, 19, or 20.

The compound of any one of paragraphs [00599]-[00602], wherein p is an integer that is 1, 2, 3, 4, 5, 6, 7, 8, 9, 10, 11, or 12.

The compound of any one of paragraphs [00599]-[00602], wherein p is an integer that is 3, 4, 5, 6, 7, 8, 9, or 10.

The compound of any one of paragraphs [00599]-[00602], wherein p is an integer that is 5, 6, 7, 8, or 9.

The compound of any one of paragraphs [00599]-[00602], wherein p is an integer that is 6, 7, or 8.

The compound of any one of paragraphs [00599]-[00602], wherein p is an integer that is 7.

The compound of any one of paragraphs [00410]-[00610], wherein PNA1 is the peptide nucleic acid sequence.

The compound of any one of paragraphs [00410]-[00611], wherein PNA2 is the peptide nucleic acid sequence.

The compound of any one of paragraphs [00410]-[00612], wherein L1 is the linker group.

The compound of any one of paragraphs [00410]-[00613], wherein the linker group of L1 is cleavable.

The compound of any one of paragraphs [00410]-[00613], wherein the linker group of L1 is non-cleavable.

The compound of any one of paragraphs [00410]-[00613], wherein the linker group of L1 is a peptide sequence.

The compound of any one of paragraphs [00410]-[00613], wherein the linker group of L1 is a polyamine sequence.

The compound of any one of paragraphs [00410]-[00613], wherein the linker group of L1 is a polyamide sequence.

The compound of any one of paragraphs [00410]-[00613], wherein the linker group of L1 is a residue of an omega-amino fatty acid.

The compound of any one of paragraphs [00410]-[00613] and [00619], wherein the linker group of L1 is a residue of an omega-amino caproic acid.

The compound of any one of paragraphs [00410]-[00613], wherein the linker group of L1 is a residue of a dicarboxylic acid.

The compound of any one of paragraphs [00410]-[00613] and [00621], wherein the linker group of L1 is a residue of oxalic acid.

The compound of any one of paragraphs [00410]-[00613] and [00621], wherein the linker group of L1 is a residue of succinic acid.

The compound of any one of paragraphs [00410]-[00613], [00616], and [00618], wherein the linker group of L1 is a peptide sequence that is -Glu-Val-Citrulline-.

The compound of any one of paragraphs [00410]-[00613], wherein the linker group of L1 is —NHCH(COOH)C(CH₃)₂S—SC(CH₃)₂CH(NH₂)C(O)—.

The compound of any one of paragraphs [00410]-[00613], wherein the linker group of L1 is —NHCH(COOH)C(CH₃)₂S—SCH₂CH(NH₂)C(O)—.

The compound of any one of paragraphs [00410]-[00613], wherein the linker group of L1 is -Arg-NH(CH₂)₅C(O)—.

The compound of any one of paragraphs [00410]-[00613], wherein the linker group of L1 is —NH(CH₂)₅C(O)—.

The compound of any one of paragraphs [00410]-[00613], wherein the linker group of L1 is —NH(CH₂)₂C(O)-Arg-NH(CH₂)₅C(O)NH(CH₂)₂C(O)—.

The compound of any one of paragraphs [00410]-[00613], wherein the linker group of L1 is —NH(CH₂)₅C(O)-Arg-NH(CH₂)₂C(O)-Arg-NH(CH₂)₅C(O)-Arg-NH(CH₂)₂C(O).

The compound of any one of paragraphs [00410]-[00613], wherein the linker group of L1 is -Arg-NH(CH₂)₅C(O)-Arg-Arg-NH(CH₂)₂C(O)-Arg-Arg-NH(CH₂)₅C(O)— (SEQ ID NO: 137).

The compound of any one of paragraphs [00410]-[00613], wherein the linker group of L1 is —NH(CH₂)₅C(O)NH(CH₂)₂-(D-arginine)-(D-arginine)-(D-arginine).

The compound of any one of paragraphs [00410]-[00613], wherein the linker group of L1 is —NH(CH₂CH₂O)₂CH₂C(O)—.

The compound of any one of paragraphs [00410]-[00613], wherein the linker group of L1 is —NH(CH₂CH₂O)₂CH₂C(O)-Arg-.

The compound of any one of paragraphs [00410]-[00613], wherein the linker group of L1 is —NH(CH₂CH₂O)₂CH₂C(O)—NH(CH₂CH₂O)₂CH₂C(O)—.

The compound of any one of paragraphs [00410]-[00613], wherein the linker group of L1 is —NH(CH₂CH₂O)₂CH₂C(O)—NH(CH₂CH₂O)₂CH₂C(O)-Arg-.

The compound of any one of paragraphs [00410]-[00613], wherein the linker group of L1 is —NH(CH₂CH₂O)₂CH₂C(O)—NH(CH₂CH₂O)₂CH₂C(O)-Arg-Arg-.

The compound of any one of paragraphs [00410]-[00613], wherein the linker group of L1 is —NH(CH₂CH₂O)₂CH₂C(O)-Arg-NH(CH₂CH₂O)₂CH₂C(O)-Arg-.

The compound of any one of paragraphs [00410]-[00613], [00616], and [00618], wherein the linker group of L1 is a peptide sequence that is -Lys-.

The compound of any one of paragraphs [00410]-[00613], [00616], and [00618], wherein the linker group of L1 is a peptide sequence that is -(D-Arg)-(D-Arg)-(D-Arg)-.

The compound of any one of paragraphs [00410]-[00640], wherein L2 is the linker group.

The compound of any one of paragraphs [00410]-[00641], wherein the linker group of L2 is cleavable.

The compound of any one of paragraphs [00410]-[00641], wherein the linker group of L2 is non-cleavable.

The compound of any one of paragraphs [00410]-[00641], wherein the linker group of L2 is a peptide sequence.

The compound of any one of paragraphs [00410]-[00641], wherein the linker group of L2 is a polyamine sequence.

The compound of any one of paragraphs [00410]-[00641] and [00644], wherein the linker group of L2 is a polyamide sequence.

The compound of any one of paragraphs [00410]-[00641], wherein the linker group of L2 is a residue of an omega-amino fatty acid.

The compound of any one of paragraphs [00410]-[00641] and [00647], wherein the linker group of L2 is a residue of an omega-amino caproic acid.

The compound of any one of paragraphs [00410]-[00641], wherein the linker group of L2 is a residue of a dicarboxylic acid.

The compound of any one of paragraphs [00410]-[00641] and [00649], wherein the linker group of L2 is a residue of oxalic acid.

The compound of any one of paragraphs [00410]-[00641] and [00649], wherein the linker group of L2 is a residue of succinic acid.

The compound of any one of paragraphs [00410]-[00641] and [00644], wherein the linker group of L2 is a peptide sequence that is -Glu-Val- Citrulline-.

The compound of any one of paragraphs [00410]-[00641], wherein the linker group of L2 is —NHCH(COOH)C(CH₃)₂S—SC(CH₃)₂CH(NH₂)C(O)—.

The compound of any one of paragraphs [00410]-[00641], wherein the linker group of L2 is —NHCH(COOH)C(CH₃)₂S—SCH₂CH(NH₂)C(O)—.

The compound of any one of paragraphs [00410]-[00641], wherein the linker group of L2 is -Arg-NH(CH₂)₅C(O)—.

The compound of any one of paragraphs [00410]-[00641], wherein the linker group of L2 is —NH(CH₂)₅C(O)—.

The compound of any one of paragraphs [00410]-[00641], wherein the linker group of L2 is —NH(CH₂)₂C(O)-Arg-NH(CH₂)₅C(O)NH(CH₂)₂C(O)—.

The compound of any one of paragraphs [00410]-[00641], wherein the linker group of L2 is —NH(CH₂)₅C(O)-Arg-NH(CH₂)₂C(O)-Arg-NH(CH₂)₅C(O)-Arg-NH(CH₂)₂C(O).

The compound of any one of paragraphs [00410]-[00641], wherein the linker group of L2 is -Arg-NH(CH₂)₅C(O)-Arg-Arg-NH(CH₂)₂C(O)-Arg-Arg-NH(CH₂)₅C(O)— (SEQ ID NO: 137).

The compound of any one of paragraphs [00410]-[00641], wherein the linker group of L2 is —NH(CH₂)₅C(O)NH(CH₂)₂-(D-arginine)-(D-arginine)-(D-arginine).

The compound of any one of paragraphs [00410]-[00641], wherein the linker group of L2 is —NH(CH₂CH₂O)₂CH₂C(O)—.

The compound of any one of paragraphs [00410]-[00641], wherein the linker group of L2 is —NH(CH₂CH₂O)₂CH₂C(O)-Arg-.

The compound of any one of paragraphs [00410]-[00641], wherein the linker group of L2 is —NH(CH₂CH₂O)₂CH₂C(O)—NH(CH₂CH₂O)₂CH₂C(O)—.

The compound of any one of paragraphs [00410]-[00641], wherein the linker group of L2 is —NH(CH₂CH₂O)₂CH₂C(O)—NH(CH₂CH₂O)₂CH₂C(O)-Arg-.

The compound of any one of paragraphs [00410]-[00641], wherein the linker group of L2 is —NH(CH₂CH₂O)₂CH₂C(O)—NH(CH₂CH₂O)₂CH₂C(O)-Arg-Arg-.

The compound of any one of paragraphs [00410]-[00641], wherein the linker group of L2 is —NH(CH₂CH₂O)₂CH₂C(O)-Arg-NH(CH₂CH₂O)₂CH₂C(O)-Arg-.

The compound of any one of paragraphs [00410]-[00641] and [00644], wherein the linker group of L2 is a peptide sequence that is -Lys-.

The compound of any one of paragraphs [00410]-[00641] and [00644], wherein the linker group of L2 is a peptide sequence that is -(D-Arg)-(D-Arg)-(D-Arg)-.

The compound of any one of paragraphs [00410]-[00668], wherein L3 is the linker group.

The compound of any one of paragraphs [00410]-[00669], wherein the linker group of L3 is cleavable.

The compound of any one of paragraphs [00410]-[00669], wherein the linker group of L3 is non-cleavable.

The compound of any one of paragraphs [00410]-[00669], wherein the linker group of L3 is a peptide sequence.

The compound of any one of paragraphs [00410]-[00669], wherein the linker group of L3 is a polyamine sequence.

The compound of any one of paragraphs [00410]-[00669] and [00672], wherein the linker group of L3 is a polyamide sequence.

The compound of any one of paragraphs [00410]-[00669], wherein the linker group of L3 is a residue of an omega-amino fatty acid.

The compound of any one of paragraphs [00410]-[00669] and [00675], wherein the linker group of L3 is a residue of an omega-amino caproic acid.

The compound of any one of paragraphs [00410]-[00669], wherein the linker group of L3 is a residue of a dicarboxylic acid.

The compound of any one of paragraphs [00410]-[00669] and [00677], wherein the linker group of L3 is a residue of oxalic acid.

The compound of any one of paragraphs [00410]-[00669] and [00677], wherein the linker group of L3 is a residue of succinic acid.

The compound of any one of paragraphs [00410]-[00669] and [00672], wherein the linker group of L3 is a peptide sequence that is -Glu-Val- Citrulline-.

The compound of any one of paragraphs [00410]-[00669], wherein the linker group of L3 is —NHCH(COOH)C(CH₃)₂S—SC(CH₃)₂CH(NH₂)C(O)—.

The compound of any one of paragraphs [00410]-[00669], wherein the linker group of L3 is —NHCH(COOH)C(CH₃)₂S—SCH₂CH(NH₂)C(O)—.

The compound of any one of paragraphs [00410]-[00669], wherein the linker group of L3 is -Arg-NH(CH₂)₅C(O)—.

The compound of any one of paragraphs [00410]-[00669], wherein the linker group of L3 is —NH(CH₂)₅C(O)—.

The compound of any one of paragraphs [00410]-[00669], wherein the linker group of L3 is —NH(CH₂)₂C(O)-Arg-NH(CH₂)₅C(O)NH(CH₂)₂C(O)—.

The compound of any one of paragraphs [00410]-[00669], wherein the linker group of L3 is —NH(CH₂)₅C(O)-Arg-NH(CH₂)₂C(O)-Arg-NH(CH₂)₅C(O)-Arg-NH(CH₂)₂C(O).

The compound of any one of paragraphs [00410]-[00669], wherein the linker group of L3 is -Arg-NH(CH₂)₅C(O)-Arg-Arg-NH(CH₂)₂C(O)-Arg-Arg-NH(CH₂)₅C(O)— (SEQ ID NO: 137).

The compound of any one of paragraphs [00410]-[00669], wherein the linker group of L3 is —NH(CH₂)₅C(O)NH(CH₂)₂-(D-arginine)-(D-arginine)-(D-arginine).

The compound of any one of paragraphs [00410]-[00669], wherein the linker group of L3 is —NH(CH₂CH₂O)₂CH₂C(O)—.

The compound of any one of paragraphs [00410]-[00669], wherein the linker group of L3 is —NH(CH₂CH₂O)₂CH₂C(O)-Arg-.

The compound of any one of paragraphs [00410]-[00669], wherein the linker group of L3 is —NH(CH₂CH₂O)₂CH₂C(O)—NH(CH₂CH₂O)₂CH₂C(O)—.

The compound of any one of paragraphs [00410]-[00669], wherein the linker group of L3 is —NH(CH₂CH₂O)₂CH₂C(O)—NH(CH₂CH₂O)₂CH₂C(O)-Arg-.

The compound of any one of paragraphs [00410]-[00669], wherein the linker group of L3 is —NH(CH₂CH₂O)₂CH₂C(O)—NH(CH₂CH₂O)₂CH₂C(O)-Arg-Arg-.

The compound of any one of paragraphs [00410]-[00669], wherein the linker group of L3 is —NH(CH₂CH₂O)₂CH₂C(O)-Arg-NH(CH₂CH₂O)₂CH₂C(O)-Arg-.

The compound of any one of paragraphs [00410]-[00669] and [00672], wherein the linker group of L3 is a peptide sequence that is -Lys-.

The compound of any one of paragraphs [00410]-[00669] and [00672], wherein the linker group of L3 is a peptide sequence that is -(D-Arg)-(D-Arg)-(D-Arg)-.

The compound of any one of paragraphs [00410]-[00696], wherein L4 is the linker group.

The compound of any one of paragraphs [00410]-[00697], wherein the linker group of L4 is cleavable.

The compound of any one of paragraphs [00410]-[00697], wherein the linker group of L4 is non-cleavable.

The compound of any one of paragraphs [00410]-[00697], wherein the linker group of L4 is a peptide sequence.

The compound of any one of paragraphs [00410]-[00697], wherein the linker group of L4 is a polyamine sequence.

The compound of any one of paragraphs [00410]-[00697] and [00700], wherein the linker group of L4 is a polyamide sequence.

The compound of any one of paragraphs [00410]-[00697], wherein the linker group of L4 is a residue of an omega-amino fatty acid.

The compound of any one of paragraphs [00410]-[00697] and [00703], wherein the linker group of L4 is a residue of an omega-amino caproic acid.

The compound of any one of paragraphs [00410]-[00697], wherein the linker group of L4 is a residue of a dicarboxylic acid.

The compound of any one of paragraphs [00410]-[00697] and [00705], wherein the linker group of L4 is a residue of oxalic acid.

The compound of any one of paragraphs [00410]-[00697] and [00705], wherein the linker group of L4 is a residue of succinic acid.

The compound of any one of paragraphs [00410]-[00697] and [00700], wherein the linker group of L4 is a peptide sequence that is -Glu-Val- Citrulline-.

The compound of any one of paragraphs [00410]-[00697], wherein the linker group of L4 is —NHCH(COOH)C(CH₃)₂S—SC(CH₃)₂CH(NH₂)C(O)—.

The compound of any one of paragraphs [00410]-[00697], wherein the linker group of L4 is —NHCH(COOH)C(CH₃)₂S—SCH₂CH(NH₂)C(O)—.

The compound of any one of paragraphs [00410]-[00697], wherein the linker group of L4 is -Arg-NH(CH₂)₅C(O)—.

The compound of any one of paragraphs [00410]-[00697], wherein the linker group of L4 is —NH(CH₂)₅C(O)—.

The compound of any one of paragraphs [00410]-[00697], wherein the linker group of L4 is —NH(CH₂)₂C(O)-Arg-NH(CH₂)₅C(O)NH(CH₂)₂C(O)—.

The compound of any one of paragraphs [00410]-[00697], wherein the linker group of L4 is —NH(CH₂)₅C(O)-Arg-NH(CH₂)₂C(O)-Arg-NH(CH₂)₅C(O)-Arg-NH(CH₂)₂C(O).

The compound of any one of paragraphs [00410]-[00697], wherein the linker group of L4 is -Arg-NH(CH₂)₅C(O)-Arg-Arg-NH(CH₂)₂C(O)-Arg-Arg-NH(CH₂)₅C(O)— (SEQ ID NO: 137).

The compound of any one of paragraphs [00410]-[00697], wherein the linker group of L4 is —NH(CH₂)₅C(O)NH(CH₂)₂-(D-arginine)-(D-arginine)-(D-arginine).

The compound of any one of paragraphs [00410]-[00697], wherein the linker group of L4 is —NH(CH₂CH₂O)₂CH₂C(O)—.

The compound of any one of paragraphs [00410]-[00697], wherein the linker group of L4 is —NH(CH₂CH₂O)₂CH₂C(O)-Arg-.

The compound of any one of paragraphs [00410]-[00697], wherein the linker group of L4 is —NH(CH₂CH₂O)₂CH₂C(O)—NH(CH₂CH₂O)₂CH₂C(O)—.

The compound of any one of paragraphs [00410]-[00697], wherein the linker group of L4 is —NH(CH₂CH₂O)₂CH₂C(O)—NH(CH₂CH₂O)₂CH₂C(O)-Arg-.

The compound of any one of paragraphs [00410]-[00697], wherein the linker group of L4 is —NH(CH₂CH₂O)₂CH₂C(O)—NH(CH₂CH₂O)₂CH₂C(O)-Arg-Arg-.

The compound of any one of paragraphs [00410]-[00697], wherein the linker group of L4 is —NH(CH₂CH₂O)₂CH₂C(O)-Arg-NH(CH₂CH₂O)₂CH₂C(O)-Arg-.

The compound of any one of paragraphs [00410]-[00697] and [00700], wherein the linker group of L4 is a peptide sequence that is -Lys-.

The compound of any one of paragraphs [00410]-[00697] and [00700], wherein the linker group of L4 is a peptide sequence that is -(D-Arg)-(D-Arg)-(D-Arg)-.

The compound of any one of paragraphs [00410]-[00724], wherein L5 is the linker group.

The compound of any one of paragraphs [00410]-[00725], wherein the linker group of L5 is cleavable.

The compound of any one of paragraphs [00410]-[00725], wherein the linker group of L5 is non-cleavable.

The compound of any one of paragraphs [00410]-[00725], wherein the linker group of L5 is a peptide sequence.

The compound of any one of paragraphs [00410]-[00725], wherein the linker group of L5 is a polyamine sequence.

The compound of any one of paragraphs [00410]-[00725] and [00728], wherein the linker group of L5 is a polyamide sequence.

The compound of any one of paragraphs [00410]-[00725], wherein the linker group of L5 is a residue of an omega-amino fatty acid.

The compound of any one of paragraphs [00410]-[00725], and [00731], wherein the linker group of L5 is a residue of an omega-amino caproic acid.

The compound of any one of paragraphs [00410]-[00725], wherein the linker group of L5 is a residue of a dicarboxylic acid.

The compound of any one of paragraphs [00410]-[00725] and [00733], wherein the linker group of L5 is a residue of oxalic acid.

The compound of any one of paragraphs [00410]-[00725] and [00733], wherein the linker group of L5 is a residue of succinic acid.

The compound of any one of paragraphs [00410]-[00725] and [00728], wherein the linker group of L5 is a peptide sequence that is -Glu-Val- Citrulline-.

The compound of any one of paragraphs [00410]-[00725], wherein the linker group of L5 is —NHCH(COOH)C(CH₃)₂S—SC(CH₃)₂CH(NH₂)C(O)—.

The compound of any one of paragraphs [00410]-[00725], wherein the linker group of L5 is —NHCH(COOH)C(CH₃)₂S—SCH₂CH(NH₂)C(O)—.

The compound of any one of paragraphs [00410]-[00725], wherein the linker group of L5 is -Arg-NH(CH₂)₅C(O)—.

The compound of any one of paragraphs [00410]-[00725], wherein the linker group of L5 is —NH(CH₂)₅C(O)—.

The compound of any one of paragraphs [00410]-[00725], wherein the linker group of L5 is —NH(CH₂)₂C(O)-Arg-NH(CH₂)₅C(O)NH(CH₂)₂C(O)—.

The compound of any one of paragraphs [00410]-[00725], wherein the linker group of L5 is —NH(CH₂)₅C(O)-Arg-NH(CH₂)₂C(O)-Arg-NH(CH₂)₅C(O)-Arg-NH(CH₂)₂C(O).

The compound of any one of paragraphs [00410]-[00725], wherein the linker group of L5 is —NH(CH₂)₅C(O)NH(CH₂)₂-(D-arginine)-(D-arginine)-(D-arginine).

The compound of any one of paragraphs [00410]-[00725], wherein the linker group of L5 is —NH(CH₂CH₂O)₂CH₂C(O)—.

The compound of any one of paragraphs [00410]-[00725], wherein the linker group of L5 is —NH(CH₂CH₂O)₂CH₂C(O)-Arg-.

The compound of any one of paragraphs [00410]-[00725], wherein the linker group of L5 is —NH(CH₂CH₂O)₂CH₂C(O)—NH(CH₂CH₂O)₂CH₂C(O)—.

The compound of any one of paragraphs [00410]-[00725], wherein the linker group of L5 is —NH(CH₂CH₂O)₂CH₂C(O)—NH(CH₂CH₂O)₂CH₂C(O)-Arg-.

The compound of any one of paragraphs [00410]-[00725], wherein the linker group of L5 is —NH(CH₂CH₂O)₂CH₂C(O)—NH(CH₂CH₂O)₂CH₂C(O)-Arg-Arg-.

The compound of any one of paragraphs [00410]-[00725], wherein the linker group of L5 is —NH(CH₂CH₂O)₂CH₂C(O)-Arg-NH(CH₂CH₂O)₂CH₂C(O)-Arg-.

The compound of any one of paragraphs [00410]-[00725] and [00728], wherein the linker group of L5 is a peptide sequence that is -Lys-.

The compound of any one of paragraphs [00410]-[00750], wherein L6 is the linker group.

The compound of any one of paragraphs [00410]-[00751], wherein the linker group of L6 is cleavable.

The compound of any one of paragraphs [00410]-[00751], wherein the linker group of L6 is non-cleavable.

The compound of any one of paragraphs [00410]-[00751], wherein the linker group of L6 is a peptide sequence.

The compound of any one of paragraphs [00410]-[00751], wherein the linker group of L6 is a polyamine sequence.

The compound of any one of paragraphs [00410]-[00751] and [00754], wherein the linker group of L6 is a polyamide sequence.

The compound of any one of paragraphs [00410]-[00751], wherein the linker group of L6 is a residue of an omega-amino fatty acid.

The compound of any one of paragraphs [00410]-[00751] and [00757], wherein the linker group of L6 is a residue of an omega-amino caproic acid.

The compound of any one of paragraphs [00410]-[00751], wherein the linker group of L6 is a residue of a dicarboxylic acid.

The compound of any one of paragraphs [00410]-[00751] and [00759], wherein the linker group of L6 is a residue of oxalic acid.

The compound of any one of paragraphs [00410]-[00751] and [00759], wherein the linker group of L6 is a residue of succinic acid.

The compound of any one of paragraphs [00410]-[00751] and [00754], wherein the linker group of L6 is a peptide sequence that is -Glu-Val- Citrulline-.

The compound of any one of paragraphs [00410]-[00751], wherein the linker group of L6 is —NHCH(COOH)C(CH₃)₂S—SC(CH₃)₂CH(NH₂)C(O)—.

The compound of any one of paragraphs [00410]-[00751], wherein the linker group of L6 is —NHCH(COOH)C(CH₃)₂S—SCH₂CH(NH₂)C(O)—.

The compound of any one of paragraphs [00410]-[00751], wherein the linker group of L6 is -Arg-NH(CH₂)₅C(O)—.

The compound of any one of paragraphs [00410]-[00751], wherein the linker group of L6 is —NH(CH₂)₅C(O)—.

The compound of any one of paragraphs [00410]-[00751], wherein the linker group of L6 is —NH(CH₂)₂C(O)-Arg-NH(CH₂)₅C(O)NH(CH₂)₂C(O)—.

The compound of any one of paragraphs [00410]-[00751], wherein the linker group of L6 is —NH(CH₂)₅C(O)-Arg-NH(CH₂)₂C(O)-Arg-NH(CH₂)₅C(O)-Arg-NH(CH₂)₂C(O).

The compound of any one of paragraphs [00410]-[00751], wherein the linker group of L6 is -Arg-NH(CH₂)₅C(O)-Arg-Arg-NH(CH₂)₂C(O)-Arg-Arg-NH(CH₂)₅C(O)— (SEQ ID NO: 137).

The compound of any one of paragraphs [00410]-[00751], wherein the linker group of L6 is —NH(CH₂)₅C(O)NH(CH₂)₂-(D-arginine)-(D-arginine)-(D-arginine).

The compound of any one of paragraphs [00410]-[00751], wherein the linker group of L6 is —NH(CH₂CH₂O)₂CH₂C(O)—.

The compound of any one of paragraphs [00410]-[00751], wherein the linker group of L6 is —NH(CH₂CH₂O)₂CH₂C(O)-Arg-.

The compound of any one of paragraphs [00410]-[00751], wherein the linker group of L6 is —NH(CH₂CH₂O)₂CH₂C(O)—NH(CH₂CH₂O)₂CH₂C(O)—.

The compound of any one of paragraphs [00410]-[00751], wherein the linker group of L6 is —NH(CH₂CH₂O)₂CH₂C(O)—NH(CH₂CH₂O)₂CH₂C(O)-Arg-.

The compound of any one of paragraphs [00410]-[00751], wherein the linker group of L6 is —NH(CH₂CH₂O)₂CH₂C(O)—NH(CH₂CH₂O)₂CH₂C(O)-Arg-Arg-.

The compound of any one of paragraphs [00410]-[00751], wherein the linker group of L6 is —NH(CH₂CH₂O)₂CH₂C(O)-Arg-NH(CH₂CH₂O)₂CH₂C(O)-Arg-.

The compound of any one of paragraphs [00410]-[00751] and [00754], wherein the linker group of L6 is a peptide sequence that is -Lys-.

The compound of any one of paragraphs [00410]-[00751] and [00754], wherein the linker group of L6 is a peptide sequence that is -(D-Arg)-(D-Arg)-(D-Arg)-.

The compound of any one of paragraphs [00410]-[00518], wherein each of L1, L2, L3, L4, L5, L6, PEP1, PEP1, SOL1, and SOL2 is absent.

The compound of any one of paragraphs [00501]-[00518], wherein the structure is:

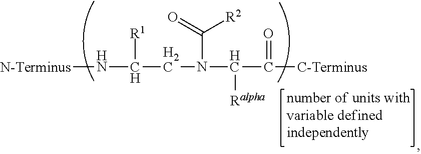

wherein the number of units with variables defined independently is at least 11;
or a pharmaceutically-acceptable salt or ionized form thereof.

The compound of any one of paragraphs [00501]-[00518] and [00780], wherein the structure is:

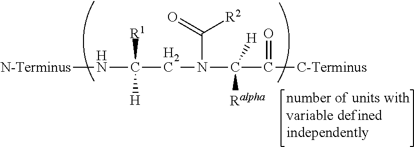

or a pharmaceutically-acceptable salt or ionized form thereof.

The compound of paragraph [00780] or paragraph [00781], wherein at least one iteration of R¹ is a hydroxyalkyl group.

The compound of any one of paragraphs [00410]-[00782], wherein N-Terminus is H.

The compound of any one of paragraphs [00410]-[00782], wherein N-Terminus is acyl.

The compound of any one of paragraphs [00410]-[00782], wherein N-Terminus is the biological agent.

The compound of paragraph [00785], wherein the biological agent is a vitamin E group.

The compound of paragraph [00785], wherein the biological agent is an O-bound tocopherol group.

The compound of any one of paragraphs [00410]-[00787], wherein C-Terminus is OH, O-alkyl, or NH₂.

The compound of any one of paragraphs [00410]-[00787], wherein C-Terminus is OH, OMe, OEt, Ot-Bu, or NH₂.

The compound of any one of paragraphs [00410]-[00787], wherein C-Terminus is NH₂.

The compound of any one of paragraphs [00410]-[00787], wherein C-Terminus is the peptide sequence.

The compound of any one of paragraphs [00410]-[00787], wherein C-Terminus is a peptide sequence according to SEQ ID NO: 1, wherein the C-terminal residue of the peptide sequence is amidated.

A compound comprising a repeating unit of formula:

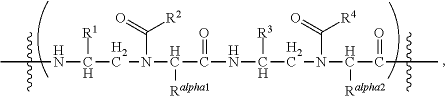

wherein:
each R¹ is independently a hydroxyalkyl group;
each R² is independently alkyl, O-alkyl, or methyl substituted with a heterocycle;
each R³ is independently a group that is not hydroxyalkyl;
each R⁴ is independently alkyl, O-alkyl, or methyl substituted with a heterocycle;
each R^{alpha1} is independently alkyl that is unsubstituted or substituted or H; and
each R^{alpha2} is independently alkyl that is unsubstituted or substituted or H,
   wherein the repeating unit occurs at least twice consecutively in the compound.

The compound of paragraph [00793], wherein each R¹ is independently an alkyl group that is unsubstituted or substituted, and each R³ is independently a group that is not substituted alkyl.

The compound of paragraph [00793], wherein each R¹ is independently a guanidinoalkyl group, and each R³ is independently a group that is not guanidinoalkyl.

The compound of paragraph [00793], wherein each R¹ is independently a hydroxyalkyl group, and each R³ is independently a group that is not hydroxyalkyl.

The compound of any one of paragraphs [00793]-[00796], wherein each R¹ is hydroxymethyl.

The compound of any one of paragraphs [00793]-[00797], wherein each R³ is H.

The compound of any one of paragraphs [00793]-[00798], wherein each R^{alpha1} and each R^{alpha2} is H.

The compound of paragraph [00793], wherein each R^{alpha1} is independently an alkyl group that is unsubstituted or substituted, and R^{alpha2} is independently a group that is not substituted alkyl.

The compound of paragraph [00793], wherein each R^{alpha1} is independently a guanidinoalkyl group, and each R^{alpha2} is independently a group that is not guanidinoalkyl.

The compound of paragraph [00793], wherein each R^{alpha1} is independently a hydroxyalkyl group, and each R^{alpha2} is independently a group that is not hydroxyalkyl.

The compound of any one of paragraphs [00800]-[00802], wherein each R^{alpha1} is hydroxymethyl.

The compound of any one of paragraphs [00800]-[00803], wherein each R^{alpha2} is H.

The compound of any one of paragraphs [00800]-[00804], wherein each R¹ and each R³ is H.

The compound of any one of paragraphs [00793]-[00805], wherein the repeating unit occurs at least five times consecutively in the compound.

The compound of any one of paragraphs [00793]-[00805], wherein the repeating unit occurs at least seven times consecutively in the compound.

The compound of any one of paragraphs [00793]-[00807], wherein at least a third of the R² groups in the structure are methyl substituted with a heterocycle.

The compound of any one of paragraphs [00793]-[00807], wherein at least half of the R² groups in the structure are methyl substituted with a heterocycle.

The compound of any one of paragraphs [00793]-[00809], wherein the heterocycles of the R² groups are nucleobases or analogues of nucleobases.

The compound of any one of paragraphs [00793]-[00810], wherein at least one of the heterocycles of the R² groups is a divalent nucleobase.

The compound of any one of paragraphs [00793]-[00811], wherein the heterocycles of the R² groups are divalent nucleobases.

The compound of any one of paragraphs [00793]-[00809], wherein the heterocycles of the R² groups are each independently:

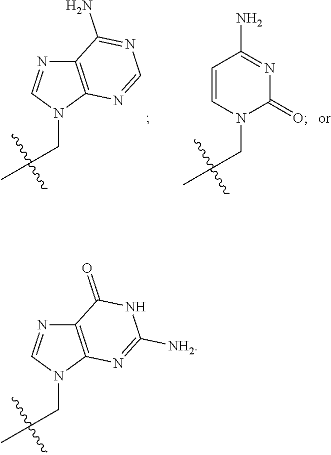

The compound of any one of paragraphs [00793]-[00809], wherein each R² is independently: methyl,

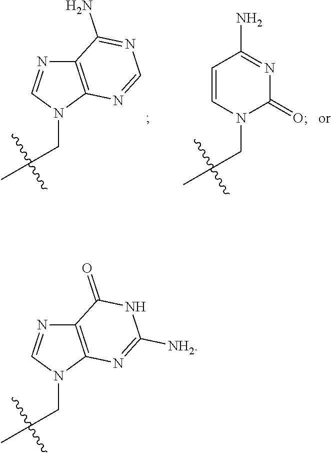

The compound of any one of paragraphs [00793]-[00814], wherein at least a third of the R⁴ groups in the structure are methyl substituted with a heterocycle.

The compound of any one of paragraphs [00793]-[00814], wherein at least half of the R⁴ groups in the structure are methyl substituted with a heterocycle.

The compound of any one of paragraphs [00793]-[00814], wherein the heterocycles of the R⁴ groups are nucleobases or analogues of nucleobases.

The compound of any one of paragraphs [00793]-[00814], wherein at least one of the heterocycles of the R⁴ groups is a divalent nucleobase.

The compound of any one of paragraphs [00793]-[00814], wherein the heterocycles of the R⁴ groups are divalent nucleobases.

The compound of any one of paragraphs [00793]-[00814], wherein the heterocycles of the R⁴ groups are each independently:

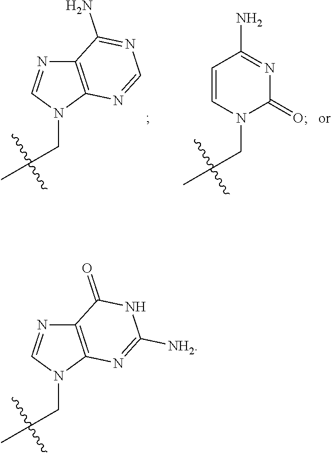

The compound of any one of paragraphs [00793]-[00814], wherein each R⁴ is independently: methyl,

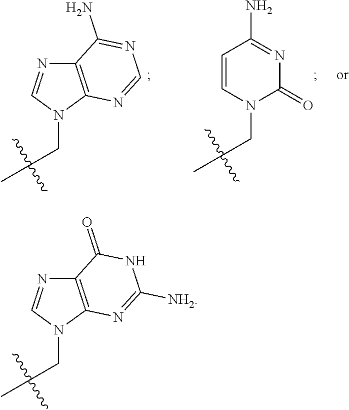 [SMILES: *Cn1cnc2c(N)ncnc21 ; *Cn1ccc(N)nc1=O ; *Cn1cnc2c(=O)[nH]c(N)nc21]

The compound of any one of paragraphs [00410]-[00821], wherein the structure binds to a mRNA sequence transcribed from a gene associated with a neuromuscular disease phenotype.

The compound of any one of paragraphs [00410]-[00821], wherein the structure binds to a mRNA sequence transcribed from a gene associated with a DM1 disease phenotype.

The compound of any one of paragraphs [00410]-[00821], wherein the structure binds to a mRNA sequence transcribed from a gene associated with a DM1 disease phenotype by interactions between the heterocycles of the R² groups and nucleobases of the DM1 gene.

The compound of paragraph [00823] or paragraph [00824], wherein the DM1 gene is a non-wild type DM1 gene.

The compound of paragraph [00825], wherein the non-wild type DM1 gene differs from a wild type DM1 gene in a repeat expansion mutation.

The compound of any one of paragraphs [00410]-[00826], wherein the structure binds to a mRNA sequence that contains a subsequence that is (CUG)_{z}, wherein z is an integer from 1-100 (SEQ ID NO: 135).

The compound of paragraph [00827], wherein the structure binds to the mRNA sequence at the subsequence that is (CUG)_{z}.

A compound comprising an oligomeric sequence, wherein the oligomeric sequence comprises a repeating unit of formula:

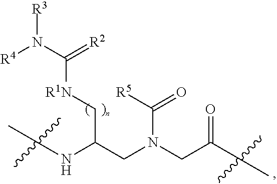

or an ionized form thereof, wherein:
R¹ is H, alkyl, or a nitrogen atom protecting group;
R² is O, NH, N(alkyl), or N(Pg^{N}), wherein Pg^{N} is a nitrogen atom protecting group;
R³ is H, alkyl, or a nitrogen atom protecting group;
R⁴ is H, alkyl, or a nitrogen atom protecting group;
R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; and
n is 1, 2, 3, 4, 5, 6, 7, 8, 9, or 10, or a pharmaceutically-acceptable salt or ionized form thereof.

The compound of paragraph [00829], wherein n is 3.

The compound of paragraph [00829], wherein n is 4.

The compound of any one of paragraphs [00829]-[00831], wherein each of R¹, R³, and R⁴ is hydrogen; and R² is NH or N(Pg^{N}).

The compound of any one of paragraphs [00829]-[00831], wherein R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, or heterocyclyl.

The compound of any one of paragraphs [00829]-[00831], wherein R⁵ is linear alkyl, branched alkyl, or cyclic alkyl.

The compound of any one of paragraphs [00829]-[00831], wherein R⁵ is linear alkyl.

The compound of any one of paragraphs [00829]-[00831], wherein R⁵ is methyl.

The compound of paragraph [00829], wherein the oligomeric sequence is connected to a region that comprises a structure that interferes with expression of a gene associated with a neuromuscular disease phenotype.

The compound of paragraph [00837], wherein the structure is a oligonucleotide or oligonucleotide analogue.

The compound of paragraph [00837], wherein the structure is a peptide nucleic acid.

A compound comprising a first chemical moiety, a second chemical moiety, and a nitrogen-containing group, wherein the first chemical moiety, the second chemical moiety, and the branched group form:

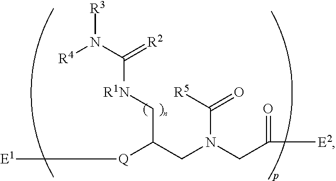

wherein:
R¹ is H, alkyl, or a nitrogen atom protecting group;
R² is O, NH, N(alkyl), or N(Pg^{N}), wherein Pg^{N} is a nitrogen atom protecting group;
R³ is H, alkyl, or a nitrogen atom protecting group;
R⁴ is H, alkyl, or a nitrogen atom protecting group;
R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
E¹ is the first chemical moiety;
E² is the second chemical moiety;
n is 1, 2, 3, 4, 5, 6, 7, 8, 9, or 10; and
p is an integer that is 1-1,000,
   or a pharmaceutically-acceptable salt or ionized form thereof.

The compound of paragraph [00840], wherein R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, or heterocyclyl.

The compound of paragraph [00840], wherein R⁵ is linear alkyl, branched alkyl, or cyclic alkyl.

The compound of paragraph [00840], wherein R⁵ is linear alkyl.

The compound of paragraph [00840], wherein R⁵ is methyl.

The compound of any one of paragraphs [00840]-[00844], wherein Q is NH or N(Pg^{N}).

The compound of any one of paragraphs [00840]-[00845], wherein n is 3.

The compound of any one of paragraphs [00840]-[00845], wherein n is 4.

The compound of any one of paragraphs [00840]-[00847], wherein each of R¹, R³, and R⁴ is hydrogen; and R² is NH or N(Pg^{N}).

The compound of any one of paragraphs [00840]-[00847], wherein p is from 1 to 100

The compound of any one of paragraphs [00840]-[00847], wherein p is 5, 6, 7, 8, or 9.

The compound of any one of paragraphs [00840]-[00850], wherein E¹ is hydrogen, acyl, a group that together with the nitrogen atom to which E¹ is bound forms a carbamate, a linker, a probe, a metal chelator, an imaging agent, or a biological agent.

The compound of any one of paragraphs [00840]-[00851], wherein E¹ is a biological agent.

The compound of any one of paragraphs [00840]-[00851], wherein E¹ is hydrogen.

The compound of any one of paragraphs [00840]-[00853], wherein E² is OH, O-alkyl, NH₂, a linker, a probe, a metal chelator, an imaging agent, or a biological agent.

The compound of any one of paragraphs [00840]-[00853], wherein E² is a biological agent.

The compound of any one of paragraphs [00840]-[00853], wherein E² is OH or NH₂.

The compound of any one of paragraphs [00851], [00852], [00854], and [00855], wherein the biological agent comprises a structure that interferes with expression of a gene associated with a neuromuscular disease phenotype.

The compound of any one of paragraphs [00851], [00852], [00854], [00855], and [00857], wherein the biological agent is a oligonucleotide or oligonucleotide analogue.

The compound of any one of paragraphs [00851], [00852], [00854], [00855], and [00857], wherein the biological agent is a peptide nucleic acid.

The compound of any one of paragraphs [00837]-[00839] and [00857], wherein the neuromuscular disease phenotype is a DM1 disease phenotype.

The compound of any one of paragraphs [00837]-[00839] and [00857], wherein the structure binds to a mRNA sequence transcribed from a gene associated with a DM1 disease phenotype.

The compound of paragraph [00861], wherein the DM1 gene is a non-wild type DM1 gene.

The compound of paragraph [00862], wherein the non-wild type DM1 gene differs from a wild type DM1 gene in a repeat expansion mutation.

The compound of any one of paragraphs [00837]-[00839] and [00857], wherein the structure binds to a mRNA sequence that contains a subsequence that is (CUG)_{z}, wherein z is an integer from 1-100 (SEQ ID NO: 135).

The compound of paragraph [00864], wherein the structure binds to the mRNA sequence at the subsequence that is (CUG)_{z}.

A composition comprising a compound according to any one of paragraphs [00410]-[00865], wherein the compound is therapeutically-effective for treatment of a neuromuscular disease.

The composition of paragraph [00866], wherein the neuromuscular disease is DM1.

A compound that is:

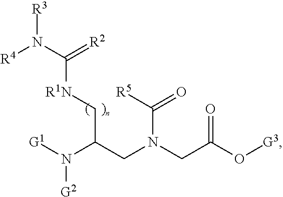

wherein:
R¹ is H, alkyl, or a nitrogen atom protecting group;
R² is NH, N(alkyl), or N(Pg^{N}), wherein Pg^{N} is a nitrogen atom protecting group;
R³ is H, alkyl, or a nitrogen atom protecting group;
R⁴ is H, alkyl, or a nitrogen atom protecting group;
R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
each of G¹ and G² is independently a nitrogen atom protecting group or hydrogen;
G³ is substituted or unsubstituted alkyl, substituted or unsubstituted alkenyl, haloalkyl, or hydrogen; and
n is 1, 2, 3, 4, 5, 6, 7, 8, 9, or 10,
   or a pharmaceutically-acceptable salt or ionized form thereof.

The compound of paragraph [00868], wherein G¹ is the nitrogen atom protecting group.

The compound of paragraph [00868], wherein G¹ is Boc, Nsc, Bsmoc, Nsmoc, ivDde, Fmoc*, Fmoc(2F), mio-Fmoc, dio-Fmoc, TCP, Pms, Esc, Sps or Cyoc.

The compound of paragraph [00868], wherein G¹ is Fmoc.

The compound of any one of paragraphs [00868]-[00871], wherein G² is hydrogen.

The compound of any one of paragraphs [00868]-[00872], wherein G³ is methyl, ethyl, n-propyl, isopropyl, n-butyl, iso-butyl, sec-butyl, or tert-butyl.

The compound of any one of paragraphs [00868]-[00872], wherein G³ is methyl, ethyl or, tert-butyl.

The compound of any one of paragraphs [00868]-[00872], wherein G³ is allyl.

The compound of any one of paragraphs [00868]-[00872], wherein G³ is 2-iodoethyl, 2-bromoethyl, 2,2,2-trichloroethyl, 2,2,2-trifluoroethyl, or 2,2,2-tribromoethyl.

The compound of any one of paragraphs [00868]-[00872], wherein G³ is tert-butyldimethylsilyl.

The compound of any one of paragraphs [00868]-[00872], wherein G³ is hydrogen.

The compound of any one of paragraphs [00868]-[00878], wherein n is 3.

The compound of any one of paragraphs [00868]-[00878], wherein n is 4.

The compound of any one of paragraphs [00868]-[00880], wherein R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, or heterocyclyl.

The compound of any one of paragraphs [00868]-[00880], wherein R⁵ is linear alkyl, branched alkyl, or cyclic alkyl.

The compound of any one of paragraphs [00868]-[00880], wherein R⁵ is linear alkyl.

The compound of any one of paragraphs [00868]-[00880], wherein R⁵ is methyl.

The compound of any one of paragraphs [00868]-[00884], wherein R¹ and R⁴ are each hydrogen.

The compound of any one of paragraphs [00868]-[00885], wherein R² is N(Pg^{N}), and R³ is the nitrogen atom protecting group.

The compound of any one of paragraphs [00868]-[00885], wherein R² is NH, NCbz, NBoc, NPmc, NPbf, NMtr, or NMts, and R³ is hydrogen, Cbz, Boc, Pmc, Pbf, Mtr, or Mts.

The compound of any one of paragraphs [00868]-[00885], wherein R² is NH, and R³ is the nitrogen atom protecting group.

The compound of paragraph [00888], wherein the nitrogen protecting group is Cbz, Boc, Pmc, Pbf, Mtr, or Mts.

The compound of any one of paragraphs [00868]-[00885], wherein R² is NCbz, and R³ is NHCbz.

The compound of any one of paragraphs [00868]-[00885], wherein R² is NBoc, and R³ is NHBoc.

The compound of any one of paragraphs [00868]-[00885], wherein R² is NH, and R³ is hydrogen.

The compound of paragraph [00868], wherein the compound is

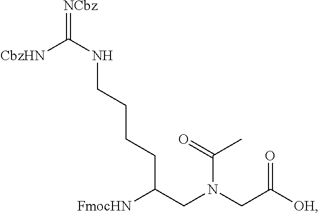

or a pharmaceutically acceptable salt thereof.

The compound of paragraph [00868], wherein the compound is

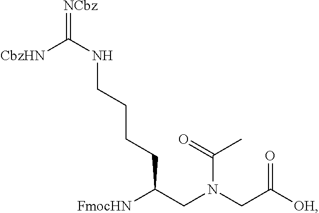

or a pharmaceutically acceptable salt thereof.

A compound comprising an oligomeric sequence, wherein the oligomeric sequence comprises a repeating unit of formula:

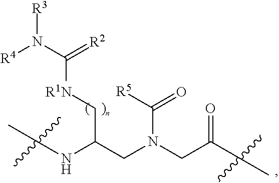

or an ionized form thereof, wherein:
R¹ is H, alkyl, or a nitrogen atom protecting group;
R² is O, NH, N(alkyl), or N(Pg^{N}), wherein Pg^{N} is a nitrogen atom protecting group;
R³ is H, alkyl, or a nitrogen atom protecting group;
R⁴ is H, alkyl, or a nitrogen atom protecting group;
R⁵ is alkyl or O-alkyl, any of which is unsubstituted or substituted; and
n is 1, 2, 3, 4, 5, 6, 7, 8, 9, or 10,
   or a pharmaceutically-acceptable salt or ionized form thereof.

The compound of paragraph [00895], wherein each of R¹, R³, and R⁴ is hydrogen; and R² is NH or N(Pg^{N}).

The compound of paragraph [00895] or paragraph [00896], wherein R⁵ is linear alkyl.

The compound of paragraph [00895] or paragraph [00896], wherein R⁵ is methyl.

The compound of any one of paragraphs [00895]-[00898], wherein n is 3.

The compound of any one of paragraphs [00895]-[00898], wherein n is 4.

The compound of paragraph [00895], further comprising a first chemical moiety attached to the oligomeric structure, and a second chemical moiety attached to the oligomeric structure, wherein the oligomeric structure, wherein the first chemical moiety, and the second chemical moiety form:

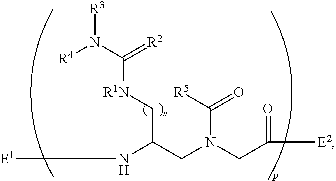

wherein:
E¹ is the first chemical moiety;
E² is the second chemical moiety; and
p is an integer that is 1-100.

The compound of paragraph [00901], wherein p is 6.

The compound of paragraph [00901], wherein p is 7.

The compound of paragraph [00901], wherein p is 8.

The compound of any one of paragraphs [00901]-[00904], wherein E¹ is hydrogen, acyl, a group that together with the nitrogen atom to which E¹ is bound forms a carbamate, a probe, a metal chelator, an imaging agent, or a biologically-active agent; and E² is OH, OMe, NH₂, a probe, a metal chelator, an imaging agent, or a biologically-active agent.

The compound of any one of paragraphs [00901]-[00904], wherein E¹ is hydrogen and E² is the biologically-active agent.

The compound of paragraph [00906], wherein the biologically-active agent comprises a structure that interferes with expression of a gene associated with a neuromuscular disease phenotype.

The compound of paragraph [00907], wherein the neuromuscular disease phenotype is a DM1 disease phenotype.

The compound of paragraph [00908], wherein the DM1 disease phenotype is associated with a non-wild-type DM1 gene that differs from a wild type DM1 gene in a repeat expansion mutation.

The compound of paragraph [00906], wherein the biologically-active agent binds to a mRNA sequence at a region that is (CUG)_{z}, wherein z is an integer from 1-100 (SEQ ID NO: 135).

The compound of any one of paragraphs [00906]-[00910], wherein the biologically-active agent is an oligonucleotide or oligonucleotide analogue.

The compound of any one of paragraphs [00906]-[00911], wherein the biologically-active agent is a peptide nucleic acid.

The compound of paragraph [00895], wherein the compound is:

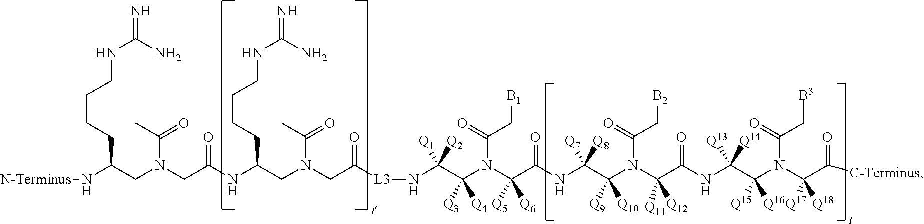

wherein:
each instance of B¹, B², and B³ is independently a heterocycle;
each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl, heteroalkyl, aryl, or heteroaryl, each of which is independently substituted or unsubstituted, or hydrogen;
L3 is a linker group or absent;
N-Terminus is H, acyl, a group that together with the nitrogen atom to which the N-Terminus is bound forms a carbamate, a probe, a fluorophore, a lipid, a metal chelator, or a biological agent;
C-Terminus is —N(H)-J, wherein J is H, acyl, a group that together with the nitrogen atom to which J is bound forms a carbamate, a probe, a fluorophore, a lipid, a metal chelator, or a biological agent;
t is an integer that is from 1 to 30; and
t′ is an integer that is from 2 to 9,
   or a pharmaceutically acceptable salt or ionized form thereof.

The compound of paragraph [00913], wherein L3 is absent.

The compound of paragraph [00913] or paragraph [00914], wherein N-Terminus is H.

The compound of any one of paragraphs [00913]-[00915], wherein C-Terminus is NH₂.

The compound of any one of paragraphs [00913]-[00916], wherein each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl that is substituted or unsubstituted, or hydrogen.

The compound of paragraph [00895], wherein the compound is:

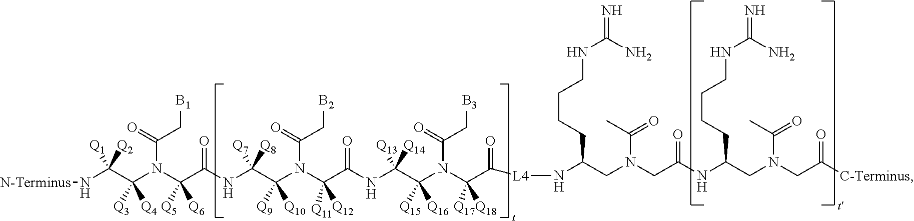

wherein:
each instance of B¹, B², and B³ is independently a heterocycle;
each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl, heteroalkyl, aryl, or heteroaryl, each of which is independently substituted or unsubstituted, or hydrogen;
L4 is a linker group or absent;
N-Terminus is H, acyl, a group that together with the nitrogen atom to which the N-Terminus is bound forms a carbamate, a probe, a fluorophore, a lipid, a metal chelator, or a biological agent;
C-Terminus is —N(H)-J, wherein J is H, acyl, a group that together with the nitrogen atom to which J is bound forms a carbamate, a probe, a fluorophore, a lipid, a metal chelator, or a biological agent;
t is an integer that is from 1 to 30; and
t′ is an integer that is from 2 to 9,
   or a pharmaceutically acceptable salt or ionized form thereof.

The compound of paragraph [00918], wherein L4 is absent.

The compound of paragraph [00918] or paragraph [00919], wherein C-Terminus is NH₂.

The compound of any one of paragraphs [00918]-[00920], wherein N-Terminus is H. The compound of any one of paragraphs [00918]-[00921], wherein each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷ and Q¹⁸ is independently an amino acid side chain, alkyl that is substituted or unsubstituted, or hydrogen.

A compound comprising a structure that is:

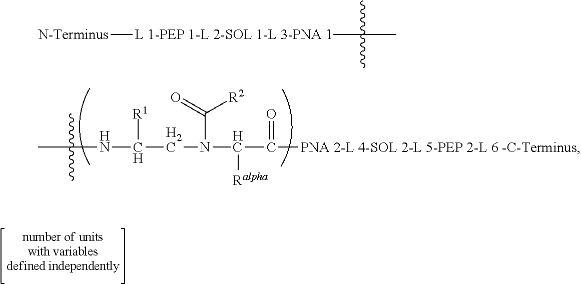

wherein:
the number of units with variables defined independently is at least 11;
N-Terminus is H, acyl, a group that together with the nitrogen atom to which the N-Terminus is bound forms a carbamate, a probe, a metal chelator, or a biological agent;
each R¹ is independently alkyl that is unsubstituted or substituted or H, wherein at least one iteration of R¹ is a hydroxyalkyl group;
each R^{alpha} is independently alkyl that is unsubstituted or substituted or H;
each R² is independently methyl substituted with a heterocycle;
C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof.

The compound of paragraph [00923], wherein the number of units with variables defined independently is 11, 12, 13, 14, 15, 16, or 17.

The compound of paragraph [00923], wherein the number of units with variables defined independently is 14.

The compound of any one of paragraphs [00923]-[00925], wherein N-Terminus is H and C-Terminus is NH₂.

The compound of any one of paragraphs [00923]-[00926], wherein each R¹ is independently H, hydroxylmethyl, or 4-guanidinobut-1-yl.

The compound of any one of paragraphs [00923]-[00926], wherein at least one iteration of R¹ is hydroxylmethyl.

The compound of any one of paragraphs [00923]-[00926], wherein at least half the iterations of R¹ are hydroxylmethyl and the other iterations of R¹ are H.

The compound of any one of paragraphs [00923]-[00929], wherein each of L1, L2, L3, L4, L5, and L6 is absent.

The compound of any one of paragraphs [00923]-[00930], wherein PEP1 and PEP2 are absent.

The compound of any one of paragraphs [00923]-[00930], wherein one of PEP1 and PEP2 is a peptide sequence that is a nuclear localization sequence and the other is absent.

The compound of any one of paragraphs [00923]-[00932], wherein SOL1 is the water-solubilizing group and SOL2 is absent.

The compound of any one of paragraphs [00923]-[00929], wherein each of L1, L2, L3, L4, L5, L6, PEP1, PEP2, and SOL2 is absent, and SOL1 is the water-solubilizing group. The compound of any one of paragraphs [00923]-[00934], wherein the water-solubilizing group is a group that contains multiple positive charges at physiological pH.

The compound of any one of paragraphs [00923]-[00934], wherein the water-solubilizing group of SOL1 is a group of formula:

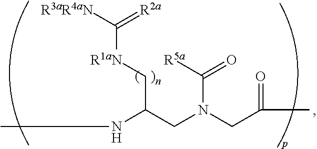

wherein
R^{1a} is H, alkyl, or a nitrogen atom protecting group;
R^{2a} is O, NH, N(alkyl), or N(Pg^{N}), wherein Pg^{N} is a nitrogen atom protecting group;
R^{3a} is H, alkyl, or a nitrogen atom protecting group;
R^{4a} is H, alkyl, or a nitrogen atom protecting group;
R^{5a} is alkyl or O-alkyl, any of which is unsubstituted or substituted;
n is 1, 2, 3, 4, 5, 6, 7, 8, 9, or 10; and
p is an integer that is 1-100.

The compound of paragraph [00936], wherein p is 5, 6, 7, or 8.

The compound of paragraph [00936], wherein p is 7.

The compound of paragraph [00936], wherein the water-solubilizing group of SOL1 is a group of formula:

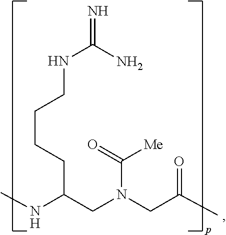

wherein p is an integer that is 5, 6, 7, or 8.

The compound of paragraph [00936], wherein the water-solubilizing group of SOL1 is a group of formula:

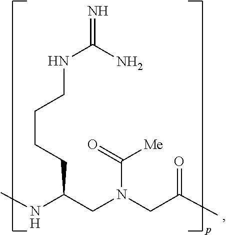

wherein p is an integer that is 5, 6, 7, or 8.

The compound of paragraph [00939] or paragraph [00940], wherein p is 7.

The compound of any one of paragraphs [00923]-[00941], wherein the heterocycles of the R² groups are nucleobases or analogues of nucleobases.

The compound of any one of paragraphs [00923]-[00942], wherein the heterocycles of the R² groups are each independently:

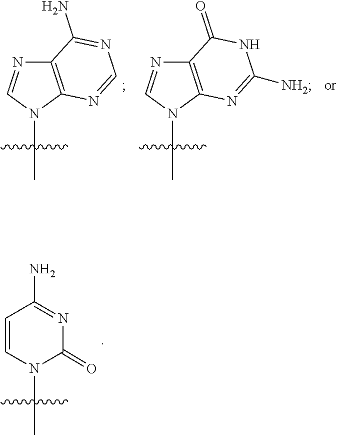

The compound of any one of paragraphs [00923]-[00942], wherein each R² group in the structure is independently:

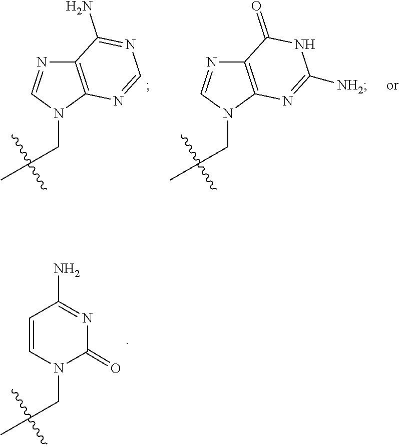 [SMILES: *Cn1cnc2c(N)ncnc21 ; *Cn1cnc2c(=O)[nH]c(N)nc21 ; *Cn1ccc(N)nc1=O]

The compound of any one of paragraphs [00923]-[00942], wherein the heterocycles of the R² groups form a sequence that repeats at least twice, wherein the sequence is, from N-Terminus to C-Terminus:

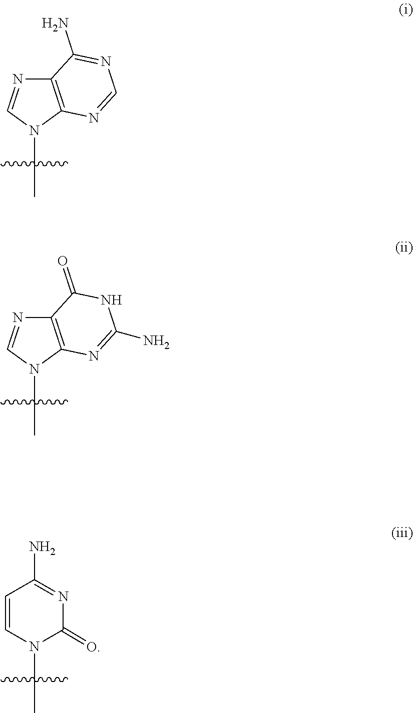 [SMILES: *n1cnc2c(N)ncnc21 ; *n1cnc2c(=O)[nH]c(N)nc21 ; *n1ccc(N)nc1=O]

The compound of any one of paragraphs [00923]-[00945], wherein the compound binds to a mRNA sequence transcribed from a gene associated with a neuromuscular disease phenotype.

The compound of any one of paragraphs [00923]-[00945], wherein the compound binds to a mRNA sequence transcribed from a gene associated with a DM1 disease phenotype.

The compound of any one of paragraphs [00923]-[00945], wherein the compound binds to a mRNA sequence transcribed from a gene associated with a DM1 disease phenotype by interactions between the heterocycles of the R² groups and nucleobases of a DM1 gene.

The compound of paragraph [00948], wherein the DM1 gene is a non-wild type DM1 gene.

The compound of paragraph [00949], wherein the non-wild type DM1 gene differs from a wild type DM1 gene in a repeat expansion mutation.

The compound of any one of paragraphs [00923]-[00945], wherein the compound binds to a mRNA sequence of (CUG)_{z}, wherein z is an integer from 1-100 (SEQ ID NO: 135).

The compound of paragraph [00923], wherein the compound is:

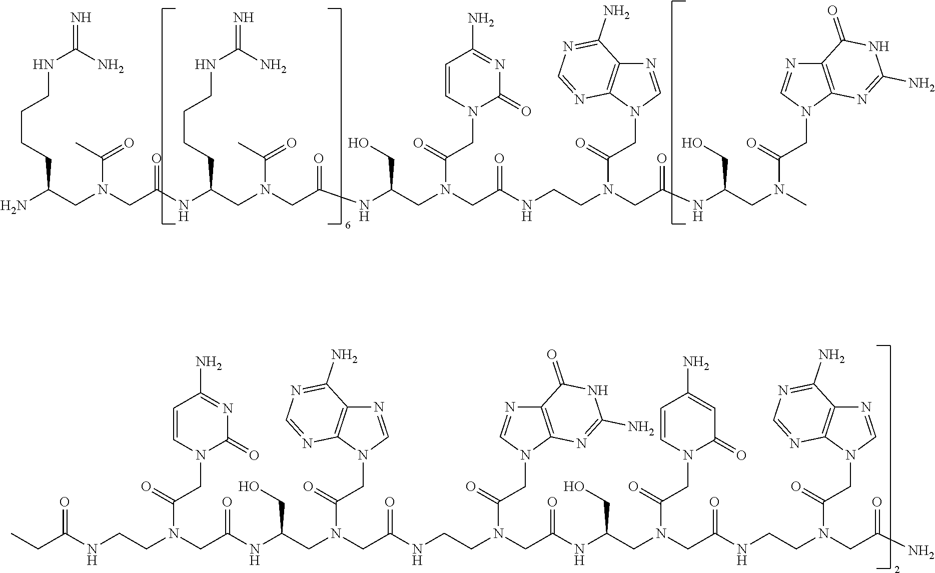

or a pharmaceutically-acceptable salt or ionized form thereof.

A compound of formula:

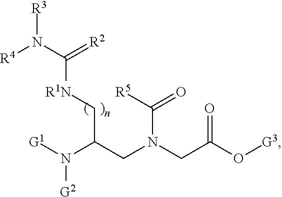

wherein:
R¹ is H, alkyl, or a nitrogen atom protecting group;
R² is NH, N(alkyl), or N(Pg^{N}), wherein Pg^{N} is a nitrogen atom protecting group;
R³ is H, alkyl, or a nitrogen atom protecting group;
R⁴ is H, alkyl, or a nitrogen atom protecting group;
R⁵ is alkyl or O-alkyl, any of which is unsubstituted or substituted;
each of G¹ and G² is independently a nitrogen atom protecting group or hydrogen;
G³ is substituted or unsubstituted alkyl, substituted or unsubstituted alkenyl, haloalkyl, or hydrogen; and
n is 1, 2, 3, 4, 5, 6, 7, 8, 9, or 10,
   or a salt or ionized form thereof.

The compound of paragraph [00953], wherein G¹ is the nitrogen atom protecting group, G² is hydrogen, and G³ is hydrogen.

The compound of paragraph [00983] or paragraph [00954], wherein G¹ is Fmoc.

The compound of any one of paragraphs [00953]-[00955], wherein n is 3.

The compound of any one of paragraphs [00953]-[00955], wherein n is 4.

The compound of any one of paragraphs [00953]-[00957], wherein R⁵ is methyl.

The compound of paragraph [00953], wherein R¹ is hydrogen, R² is N(Pg^{N}), R³ is a nitrogen atom protecting group, and R⁴ is hydrogen.

The compound of paragraph [00953], wherein the compound is

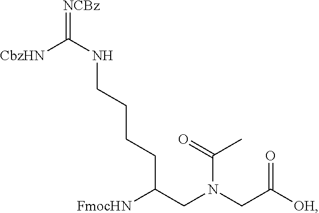

or a salt or ionized form thereof.

The compound of paragraph [00953], wherein the compound is

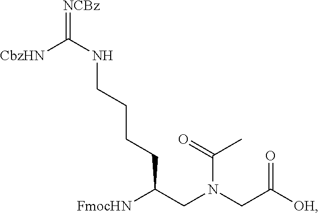

or a salt or ionized form thereof.

A compound of formula:

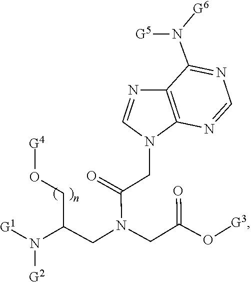

wherein:
G¹ is H or a nitrogen atom protecting group;
G² is H or a nitrogen atom protecting group;
G³ is H or carboxylate protecting group;
G⁴ is H or a hydroxyl protecting group;
G⁵ is H;
G⁶ is H or a nitrogen atom protecting group; and
n is 1, 2, 3, or 4,
   or a salt or ionized form thereof.

The compound of paragraph [00962], wherein:
G¹ is a nitrogen atom protecting group; G² is H; G³ is H; G⁴ is a hydroxyl protecting group; G⁶ is a nitrogen atom protecting group; and n is 1.

The compound of paragraph [00962], wherein the compound is:

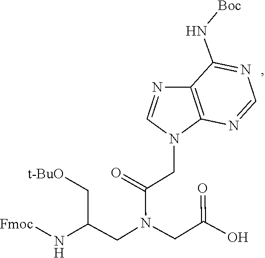

or a salt or ionized form thereof.

A compound of formula:

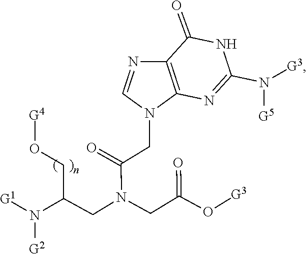

wherein:
G¹ is H or a nitrogen atom protecting group;
G² is H or a nitrogen atom protecting group;
G³ is H or carboxylate protecting group;
G⁴ is H or a hydroxyl protecting group;
G⁵ is H or a nitrogen atom protecting group;
G⁶ is H or a nitrogen atom protecting group; and
n is 1, 2, 3, or 4,
   or a salt or ionized form thereof.

The compound of paragraph [00965], wherein: G¹ is a nitrogen atom protecting group; G² is H; G³ is H; G⁴ is a hydroxyl protecting group; G⁵ is H; G⁶ is a nitrogen atom protecting group; and n is 1.

The compound of paragraph [00965], wherein the compound is:

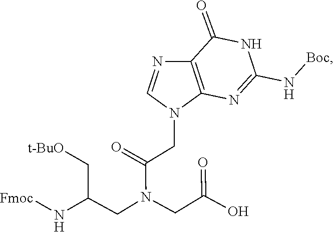

or a salt or ionized form thereof.

A method of treating a condition, the method comprising administering to a subject in need thereof a therapeutically-effective amount of a compound of any one of paragraphs [00923]-[00952].

The method of paragraph [00968], wherein the condition is a neuromuscular disease.

The method of paragraph [00968] or paragraph [00969], wherein the condition is associated with a gene having a repeat expansion mutation.

The method of any one of paragraphs [00968]-[00970], wherein the condition is DM1.

The method of any one of paragraphs [00968]-[00971], wherein the subject is human.

### EXAMPLES

### Example 1: Reduction in Nuclear Inclusions in HSA^{LR} Mouse Muscle Using Compound 1

The ability of Compound 1 to reduce nuclear aggregates/inclusions caused by mRNA with CUG repeats was assessed in a HSA^{LR} mouse model via Fluorescence in situ hybridization (FISH). The HSA^{LR} mouse model of myotonic dystrophy utilizes transgenic mice that have a CUG repeat in the 3′ UTR of hACTA1 mRNA. The CUG repeat leads to the formation of toxic hairpins and impaired splicing due to MBNL1 sequestration, as seen in myotonic dystrophy.

HSA-LR mice were: (i) treated with a single dose of Compound 1 by tail vein (IV) injection at 29 mg/kg, (ii) treated with daily doses of 3 mg/kg Compound 1 for 7 days by tail vein (IV) injection at 3 mg/kg, or (iii) injected with PBS as an untreated control. The mice were then sacrificed 20 or 21 days after the first injection and the quadricep was harvested and snap frozen in liquid nitrogen. The frozen tissue was then fixed and sectioned in preparation for evaluation of nuclear inclusions.

Fluorescence in situ hybridization (FISH) was performed on 6 micron sections using a CAG repeat probe having the sequence: /5Cy3/CAGCAGCAGCAGCAGCAGCA (SEQ ID NO: 111). The probe (1 ng/μl final concentration) was hybridized to sections overnight. After washing, sections were imaged using confocal fluorescence microscopy, with DAPI as a nuclear counterstain for localization.

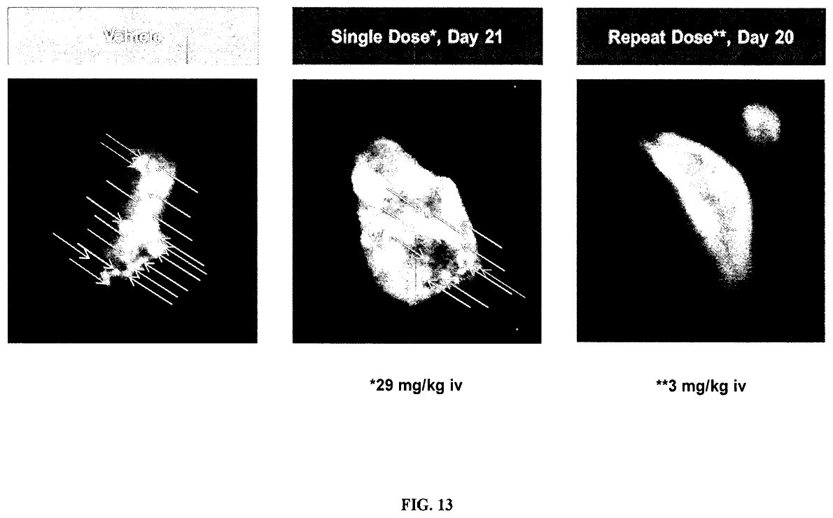

Reductions in nuclear inclusions were observed for Animals treated with Compound 1 as shown in FIG. 13, where intensities attributable to probe are denoted with white arrows.

### Example 2: In Vivo Mis-Splicing Correction Using Compound 1

In vivo mis-splicing correction of the myotonia-causing transcripts by Compound 1 was assessed. Single doses of Compound 1 were administered IV at 29 mg/kg to the HSA^{LR} mice, and RNA seq performed on tibialis anterior muscle. Untreated HSA^{LR} mice and healthy FVB background mice were included as controls.

DSI calculated as below was used as a composite score of global splice rescue 13 days after treatment. The DSI provides a composite view of splice correction across only mis-spliced transcripts.

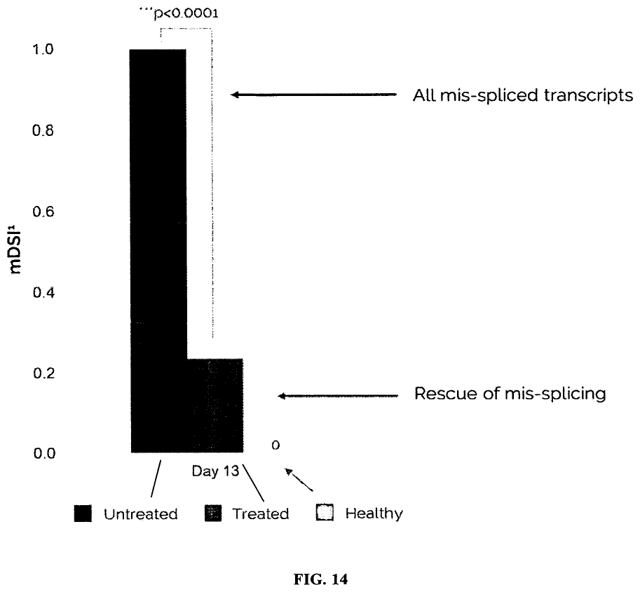

As shown in FIG. 14, a statistically significant broad correction across many transcripts was observed for HSA^{LR} mice treated with the single dose of Compound 1 (p<0.0001). Approximately 75% of mis-spliced transcripts were rescued within 13 days relative to untreated animals that harbor the mutation, which animals exhibit a set of inappropriately spliced genes that lead to the disease. Among the genes exhibiting rescued splicing was the chloride channel CLCN1 gene, mis-splicing of which can cause the myotonia phenotype, insulin receptor (IR), and others.

### RNA Sequencing Analyses.

A. Measurement of Aberrant Splicing (hDSI/mDSI).

Tissues were collected and processed for RNA sequencing. Reads were aligned to reference data (see below) with STAR v2.7.1a using the STAR options from rMATS v4.1.0. Duplicates were removed with Picard MarkDuplicates v2.18.7. Aberrant splice activity was measured with rMATS v4.1.0 run on all pairwise combinations of conditions (healthy-untreated, disease-treated, and disease-untreated) with standard options. The top events from the “Skipped Exons Junction Counts Exon Counts” (‘SE.MATS.JCEC.txt’) from the three outputs were then merged together. Any event not present in the healthy-untreated vs. disease-treated comparison was dropped, and only those events with a minimum absolute inclusion difference greater than 0.2 were included.

Mouse/human differential splice inclusion (mDSI/hDSI) were calculated by first selecting a set of cassette exons with:
a) delta-PSI (absolute difference in Percent Splice Inclusion) between healthy-unrelated and disease-untreated samples, as calculated by rMATS, of >=20%;
b) p-value of <=0.05 for differential splicing as calculated by rMATS; and
c) read depth >=5× in all healthy-untreated, disease-untreated and disease-treated samples.

For each cassette exon a scale was calibrated from 0-1, where 0 is the mean PSI of healthy-untreated samples and 1 is the mean PSI of disease-untreated samples. For each sample, the PSI for each cassette exon was normalized into the scale, and then values for all selected cassette exons were averaged to yield mDSI/hDSI.

### B. Transcript Splice Correction.

Reads were aligned to the FASTA file using BWA with a clipping penalty of “100,100”. Junctions were identified by aligning the exclusion isoform against the inclusion isoform. Reads overlapping the identified splice junctions were then counted using sambamba v0.6.6 with the following filters: ‘“mapping_quality >=60 and not (secondary_alignment or failed_quality_control or duplicate or supplementary or chimeric)”’.

### Reference Data:

Mouse—GCF_000001635.27 GRC39
Human—GCF_000001405.39 GRCh38.019

### Example 3: Restoration of Skeletal Muscle Chloride Channel (CLCN1) In Vivo Using Compound 1

The ability of Compound 1 to correct Clcn1 splicing and expression was assessed in vivo. HSA^{LR} mice were treated intravenously (IV) with: (i) 3 mg/kg doses of Compound 1, daily for seven days, or (ii) a single dose of Compound 1 at 30 mg/kg. Untreated HSA^{LR} mice and healthy untreated FVB background mice were included as controls (n=3 per group).

20 days after the first dose, muscles were harvested and processed for RNA seq analysis and western blotting.

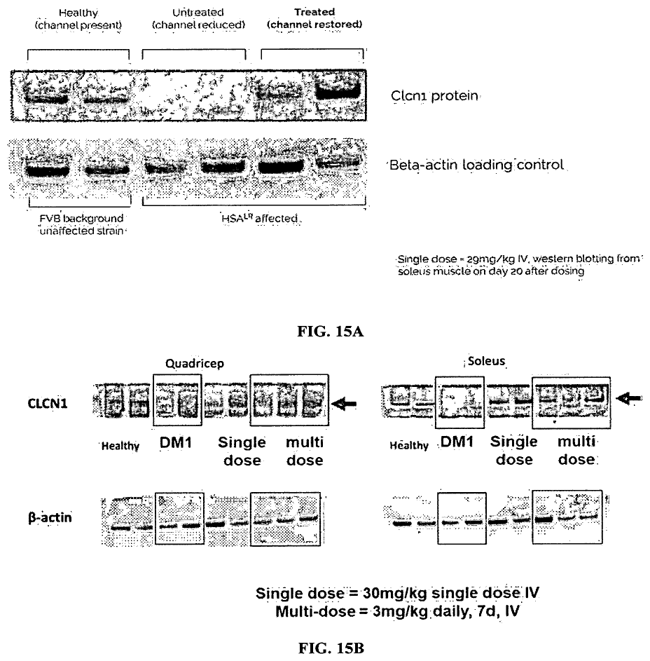

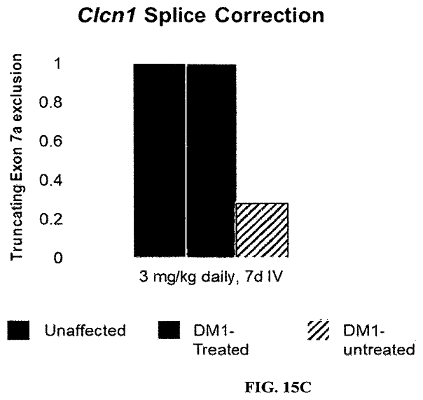

Levels of the chloride channel were restored at protein level by day 20 (FIG. 15A, showing data from soleus muscle for the group that received the single dose regimen, and FIG. 15B, showing data from quadricep and soleus for both dosing groups. As shown in FIG. 15C, approximately 99% of normal Clcn1 transcript splicing was corrected by day 20 for mice treated daily with 3 mg/kg of compound 1.

### Western Blot Methods.

About 50-100 mg of muscle tissue was homogenized and lysed using 300 μM-Per™ buffer (Thermo Scientific™, Waltham, MA, cat. n #78503). Protein extracts were quantified using BCA assay (Pierce™ BCA protein Assay Kit cat. n #23225, Thermo Scientific, Waltham, MA). Protein extracts were resolved on SDS-PAGE 4-12% gradient gels (Invitrogen™, Carlsbad, CA, cat #28451) with Tris-SDS IVIES Buffer (Invitrogen™, Carlsbad, CA cat #22341). Gels were run at 150 V for 2 hours. After gel electrophoresis, proteins were transferred to a nitrocellulose membrane (Invitrogen™, Carlsbad, CA, cat #PB3310). Primary antibodies specific for CLCN1 (1:1000, Abcam™, Cambridge UK, cat. n #ab189857) and Beta-actin (1:5000, Abcam™, Cambridge, UK, cat. n #ab8227) were used. HRP conjugate anti-mouse or anti-rabbit secondary antibody (1:10,000, Jackson ImmunoResearch Laboratories, West Grove, PA, cat. n #315-035-0003 and 111-035-045) were used for visualizing proteins using SuperSignal™ West Pico Plus Chemiluminescent Substrate (Thermo Scientific™, Waltham, MA, cat. n #34577). Protein bands were quantified using iBright™ Analysis Software (Thermo Scientific, Waltham, MA). Bands were normalized according to beta-actin expression and the CLCN1 expression inhibition was calculated as a relative value to untreated control cells.

### RNA Seq Methods.

Total RNA was collected using RNAeasy kit (Qiagen cat #74106) following instruction manual. RNA was eluted in 30 μl and quantify. Libraries for RNA sequencing were created from polyA-selected RNA using a kit from Illumina cat #20040532 and Index plate cat #20040553. The protocol was adjusted for 300 ng total RNA. Libraries were amplified for 13 cycles and after clean up, concentration and quality were checked using Qubit and Tape station (D1000 ScreenTape cat #5067-5582, D1000 Reagents cat #5067-5583, Agilent). Library were run on Illumima NextSeq500. 2×150 bp sequencing was performed with read depth of approximately 50M. To analyze the data, reads were aligned to reference data with STAR v2.7.1a using the STAR options from rMATS v4.1.0. Duplicates were removed with Picard MarkDuplicates v2.18.7. Aberrant splice activity was measured with rMATS v4.1.0 run on all pairwise combinations of conditions (healthy-untreated, disease-treated, and disease-untreated) with standard options. The top events from the “Skipped Exons Junction Counts Exon Counts” (‘SE.MATS.JCEC.txt’) from the three outputs were then merged together using a custom tool. Any event not present in the healthy-untreated vs. disease-treated comparison was dropped, and only those events with a minimum absolute inclusion difference greater than 0.2 were included.

### Example 4: Functional Rescue of Myotonia In Vivo

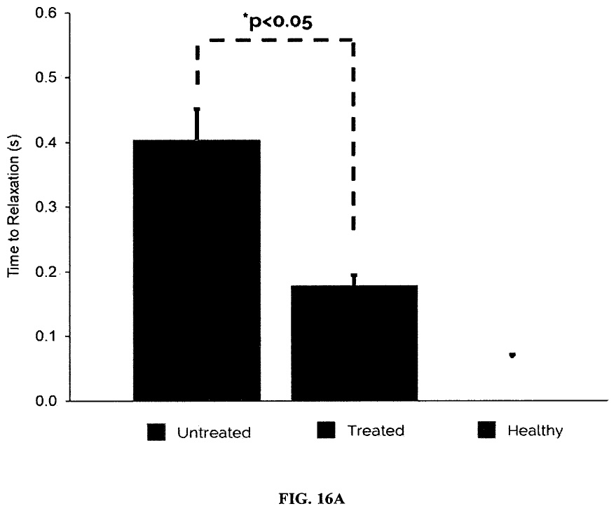

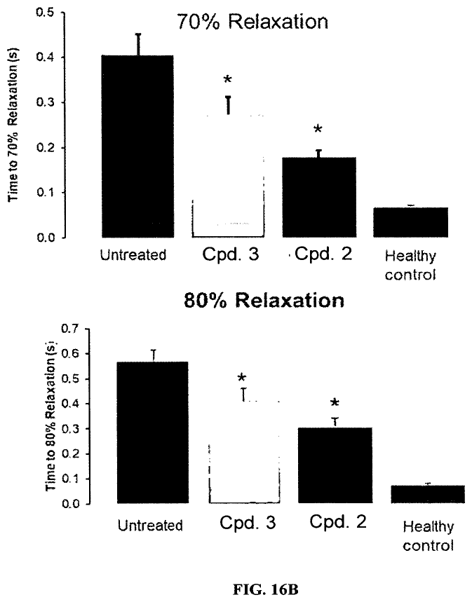

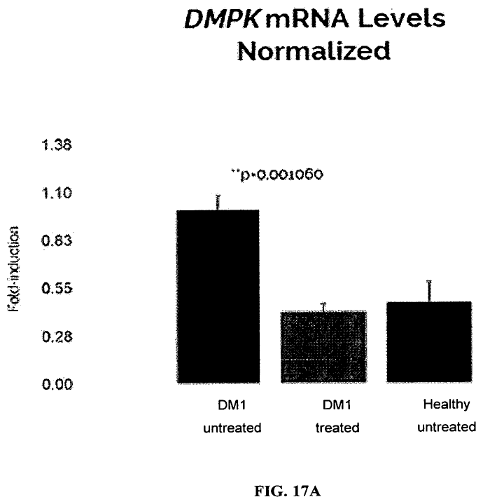

In vivo functional rescue of myotonia was assessed by muscle relaxation tests using Compound 2 and Compound 3. HSA^{LR} mice were subcutaneously dosed weekly for four weeks with 3 mg/kg of Compound 2 or Compound 3. As shown in FIG. 16A, a ˜70% reduction of myotonia was observed at 35 days after the first dose for mice dosed with Compound 2. FIG. 16B provides additional data for both Compound 2 and Compound 3, including data calculated with a relaxation threshold of 70% and data calculated with a relaxation threshold of 80%, with significance indicated for a 2-way ANOVA. These experiments demonstrated significant reduction in myotonia in vivo when compounds of the disclosure are administered via the subcutaneous route. Myotonia was assessed by a plantar flexor torque assay as described below.

Muscle performance was measured in vivo using a nerve-evoked muscle function in vivo assay that provides a measurement of the specific force of the plantarflexor muscle group. The assay evokes muscle contraction by direct electrical stimulation. Myotonia is measured as the delay of relaxation muscle after the measure of the maximal isometric tetanic force.

Muscle performance was measured with a 305C muscle lever system (Aurora Scientific Inc., Aurora, CAN) on anesthetized mice. The mice were placed on a thermostatically controlled table and anesthesia maintained via nose-cone (˜3% isoflurane, or to effect). The knee was isolated using a pin pressed against the tibial head and the foot firmly fixed to a footplate on the motor shaft. For the plantar flexor muscle group, contractions were elicited by percutaneous electrical stimulation of the sciatic nerve. No stimulations were performed prior to initiation of the test contractions. Optimal stimulus intensity was determined using a wild-type FVB/N mouse, then increased 30%. Ten (10) contractions (0.2 ms pulse, 500 ms train duration) were elicited at 30 second intervals and relaxation was recorded for 5 seconds. The force and relaxation curves were normalized to maximal force prior to analysis.

### Example 5: Correction of Splicing and Normalization of DMPK mRNA Expression in DM1 Patient Fibroblasts Treated with Compound 1

The ability of Compound 1 to correct splicing in human DM1 patient fibroblasts was evaluated.

DM1 human patient-derived fibroblast cell line GM03989 and normal fibroblast control cell line GM7492 were maintained at 37° C. and 5% CO₂ in MEM supplemented with 15% heat inactivated FBS. Cells were plated in 24-well plates at 100,000 cells/well in 1 mL of medium one day before treatment. Before treatment, medium was replaced with MEM with 2% heat-inactivated FBS. Stock solutions of Compound 1 were heated at 80° C. for 10 min before use and were added to the cell cultures at a final concentration of 1 μM. Cells were incubated for 5 days in the presence of Compound 1. All treatments were performed in triplicate.

Total RNA was collected using an RNAeasy kit on days 1, 2, 3, 4, 5, and 7. RNA was eluted in 30 μL and quantified.

For quantification of DMPK mRNA via qPCR, cDNA synthesis was performed using an Invitrogen Super Script III kit with priming by random hexamers. DMPK expression level was analyzed via SYBR green rt-qPCR using the ΔΔCt method, normalizing to GAPDH, with the following primers: DMPK_FWD: CACTGTCGGACATTCGGGAAGGTGC (SEQ ID NO: 115); DMPK_REV: GCTTGCACGTGTGGCTCAAGCAGCTG (SEQ ID NO: 116); GAPDH_FWD: GTCTCCTCTGACTTCAACAGCG (SEQ ID NO: 117) GAPDH_Rev: ACCACCCTGTTGCTGTAGCCAA (SEQ ID NO: 118).

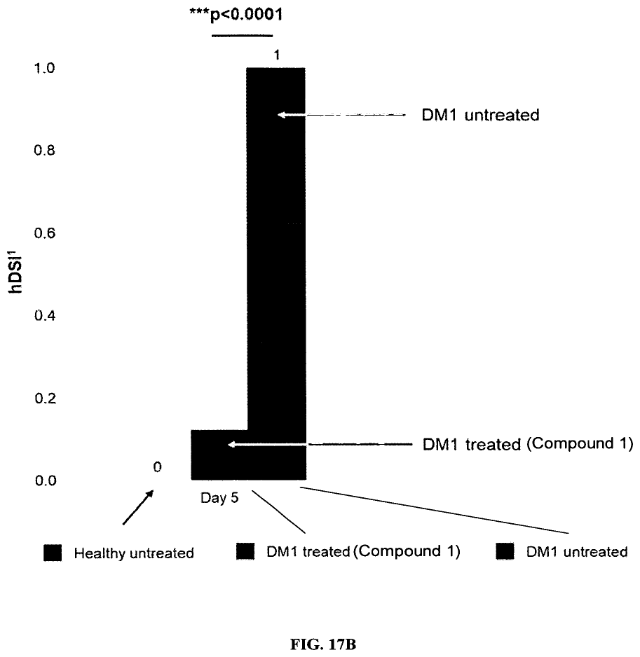

As shown in FIG. 17A, treatment with Compound 1 at 1 μM reduced the level of DMPK transcript to that of the normal control cell line within 24 hours.

For RNA sequencing, libraries were created from polyA-selected RNA using a kit from Illumina cat #20040532 and Index plate cat #20040553. The protocol was adjusted for 300 ng total RNA. Libraries were amplified for 13 cycles and after clean up, concentration and quality were checked using Qubit and Tape station. Sequencing was performed with an Illumima NextSeq500. To analyze the data, reads were aligned to reference data with STAR v2.7.1a using the STAR options from rMATS v4.1.0. Duplicates were removed with Picard MarkDuplicates v2.18.7.

Aberrant splice activity was measured with rMATS v4.1.0 run on all pairwise combinations of conditions (healthy-untreated, disease-treated, and disease-untreated) with standard options. The top events from the “Skipped Exons Junction Counts Exon Counts” (‘SE.MATS.JCEC.txt’) from the three outputs were then merged together. Any event not present in the healthy-untreated vs. disease-treated comparison was dropped, and only those events with a minimum absolute inclusion difference greater than 0.2 were included.

Human differential splice inclusion (hDSI) was calculated by first selecting a set of cassette exons with:
a) delta-PSI (absolute difference in Percent Splice Inclusion) between healthy-unrelated and disease-untreated samples, as calculated by rMATS, of >=20%
b) p-value of <=0.05 for differential splicing as calculated by rMATS
c) read depth >=5× in all healthy-untreated, disease-untreated and disease-treated samples.

For each cassette exon a scale was calibrated from 0-1 where 0 is the mean PSI of healthy-untreated samples and 1 is the mean PSI of disease-untreated samples. For each sample the PSI for each cassette exon was normalized into the scale, and then values for all selected cassette exons were averaged to yield hDSI.

The hDSI calculated as above was used as a composite score of global splice rescue after 5 days of treatment. As shown in FIG. 17B, a statistically significant broad correction across many transcripts was observed for DM1 cells treated with Compound 1 for 5 days (p<0.0001). Approximately 90% of mis-spliced transcripts were rescued. Treatment resulted in rescue of appropriate adult cassette exon use across many mis-spliced transcripts, including those known to rescue the phenotype including Clcn1, IR, and others.

### Example 6: Intramuscular Administration of Compounds of the Disclosure Reduces Pathogenic ACTA1 mRNA in the HSA^{LR} Mouse Model of Myotonic Dystrophy

The ability of compounds of the disclosure to reduce pathogenic mRNA comprising CUG repeats upon intramuscular administration was evaluated.

Mice were administered a single dose of Compound 1, Compound 2, or Compound 3 at a dose of 0.03 mg/kg via the intramuscular route. Muscle tissue was harvested six weeks after dosing and processed for qPCR to quantify transcript levels.

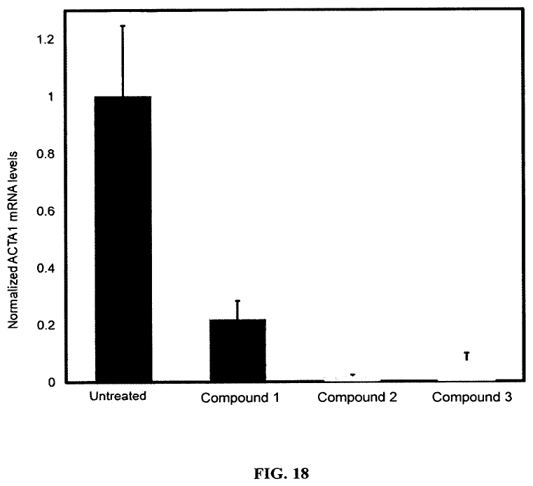

As shown in FIG. 18, a single 0.03 mg/kg dose via the intramuscular route of any of compounds 1, 2, or 3 reduced levels of pathogenic mRNA (hACTA1 mRNA, which comprises a CUG repeat in the 3′ UTR that leads to the formation of toxic hairpins and impaired splicing due to MBNL1 sequestration in the HSA^{LR} mouse model of myotonic dystrophy).

### RNA Isolation and RT-qPCR Methods.

All muscles were weighed before RNA isolation. For 100 mg tissue, total RNA was isolated using 1 mL of Trizol. Tissues was homogenized with OMNI TH homogenizer in Trizol. RNA was extracted using BCP, precipitated using Isopropanol, and resuspended in 100 μL RNase free water. Five μg of the isolated RNA was treated with DNase to remove genomic DNA, and RNA re-purified using an RNeasy Mini kit.

300 ng of purified total RNA was reverse transcribed using a SuperScriptIII First-Strand Synthesis System for RT-PCR. qPCR reactions were carried out using TaqMan Fast Advanced Master Mix. For ACTA1 qPCR, forward primer was GTAGCTACCCGCCCAGAAACT (SEQ ID NO: 112), reverse primer was CCAGGCCGGAGCCATT (SEQ ID NO: 113), and custom TaqMan probe was ACCACCGCCCTCGTGTGCG (3′ MGBNFQ quencher and 5′ 6FAM dye, IDT) (SEQ ID NO: 114) and a 1:10 dilution of cDNA was used as input. For Gtf2b reference gene qPCR, Gene Expression Assay Mm00663250 ml (4331182, Vic, Applied Biosystems) was used and 2 μL of cDNA. qPCR was performed on QuantaStudio5 using fast ramp speed. ACTA1 transgene expression relative to Gtf2b was calculated using the ΔΔCt method.

### Example 7: Pharmacologic Durability of Compound 1 Administered by Intramuscular Injection in the HSA^{LR} Mouse Model of Myotonic Dystrophy

The ability of Compound 1 to reduce pathogenic mRNA comprising CUG repeats and rescue splicing upon intramuscular administration was evaluated.

Mice were administered four 0.3 mg/kg doses of Compound 1, seven days apart, via the intramuscular route. Tibialis anterior muscle tissue was harvested six weeks after the final dose and processed for qPCR to quantify hACTA1 mRNA transcript levels, and splicing rescue of Mbln1 and Clcn1.

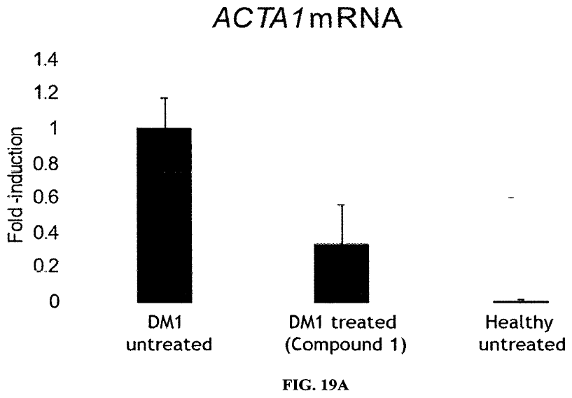

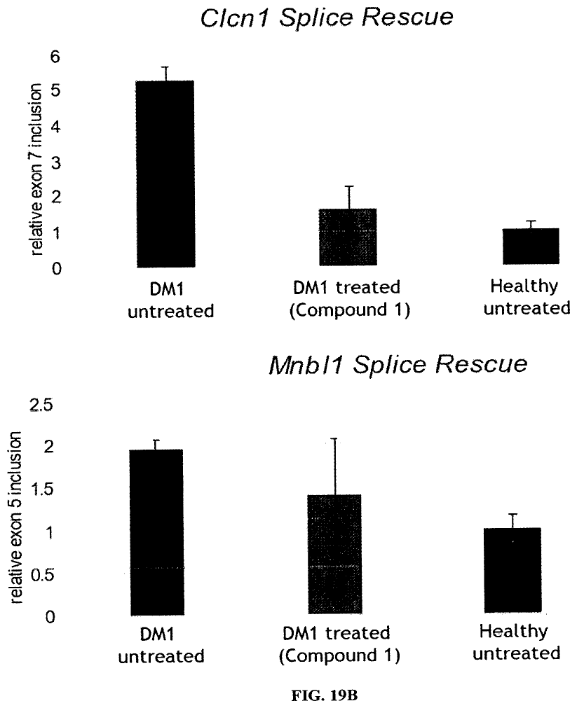

As shown in FIG. 19A, Compound 1 reduced levels of pathogenic hACTA1 mRNA to less than 40% of the untreated level. hACTA1 mRNA comprises a CUG repeat in the 3′ UTR that leads to the formation of toxic hairpins and impaired splicing due to MBNL1 sequestration in the HSA^{LR} mouse model of myotonic dystrophy. FIG. 19B shows that Compound 1 also resulted in near complete splicing rescue of Clcn1. Clcn1 mis-splicing can cause the myotonia phenotype in animal models and humans, and splicing rescue such as that observed in this study can be translationally relevant to human disease.

### RNA Isolation and RT-qPCR Methods.

All muscles were weighed before RNA isolation. For 100 mg tissue, total RNA was isolated using 1 mL of Trizol. Tissues was homogenized with OMNI TH homogenizer in Trizol. RNA was extracted using BCP, precipitated using Isopropanol, and resuspended in 100 μL RNase free water. Five μg of the isolated RNA was treated with DNase to remove genomic DNA, and RNA re-purified using an RNeasy Mini kit.

300 ng of purified total RNA was reverse transcribed using a SuperScriptIII First-Strand Synthesis System for RT-PCR.

For quantification of ACTA1, qPCR reactions were carried out using TaqMan Fast Advanced Master Mix. For ACTA1, forward primer was GTAGCTACCCGCCCAGAAACT (SEQ ID NO: 112), reverse primer was CCAGGCCGGAGCCATT (SEQ ID NO: 113), and custom TaqMan probe was ACCACCGCCCTCGTGTGCG (3′ MGBNFQ quencher and 5′ 6FAM dye, IDT) (SEQ ID NO: 114) and a 1:10 dilution of cDNA was used as input. For Gtf2b reference gene, Gene Expression Assay Mm00663250_ml (4331182, Vic, Applied Biosystems) was used with 2 μL of cDNA. qPCR was performed on QuantaStudio5 using fast ramp speed. ACTA1 transgene expression relative to Gtf2b was calculated using the ΔΔCt method.

To evaluate alternative splicing of MBNL1 exon 5 and Clcn1 exon 7, qPCR was performed using Power Up Sybr Green 2× master mix with 3 μL of cDNA and the following primers:

Mbnl1 exon 5
forward
(SEQ ID NO: 119)
5′-CTGTCAAATCACTGAAGCGACCC-3′;
reverse
(SEQ ID NO: 120)
5′-TGGTGGGAGAAATGCTGTATGC-3′.
Clcn1 exon 7
forward
(SEQ ID NO: 121)
5′-GATGCTACTTTGCAGCCCCTG-3′;
(SEQ ID NO: 122)
reverse
5′-CACGGAACACAAAGGCACTG-3′.
GAPDH
forward
(SEQ ID NO: 123)
5′-CATCACTGCCACCCAGAAGACTG-3′;
reverse
(SEQ ID NO: 124)
5′-ATGCCAGTGAGCTTCCCGTTCAG-3′.

qPCR was performed on QuantaStudio5. MBNL1 exon 5 and Clcn1 exon 7 inclusion were expressed relative to GAPDH and calculated using the ΔΔCt method.

### Example 8: Synthesis and Purification of Compounds of the Disclosure

Synthesis of crude PNA: PNA synthesis was performed using Fmoc solid-phase peptide chemistry on an Intavis MultiPep RSi (25 μmol scale) at ambient temperature. TentaGel® R RAM resin (0.18 meq/g) was swelled in N,N-dimethylformamide (DMF) (1 mL, 3×10 min). Fmoc groups were deprotected with 20% piperidine (v/v) in DMF (0.8 mL, 2×10 minutes). Following deprotection, the resin was washed with DMF (6×1 mL) and the next PNA residue was coupled to the N-terminus of resin-bound PNA upon treatment with a mixture of Fmoc-protected monomer (85 μmol, 3.4×, 0.2 M in NMP), HATU (80 μmol, 3.2×, 0.5 M in DMF), and N,N-diisopropylethylamine (DIPEA, 83 μmol, 3.3×, 0.52 M in DMF) (double coupling: 2 min pre-activation and 30 min coupling). After coupling, the resin was acetyl capped with 5% acetic anhydride and 6% 2,6-lutidine (v/v) in DMF (5 min), and subsequently washed with DMF (6×1 mL). Upon completion of all synthetic cycles, the resin was washed with DMF (5×) and DCM (5×). PNA was simultaneously cleaved and deprotected upon treatment with a mixture of trifluoroacetic acid/triflic acid/thioanisol/m-cresol (5 mL, 6:2:1:1) under agitation for 90 minutes. PNA was precipitated in ice-cold diethyl ether (45 mL), centrifuged, isolated from the supernatant, washed with ice-cold diethyl ether (50 mL), and dried under vacuum.

Purification of PNA: Crude PNA was dissolved in Milli-Q water (5 mL) and filtered through a nylon syringe-tip filter (0.45 μm pore size). Preparative reversed-phase high-pressure liquid chromatography (RP-HPLC) was performed on a Dionex Ultimate 3000 equipped with a Hypersil Gold C18aq 30×250 mm column, with a 5 μm particle size and 175 Å pore size. A 30-minute linear gradient of acetonitrile (5-25%) in water with 0.1% TFA was used as the mobile phase. HPLC eluate was fractionated based on UV absorbance (λ=254 nm) and fractional purity was assessed via MALDI-TOF in linear positive mode with a matrix of CHCA. Pure fractions were lyophilized, dissolved in water, combined, and re-lyophilized. Pure PNA was dissolved in PBS and the concentration of the PNA solution was determined via absorbance spectroscopy (ε(λ=260 nm)=148,300 cm⁻¹M⁻¹). Selected compounds were characterized by matrix-assisted laser desorption/ionization mass spectroscopy (MALDI-MS) or ESI+MS. Observed masses are presented in TABLE 8.

TABLE 8

| Cpd | | Observed | SEQ |
|---|---|---|---|
| # | Structure Code(N to C terminus)* | Mass [M + 1] | ID NO^{c} |
| 1 | YgYgYgYgYgYgYgCsAnGsCnAsGnCsAnGsCnAsGnCsAn | 5812.8 | — |
| 2 | {RHxRRBxRRHxRFQILYRHxRBxRHxRBx}CsAnGsCnAsGnC | 6912.4, | 128 |
| | sAnGsCnAsGnCsAn | 6919.6, | |
| | | 6915.7 | |
| 3 | {ASSLNIAHxBxR*R*R*}CsAnGsCnAsGnCsAnGsCnAsGnC | 5333.7 | 129 |
| | sAn | | |
| 4 | VeSxCsAnGsCnAsGnCsAnGsCnAsGnCsAn{RHxRRBxRRHx | 6340.5 | 130 |
| | RRBxRHxBx} | | |
| 5 | VeSxCsAnGsCnAsGnCsAnGsCnAsGnCsAn{RHxRRBxRRHx | 7427.5 | 128 |
| | RFQILYRHxRBxRHxRBx} | | |
| 6 | YgYgYgYgYgYgYgPe&&PeCsAnGsCnAsGnCsAnGsCnAsGn | 6955.6 | 1 |
| | CsAn{PKKKRKV} | | |
| 7 | YgYgYgYgYgYgYgPe&&PeCsAnGsCnAsGnCsAnGsCnAsGn | 6089.7 | — |
| | CsAn | | |
| 8 | YgYgYgYgYgYgYgEVCtCsAnGsCnAsGnCsAnGsCnAsGnCs | 6190.3 | — |
| | An | | |
| 11 | YgYgYgYgYgYgYgCsAnGsCnAsGnCsAnGsCnAsGnCsAnGs | 6130.1 | — |

^{c}Portion of structure code within braces(e.g., {PKKKRKV}” (SEQ ID NO: 1)), when
present, corresponds to SEQ ID NO provided in this column.

### Example 9A: Synthesis of A-(t-BuSer)-(Boc)C Monomer

### Step 1.

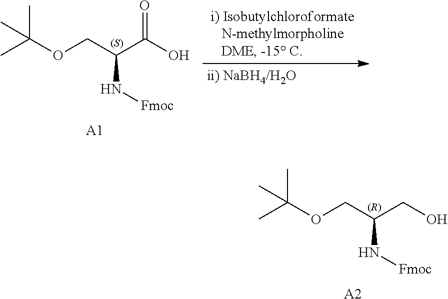

A solution of A1 (3.2 kg, 1.0 eq) in 1,2-dimethoxyethane (8.2 L, 2.6 V) was cooled to −20° C. in a liquid nitrogen/ethanol bath. N-methylmorpholine (1008 mL, 1.1 eq) was added dropwise at a rate such that the internal temperature did not exceed −10° C., and upon completion of the addition the solution was stirred for an additional 10 min. Isobutylchloroformate (1200 mL, 1.1 eq) was then added dropwise at a rate such that the internal temperature did not exceed −10° C., and upon completion of the addition the solution was stirred in the cold bath for an additional 1 h. The precipitate formed was removed via vacuum filtration and the precipitate was washed with 1,2-dimethoxyethane (2×2.6 L). The filtrate was cooled to −15° C. and a solution of sodium borohydride in water (800 g, 2.6 L) was added at a rate such that the internal temperature did not exceed −10° C. Upon completion of the addition the solution was stirred at −10° C. for 1 h. The reaction was quenched through the addition of water (16 L) and upon completion of the addition the solution was stirred for 30 min. The solution was filtered, and the filtrate was diluted with ethyl acetate (30 L). The ethyl acetate solution was washed with brine and the solvent was removed in vacuo to afford A2 as a white solid (2.31 kg, 98.9% yield).

### Step 2.

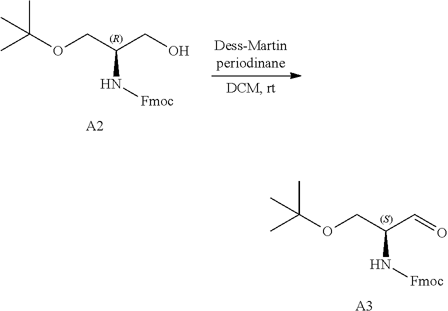

A solution of A2 (2.3 kg, 1.0 eq) in dichloromethane (23 L, 10V) was cooled in an ice-bath to 10° C. Dess-Martin periodinane (3.43 kg, 1.3 eq) was added in four portions, and upon completion of the addition the solution was allowed to warm to rt, and stirring was maintained for 2 h. The reaction was quenched through the addition of sodium thiosulphate solution (1.0N), and the layers were separated. The organic layer was washed with saturated sodium bicarbonate solution (20 L) and brine (2×10L) and the solution was dried over anhydrous sodium sulphate, filtered, and the solvent was removed in vacuo. The crude A3 was obtained as a pale yellow oil (2.7 kg) was used in the subsequent reaction without further purification.

### Step 3.

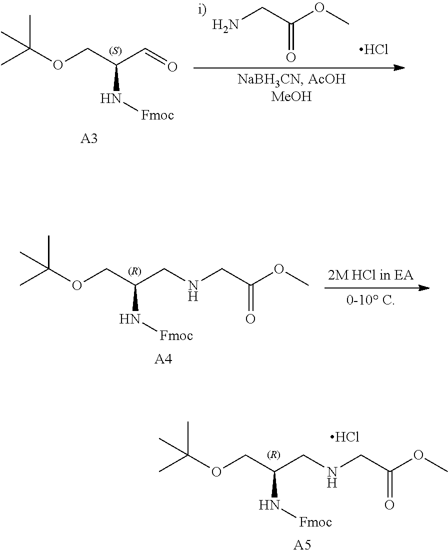

A solution of crude A3 (2.29 kg theoretical weight, 1.0 eq) was dissolved in methanol (18 L, 8V) and was cooled in an ice-bath to 10° C. H-Gly-OMe·HCl (1.56 kg, 2.0 eq) was added and the solution was stirred for 5 min. Sodium cyanoborohydride (782 g, 2.0 eq) and acetic acid (747 g, 2.0 eq) were added and the solution was allowed to warm to rt and stirring was maintained for 8 h. Upon completion of the reaction the solution was cooled to 10° C. and ethyl acetate (25 L, 10 V) was added, followed by saturated sodium bicarbonate solution (20 L). The layers were separated and the organic layer was washed with brine (2×10 L), dried over anhydrous sodium sulphate, filtered, and the solvent was removed in vacuo to afford a pale yellow oil (2.74 kg). tert-Butyl methyl ether (2.7 L) was added and the solution was cooled to 10° C. A solution of hydrochloric acid in ethyl acetate (2 N, 2.7 L) was added and the resulting white precipitate was collected via vacuum filtration to afford A5 as a white solid (1.70 kg, HPLC 94.6%).

### Step 4.

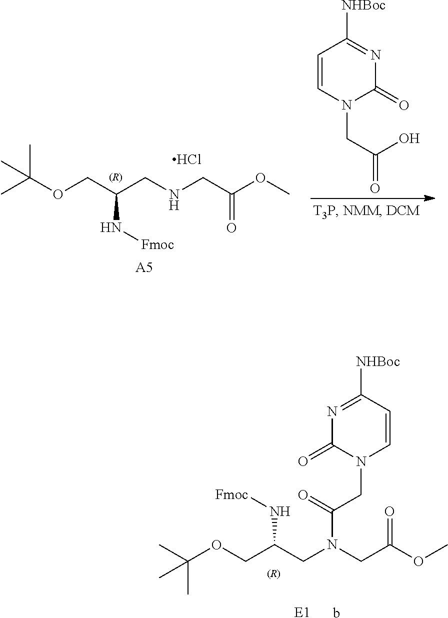

A solution of A5 (1.44 kg, 1.0 eq) and T₃P (3.60 kg, 2.0 eq) in dichloromethane (14.0 L, 10V) was cooled in an ice-bath to 15° C. under nitrogen. N-methylmorpholine (2.30 kg, 8.0 eq) was added and the solution was stirred for 5 min. 2-(4-((tert-Butoxycarbonyl)amino)pyrimidin-1(2H)-yl)acetic acid (1050 g, 1.3 eq) was added, and upon completion of the addition the solution was stirred at rt for 45 h. The solution was diluted with dichloromethane (7 L) and hydrochloric acid (10L, 1N) and the layers were separated. The organic layer was washed with brine (2×10 L), dried over anhydrous sodium sulphate, filtered, and the solvent was removed in vacuo to obtain E1 as a brown oil which was used in the subsequent step without further purification.

### Step 5.

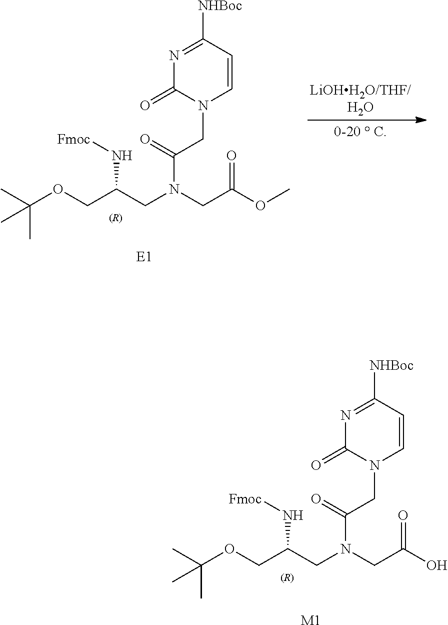

A solution of E1 (2.1 kg theoretical weight, 1.0 eq) in tetrahydrofuran (15 L, 7V) was cooled in an ice-bath to 10° C. A solution of lithium hydroxide (5.5 L, 1N, 1.8 eq) was added dropwise such that the internal temperature did not exceed 10° C. Upon completion of the addition the solution was allowed to warm to rt, and stirring was maintained for 3 h. N-(9-Fluorenylmethoxycarbonyloxy)succinimide (512 g, 0.5 eq) was added and the solution was stirred at rt for 12 h. Upon completion of the reaction the solution was diluted with water (15 L) and petroleum ether (15 L). The layers were separated and the aqueous phase was adjusted to pH=4 through the addition of hydrochloric acid solution (1N), and the mixture was extracted with ethyl acetate (25 L). The layers were separated and the organic layer was washed with brine (10 L), dried over anhydrous sodium sulphate, filtered, and the solvent was removed in vacuo to afford a yellow solid. The crude material was purified by column chromatography over silica, eluting with dichloromethane:methanol mixtures (30:1) to afford M1 as an off-white solid (1197 g, 59% yield, HPLC 98.0%). Enantiomeric ratio was measured by chiral phase HPLC (Superchiral S-AD, 0.46×15 cm, 5 μm, 70/30/0.5 (v/v/v) Hexane/EtOH/TFA, 0.9 mL/min, 254 nm, 40° C.) Rt (major)=7.2 min, Rt (minor)=11.1 min; er=99.9:0.1. LC-MS (ESI+) m/z: [M+1]+678.31.

### Example 9B: Synthesis of A-(t-BuSer)-(Boc)A Monomer

### Step 1.

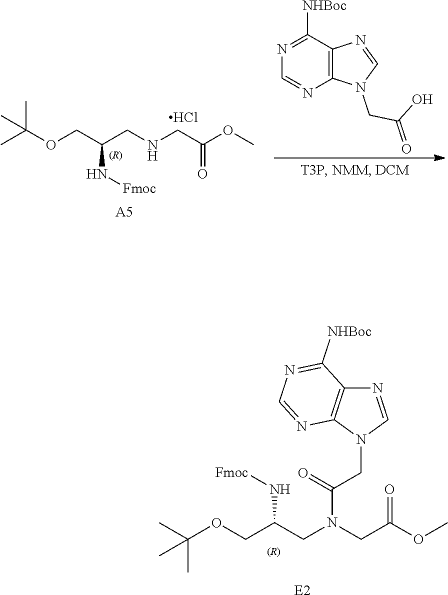

A5 was prepared according to Steps 1-3 of Example 9A. A solution of A5 (940 g, 1.0 eq) and T₃P (2.508 kg, 2.0 eq) in dichloromethane (9.4 L, 10V) was cooled in an ice-bath to 15° C. under nitrogen. N-methylmorpholine (1594 g, 8.0 eq) was added and the solution was stirred for 5 min. 2-(6-((tert-Butoxycarbonyl)amino)-9H-purin-9-yl)acetic acid (289 g+231 g+116 g+116 g, 0.5 eq+0.4 eq+0.2 eq+0.2 eq) was added at 20 min intervals, and upon completion of the addition the solution was stirred for 4 h. The solution was diluted with dichloromethane (5 L) and hydrochloric acid (9L, 1N) and the layers were separated. The aqueous layer was extracted with dichloromethane (4 L) and the combined organic layers were washed with brine (10 L), dried over anhydrous sodium sulphate, filtered, and the solvent was removed in vacuo to obtain E2 as a brown oil which was used in the subsequent step without further purification.

### Step 2.

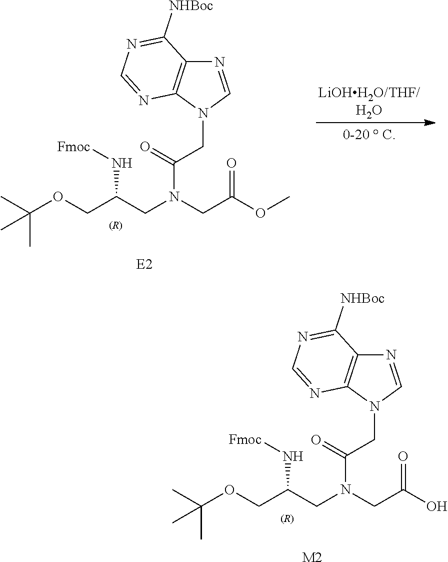

A solution of E2 (1.56 kg theoretical weight, 1.0 eq) in tetrahydrofuran (11 L, 7V) was cooled in an ice-bath to 10° C. A solution of lithium hydroxide (3.92 L, 1N, 1.8 eq) was added dropwise such that the internal temperature did not exceed 20° C. Upon completion of the addition the solution was allowed to warm to rt, and stirring was maintained for 3 h. N-(9-Fluorenylmethoxycarbonyloxy)succinimide (367 g, 0.5 eq) was added and the solution was stirred at rt for 12 h. Upon completion of the reaction the solution was diluted with water (5 L) and petroleum ether (11 L). The layers were separated and the aqueous phase was adjusted to pH=4 through the addition of hydrochloric acid solution (1N), and the mixture was extracted with ethyl acetate (15 L). The layers were separated and the organic layer was washed with brine (10 L), dried over anhydrous sodium sulphate, filtered, and the solvent was removed in vacuo to afford a yellow solid. The crude material was purified by column chromatography over silica, eluting with dichloromethane:methanol mixtures (30:1) to afford M2 as an off-white solid (1030 g, 67% yield, HPLC 98.5%). (Superchiral S-AD, 0.46×15 cm, 5 μm, 70/30/0.5 (v/v/v) Hexane/EtOH/TFA, 0.9 mL/min, 254 nm, 40° C.) Rt (major)=5.5 min, Rt (minor)=9.2 min; er=99.5:0.5. LC-MS (ESI+) m/z: [M+1]+702.32.

### Example 9C: Synthesis of A-(t-BuSer)-(Boc)G Monomer

### Step 1.

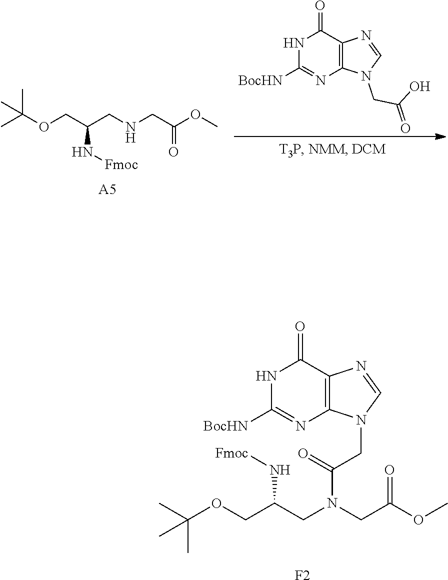

A5 was prepared according to Steps 1-3 of Example 9A. A solution of A5 (940 g, 1.0 eq) and T₃P (7.60 kg, 7.0 eq) in dichloromethane (9.4 L, 10V) was cooled in an ice-bath to 15° C. under nitrogen. N-methylmorpholine (3000 g, 18.0 eq) was added and the solution was stirred for 5 min. 2-(2-((tert-Butoxycarbonyl)amino)-6-oxo-1,6-dihydro-9H-purin-9-yl)acetic acid (1040 g, 1.8 eq) was added and the solution was stirred for 4 h. The solution was diluted with dichloromethane (5 L) and hydrochloric acid (9L, 1N) and the layers were separated. The organic layer was washed with brine (10 L), dried over anhydrous sodium sulphate, filtered, and the solvent was removed in vacuo to obtain F2 as a brown oil which was used in the subsequent step without further purification.

### Step 2

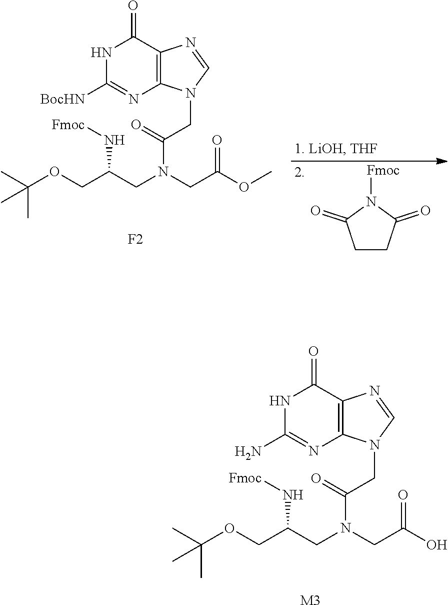

A solution of F2 (1.36 kg theoretical weight, 1.0 eq) in tetrahydrofuran (10 L, 7V) was cooled in an ice-bath to 10° C. A solution of lithium hydroxide (3.92 L, 1N, 2.5 eq) was added dropwise such that the internal temperature did not exceed 20° C. Upon completion of the addition the solution was allowed to warm to rt, and stirring was maintained for 3 h. N-(9-Fluorenylmethoxycarbonyloxy)succinimide (313 g, 0.5 eq) was added and the solution was stirred at rt for 12 h. Upon completion of the reaction the solution was diluted with water (5 L) and petroleum ether (11 L). The layers were separated and the aqueous phase was adjusted to pH=4 through the addition of hydrochloric acid solution (1N), and the mixture was extracted with ethyl acetate (15 L). The layers were separated and the organic layer was washed with brine (10 L), dried over anhydrous sodium sulphate, filtered, and the solvent was removed in vacuo to afford a yellow solid. The crude material was purified by column chromatography over silica, eluting with dichloromethane:methanol mixtures (30:1) to afford M3 as an off-white solid (710 g, 53% yield, HPLC 98.0%).

### Example 10: Synthesis of FmocN-(Z₂)Yg Monomer

### Step 1.

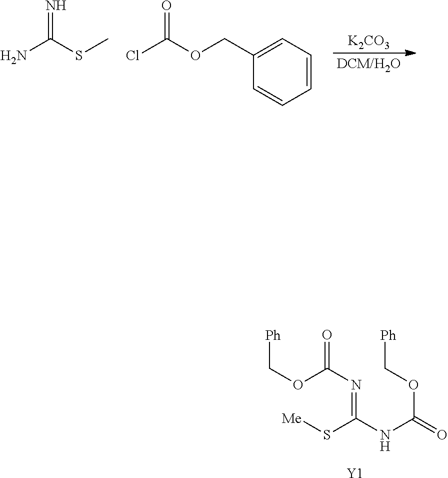

Methyl carbamimidothioate (5.0 kg, 55.5 mol) was dissolved in dichloromethane (75 L), then charged with potassium carbonate (11.5 kg, 83.2 mol) and deionized water (40 L). The reaction mixture was cooled to 10° C., charged with benzyl chloroformate (50% in toluene) (25.5 kg, 74.7 mol), and stirred at rt for 24 h. Upon completion of the reaction, deionized water (55 L) was added and the reaction mixture was stirred for 30 minutes. The phases were separated, and the aqueous layer was washed with dichloromethane (50 L). The organic layers were combined and washed with saturated aqueous sodium bicarbonate (35 L×3), water (35 L), dried over sodium sulfate, and concentrated in vacuo. The crude product was stirred in n-heptane (30 L) at 5° C. for 4 hours. The precipitate was collected by filtration, washed with n-heptane (10 L), and dried in vacuo to yield Y1 as a white solid (9.6 kg, 97% yield).

### Step 2.

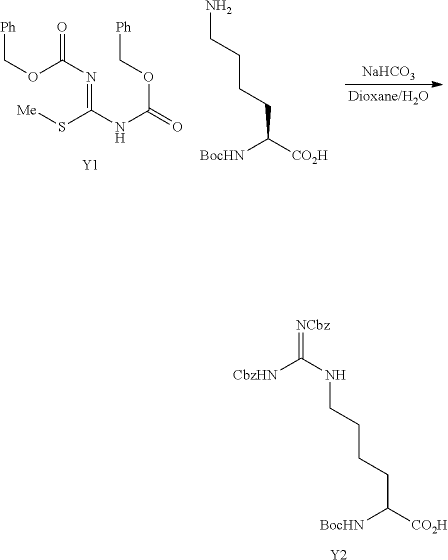

A solution of sodium bicarbonate (1.36 kg, 16.2 mol) in dioxane:water (1:1, 49 L) was charged with Y1 (2.93 kg, 11.9 mol), then with (tert-butoxycarbonyl)-L-lysine (3.5 kg, 9.77 mol) and stirred at 45° C. for 48 hours. Excess solvent was removed in vacuo. The reaction mixture was diluted with water (18 L) and adjusted to a pH of 7 with aqueous HCl (0.3 M, 42 L). The reaction mixture was extracted into ethyl acetate (90 and 35 L). The combined organic layers were washed with brine (20 L), dried over anhydrous sodium sulphate, and concentrated in vacuo. The crude material was purified by flash chromatography over silica gel, eluting with methanol:dichloromethane (0:1 to 1:9) to afford Y2 as a colorless, sticky oil (4.4 kg, 81% yield).

### Step 3.

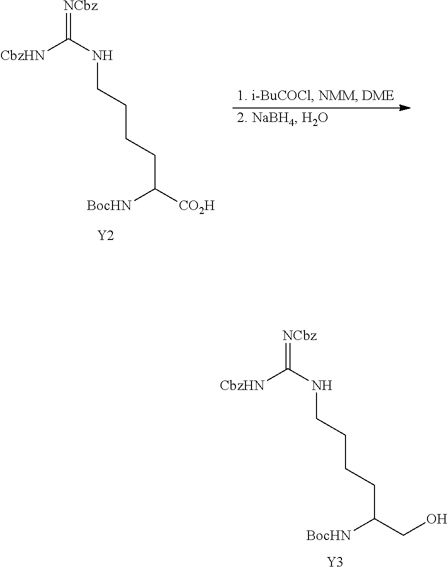

A solution of Y2 (750 g, 1.35 mol) in 1,2-dimethoxyethane (2.25 L) was cooled to −15° C. The reaction mixture was stirred under a nitrogen atmosphere and charged with N-methylmorpholine (150 g, 1.48 mol), followed by isobutyl chloroformate (202 g, 1.48 mol), and stirred at −15° C. for 15 min. The solution was filtered through a Hyflo bed and the precipitate was washed with DME (1.5 L). The filtrate was cooled to −15° C. A solution of sodium borohydride (204 g, 5.39 mol) in water (750 mL) was added dropwise over a period of 60 minutes to the filtrate at −15° C., then stirred vigorously for 30 min at −15° C. Upon completion of the reaction, the solution was diluted with water (15 L) and filtered to collect the precipitate. The precipitate was washed with water (4.6 L), diethyl ether (4.6 L), and dried in vacuo to afford Y3 as a white powder (475 g, 65% yield), which was used in the subsequent reaction without further purification.

### Step 4.

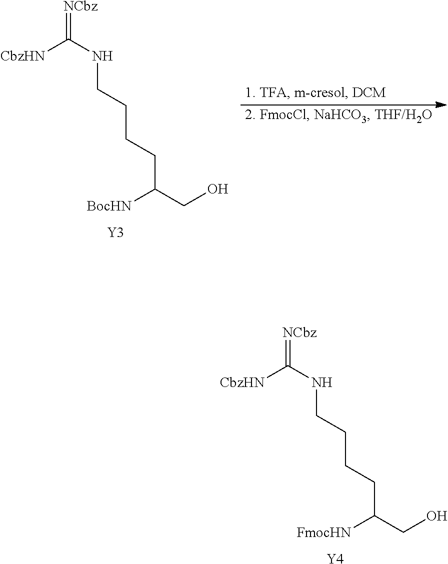

A solution of Y3 (500 g, 921 mmol) in anhydrous dichloromethane (2.95 L) was cooled to −15° C. A solution of trifluoroacetic acid:m-cresol (2.68:0.14 L) was slowly added to the −15° C. reaction mixture over a period of 90 minutes, then stirred for 1 hour until the reaction was complete. Both the dichloromethane and trifluoroaceitic acid were removed by vacuum distillation vacuum at 45° C., with two additions of toluene (2.5 L) to break the azeotrope. The concentrated reaction mixture was dissolved in water (3.68 L) and tetrahydrofuran (2.38 L) and cooled to 0° C. An aqueous solution of saturated sodium bicarbonate (8.25 L) was added to adjust the reaction mixture to a pH of ˜8 and the mixture was stirred for 15 minutes at 0° C. A solution of fluorenylmethyloxycarbonyl chloride (239 g, 921 mmol) in anhydrous tetrahydrofuran (2.0 L) was added dropwise to the stirred reaction mixture over a period of 30 minutes. The reaction mixture was warmed to room temperature and stirred for 1 hour until the reaction was complete. The reaction mixture extracted into ethyl acetate (5 L×2) and the combined the organic layers were washed with brine (5 L), dried over sodium sulfate, and concentrated in vacuo. The crude was purified by flash chromatography over silica, eluting with ethyl acetate:heptanes (0:1 to 1:0) to afford Y4 as a white solid (252 g, 41% yield).

### Step 5.

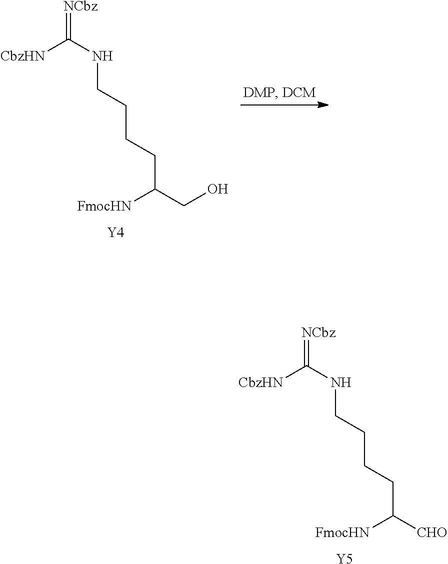

A solution of Y4 (400 g, 602 mmol) in dichloromethane (1.6 L) was cooled to 0° C. Dess-Martin periodinane (332 g, 782 mmol) was added portion-wise and the solution was stirred at 0° C. for 30 min, then at rt for an additional 2 h. The solution was diluted with diethyl ether (3.5 L) and washed with saturated sodium thiosulphate (5 L), saturated sodium bicarbonate (5 L), water (5 L), dried over anhydrous sodium sulphate, filtered, and concentrated in vacuo to afford Y5 (236 g crude) as a gummy yellow oil, which was used in the subsequent reaction without further purification.

### Step 6.

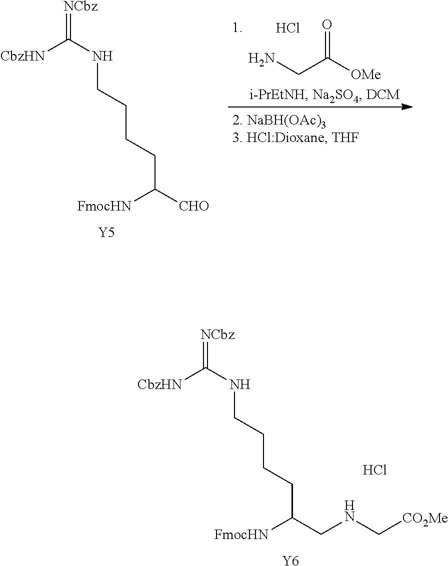

A mixture of Y5 (236 g, 356 mmol), glycine methyl ester hydrochloride (136 g, 1.08 mol), diisopropylethylamine (194 g, 129 mmol), and sodium sulphate (513 g, 3.61 mol) in dichloromethane (1.6 L) was stirred at rt for 1 hour. Sodium triacetoxyborohydride (280 g, 1.32 mol) in dichloromethane (3.2 L) was added to the reaction mixture and stirred overnight at rt. The solution was diluted with dichloromethane (4 L) and vigorously stirred. Saturated sodium bicarbonate (4 L) was added portion-wise and stirring was maintained until effervescence ceased. The layers were separated, and the organic layer was washed with water (4 L), dried over anhydrous sodium sulphate, filtered, and concentrated in vacuo. The crude was purified by flash chromatography over silica, eluting with ethyl acetate:heptane (0:1 to 1:0) to afford a gummy yellow solid, which was immediately dissolved in tetrahydrofuran (2.0 L), which was then cooled to 0° C. A solution of hydrochloric acid in 1,4-dioxane (52.0 mL, 4N) was added dropwise to the reaction mixture and stirred at 0° C. for 2 h. Diethyl ether (8 L) was added with vigorous stirring and the precipitate that formed was collected via vacuum filtration to afford Y6 as an off-white powder (192 g, 41% yield).

### Step 7.

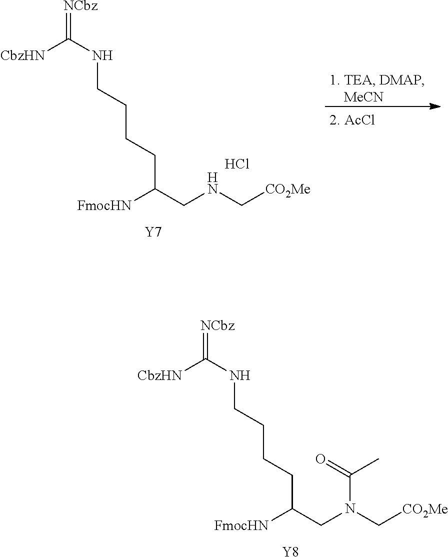

A solution of Y7 (150 g, 194 mmol) in acetonitrile (1.5 L) was stirred under a nitrogen atmosphere at rt and charged with triethylamine (39.3 g, 388 mmol) and 4-dimethylaminopyridine (4.70 g, 38.9 mmol). The reaction mixture was cooled to 0° C., charged with acetyl chloride (30.5 g, 388 mmol), and allowed to warm to rt. After 30 minutes, the completion of the reaction was confirmed by TLC. The reaction mixture was quenched with water (3 L) and extracted into ethyl acetate (2 L×2). The combined organic layers were washed with water (1.5 L×2), brine (1.5 L), dried over anhydrous sodium sulphate, and concentrated in vacuo. The crude product was purified by flash chromatography over silica, eluting with ethyl acetate:n-heptane (1:4 to 4:1) to afford Y8 as an oil (143 g, 95% yield).

### Step 8.

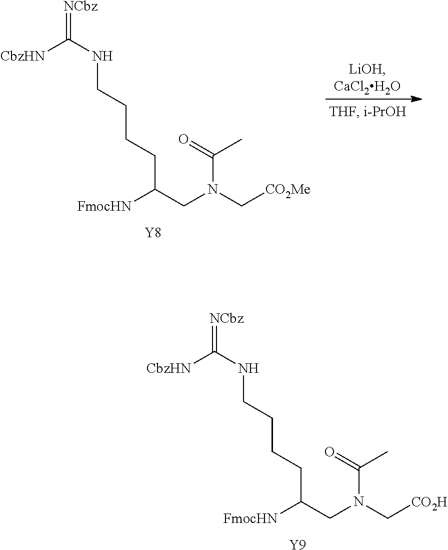

A solution of Y8 (143 g, 184 mmol) in tetrahydrofuran (1.85 L) and isopropanol (2.71 L) was cooled to 0° C. Calcium chloride dihydrate (405 g, 2.76 mol) was added with vigorous stirring, followed by the portion-wise addition of a solution of lithium hydroxide monohydrate (46.3 g, 1.10 mol) in water (0.43 L). The solution was stirred at 0° C. for 24 h. The reaction mixture was adjusted to a pH of 4 through the addition of aqueous hydrochloric acid (2 M, 572 mL). The reaction mixture was diluted with water (2 L) and extracted into ethyl acetate (3 L×2). The combined organic phases were washed with brine (2 L), dried over anhydrous sodium sulphate, and concentrated in vacuo. The crude product was purified by flash chromatography over silica, eluting with methanol:dichloromethane (0:1 to 1:4) to afford Y9 as an off-white solid (42.9 g, 31% yield, HPLC 98.2% LC-MS (ESI+) m/z: [M+1]+764.50).

### Example 11: Normalization of DMPK mRNA Expression in DM1 Patient Fibroblasts Treated with Compound 1

DM1 patient-derived human fibroblast cells that carry the DMPK 3′-UTR repeat expansion were maintained at 37° C. and 5% CO₂ in MEM supplemented with 15% heat inactivated FBS. Cells were plated in 24-well plates at 30,000 cells/well in 1 mL of medium one day before treatment. Before treatment, medium was replaced with MEM with 2% heat-inactivated FBS. Stock solutions of Compound 1 were heated at 80° C. for 10 min before use and were added to the cell cultures at a final concentration of 111M. Cells were incubated for 7 days in the presence of Compound 1. All treatments were performed in triplicate.

Total RNA was extracted using an RNAeasy kit. RNA was eluted in 30 μL. For quantification of DMPK mRNA via qPCR, cDNA synthesis was performed using an Invitrogen Super Script III kit with priming by random hexamers.

DMPK expression level was analyzed by SYBR Green rt-qPCR using the ΔΔCt method, normalizing to GAPDH, using the following primers: DMPK_FWD: CACTGTCGGACATTCGGGAAGGTGC (SEQ ID NO: 115); DMPK_REV: GCTTGCACGTGTGGCTCAAGCAGCTG (SEQ ID NO: 116); GAPDH_FWD: GTCTCCTCTGACTTCAACAGCG (SEQ ID NO: 117) GAPDH_Rev: ACCACCCTGTTGCTGTAGCCAA (SEQ ID NO: 118).

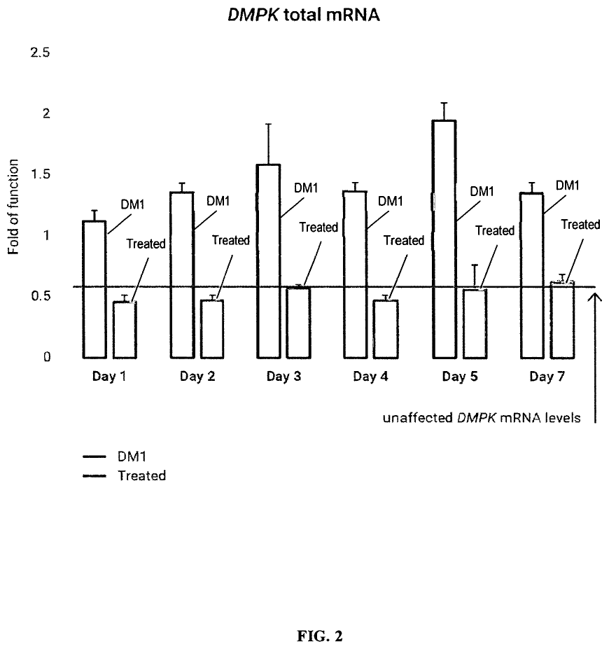

PCR samples were analyzed using Quanti Studio 5 System. As shown in FIG. 2, treatment with Compound 1 reduced the level of DMPK transcript to that of the normal control cells within 24 hours. The horizontal line indicates the level of DMPK transcript in normal human cells. These results indicate that Compound 1 crossed the cell membrane, escaped the endosome, penetrated the nucleus, and disrupted the nuclear aggregates to allow the mutant transcript to translocate to the cytoplasm and normalize the expression of DMPK.

### Example 12: Splice Rescue of MBNL1 and MBNL2 in DM1 Patient Fibroblasts Treated with Compound 1

DM1 patient-derived human fibroblast cells that carry the DMPK 3′-UTR repeat expansion were maintained at 37° C. and 5% CO₂ in MEM supplemented with 15% heat inactivated FBS. Cells were plated in 24-well plates at 30,000 cells/well in 1 mL of medium one day before treatment. Before treatment, medium was replaced with MEM with 2% heat-inactivated FBS. Stock solutions of Compound 1 were heated at 80° C. for 10 min before use and were added to the cell cultures at a final concentration of 111M. Cells were incubated for 9 days in the presence of Compound 1. All treatments were performed in triplicate. Healthy cells that do not carry the DMPK 3′-UTR repeat expansion were included as controls.

After 2-9 days of treatment with Compound 1, total RNA was extracted using an RNAeasy kit. RNA was eluted in 30 μL. For quantification of DMPK mRNA via qPCR, cDNA synthesis was performed using an Invitrogen Super Script III kit with priming by random hexamers.

Alternative splicing for the MBNL1 exon 5 and MBNL2 exon 7 were assessed by PCR using DreamTaq PCR MasterMix 2× (Thermo #K1071) and the following primers:

MBNL1_F
(SEQ ID NO: 125)
5′-GCTGCCCAATACCAGGTCAAC-3′
MBNL1_R
(SEQ ID NO: 120)
5′-TGGTGGGAGAAATGCTGTATGC-3′
MBNL2_F
(SEQ ID NO: 126)
5′-TCCTTTACCAAAGAGACAAGCAC-3′
MBNL2_R
(SEQ ID NO: 127)
5′-CTCAATGCAGATTCTTGGCATTCC-3′

PCR samples were analyzed using D1000 ScreenTape Assay on a Tape Station 4200. PCR bands were quantified using iBright Analysis Software. Exon 5/7 inclusion was calculated as the % of the upper band over total gene transcripts.

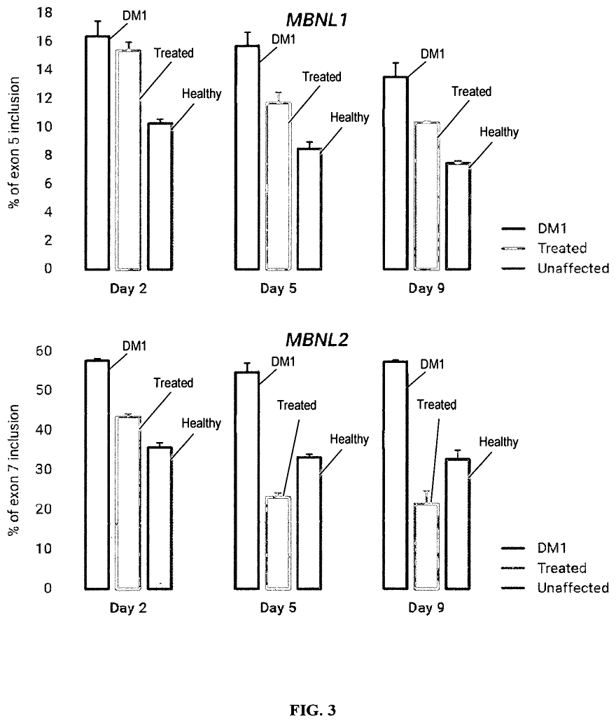

By day 2 post treatment, an effect on splicing rescue was observed, with the correct adult exon usage further improved at later time points and approaching full restoration by day 9 in each case (FIG. 3).

### Example 13: Correction of Splicing in DM1 Patient Fibroblasts Treated with Compound 1

DM1 patient-derived human fibroblast cells that carry the DMPK 3′-UTR repeat expansion were maintained at 37° C. and 5% CO₂ in MEM supplemented with 15% heat inactivated FBS. Cells were plated in 24-well plates at 30,000 cells/well in 1 mL of medium one day before treatment. Before treatment, medium was replaced with MEM with 2% heat-inactivated FBS. Stock solutions of Compound 1 were heated at 80° C. for 10 min before use and were added to the cell cultures at a final concentration of 1 μM. Cells were incubated for 5 days in the presence of Compound 1, assayed for splice rescue, and compared to untreated DM1 patient-derived fibroblast cells and healthy fibroblasts that do not contain the DMPK 3′-UTR repeat expansion. All treatments were performed in triplicate.

Transcriptomes of the cells were sequenced, and the change in transcript percent splice inclusion (ΔPSI) between affected cells and healthy cells at the p<0.05 level was determined.

Total RNA was collected using RNAeasy kit (Qiagen cat #74106) following instruction manual. RNA was eluted in 3011.1 and quantified.

For RNA sequencing, libraries were created from polyA-selected RNA using a kit from Illumina cat #20040532 and Index plate cat #20040553. The protocol was adjusted for 300 ng total RNA. Libraries were amplified for 13 cycles and after clean up, concentration and quality were checked using Qubit and Tape station. Sequencing was performed with an Illumima NextSeq500. To analyze the data, reads were aligned to reference data with STAR v2.7.1a using the STAR options from rMATS v4.1.0. Duplicates were removed with Picard MarkDuplicates v2.18.7.

Aberrant splice activity was measured with rMATS v4.1.0 run on all pairwise combinations of conditions (healthy-untreated, disease-treated, and disease-untreated) with standard options. The top events from the “Skipped Exons Junction Counts Exon Counts” (‘SE.MATS.JCEC.txt’) from the three outputs were then merged together. Any event not present in the healthy-untreated vs. disease-treated comparison was dropped, and only those events with a minimum absolute inclusion difference greater than 0.2 were included.

Human differential splice inclusion (hDSI) was calculated by first selecting a set of cassette exons with:
a) delta-PSI (absolute difference in Percent Splice Inclusion) between healthy-unrelated and disease-untreated samples, as calculated by rMATS, of >=20%
b) p-value of <=0.05 for differential splicing as calculated by rMATS
c) read depth >=5× in all healthy-untreated, disease-untreated and disease-treated samples.

For each cassette exon a scale was calibrated from 0-1 where 0 is the mean PSI of healthy-untreated samples and 1 is the mean PSI of disease-untreated samples. For each sample the PSI for each cassette exon was normalized into the scale, and then values for all selected cassette exons were averaged to yield hDSI.

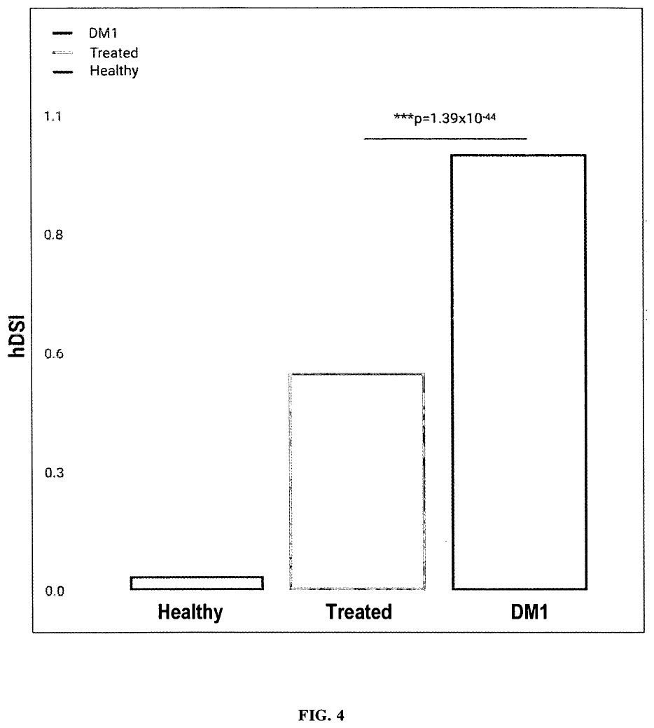

The hDSI calculated as above was used as a composite score of global splice rescue after 5 days of treatment. Correction of global splicing across multiple transcripts (hDSI) was observed at day 5 (FIG. 4). Calculation of hDSI (degree of average ΔPSI across all mis-spliced transcripts) indicated that treatment rescued normal splicing at a level of p=1.39 ×10⁻⁴⁴ over untreated DM1 cells.

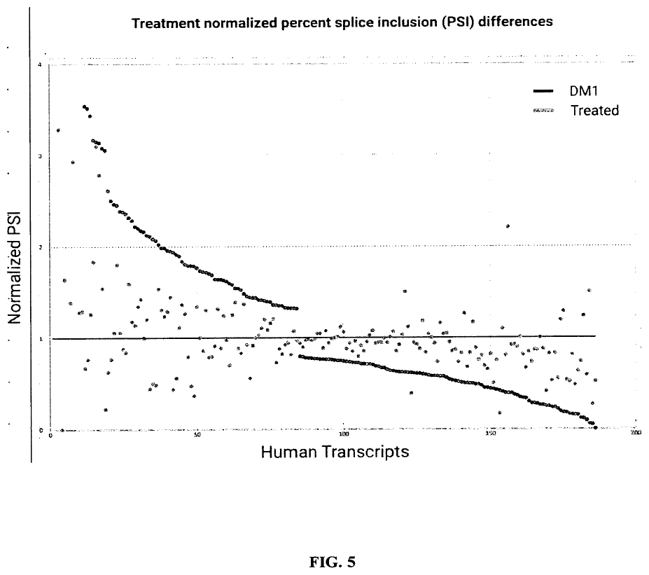

FIG. 5 is a chart that depicts normalized percent splice inclusion (PSI) between treated vs. healthy human cells for 188 unique human transcripts (x-axis) at 5 days post treatment with Compound 1. A statistically-significant difference in PSI was observed between healthy and DM1 splice inclusion, greater than >20% PSI and robust fold-coverage. A statistically-significant p-value was observed between treated and untreated (DM1) PSI. Restoration of normal splicing was observed after Compound 1 treatment across 188 significantly mis-spliced human transcripts by day 5.

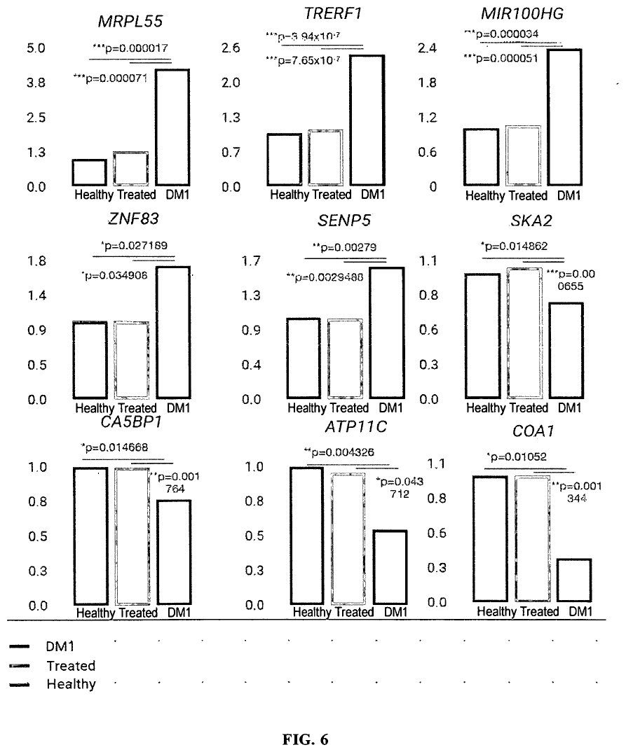

Treatment resulted in rescue of appropriate adult cassette exon use across many mis-spliced transcripts (FIG. 6, summarizing percent splice inclusion levels (PSI) for selected transcripts in cells 5 days after treatment with Compound 1).

### Example 14: Intravascular Administration of Compound 1 Reduces Pathogenic ACTA1 mRNA in the HSA^{LR} Mouse Model of Myotonic Dystrophy

The ability of Compound 1 to reduce pathogenic mRNA comprising CUG repeats upon intramuscular administration was evaluated.

HSA^{LR} mice were administered a single dose of Compound 1 at a dose of 29 mg/kg via the intravenous injection into the tail vein. Muscle tissue was harvested one day after dosing and processed for qPCR to quantify transcript levels.

All muscles were weighed before RNA isolation. For 100 mg tissue, total RNA was isolated using 1 mL of Trizol. Tissues was homogenized with OMNI TH homogenizer in Trizol. RNA was extracted using BCP, precipitated using Isopropanol, and resuspended in 100 μL RNase free water. Five μg of the isolated RNA was treated with DNase to remove genomic DNA, and RNA re-purified using an RNeasy Mini kit.

300 ng of purified total RNA was reverse transcribed using a SuperScriptIII First-Strand Synthesis System for RT-PCR. qPCR reactions were carried out using TaqMan Fast Advanced Master Mix.

For ACTA1 qPCR, forward primer was GTAGCTACCCGCCCAGAAACT (SEQ ID NO: 112), reverse primer was CCAGGCCGGAGCCATT (SEQ ID NO: 113), and custom TaqMan probe was ACCACCGCCCTCGTGTGCG (3′ MGBNFQ quencher and 5′ 6FAM dye, IDT) (SEQ ID NO: 114) and a 1:10 dilution of cDNA was used as input. For Gtf2b reference gene qPCR, Gene Expression Assay Mm00663250 ml (4331182, Vic, Applied Biosystems) was used and 2 μL of cDNA. qPCR was performed on QuantaStudio5 using fast ramp speed. ACTA1 transgene expression relative to Gtf2b was calculated using the ΔΔCt method.

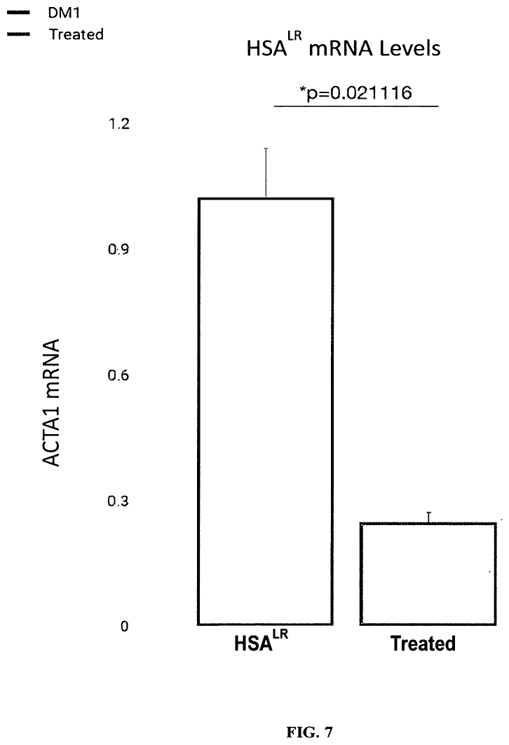

Treatment via the intravenous route with 29 mg/kg of Compound 1 reduced levels of pathogenic mRNA (FIG. 7; ACTA1 mRNA, which comprises a CUG repeat in the 3′ UTR that leads to the formation of toxic hairpins and impaired splicing due to MBNL1 sequestration in the HSA^{LR} mouse model of myotonic dystrophy).

These results indicate that Compound 1 can exit the vasculature and penetrate into tibialis anterior muscle nuclei to engage target HSA (skeletal muscle actin) mRNA within 24 hours. mRNA levels remained decreased until 13 days (last timepoint tested).

### Example 15: Correction of Splicing Upon Administration of Compound 1 in the HSA^{LR} Mouse Model of Myotonic Dystrophy

The ability of Compound 1 to rescue splicing was assessed in the HSA^{LR} mouse model of myotonic dystrophy.

Compound 1 was administered intravenously by tail vein injection in a single dose at 29 mg/kg. Tibialis anterior muscle was harvested 13 days after treatment and processed for RNA sequencing. FVB background strain mice were included as healthy controls.

Total RNA was collected using RNAeasy kit. RNA was eluted in 10004, and quantified. For RNA sequencing, libraries were created from polyA-selected RNA using a kit from Illumina cat #20040532 and Index plate cat #20040553. The protocol was adjusted for 300 ng total RNA. Libraries were amplified for 13 cycles and after clean up, concentration and quality were checked using Qubit and Tape station. Sequencing was performed with an Illumima NextSeq500. To analyze the data, reads were aligned to reference data with STAR v2.7.1a using the STAR options from rMATS v4.1.0. Duplicates were removed with Picard MarkDuplicates v2.18.7.

Aberrant splice activity was measured with rMATS v4.1.0 run on all pairwise combinations of conditions (healthy-untreated, disease-treated, and disease-untreated) with standard options. The top events from the “Skipped Exons Junction Counts Exon Counts” (‘SE.MATS.JCEC.txt’) from the three outputs were then merged together. Any event not present in the healthy-untreated vs. disease-treated comparison was dropped, and only those events with a minimum absolute inclusion difference greater than 0.2 were included.

Mouse differential splice inclusion (mDSI) was calculated by first selecting a set of cassette exons with:
a) delta-PSI (absolute difference in Percent Splice Inclusion) between healthy-unrelated and disease-untreated samples, as calculated by rMATS, of >=20%
b) p-value of <=0.05 for differential splicing as calculated by rMATS
c) read depth >=5× in all healthy-untreated, disease-untreated and disease-treated samples.

For each cassette exon a scale was calibrated from 0-1 where 0 is the mean PSI of healthy-untreated samples and 1 is the mean PSI of disease-untreated samples. For each sample the PSI for each cassette exon was normalized into the scale, and then values for all selected cassette exons were averaged to yield mDSI.

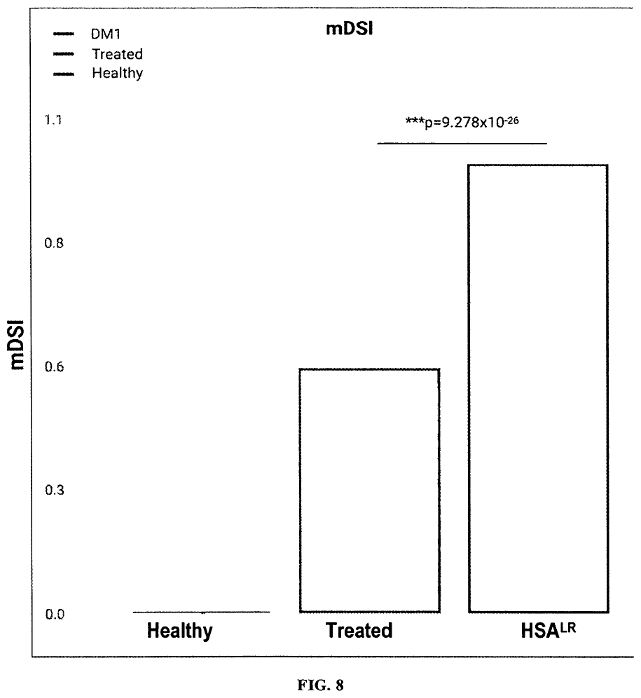

The hDSI calculated as above was used as a composite score of global splice rescue 13 days after treatment. Correction of global splicing across multiple transcripts (mDSI) was observed at day 13 (FIG. 8).

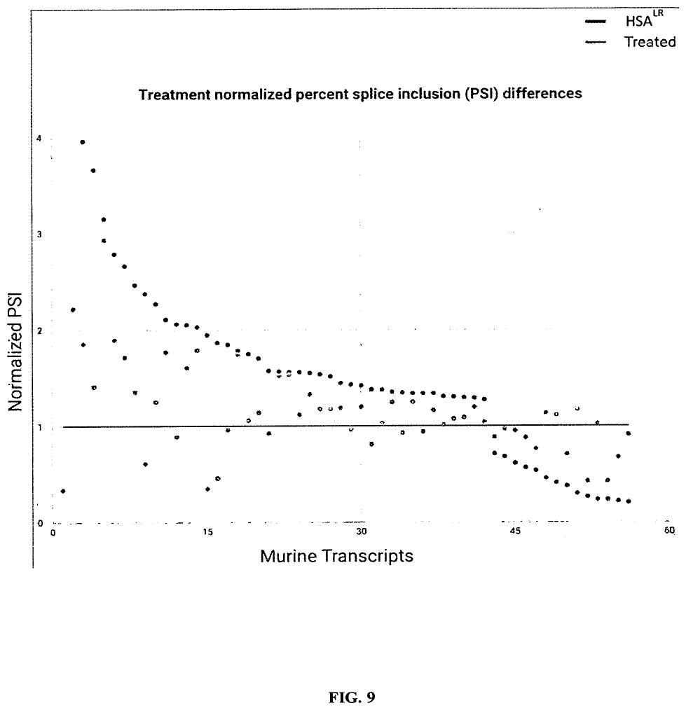

FIG. 9 illustrates normalized percent splice inclusion (PSI) between treated HSA^{LR} mice vs. FVB background strain (healthy control) mice for 56 unique murine Tibialis anterior muscle transcripts (x-axis) at 13 days post IV administration of Compound 1. Observations included (i) a statistically-significant difference in PSI between healthy and untreated HSA^{LR} exon inclusion, (ii) greater than >20% PSI fold-coverage, and (iii) a statistically-significant p-value between treated and untreated HSA^{LR} PSI. Restoration of normal splicing was observed across 56 mis-spliced murine Tibialis anterior skeletal muscle transcripts at 13 days post intravenous administration of Compound 1.

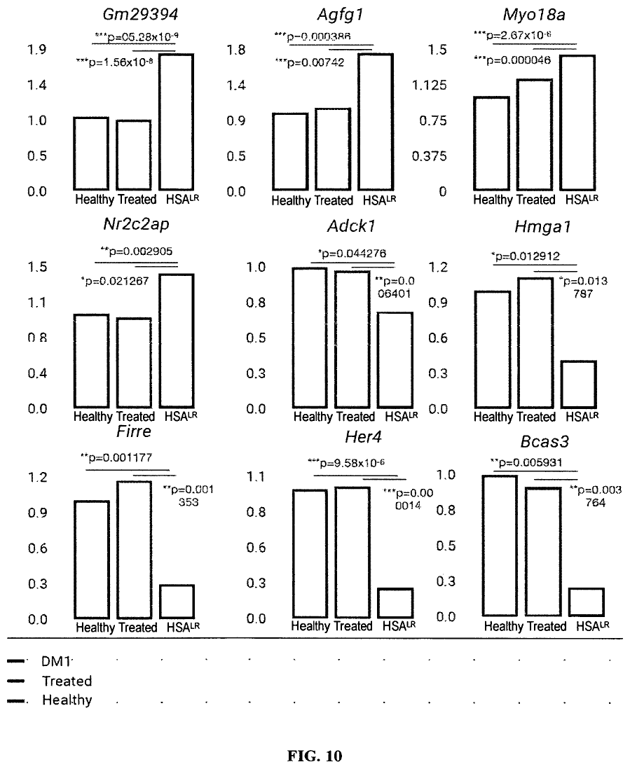

FIG. 10 illustrates percent splice inclusion (PSI) in selected murine Tibialis anterior muscle transcripts at day 13 after intravenous administration of Compound 1.

### Example 16: DMPK Protein Levels in Response to Treatment with Compound 1

DM1 patient-derived human fibroblast cells were maintained at 37° C. and 5% CO₂ in MEM supplemented with 15% heat inactivated FBS. Cells were plated in 24-well plates at 30,000 cells/well in 1 mL of medium one day before treatment. Before treatment, medium was replaced with MEM with 2% heat-inactivated FBS. Stock solutions of Compound 1 were heated at 80° C. for 10 min before use and were added to the cell cultures at a final concentration of 1 μM. Cells were incubated for 5 days in the presence of Compound 1, assayed for levels of DMPK protein and compared to untreated DM1 patient-derived fibroblast cells. All treatments were performed in triplicate.

Cells were lysed 100 μl M-Per buffer. Protein extracts were quantified using BCA assay (Pierce™ BCA protein Assay Kit cat. n #23225). Protein extracts were resolved on SDS-PAGE 4-12% gradient gels (Invitrogen™, Carlsbad, CA, cat #28451) with Tris-SDS MES Buffer (Invitrogen™, Carlsbad, CA cat #22341). Gels were run at 150 V for 1 hour. After gel electrophoresis, proteins were transferred to a nitrocellulose membrane (Invitrogen™, Carlsbad, CA, cat #PB3310). Primary antibodies specific for DMPK (1:500, Abcam, Cambridge UK, cat. n #ab102804) and Beta-actin (1:5000, Abcam Cambridge, UK, cat. n #ab8227) were used.

HRP conjugate anti-mouse or anti-rabbit secondary antibody (1:10,000, Jackson ImmunoResearch Laboratories, cat. n #315-035-0003 and 111-035-045) were used for visualizing proteins using SuperSignal™ West Pico Plus Chemiluminescent Substrate (cat. n #34577). Protein bands were quantified using iBright™ Analysis Software (Thermo Scientific™, Waltham, MA). Protein bands were quantified using iBright analysis software and normalized according to beta-actin expression and the DMPK expression level was calculated as a relative value to untreated.

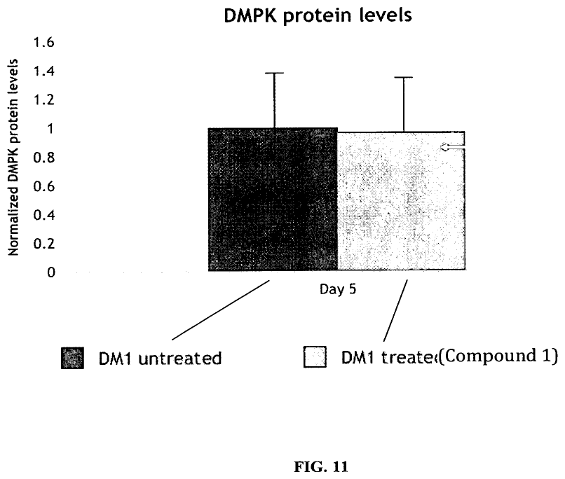

DMPK protein levels in untreated and treated DM1 patient-derived cells were similar (FIG. 11).

### Example 17: Reduction of Pathogenic ACTA1 Transcript in Response to Treatment with Compound 1

The ability of Compound 1 to reduce pathogenic mRNA comprising CUG repeats was evaluated. HSA^{LR} mice were administered a 29 mg/kg single IV dose of Compound 1. 24 hours after administration, the tibialis anterior muscle was harvested and processed or qPCR gene expression analysis.

All muscles were weighed before RNA isolation. For 100 mg tissue, total RNA was isolated using 1 mL of Trizol. Tissues was homogenized with OMNI TH homogenizer in Trizol. RNA was extracted using BCP, precipitated using Isopropanol, and resuspended in 100 μL RNase free water. Five μg of the isolated RNA was treated with DNase to remove genomic DNA, and RNA re-purified using an RNeasy Mini kit.

300 ng of purified total RNA was reverse transcribed using a SuperScriptIII First-Strand Synthesis System for RT-PCR. qPCR reactions were carried out using TaqMan Fast Advanced Master Mix.

For ACTA1 qPCR, forward primer was GTAGCTACCCGCCCAGAAACT (SEQ ID NO: 112), reverse primer was CCAGGCCGGAGCCATT (SEQ ID NO: 113), and custom TaqMan probe was ACCACCGCCCTCGTGTGCG (3′ MGBNFQ quencher and 5′ 6FAM dye, IDT) (SEQ ID NO: 114) and a 1:10 dilution of cDNA was used as input. For Gtf2b reference gene qPCR, Gene Expression Assay Mm00663250_ml (4331182, Vic, Applied Biosystems) was used and 2 μL of cDNA. qPCR was performed on QuantaStudio5 using fast ramp speed. ACTA1 transgene expression relative to Gtf2b was calculated using the ΔΔCt method.

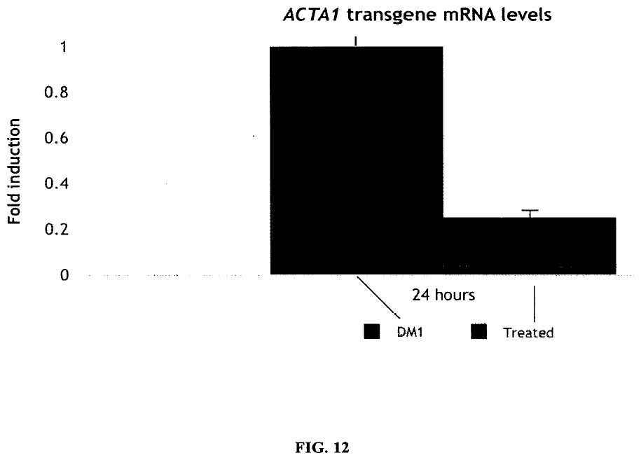

Treatment with Compound 1 reduced levels of pathogenic mRNA (FIG. 12; ACTA1 mRNA, which comprises a CUG repeat with ˜220 CUG copies in the 3′ UTR that leads to the formation of toxic hairpins and impaired splicing due to MBNL1 sequestration in the HSA^{LR} mouse model of myotonic dystrophy, resulting in a similar muscular phenotype as DM1). These results show that ACTA1 transgene mRNA levels were lower (p=0.021) in mice treated with Compound 1, and further indicate that Compound 1 penetrated myofiber, escaped the endosome, localized to nuclei, and invaded ACTA1 mRNA 3′ UTR hairpins.

### Example 18: Reduction of DMPK mRNA in a DM1 Cell Culture Model as a Function of Shuttle (Poly-Yg Tail) Length

To assess reduction of DMPK mRNA in a DM1 cell culture model with PNA of varying of poly-Yg tail lengths, DM1 fibroblast cells (3989 fibroblast cells) were treated with increasing dose concentrations of Compounds 1, 34, 35, 36 and 90. All five Compounds share an identical 14-mer pharmacophore but have variable shuttle (poly-Yg tail) lengths. After five days of treatment, total cellular RNA was extracted from cells and utilized as template for cDNA synthesis and qRT-PCR reaction using commercially available primers for DMPK. The comparative ΔΔCt method was used to measure the DMPK induction relative to untreated DM1 fibroblast cells with expression normalized to GAPDH. FIG. 20 shows that all five compounds reduced DMPK mRNA. Quantitative results are provided in TABLE 9.

TABLE 9

| | Compound | | Mean DMPK |
|---|---|---|---|
| Compound | concentration | | expression |
| Untreated Control | — | | 1 |
| 1 | 1 | μM | 0.42 |
| 1 | 300 | nM | 0.31 |
| 1 | 100 | nM | 0.57 |
| 34 | 1 | μM | 0.50 |
| 34 | 300 | nM | 0.38 |
| 34 | 100 | nM | 0.52 |
| 35 | 1000 | nM | 0.51 |
| 35 | 300 | nM | 0.51 |
| 35 | 100 | nM | 0.73 |
| 36 | 1000 | nM | 0.55 |
| 36 | 300 | nM | 0.75 |
| 36 | 100 | nM | 1.02 |
| 90 | 1000 | nM | 0.44 |
| 90 | 300 | nM | 0.54 |
| 90 | 100 | nM | 0.87 |

1. A compound comprising an oligomeric structure, wherein the oligomeric structure comprises a repeating unit of formula: 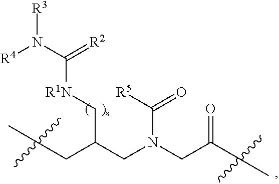
or an ionized form thereof, wherein:
R¹ is H, alkyl, or a nitrogen atom protecting group;
R² is O, NH, N(alkyl), or N(Pg^{N}), wherein Pg^{N} is a nitrogen atom protecting group;
R³ is H, alkyl, or a nitrogen atom protecting group;
R⁴ is H, alkyl, or a nitrogen atom protecting group;
R⁵ is alkyl or O-alkyl, any of which is unsubstituted or substituted; and
n is 1, 2, 3, 4, 5, 6, 7, 8, 9, or 10,
or a pharmaceutically-acceptable salt or ionized form thereof.

2. The compound of claim 1, wherein each of R¹, R³, and R⁴ is hydrogen; and R² is NH or N(Pg^{N}).

3. The compound of claim 1, wherein R⁵ is linear alkyl.

4. The compound of claim 1, wherein R⁵ is methyl.

5. The compound of claim 1, wherein n is 3.

6. The compound of claim 1, wherein n is 4.

7. The compound of claim 1, further comprising a first chemical moiety attached to the oligomeric structure, and a second chemical moiety attached to the oligomeric structure, wherein the oligomeric structure, wherein the first chemical moiety, and the second chemical moiety form: 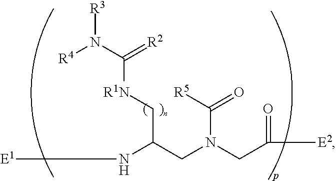
wherein:
E¹ is the first chemical moiety;
E² is the second chemical moiety; and
p is an integer that is 1-100.

8. The compound of claim 7, wherein p is 6.

9. The compound of claim 7, wherein p is 7.

10. The compound of claim 7, wherein p is 8.

11. The compound of claim 7, wherein E¹ is hydrogen, acyl, a group that together with the nitrogen atom to which E¹ is bound forms a carbamate, a probe, a metal chelator, an imaging agent, or a biologically-active agent; and E² is OH, OMe, NH₂, a probe, a metal chelator, an imaging agent, or a biologically-active agent.

12. The compound of claim 11, wherein E¹ is hydrogen and E² is the biologically-active agent.

13. The compound of claim 12, wherein the biologically-active agent comprises a structure that interferes with expression of a gene associated with a neuromuscular disease phenotype.

14. The compound of claim 13, wherein the neuromuscular disease phenotype is a DM1 disease phenotype.

15. The compound of claim 14, wherein the DM1 disease phenotype is associated with a non-wild-type DM1 gene that differs from a wild type DM1 gene in a repeat expansion mutation.

16. The compound of claim 12, wherein the biologically-active agent binds to a mRNA sequence at a region that is (CUG)_{z}, wherein z is an integer from 1-100 (SEQ ID NO: 135).

17. The compound of claim 12, wherein the biologically-active agent is an oligonucleotide or oligonucleotide analogue.

18. The compound of claim 12, wherein the biologically-active agent is a peptide nucleic acid.

19. The compound of claim 1, wherein the compound is: 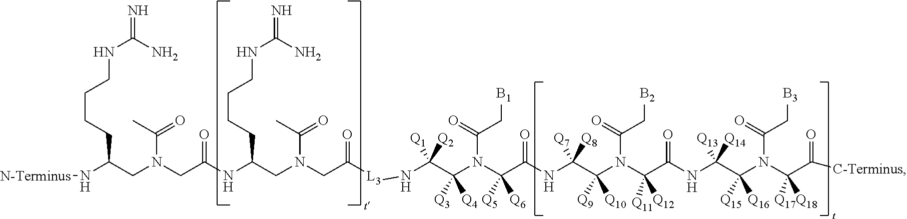
wherein:
each instance of B¹, B², and B³ is independently a heterocycle;
each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl, heteroalkyl, aryl, or heteroaryl, each of which is independently substituted or unsubstituted, or hydrogen;
L3 is a linker group or absent;
N-Terminus is H, acyl, a group that together with the nitrogen atom to which the N-Terminus is bound forms a carbamate, a probe, a fluorophore, a lipid, a metal chelator, or a biological agent;
C-Terminus is —N(H)-J, wherein J is H, acyl, a group that together with the nitrogen atom to which J is bound forms a carbamate, a probe, a fluorophore, a lipid, a metal chelator, or a biological agent;
t is an integer that is from 1 to 30; and
t′ is an integer that is from 2 to 9,
or a pharmaceutically acceptable salt or ionized form thereof.

20. The compound of claim 19, wherein L3 is absent.

21. The compound of claim 19, wherein each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl that is substituted or unsubstituted, or hydrogen.

22. The compound of claim 1, wherein the compound is: 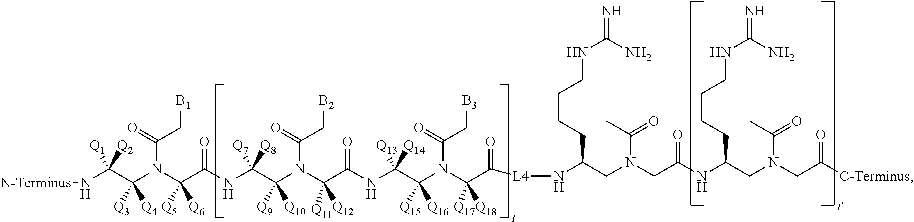
wherein:
each instance of B¹, B², and B³ is independently a heterocycle;
each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl, heteroalkyl, aryl, or heteroaryl, each of which is independently substituted or unsubstituted, or hydrogen;
L4 is a linker group or absent;
N-Terminus is H, acyl, a group that together with the nitrogen atom to which the N-Terminus is bound forms a carbamate, a probe, a fluorophore, a lipid, a metal chelator, or a biological agent;
C-Terminus is —N(H)-J, wherein J is H, acyl, a group that together with the nitrogen atom to which J is bound forms a carbamate, a probe, a fluorophore, a lipid, a metal chelator, or a biological agent;
t is an integer that is from 1 to 30; and
t′ is an integer that is from 2 to 9,
or a pharmaceutically acceptable salt or ionized form thereof.

23. The compound of claim 22, wherein L4 is absent.

24. The compound of claim 19, wherein N-Terminus is H.

25. The compound of claim 19, wherein C-Terminus is NH₂.

26. The compound of claim 23, wherein each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷ and Q¹⁸ is independently an amino acid side chain, alkyl that is substituted or unsubstituted, or hydrogen.

27-55. (canceled)

56. The compound of claim 19, wherein the compound is: 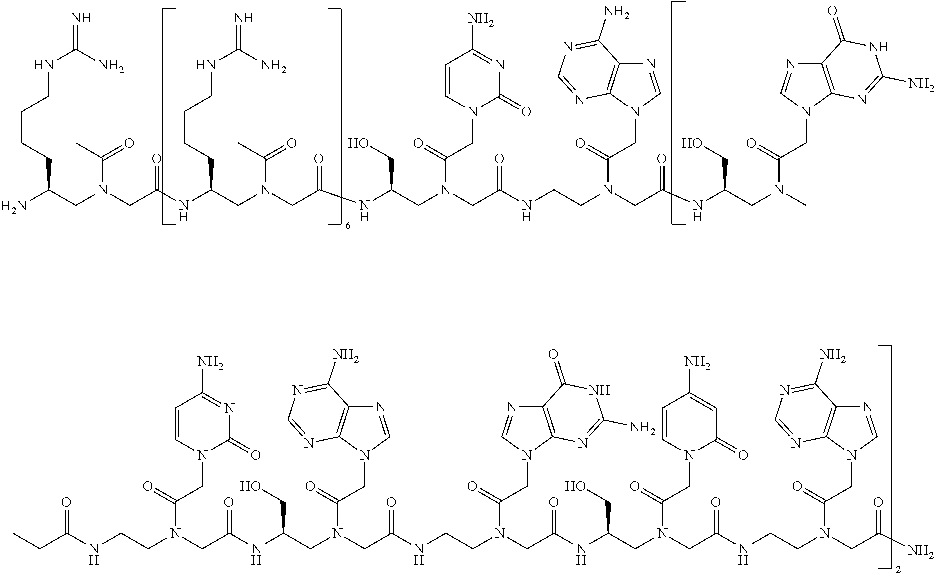
or a pharmaceutically-acceptable salt or ionized form thereof.

57-76. (canceled)


SEQUENCE LISTING (140 sequences)

SEQ ID NO: 1 - protein, 7 aa, Artificial Sequence
  Description of Artificial Sequence Synthetic peptide
PKKKRKV

SEQ ID NO: 2 - protein, 9 aa, Artificial Sequence
  Description of Artificial Sequence Synthetic peptide
PAAKRVKLD

SEQ ID NO: 3 - protein, 16 aa, Artificial Sequence
  Description of Artificial Sequence Synthetic peptide
KRPAATKKAGQAKKKL

SEQ ID NO: 4 - protein, 10 aa, Artificial Sequence
  Description of Artificial Sequence Synthetic peptide
VQRKRQKLMP

SEQ ID NO: 5 - protein, 8 aa, Artificial Sequence
  Description of Artificial Sequence Synthetic peptide
SKKKKTKV

SEQ ID NO: 6 - protein, 8 aa, Artificial Sequence
  Description of Artificial Sequence Synthetic peptide
GRKRKKRT

SEQ ID NO: 7 - protein, 8 aa, Artificial Sequence
  Description of Artificial Sequence Synthetic peptide
ERKKRRRE

SEQ ID NO: 8 - protein, 7 aa, Artificial Sequence
  Description of Artificial Sequence Synthetic peptide
FKKFRKF

SEQ ID NO: 9 - protein, 16 aa, Artificial Sequence
  Description of Artificial Sequence Synthetic peptide
RKKRRRESRKKRRRES

SEQ ID NO: 10 - protein, 17 aa, Artificial Sequence
  Description of Artificial Sequence Synthetic peptide
GRPRESGKKRKRKRLKP

SEQ ID NO: 11 - protein, 15 aa, Artificial Sequence
  Description of Artificial Sequence Synthetic peptide
GKRKKKGKLGKKRDP

SEQ ID NO: 12 - protein, 17 aa, Artificial Sequence
  Description of Artificial Sequence Synthetic peptide
GKRKKKGKLGKKRPRSR

SEQ ID NO: 13 - protein, 18 aa, Artificial Sequence
  Description of Artificial Sequence Synthetic peptide
RKKRRRESRRARRSPRHL

SEQ ID NO: 14 - protein, 19 aa, Artificial Sequence
  Description of Artificial Sequence Synthetic peptide
SRRARRSPRESGKKRKRKR

SEQ ID NO: 15 - protein, 19 aa, Artificial Sequence
  Description of Artificial Sequence Synthetic peptide
VKRGLKLRHVRPRVTRMDV

SEQ ID NO: 16 - protein, 14 aa, Artificial Sequence
  Description of Artificial Sequence Synthetic peptide
SRRARRSPRHLGSG

SEQ ID NO: 17 - protein, 16 aa, Artificial Sequence
  Description of Artificial Sequence Synthetic peptide
LRRERQSRLRRERQSR

SEQ ID NO: 18 - protein, 22 aa, Artificial Sequence
  Description of Artificial Sequence Synthetic peptide
GAYDLRRRERQSRLRRRERQSR

SEQ ID NO: 19 - protein, 9 aa, Artificial Sequence
  Description of Artificial Sequence Synthetic peptide
RKKRRQRRR

SEQ ID NO: 20 - protein, 15 aa, Artificial Sequence
  Description of Artificial Sequence Synthetic peptide
RRRRNRTRRNRRRVR

SEQ ID NO: 21 - protein, 17 aa, Artificial Sequence
  Description of Artificial Sequence Synthetic peptide
TRQARRNRRRRWRERQR

SEQ ID NO: 22 - protein, 13 aa, Artificial Sequence
  Description of Artificial Sequence Synthetic peptide
TRRQRTRRARRNR

SEQ ID NO: 23 - protein, 19 aa, Artificial Sequence
  Description of Artificial Sequence Synthetic peptide
KMTRAQRRAAARRNRWTAR

SEQ ID NO: 24 - protein, 17 aa, Artificial Sequence
  Description of Artificial Sequence Synthetic peptide
NAKTRRHERRRKLAIER

SEQ ID NO: 25 - protein, 22 aa, Artificial Sequence
  Description of Artificial Sequence Synthetic peptide
MDAQTRRRERRAEKQAQWKAAN

SEQ ID NO: 26 - protein, 18 aa, Artificial Sequence
  Description of Artificial Sequence Synthetic peptide
TAKTRYKARRAELIAERR

SEQ ID NO: 27 - protein, 16 aa, Artificial Sequence
  Description of Artificial Sequence Synthetic peptide
TRRNKRNRIQEQLNRK

SEQ ID NO: 28 - protein, 32 aa, Artificial Sequence
  Description of Artificial Sequence Synthetic polypeptide
PRRRRSSSRPVRRRRRPRVSRRRRRRGGRRRR

SEQ ID NO: 29 - protein, 28 aa, Artificial Sequence
  Description of Artificial Sequence Synthetic peptide
RIKAERKRMRNRIAASKSRKRKLERIAR

SEQ ID NO: 30 - protein, 26 aa, Artificial Sequence
  Description of Artificial Sequence Synthetic peptide
KRRIRRERNKMAAAKSRNRRRELTDT

SEQ ID NO: 31 - protein, 22 aa, Artificial Sequence
  Description of Artificial Sequence Synthetic peptide
KRARNTEAARRSRARKLQRMKQ

SEQ ID NO: 32 - protein, 16 aa, Artificial Sequence
  Description of Artificial Sequence Synthetic peptide
RQIKIWFQNRRMKWKK

SEQ ID NO: 33 - protein, 16 aa, Artificial Sequence
  Description of Artificial Sequence Synthetic peptide
RVIRVWFQNKRCKDKK

SEQ ID NO: 34 - protein, 60 aa, Artificial Sequence
  Description of Artificial Sequence Synthetic polypeptide
SKRTRQTYTRYQTLELEKEFHFNRYITRRRRIDIANALSLSERQIKIWFQNRRMKSKKDR

SEQ ID NO: 35 - protein, 16 aa, Artificial Sequence
  Description of Artificial Sequence Synthetic peptide
SQIKIWFQNKRAKIKK

SEQ ID NO: 36 - protein, 16 aa, Artificial Sequence
  Description of Artificial Sequence Synthetic peptide
RQVTIWFQNRRVKEKK

SEQ ID NO: 37 - protein, 16 aa, Artificial Sequence
  Description of Artificial Sequence Synthetic peptide
KQINNWFINQRKRHWK

SEQ ID NO: 38 - protein, 16 aa, Artificial Sequence
  Description of Artificial Sequence Synthetic peptide
RHIKIWFQNRRMKWKK

SEQ ID NO: 39 - protein, 27 aa, Artificial Sequence
  Description of Artificial Sequence Synthetic peptide
GLAFLGFLGAAGSTMGAWSQPKKKRKV

SEQ ID NO: 40 - protein, 24 aa, Artificial Sequence
  Description of Artificial Sequence Synthetic peptide
RRIRPRPPRLPRPRPRPLPFPRPG

SEQ ID NO: 41 - protein, 20 aa, Artificial Sequence
  Description of Artificial Sequence Synthetic peptide
ALWKTLLKKVLKAPKKKRKV

SEQ ID NO: 42 - protein, 10 aa, Artificial Sequence
  Description of Artificial Sequence Synthetic peptide
RRIPNRRPRR

SEQ ID NO: 43 - protein, 20 aa, Artificial Sequence
  Description of Artificial Sequence Synthetic peptide
HARIKPTFRRLKWKYKGKFW

SEQ ID NO: 44 - protein, 26 aa, Artificial Sequence
  Description of Artificial Sequence Synthetic peptide
GIGAVLKVLTTGLPALISWIKRKRQQ

SEQ ID NO: 45 - protein, 18 aa, Artificial Sequence
  Description of Artificial Sequence Synthetic peptide
RGGRLSYSRRRFSTSTGR

SEQ ID NO: 46 - protein, 15 aa, Artificial Sequence
  Description of Artificial Sequence Synthetic peptide
KLWMRWYSPTTRRYG

SEQ ID NO: 47 - protein, 5 aa, Artificial Sequence
  Description of Artificial Sequence Synthetic peptide
RLRWR

SEQ ID NO: 48 - protein, 9 aa, Artificial Sequence
  Description of Artificial Sequence Synthetic peptide
CAYHRLRRC

SEQ ID NO: 49 - protein, 19 aa, Artificial Sequence
  Description of Artificial Sequence Synthetic peptide
LYKKGPAKKGRPPLRGWFH

SEQ ID NO: 50 - protein, 16 aa, Artificial Sequence
  Description of Artificial Sequence Synthetic peptide
KGTYKKKLMRIPLKGT

SEQ ID NO: 51 - protein, 18 aa, Artificial Sequence
  Description of Artificial Sequence Synthetic peptide
RASKRDGSWVKKLHRILE

SEQ ID NO: 52 - protein, 21 aa, Artificial Sequence
  Description of Artificial Sequence Synthetic peptide
TRSSRAGLQWPVGRVHRLLRK

SEQ ID NO: 53 - protein, 15 aa, Artificial Sequence
  Description of Artificial Sequence Synthetic peptide
FKIYDKKVRTRVVKH

SEQ ID NO: 54 - protein, 18 aa, Artificial Sequence
  Description of Artificial Sequence Synthetic peptide
VRLPPPVRLPPPVRLPPP

SEQ ID NO: 55 - protein, 13 aa, Artificial Sequence
  Description of Artificial Sequence Synthetic peptide
GPFHFYQFLFPPV

SEQ ID NO: 56 - protein, 12 aa, Artificial Sequence
  Description of Artificial Sequence Synthetic peptide
PLILLRLLRGQF

SEQ ID NO: 57 - protein, 16 aa, Artificial Sequence
  Description of Artificial Sequence Synthetic peptide
YTAIAWVKAFIRKLRK

SEQ ID NO: 58 - protein, 20 aa, Artificial Sequence
  Description of Artificial Sequence Synthetic peptide
KETWWETWWTEWSQPKKRKV

SEQ ID NO: 59 - protein, 23 aa, Artificial Sequence
  Description of Artificial Sequence Synthetic peptide
LIRLWSHLIHIWFQNRRLKWKKK

SEQ ID NO: 60 - protein, 20 aa, Artificial Sequence
  Description of Artificial Sequence Synthetic peptide
VDKGSYLPRPTPPRPIYNRN

SEQ ID NO: 61 - protein, 13 aa, Artificial Sequence
  Description of Artificial Sequence Synthetic peptide
GSPWGLQHHPPRT

SEQ ID NO: 62 - protein, 17 aa, Artificial Sequence
  Description of Artificial Sequence Synthetic peptide
KLALKALKALKAALKLA

SEQ ID NO: 63 - protein, 5 aa, Artificial Sequence
  Description of Artificial Sequence Synthetic peptide
IPALK

SEQ ID NO: 64 - protein, 5 aa, Artificial Sequence
  Description of Artificial Sequence Synthetic peptide
VPALR

SEQ ID NO: 65 - protein, 18 aa, Artificial Sequence
  Description of Artificial Sequence Synthetic peptide
LLIILRRRIRKQAHAHSK

SEQ ID NO: 66 - protein, 17 aa, Artificial Sequence
  Description of Artificial Sequence Synthetic peptide
IAWVKAFIRKLRKGPLG

SEQ ID NO: 67 - protein, 21 aa, Artificial Sequence
  Description of Artificial Sequence Synthetic peptide
AAVLLPVLLAAPVQRKRQKLP

SEQ ID NO: 68 - protein, 12 aa, Artificial Sequence
  Description of Artificial Sequence Synthetic peptide
TSPLNIHNGQKL

SEQ ID NO: 69 - protein, 5 aa, Artificial Sequence
  Description of Artificial Sequence Synthetic peptide
VPTLK

SEQ ID NO: 70 - protein, 5 aa, Artificial Sequence
  Description of Artificial Sequence Synthetic peptide
VSALK

SEQ ID NO: 71 - protein, 26 aa, Artificial Sequence
  Description of Artificial Sequence Synthetic peptide
DPKGDPKGVTVTVTVTVTGKGDPKPD

SEQ ID NO: 72 - protein, 21 aa, Artificial Sequence
  Description of Artificial Sequence Synthetic peptide
AGYLLGKINLKALAALAKKIL

SEQ ID NO: 73 - protein, 18 aa, Artificial Sequence
  Description of Artificial Sequence Synthetic peptide
VELPPPVELPPPVELPPP

SEQ ID NO: 74 - protein, 20 aa, Artificial Sequence
  Description of Artificial Sequence Synthetic peptide
GLWRALWRLLRSLWRLLWRA

SEQ ID NO: 75 - protein, 12 aa, Artificial Sequence
  Description of Artificial Sequence Synthetic peptide
PLSSIFSRIGDP

SEQ ID NO: 76 - protein, 9 aa, Artificial Sequence
  Description of Artificial Sequence Synthetic peptide
RRWWRRWRR

SEQ ID NO: 77 - protein, 9 aa, Artificial Sequence
  Description of Artificial Sequence Synthetic peptide
AKASSLNIA

SEQ ID NO: 78 - protein, 7 aa, Artificial Sequence
  Description of Artificial Sequence Synthetic peptide
ASSLNIA

SEQ ID NO: 79 - protein, 12 aa, Artificial Sequence
  Description of Artificial Sequence Synthetic peptide
VQLRNGFPYFSY

SEQ ID NO: 80 - protein, 12 aa, Artificial Sequence
  Description of Artificial Sequence Synthetic peptide
THRPPMWSPVWP

SEQ ID NO: 81 - protein, 7 aa, Artificial Sequence
  Description of Artificial Sequence Synthetic peptide
HAIYPRH

SEQ ID NO: 82 - protein, 9 aa, Artificial Sequence
  Description of Artificial Sequence Synthetic peptide
  SITE (1)..(9): Intrachain disulfide bond
CRTIGPSVC

SEQ ID NO: 83 - protein, 12 aa, Artificial Sequence
  Description of Artificial Sequence Synthetic peptide
  MOD_RES (1)..(12): D-amino acid
PWVPSWMPPRHT

SEQ ID NO: 84 - protein, 7 aa, Artificial Sequence
  Description of Artificial Sequence Synthetic peptide
  MOD_RES (1)..(7): D-amino acid
HRPYIAH

SEQ ID NO: 85 - protein, 12 aa, Artificial Sequence
  Description of Artificial Sequence Synthetic peptide
  MOD_RES (1)..(12): D-amino acid
THRPPMWSPVWP

SEQ ID NO: 86 - protein, 7 aa, Artificial Sequence
  Description of Artificial Sequence Synthetic peptide
RFQILYR

SEQ ID NO: 87 - protein, 7 aa, Artificial Sequence
  Description of Artificial Sequence Synthetic peptide
RYQFLIR

SEQ ID NO: 88 - protein, 7 aa, Artificial Sequence
  Description of Artificial Sequence Synthetic peptide
RIQFLIR

SEQ ID NO: 89 - protein, 5 aa, Artificial Sequence
  Description of Artificial Sequence Synthetic peptide
RRWQW

SEQ ID NO: 90 - protein, 4 aa, Artificial Sequence
  Description of Artificial Sequence Synthetic peptide
GWWG

SEQ ID NO: 91 - protein, 5 aa, Artificial Sequence
  Description of Artificial Sequence Synthetic peptide
GFWFG

SEQ ID NO: 92 - protein, 12 aa, Artificial Sequence
  Description of Artificial Sequence Synthetic peptide
GRKKRRQRRRPQ

SEQ ID NO: 93 - protein, 14 aa, Artificial Sequence
  Description of Artificial Sequence Synthetic peptide
RGRGRGRGRGRGRG

SEQ ID NO: 94 - protein, 12 aa, Artificial Sequence
  Description of Artificial Sequence Synthetic peptide
RRRRRRRRRRRR

SEQ ID NO: 95 - protein, 12 aa, Artificial Sequence
  Description of Artificial Sequence Synthetic peptide
PPRPPRPPRPPR

SEQ ID NO: 96 - RNA, 10 nt, Artificial Sequence
  Description of Artificial Sequence Synthetic oligonucleotide
cugcugcugc

SEQ ID NO: 97 - RNA, 11 nt, Artificial Sequence
  Description of Artificial Sequence Synthetic oligonucleotide
cugcugcugcu

SEQ ID NO: 98 - RNA, 12 nt, Artificial Sequence
  Description of Artificial Sequence Synthetic oligonucleotide
cugcugcugcug

SEQ ID NO: 99 - RNA, 13 nt, Artificial Sequence
  Description of Artificial Sequence Synthetic oligonucleotide
cugcugcugcugc

SEQ ID NO: 100 - RNA, 14 nt, Artificial Sequence
  Description of Artificial Sequence Synthetic oligonucleotide
cugcugcugcugcu

SEQ ID NO: 101 - RNA, 15 nt, Artificial Sequence
  Description of Artificial Sequence Synthetic oligonucleotide
cugcugcugcugcug

SEQ ID NO: 102 - RNA, 16 nt, Artificial Sequence
  Description of Artificial Sequence Synthetic oligonucleotide
cugcugcugcugcugc

SEQ ID NO: 103 - RNA, 17 nt, Artificial Sequence
  Description of Artificial Sequence Synthetic oligonucleotide
cugcugcugcugcugcu

SEQ ID NO: 104 - RNA, 18 nt, Artificial Sequence
  Description of Artificial Sequence Synthetic oligonucleotide
cugcugcugcugcugcug

SEQ ID NO: 105 - RNA, 19 nt, Artificial Sequence
  Description of Artificial Sequence Synthetic oligonucleotide
cugcugcugcugcugcugc

SEQ ID NO: 106 - RNA, 20 nt, Artificial Sequence
  Description of Artificial Sequence Synthetic oligonucleotide
cugcugcugcugcugcugcu

SEQ ID NO: 107 - RNA, 21 nt, Artificial Sequence
  Description of Artificial Sequence Synthetic oligonucleotide
cugcugcugcugcugcugcug

SEQ ID NO: 108 - RNA, 22 nt, Artificial Sequence
  Description of Artificial Sequence Synthetic oligonucleotide
cugcugcugcugcugcugcugc

SEQ ID NO: 109 - RNA, 23 nt, Artificial Sequence
  Description of Artificial Sequence Synthetic oligonucleotide
cugcugcugcugcugcugcugcu

SEQ ID NO: 110 - RNA, 24 nt, Artificial Sequence
  Description of Artificial Sequence Synthetic oligonucleotide
cugcugcugcugcugcugcugcug

SEQ ID NO: 111 - DNA, 20 bp, Artificial Sequence
  Description of Artificial Sequence Synthetic probe
cagcagcagcagcagcagca

SEQ ID NO: 112 - DNA, 21 bp, Artificial Sequence
  Description of Artificial Sequence Synthetic primer
gtagctacccgcccagaaact

SEQ ID NO: 113 - DNA, 16 bp, Artificial Sequence
  Description of Artificial Sequence Synthetic primer
ccaggccggagccatt

SEQ ID NO: 114 - DNA, 19 bp, Artificial Sequence
  Description of Artificial Sequence Synthetic probe
accaccgccctcgtgtgcg

SEQ ID NO: 115 - DNA, 25 bp, Artificial Sequence
  Description of Artificial Sequence Synthetic primer
cactgtcggacattcgggaaggtgc

SEQ ID NO: 116 - DNA, 26 bp, Artificial Sequence
  Description of Artificial Sequence Synthetic primer
gcttgcacgtgtggctcaagcagctg

SEQ ID NO: 117 - DNA, 22 bp, Artificial Sequence
  Description of Artificial Sequence Synthetic primer
gtctcctctgacttcaacagcg

SEQ ID NO: 118 - DNA, 22 bp, Artificial Sequence
  Description of Artificial Sequence Synthetic primer
accaccctgttgctgtagccaa

SEQ ID NO: 119 - DNA, 23 bp, Artificial Sequence
  Description of Artificial Sequence Synthetic primer
ctgtcaaatcactgaagcgaccc

SEQ ID NO: 120 - DNA, 22 bp, Artificial Sequence
  Description of Artificial Sequence Synthetic primer
tggtgggagaaatgctgtatgc

SEQ ID NO: 121 - DNA, 21 bp, Artificial Sequence
  Description of Artificial Sequence Synthetic primer
gatgctactttgcagcccctg

SEQ ID NO: 122 - DNA, 20 bp, Artificial Sequence
  Description of Artificial Sequence Synthetic primer
cacggaacacaaaggcactg

SEQ ID NO: 123 - DNA, 23 bp, Artificial Sequence
  Description of Artificial Sequence Synthetic primer
catcactgccacccagaagactg

SEQ ID NO: 124 - DNA, 23 bp, Artificial Sequence
  Description of Artificial Sequence Synthetic primer
atgccagtgagcttcccgttcag

SEQ ID NO: 125 - DNA, 21 bp, Artificial Sequence
  Description of Artificial Sequence Synthetic primer
gctgcccaataccaggtcaac

SEQ ID NO: 126 - DNA, 23 bp, Artificial Sequence
  Description of Artificial Sequence Synthetic primer
tcctttaccaaagagacaagcac

SEQ ID NO: 127 - DNA, 24 bp, Artificial Sequence
  Description of Artificial Sequence Synthetic primer
ctcaatgcagattcttggcattcc

SEQ ID NO: 128 - protein, 22 aa, Artificial Sequence
  Description of Artificial Sequence Synthetic peptide
  MOD_RES (2)..(2): 6-aminohexanoic acid
  MOD_RES (5)..(5): 3-aminopropanoic acid
  MOD_RES (8)..(8): 6-aminohexanoic acid
  MOD_RES (16)..(16): 6-aminohexanoic acid
  MOD_RES (18)..(18): 3-aminopropanoic acid
  MOD_RES (20)..(20): 6-aminohexanoic acid
  MOD_RES (22)..(22): 3-aminopropanoic acid
RXRRXRRXRFQILYRXRXRXRX

SEQ ID NO: 129 - protein, 12 aa, Artificial Sequence
  Description of Artificial Sequence Synthetic peptide
  MOD_RES (8)..(8): 6-aminohexanoic acid
  MOD_RES (9)..(9): 3-aminopropanoic acid
  MOD_RES (10)..(10): D-amino acid
  MOD_RES (11)..(11): D-amino acid
  MOD_RES (12)..(12): D-amino acid
ASSLNIAXXRRR

SEQ ID NO: 130 - protein, 14 aa, Artificial Sequence
  Description of Artificial Sequence Synthetic peptide
  MOD_RES (2)..(2): 6-aminohexanoic acid
  MOD_RES (5)..(5): 3-aminopropanoic acid
  MOD_RES (8)..(8): 6-aminohexanoic acid
  MOD_RES (11)..(11): 3-aminopropanoic acid
  MOD_RES (13)..(13): 6-aminohexanoic acid
  MOD_RES (14)..(14): 3-aminopropanoic acid
RXRRXRRXRRXRXX

SEQ ID NO: 131 - protein, 22 aa, Artificial Sequence
  Description of Artificial Sequence Synthetic peptide
  MOD_RES (2)..(2): 6-aminohexanoic acid
  MOD_RES (5)..(5): 3-aminopropanoic acid
  MOD_RES (8)..(8): 6-aminohexanoic acid
  MOD_RES (16)..(16): 6-aminohexanoic acid
  MOD_RES (18)..(18): 3-aminopropanoic acid
  MOD_RES (20)..(20): 6-aminohexanoic acid
  MOD_RES (22)..(22): 3-aminopropanoic acid
RXRRXRRXRYQFLIRXRXRXRX

SEQ ID NO: 132 - protein, 22 aa, Artificial Sequence
  Description of Artificial Sequence Synthetic peptide
  MOD_RES (2)..(2): 6-aminohexanoic acid
  MOD_RES (5)..(5): 3-aminopropanoic acid
  MOD_RES (8)..(8): 6-aminohexanoic acid
  MOD_RES (16)..(16): 6-aminohexanoic acid
  MOD_RES (18)..(18): 3-aminopropanoic acid
  MOD_RES (20)..(20): 6-aminohexanoic acid
  MOD_RES (22)..(22): 3-aminopropanoic acid
RXRRXRRXRIQFLIRXRXRXRX

SEQ ID NO: 133 - protein, 21 aa, Artificial Sequence
  Description of Artificial Sequence Synthetic peptide
  MOD_RES (2)..(2): 6-aminohexanoic acid
  MOD_RES (5)..(5): 3-aminopropanoic acid
  MOD_RES (8)..(8): 6-aminohexanoic acid
  MOD_RES (15)..(15): 6-aminohexanoic acid
  MOD_RES (17)..(17): 3-aminopropanoic acid
  MOD_RES (19)..(19): 6-aminohexanoic acid
  MOD_RES (21)..(21): 3-aminopropanoic acid
RXRRXRRXYRFLIRXRXRXRX

SEQ ID NO: 134 - protein, 9 aa, Artificial Sequence
  Description of Artificial Sequence Synthetic peptide
  SITE (2)..(8): Intrachain disulfide bond
GCGGGGGCG

SEQ ID NO: 135 - RNA, 300 nt, Artificial Sequence
  Description of Artificial Sequence Synthetic polynucleotide
  misc_feature (1)..(300): This sequence may encompass 1-100 “cug” repeating units
  See specification as filed for detailed description of substitutions and preferred embodiments
cugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcug

SEQ ID NO: 136 - protein, 5 aa, Artificial Sequence
  Description of Artificial Sequence Synthetic peptide
  MOD_RES (3)..(3): 3-aminopropanoic acid
RRXRR

SEQ ID NO: 137 - protein, 8 aa, Artificial Sequence
  Description of Artificial Sequence Synthetic peptide
  MOD_RES (2)..(2): 6-aminohexanoic acid
  MOD_RES (5)..(5): 3-aminopropanoic acid
  MOD_RES (8)..(8): 6-aminohexanoic acid
RXRRXRRX

SEQ ID NO: 138 - protein, 100 aa, Artificial Sequence
  Description of Artificial Sequence Synthetic polypeptide
  SITE (1)..(100): This sequence may encompass 1-50 “Arg Gly” repeating units
RGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRG

SEQ ID NO: 139 - protein, 100 aa, Artificial Sequence
  Description of Artificial Sequence Synthetic polypeptide
  SITE (1)..(100): This sequence may encompass 2-100 residues
RRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRRR

SEQ ID NO: 140 - protein, 150 aa, Artificial Sequence
  Description of Artificial Sequence Synthetic polypeptide
  SITE (1)..(150): This sequence may encompass 2-50 “Pro Pro Arg” repeating units
PPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPRPPR